(19) United States
(12) Patent Application Publication
(10) Pub. No.: US 2026/0070982 A1
(43) Pub. Date: Mar. 12, 2026

(54) ANTIGEN BINDING MOLECULES TO CD3 AND CD19

(71) Applicant: XENCOR, INC., Pasadena, CA (US)

(72) Inventors: Matthew Bernett, Pasadena, CA (US); Gregory L. Moore, Pasadena, CA (US); Viralkumar Rameshkumar Davra, Pasadena, CA (US); Katrina Bykova, Pasadena, CA (US); Seung Y. Chu, Pasadena, CA (US); Jing Qi, Pasadena, CA (US); Su-Shin Hao, Pasadena, CA (US); Kendra Avery, Pasadena, CA (US); Jitendra Kanodia, Pasadena, CA (US); Juan Diaz, Pasadena, CA (US)

(21) Appl. No.: 19/321,270

(22) Filed: Sep. 7, 2025

Related U.S. Application Data
(60) Provisional application No. 63/692,069, filed on Sep. 7, 2024.
(60) Provisional application No. 63/793,511, filed on Apr. 23, 2025.

(51) Int. Cl. C07K 16/28; A61K 39/00; A61P 35/00; A61P 37/06

(52) U.S. Cl. CPC C07K 16/2809; A61P 35/00; A61P 37/06; C07K 16/2803; A61K 2039/505; C07K 2317/31; C07K 2317/51; C07K 2317/522; C07K 2317/524; C07K 2317/526; C07K 2317/53; C07K 2317/55; C07K 2317/565; C07K 2317/567; C07K 2317/622

(57) ABSTRACT

Disclosed herein are anti-CD3 antigen binding molecules, anti-CD19 antigen binding molecules, anti-CD19 and anti-CD3 bi-specific antigen binding molecules, and methods for the use thereof.

### CROSS REFERENCE TO RELATED APPLICATIONS

This application claims priority to U.S. Provisional Application No. 63/793,511, filed on Apr. 23, 2025, and U.S. Provisional Application No. 63/692,069, filed on Sep. 7, 2024, the disclosures of each of which are hereby incorporated by reference in their entireties.

### SEQUENCE LISTING

The contents of the electronic sequence listing (121168000407_Sequence_Listing_ST26.xml; Size: 3,392,961 bytes; and Date of Creation: Aug. 25, 2025) is herein incorporated by reference in its entirety.

### TECHNICAL FIELD

Disclosed herein are antigen binding molecules binding CD3 and CD19, and methods for the use thereof.

### BACKGROUND

CD3 (Cluster of Differentiation 3) is a T-cell co-receptor that is present on both cytotoxic and helper T-cells. CD3 is involved in activating T-cells making CD3 a potential target to stimulate or block T cell activation. CD19 (Cluster of Differentiation 19) is a signaling glycoprotein that is present on all B-cells, which makes CD19 an important marker and potential target for therapies aimed to modulate for B-cells.

### SUMMARY

In a first aspect, the disclosure is directed to a bi-specific anti-CD19 and anti-CD3 antigen binding molecule comprising an anti-CD19 antigen binding domain and an anti-CD3 antigen binding domain.

In some variations, the anti-CD19 antigen binding domains comprise a first binding subunit comprising the CDR sequences of SEQ ID NOs: 1, 2, and 3, and a second binding subunit comprising the CDR sequences of SEQ ID NOs: 4, 5, and 6. The anti-CD3 antigen binding domains comprise a first binding subunit comprising the CDR sequences of SEQ ID NO: 9, SEQ ID NO: 10 or 11, and SEQ ID NO: 12, 13, 14, 15, or 16, and a second binding subunit comprising the CDR sequences of SEQ ID NOs: 17, 18, and 19.

In some variations, the bi-specific anti-CD19 and anti-CD3 antigen binding molecules include a first monomer, second monomer, and light chain. The first monomer comprises a scFv-domain linker-CH2-CH3 domain in which the scFv comprises an antigen binding domain that binds CD3, and the CH2-CH3 is a first variant Fc domain. The second monomer comprises a VH-CH1-hinge-CH2-CH3, in which the CH2-CH3 is a second variant Fc domain complementary to the first variant Fc domain. The first variant Fc domain and second variant Fc domain form a heterodimeric backbone. The light chain together with the second monomer forms a Fab domain that binds CD19. In some variations, the Fab domain is a Fv domain.

In some variations, the bi-specific anti-CD19 and anti-CD3 antigen binding molecules include first monomer, a second monomer, a first light chain, and a second light chain. The first monomer comprises a VH-CH1-first domain linker-scFv-second domain linker-CH2-CH3 in which the the scFv comprises an antigen binding domain that binds CD3, and the CH2-CH3 is a first variant Fc domain. The second monomer comprises a VH-CH1-hinge-CH2-CH3, in which the CH2-CH3 is a second variant Fc domain complementary to the first variant Fc domain. The first variant Fc domain and second variant Fc domain form a heterodimeric backbone. The first light chain together with the first monomer forms a Fab domain that binds CD19. The second light chain together with the second monomer forms a Fab domain that binds CD19. In some variations, the Fab domain is a Fv domain.

In a second aspect, the disclosure is directed to an anti-CD19 antigen binding molecules comprising an antigen binding domain. The antigen binding domain comprises a first binding subunit comprising the CDR sequences of SEQ ID NOs: 1, 2, and 3, and a second binding subunit comprising the CDR sequences of SEQ ID NOs: 4, 5, and 6.

In some variations, the anti-CD19 antigen binding molecules, comprising an antigen binding domain. The antigen binding domain comprises a first binding subunit, a second binding subunit, and a framework region comprising at least 95% identity to an amino acid sequence selected from the group consisting of SEQ ID NOs: 20, 21, 22, 23, 24, 25, 26, and 27.

In some variations, the anti-CD19 antigen binding molecules, comprising an antigen binding domain. The antigen binding domain comprises an amino acid sequence comprising at least 95% identity to SEQ ID NO: 20 N-terminal to a first binding subunit CDR1, an amino acid sequence comprising at least 95% identity to SEQ ID NO: 21 between the first binding subunit CDR1 and a first binding subunit CDR2, an amino acid sequence comprising at least 95% identity to SEQ ID NO: 22 between the first binding subunit CDR2 and a first binding subunit CDR3, an amino acid sequence comprising at least 95% identity to SEQ ID NO: 23 C-terminal to the first binding subunit CDR3, an amino acid sequence comprising at least 95% identity to SEQ ID NO: 24 N-terminal to a second binding subunit CDR1, an amino acid sequence comprising at least 95% identity to SEQ ID NO: 25 between the second binding subunit CDR1 and a second binding subunit CDR2, an amino acid sequence comprising at least 95% identity to SEQ ID NO: 26 between the second binding subunit CDR2 and a second binding subunit CDR3, and an amino acid sequence comprising at least 95% identity to SEQ ID NO: 27 C-terminal to the second binding subunit CDR3.

In a third aspect, the disclosure is directed to an anti-CD3 antigen binding molecules comprising an antigen binding domain. The antigen binding domain comprises a first binding subunit comprising the CDR sequences of SEQ ID NO: 9, SEQ ID NO: 10 or 11, and SEQ ID NO: 12, 13, 14, 15, or 16, and a second binding subunit comprising the CDR sequences of SEQ ID NOs: 17, 18, and 19.

In some variations, the anti-CD3 antigen binding molecules, comprising a first binding subunit and a second binding subunit, and a framework region comprising at least 95% identity to an amino acid sequence selected from the group consisting of SEQ ID NOs: 28, 29, 30, 31, 32, 33, 34, and 35.

In some variations, the anti-CD3 antigen binding molecules, comprising an amino acid sequence comprising at least 95% identity to SEQ ID NO: 28 N-terminal to a first binding subunit CDR1, an amino acid sequence comprising at least 95% identity to SEQ ID NO: 29 between the first binding subunit CDR1 and a first binding subunit CDR2, an amino acid sequence comprising at least 95% identity to SEQ ID NO: 30 between the first domain CDR2 and a first domain CDR3, an amino acid sequence comprising at least 95% identity to SEQ ID NO: 31 C-terminal to the first domain CDR3, an amino acid sequence comprising at least 95% identity to SEQ ID NO: 32 N-terminal to a second domain CDR1, an amino acid sequence comprising at least 95% identity to SEQ ID NO: 33 between the second domain CDR1 and a second domain CDR2, an amino acid sequence comprising at least 95% identity to SEQ ID NO: 34 between the second domain CDR2 and a second domain CDR3, and an amino acid sequence comprising at least 95% identity to SEQ ID NO: 35 C-terminal to the second domain CDR3.

### BRIEF DESCRIPTION OF THE DRAWINGS

The summary, as well as the following detailed description, is further understood when read in conjunction with the appended drawings. For the purpose of illustrating the disclosed compositions and methods, the drawings show example embodiments of the compositions and methods; however, the compositions and methods are not limited to the specific embodiments disclosed. In the drawings:

FIG. 1 depicts sequences for human, mouse, and cynomolgus CD3. Such CD3 sequences are useful for the development of cross-reactive CD3 antigen binding domains for ease of clinical development.

FIG. 2 depicts sequences for human, mouse, and cynomolgus CD19. Such CD19 sequences are useful for the development of cross-reactive CD19 antigen binding domains for ease of clinical development.

FIG. 3A-FIG. 3F depict useful pairs of heterodimerization variant sets (including skew and pI variants). There are variants for which there are no corresponding “monomer 2” variants (FIG. 3F). Such variants are pI variants that can be used alone on either monomer of a bispecific antibody (e.g., CD19×CD3 bsAb), or included, for example, on the non-scFv side of a format that utilizes an scFv as a component and an appropriate charged scFv linker can be used on the second monomer that utilizes an scFv as the CD3 binding domain. Suitable charged linkers are shown in FIG. 6. Skew variants and pI variants are described, for example, in U.S. Pat. Nos. 11,299,554, 9,850,320 U.S. Patent Publication No. 2023/0048390, PCT Publication WO2014/145806, and U.S. Pat. No. 9,650,446, each of which is hereby incorporated by reference in its entirety.

FIG. 4 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein, according to an illustrative embodiment.

FIG. 5A and FIG. 5B depict useful ablation variants that ablate FcTR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 6 depicts a number of charged scFv linkers that find use in increasing or decreasing the pI of the subject heterodimeric bispecific antibodies that utilize one or more scFv as a component, as described herein (e.g., CD19×CD3 bispecific antibodies (bsAbs)). The (+H) positive linker finds particular use herein, particularly with anti-CD3 VL and VH sequences shown herein. A single prior art scFv linker with a single charge is referenced as “Whitlow,” from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc and 2+1 Fab2-scFv-Fc formats).

FIG. 7A and FIG. 7B depict a number of example domain linkers. In some embodiments, these linkers find use linking a single-chain Fv to an Fc chain. In some embodiments, these linkers may be combined in any orientation. For example, a GGGGS linker may be combined with a “lower half hinge” linker at the N-terminus or at the C-terminus.

FIG. 8 shows a particularly useful bispecific antibody backbone for the CD19×CD3 bispecific antibodies (bsAbs) of the disclosure. Although the backbone is described here in the context of the 1+1 Fab-scFv-Fc format, they can be adapted for use in other bispecific antibody formats.

FIG. 9 depicts various heterodimeric skew variant amino acid substitutions that can be used with the heterodimeric antibodies described herein.

FIG. 10A-FIG. 10E show the sequences of several heterodimeric CD19×CD3 bsAb backbones based on human IgG1, without the variable domains. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants on both chains. Heterodimeric Fc backbone 14 is based on human IgG1 (356E/358M allotype), and includes the T366W skew variant on a first heterodimeric Fc chain, the T366S/L368A/Y407V skew variants and H435R/Y436F purification variants on a second heterodimeric Fc chain, and the L234A/L235A/D265S ablation variants and M252Y/S254T/T256E half-life extension variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 16 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 17 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (G446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage. As used herein, a “heterodimeric Fc chain” is also referred to as a “variant Fc domain.”

FIG. 11 depicts sequences for “CH1” that find use in embodiments of CD19×CD3 bsAbs.

FIG. 12 depicts sequences for “hinge” that find use in embodiments of CD19×CD3 bsAbs.

FIG. 13 depicts the constant domain of the cognate light chains that find use in the subject CD19×CD3 bsAbs that utilize a Fab binding domain.

FIG. 14 depicts the variable heavy and variable light chain sequences for CD19-OG (VH=SEQ ID NO: 151/VL=SEQ ID NO: 152), an example CD19 binding domain. CDRs are underlined and slashes indicate the border(s) between the variable regions and constant domain. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 15 depicts the variable heavy and variable light chain sequences for CD19-NEW (VH=SEQ ID NO: 7/VL=SEQ ID NO: 8), an example CD19 binding domain that is a re-engineered variant of CD19-OG. CDRs are underlined and slashes indicate the border(s) between the variable regions and constant domain. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 16A-FIG. 16F depict the variable heavy and variable light chain sequences for illustrative CD3 binding domains which find use in the CD19×CD3 bsAbs of the disclosure. The CDRs are underlined, the scFv linker is double underlined (in the sequences, the scFv linker is a positively charged scFv (GKPGS)₄ linker (SEQ ID NO: 36), although as will be appreciated by those in the art, this linker can be replaced by other linkers, including uncharged or negatively charged linkers, some of which are depicted in FIG. 6), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 17A-FIG. 17J depict the variable heavy and variable light chain sequences for example CD3 binding domains re-engineered to improve stability while retaining an affinity ladder. The CDRs are underlined, the scFv linker is double underlined (in the sequences, the scFv linker is a positively charged scFv (GKPGS)₄ linker (SEQ ID NO: 36), although as will be appreciated by those in the art, this linker can be replaced by other linkers, including uncharged or negatively charged linkers, some of which are depicted in FIG. 6), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 1, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 18A-FIG. 18N depict bispecific formats of the present disclosure. FIG. 18A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 18B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 18C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 18D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 18E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. FIG. 18F depicts the “2+1 mAb-scFv” format which comprises a first monomer comprising from N-terminal to C-terminal VH1-CH1-linker-VH2-CH1-hinge-CH2-CH3 wherein CH2-CH3 is a first heterodimeric Fc domain; a second monomer comprising from N-terminal to C-terminal scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising from N-terminal to C-terminal VL-CL wherein the VL pairs with VH1 and VH2 of the first monomer to form two antigen binding domains each having a specificity for a second antigen binding domain. Additional bispecific formats include dual scFv (FIG. 18G), one-arm scFv-mAb (FIG. 18H), scFv-mAb (FIG. 18I), bispecific mAb (FIG. 18J), one-arm central-scFv (FIG. 18K), mAb-Fv (FIG. 18L), central-Fv (FIG. 18M), and trident (FIG. 18N).

FIG. 19A-FIG. 19C depict the sequences for illustrative CD19×CD3 bsAbs in the 1+1 Fab-scFv-Fc format. It should be noted that any other suitable backbones including those depicted in FIG. 10A-FIG. 10E may be used. CDRs are underlined and slashes indicate the border(s) between the variable regions and other domains. It should be noted that the CD19×CD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S, M428L/N434A, or M252Y/S254T/T256E variants in one or preferably both Fc domains, which including the variants results in longer half-life in serum.

FIG. 20A-FIG. 20AD depict the sequences for illustrative CD19×CD3 bsAbs in the 2+1 Fab2-scFv-Fc format. It should be noted that any other suitable backbones including those depicted in FIG. 10A-FIG. 10E may be used. CDRs are underlined and slashes indicate the border(s) between the variable regions and other domains. It should be noted that the CD19×CD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S, M428L/N434A, or M252Y/S254T/T256E variants in one or preferably both Fc domains, which including the variants results in longer half-life in serum.

FIG. 21 depicts in vitro depletion of CD19⁺ Ramos cells by CD19×CD3 bsAbs (in 1+1 or 2+1 formats with either high or intermediate affinity CD3 binding domains).

FIG. 22 depicts in vivo depletion of peripheral B cells in cynomolgus monkeys dosed with CD19×CD3 bsAbs (in 1+1 or 2+1 formats with either high or intermediate affinity CD3 binding domains).

FIG. 23 depicts in vivo depletion of peripheral B cells in cynomolgus monkeys dosed with CD19×CD3 bsAbs (in 1+1 or 2+1 formats with either high or intermediate affinity CD3 binding domains) with log₁₀ y-axis.

FIG. 24 depicts peripheral T cell activation in cynomolgus monkeys dosed with CD19×CD3 bsAbs (in 1+1 or 2+1 formats with either high or intermediate affinity CD3 binding domains).

FIG. 25A depicts in vivo depletion of bone marrow B cell and FIG. 25B depicts lymph node B cell in cynomolgus monkeys dosed with CD19×CD3 bsAbs (in 1+1 or 2+1 formats with either high or intermediate affinity CD3 binding domains).

FIG. 26 depicts serum concentration of CD19×CD3 bsAbs (in 1+1 or 2+1 formats with either high or intermediate affinity CD3 binding domains) over time in cynomolgus monkeys.

FIG. 27A depicts B cell depletion by XENP26982 and FIG. 27B serum concentration of XENP26982 in cynomolgus monkeys over time.

FIG. 28A and FIG. 28B depict comparison of CD19-OG and re-engineered CD19-NEW binding domains.

FIG. 29 depicts improvements in re-engineered CD19-NEW binding domain relative to CD19-OG.

FIG. 30 depicts affinity of original CD3 binding domains and CD3 binding domains re-engineered with disulfide bridges and deamidation fix.

FIG. 31 depicts induction of CD19⁺ Ramos cell kill by CD19×CD3 bsAbs with CD19-OG and CD19-NEW binding domains (in 1+1 and 2+1 formats with varied CD3 affinity).

FIG. 32A-FIG. 32D depict induction of T cell activation by CD19×CD3 bsAbs having original and re-engineered CD3 binding domains in the presence of human PBMC (FIG. 32A), cyno PBMC (FIG. 32B), and CD19+ Ramos cell kill (FIG. 32C).

FIG. 33 depicts induction of CD19+ Ramos cell kill, T cell activation, and IFNγ release by CD19×CD3 bsAbs with CD19-OG and CD19-NEW binding domains and original and re-engineered CD3 binding domains (in 1+1 and 2+1 formats).

FIG. 34 depicts induction of human B cell kill by CD19×CD3 bsAbs with CD19-NEW and the stability and affinity-enhanced CD3 binding domains.

FIG. 35 depicts the average pharmacokinetics at various relative doses of various CD19×CD3 bsAbs (along with ADA status), non-compartmental analysis.

FIG. 36 depicts depletion of cyno B cells in peripheral blood over time after dosing with various CD19×CD3 bsAbs at indicated relative doses. Certain of the points denoted 0.01 are 0 B cells per μL as shown in FIG. 41.

FIG. 37 depicts depletion of cyno B cells among lymphocytes over time after dosing with various CD19×CD3 bsAbs at indicated relative doses.

FIG. 38 depicts depletion of cyno B cells in lymph nodes over time after dosing with various CD19×CD3 bsAbs at indicated relative doses.

FIG. 39 depicts depletion of cyno B cells among bone marrow lymphocytes over time after dosing with various CD19×CD3 bsAbs at indicated relative doses.

FIG. 40 depicts depletion of B cell over time in all cynomolgus monkeys dosed with XENP49657 at 1× (top graph) or 3× dose level (bottom graph).

FIG. 41 depicts B cell depletion in periphery, bone marrow, and lymph nodes of cynomolgus monkeys dosed with 1× or 3× dose XENP49657. * indicates point is peripheral B cells <1 B cell per μL and +indicates data point is 0 B cells per μL. B cells were gated as CD45+CD2−/lowCD20+CD4−CD8a−CD159a−

FIG. 42 depicts plasma deletion in the bone marrow of cynomolgus monkeys dosed with 1× (top graph) or 3× dose (bottom graph) XENP49657. Plasma cells defined as SSlow/CD45+CD2−/lowCD20−/lowCD4−CD8a−CD159a−CD27−CD38high.

FIG. 43 depicts reduction in serum immunoglobulin in cynomolgus monkeys dosed with 1× or 3×XENP49657 that were not impacted by ADA.

FIG. 44 depicts affinity for human and cyno CD19 (apparent dissociation constant to account for bivalent binding, except in the case of 49653 which has monovalent CD19 binding), human and cyno CD3, and melting temperatures of CD19×CD3 bsAbs having the old CD3 and new stability-enhanced CD3 binding domains.

FIG. 45 depicts depletion of bone marrow plasma cells over time in cynomolgus monkeys dosed with 1× or 3× dose XENP49657.

FIG. 46 depicts pharmacokinetics of 1× and 3× intravenous dose XENP49657 in cynomolgus monkeys, half life ˜15 days.

FIG. 47 depicts pharmacokinetics of 3× subcutaneous dose XENP49657 in cynomolgus monkeys, half-life ˜15 days.

FIG. 48 depicts depletion of B cell over time in cynomolgus monkeys dosed subcutaneously with XENP49657 3× dose level.

FIG. 49 depicts deep B-depletion sustained for at least 42 days in cynomolgus monkeys dosed intravenously with XENP49657 at 3× dose level.

FIG. 50 depicts depletion of CD19+ plasma cells in bone marrow in cynomolgus monkeys dosed with CD19×CD3 bsAbs.

FIG. 51 depicts melting temperature of bispecific antibodies in the 2+1 Fab2-scFv-Fc format having original CD3 binding domains and CD3 binding domains re-engineered with disulfide bridges (as denoted by S-S).

FIG. 52 depicts affinity of original CD3 binding domains and CD3 binding domains re-engineered with disulfide bridges (as denoted by S-S) and deamidation fix (in the context of 2+1 Fab2-scFv-Fc bispecific antibodies).

FIG. 53 depicts induction of B cell killing (top panel), IFNγ secretion (middle panel), and IL-6 secretion (bottom panel) by XENP49657 in human PBMCs from healthy donors (average of 4 donors) vs. donors with rheumatoid arthritis (average of 7 donors).

FIG. 54 depicts induction of A) B cell killing, B) IFNγ secretion, and C) IL-6 secretion by XENP49657 in human PBMCs from healthy donors (average of 4 donors) vs. donors with systemic lupus erythematosus (average of 8 donors). depicts induction of A) B cell killing, B) IFNγ secretion, and C) IL-6 secretion by XENP49657 in human PBMCs from healthy donors (average of 4 donors) vs. donors with systemic lupus erythematosus (average of 8 donors).

### DETAILED DESCRIPTION

### I. Overview

Disclosed herein are anti-CD3 and anti-CD19 bispecific antigen binding molecules comprising an anti-CD19 antigen binding domain and an anti-CD3 antigen binding domain, such that the binding molecules bind to the extracellular domains of human CD19 and CD3.

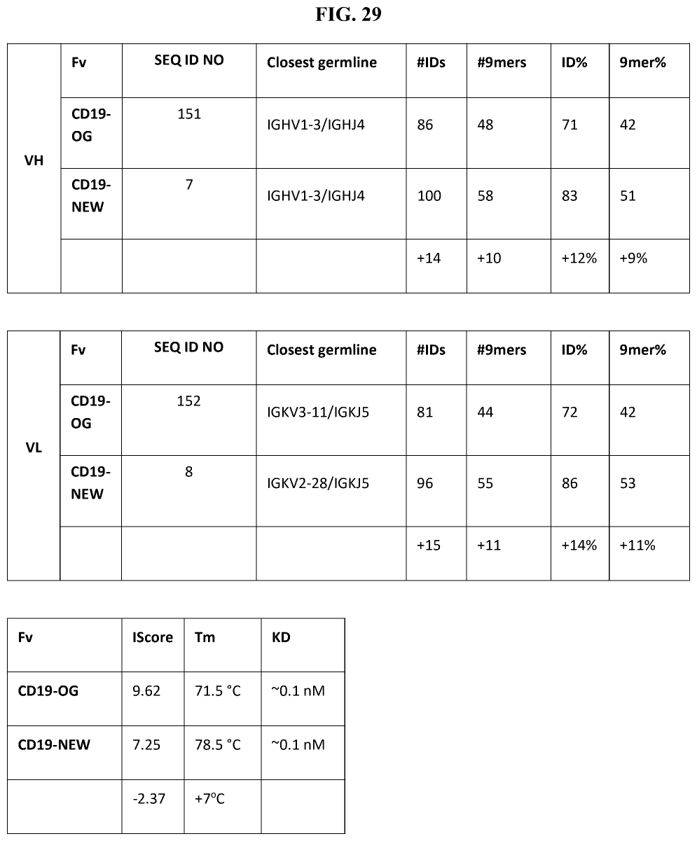

In some variations, the anti-CD19 antigen binding domain, anti-CD3 antigen binding domain, or both have improved stability while maintaining binding affinity. For example, anti-CD3 antigen binding domain variants of the bispecific antigen binding molecules are engineered to fix deamidation and incorporate disulfide bridges, while maintaining affinity and improved stability of the anti-CD3 antigen binding domain. The anti-CD3 antigen binding domain can have binding affinity similar to a non-engineered anti-CD3 antigen binding molecule. Likewise, the anti-CD19 antigen binding domain of the bispecific antigen binding molecules can be, for example, re-humanized and demonstrate enhanced stability, such as a +7° C. improved Tₘ (FIG. 29). The herein disclosed anti-CD3 and anti-CD19 bispecific antigen binding molecules exhibit comparable CD19+ Ramos cell killing and T-cell activation compared to the reference bispecific molecules that lack the sequence changes that mediate the improvements described herein.

The disclosed compositions and methods may be understood more readily by reference to the following detailed description taken in connection with the accompanying figures, which form a part of this disclosure. It is to be understood that the disclosed compositions and methods are not limited to the specific compositions and methods described and/or shown herein, and that the terminology used herein is for the purpose of describing particular embodiments by way of example only and is not intended to be limiting of the claimed compositions and methods.

Unless specifically stated otherwise, any description as to a possible mechanism or mode of action or reason for improvement is meant to be illustrative only, and the disclosed compositions and methods are not to be constrained by the correctness or incorrectness of any such suggested mechanism or mode of action or reason for improvement.

Throughout this text, the descriptions refer to compositions and methods of using said compositions. Where the disclosure describes or claims a feature or embodiment associated with a composition, such a feature or embodiment is equally applicable to the methods of using said composition. Likewise, where the disclosure describes or claims a feature or embodiment associated with a method of using a composition, such a feature or embodiment is equally applicable to the composition.

Where a range of numerical values is recited or established herein, the range includes the endpoints thereof and all the individual integers and fractions within the range, and also includes each of the narrower ranges therein formed by all the various possible combinations of those endpoints and internal integers and fractions to form subgroups of the larger group of values within the stated range to the same extent as if each of those narrower ranges was explicitly recited. Where a range of numerical values is stated herein as being greater than a stated value, the range is nevertheless finite and is bounded on its upper end by a value that is operable within the context of the herein disclosure. Where a range of numerical values is stated herein as being less than a stated value, the range is nevertheless bounded on its lower end by a non-zero value. It is not intended that the scope of the compositions and methods be limited to the specific values recited when defining a range. All ranges are inclusive and combinable.

When values are expressed as approximations, by use of the antecedent “about,” it will be understood that the particular value forms another embodiment. Reference to a particular numerical value includes at least that particular value, unless the context clearly dictates otherwise.

It is to be appreciated that certain features of the disclosed compositions and methods which are, for clarity, described herein in the context of separate embodiments, may also be provided in combination in a single embodiment. Conversely, various features of the disclosed compositions and methods that are, for brevity, described in the context of a single embodiment, may also be provided separately or in any subcombination.

The disclosures of each patent, patent application, and publication cited or described in this document are hereby incorporated herein by reference, in its entirety.

### III. Definitions

As used herein, the singular forms “a,” “an,” and “the” include the plural.

When a list is presented, unless stated otherwise, it is to be understood that each individual element of that list, and every combination of that list, is a separate embodiment. For example, a list of embodiments presented as “A, B, or C” is to be interpreted as including the embodiments: “A;” “B;” “C;” “A or B;” “A or C;” “B or C;” or “A, B, or C.”

Various terms relating to aspects of the description are used throughout the specification and claims. Such terms are to be given their ordinary meaning in the art unless otherwise indicated. Other specifically defined terms are to be construed in a manner consistent with the definitions provided herein.

The term “comprising” is intended to include examples encompassed by the terms “consisting essentially of” and “consisting of”; similarly, the term “consisting essentially of” is intended to include examples encompassed by the term “consisting of.”

“Antigen binding molecule” refers to a protein or peptide that specifically binds to an antigenic determinant. Examples of antigen binding molecules are immunoglobulin and derivatives or fragments thereof, e.g., Fab, scFv, 1+1 Fab-scFv-Fc, 2+1 Fab2-scFv-Fc, 1+1 Common Light Chain, 2+1 Common Light Chain, 2+1 mAb-scFv, 2+1 stackFab2-scFv-Fc, Dual scFv, One-arm scFv-mAb, scFv-mAb, Bispecific mAb, One-arm central-scFv, mAb-Fv, central-Fv, and Trident. Additional examples of antigen binding molecules are receptors and ligands.

“Binding subunit” as used herein refers to a protein subunit that comprises three “Complementarity Determining Regions” (CDRs). More than one binding subunit can be combined to form an antigen binding domain, which has specificity to an antigenic determinant. Multiple binding units may be linked directly or indirectly. A binding subunit can be a variable heavy domain or a variable light domain; that is, a variable heavy binding subunit and a variable light binding subunit.

“Antigen binding domain” or “ABD” herein is meant the part of an antigen binding molecule which confers its binding specificity to an antigenic determinant. Herein, the “antigen binding domain” refers to a set of six “Complementarity Determining Regions” (CDRs) that, when present as part of a polypeptide sequence, specifically binds a target antigen as discussed herein. The antigen binding domain can include a first binding subunit comprising three CDRs, and a second binding subunit comprising three CDRs. In some variations, the first binding subunit can correspond to a first set of variable heavy CDRs (vhCDRs or VHCDRs), while the second binding subunit can correspond to a second set of variable light CDRs (vlCDRs or VLCDRs). For example, as is known in the art, CDRs can be present as a first set of variable heavy CDRs and a second set of variable light CDRs, each comprising three CDRs: vhCDR1, vhCDR2, vhCDR3 for the heavy chain and vlCDR1, vlCDR2 and vlCDR3 for the light chain. The CDRs can be present in the variable heavy and variable light domains, respectively. In an antigen binding domain, when a first binding subunit is a variable heavy domain and a second binding subunit is a variable light domain, together the antigen binding domain can form an Fv region. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CHI domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh (including optional domain linkers on each side. In general, the C-terminus of the scFv domain is attached to the N-terminus of the hinge in the second monomer.

TABLE 1
CDR Positions

| | Kabat + | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

The term “antibody,” and like terms are a category of antigen binding molecule. In a broad sense antibody refers to immunoglobulin molecules including, monoclonal antibodies, antibody fragments, bispecific or multispecific antibodies, dimeric, tetrameric or multimeric antibodies, and single chain antibodies. Antibody formats include, but are not limited to Fab, scFv, 1+1 Fab-scFv-Fc, 2+1 Fab2-scFv-Fc, 1+1 Common Light Chain, 2+1 Common Light Chain, 2+1 mAb-scFv, 2+1 stackFab2-scFv-Fc, Dual scFv, One-arm scFv-mAb, scFv-mAb, Bispecific mAb, One-arm central-scFv, mAb-Fv, central-Fv, and Trident. Antibodies of the invention are generally derived or use components from human IgG, which has four isotypes IgG1, IgG2, IgG3 and IgG4. Of particular use herein are heavy chain constant regions from IgG1, IgG2 and IgG4, with IgG1 finding particular use in many embodiments. Antibody light chains of any vertebrate species can be assigned to one of two clearly distinct types, namely kappa (κ) and lambda (λ), based on the amino acid sequences of their constant domains.

“Bispecific” and “bi-specific” refer to the characteristic of a molecule that binds two different antigens or epitopes at the same time. A “bispecific antigen binding molecule,” or a “bispecific antibody” refers to a protein comprising at least two antigen-binding domains that are capable of simultaneously binding two different antigens (targets). Bispecific antibodies (also referred to as “bsAbs”) can be heterodimeric antibodies. Bispecific antigen binding molecules can, for example, be used to form a link between T cells and target cells, leading to the activation of T cells and generation of an immunological synapse. As shown herein, bispecific antigen binding constructs can be bivalent and bispecific (e.g. containing two antigen binding domains, each one binding a different target), or trivalent and bispecific (e.g. where the construct includes three antigen binding domains, two of which bind to one target (e.g. CD19) and one ABD that binds a separate target (e.g. CD3).

By “Fc” or “Fc region” or “Fc domain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus, the definition of “Fc domain” includes both amino acids 231-447 (CH2-CH3) or 216-447 (hinge-CH2-CH3), or fragments thereof. An “Fc fragment” in this context may contain fewer amino acids from either or both of the N- and C-termini but still retains the ability to form a dimer with another Fc domain or Fc fragment as can be detected using standard methods, generally based on size (e.g., non-denaturing chromatography, size exclusion chromatography, etc.) Human IgG Fc domains are of particular use in the methods provided herein, and can be the Fc domain from human IgG1, IgG2 or IgG4. Two Fc domains can self-assemble to form the full constant region or backbone.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region, and more particularly the ability to form a dimeric Fc domain.

“Heterodimeric antibody” or “heterodimeric domain,” means that the protein has at least two distinct sequences that associate to form a domain. For example, at least distinct two Fc sequences can assemble into a heterodimeric Fc domain, which can further comprise at least two Fv regions.

“Antibody fragment” refers to a portion of an immunoglobulin molecule that retains the specific antigen binding properties of the parental full length antibody (i.e. antigen-binding fragment thereof). By way of example and not limitation, antibody fragments comprise two binding domains, wherein each binding domain comprises a CDR1, a CDR2, and a CDR3. In some embodiments, the first binding domain can be a heavy chain comprising heavy chain complementarity determining regions (HCDR) 1, 2, and 3, and the second binding domain can be a light chain comprising complementarity determining regions (LCDR) 1, 2, and 3. Other example antibody fragments comprise a heavy chain variable region (VH) and a light chain variable region (VL). By “Fab” or “Fab region” as used herein is meant the polypeptide that comprises the VH, CH1, VL, and CL immunoglobulin domains. By “Fv” or “Fv fragment” or “Fv region” as used herein is meant a polypeptide that comprises the VL and VH domains of a single antibody. For example, antibody fragments include without limitation: a Fab fragment, a monovalent fragment consisting of the VL, VH, constant light (CL), and constant heavy 1 (CH1) domains; a F(ab)₂ fragment, a bivalent fragment comprising two Fab fragments linked by a disulfide bridge at the hinge region; and a Fv fragment consisting of the VL and VH domains of a single arm of an antibody. Further, VH and VL domains can be engineered and linked together via a synthetic linker to form various types of single chain antibody designs where the VH/VL domains pair intramolecularly, or intermolecularly in those cases when the VH and VL domains are expressed by separate single chain antibody constructs, to form a monovalent antigen binding site, such as single chain Fv (scFv) or diabody. These antibody fragments are obtained using techniques well known to those of skill in the art, and the fragments are screened for utility in the same manner as are full length antibodies.

An antigen binding molecule, antigen binding region, or antibody binding domain can have up to four “framework” regions (FRs) flanking CDRs in each binding subunit. In some variations, FRs alternate with three CDRs, in the sequence FR1, CDR1, FR2, CDR2, FR3, CDR3, FR4 (from amino to carboxy termini). If the binding subunit is a heavy chain variable region (VH), the three CDRs are identified as HCDR1, HCDR2, HCDR3 and the FRs are VHFR1, VHFR2, VHFR3 and VHFR4, such that the variable heavy domain comprises, from N- to C-terminal, VHFR1-VHCDR1-VIFR2-VHCDR2-VIFR3-VHCDR3-VHFR4. If the binding subunit is a light chain variable region (VL), the three CDRs are identified as LCDR1, LCDR2, LCDR3, the FRs are VLFR1, VLFR2, VLFR3 and VLFR4, such that the variable heavy domain comprises, from N- to C-terminal, VLFR1-VLCDR1-VLFR2-VLCDR2-VLFR3-VLCDR3-VLFR4. The location and size of the CDRs are defined based on rules which identify regions of sequence variability within the immunoglobulin variable regions (Wu and Kabat J Exp Med 132:211-50, 1970; Kabat et al. Sequences of Proteins of Immunological Interest, 5th Ed. Public Health Service, National Institutes of Health, Bethesda, Md., 1991).

“Humanized antibody” refers to an antibody in which the antigen binding sites are derived from non-human species and the framework regions are derived from human immunoglobulin sequences. Humanized antibodies may include substitutions in the framework regions so that the framework may not be an exact copy of expressed human immunoglobulin or germline gene sequences. If the antibody contains a constant region, the constant region is also derived from sequences of human origin. Methods of antibody humanization include, for example, string content optimization as described in U.S. Pat. No. 7,657,380.

“Human antibody” refers to an antibody having heavy and light chain variable regions in which both the framework and the antigen binding sites are derived from sequences of human origin. If the antibody contains a constant region, the constant region also is derived from sequences of human origin. A human antibody comprises heavy or light chain variable regions that are “derived from” sequences of human origin if the variable regions of the antibody are obtained from a system that uses human germline immunoglobulin or rearranged immunoglobulin genes. Such systems include human immunoglobulin gene libraries displayed on phage, and transgenic non-human animals such as mice or rats carrying human immunoglobulin loci. “Human antibody” may contain amino acid differences when compared to the human germline or rearranged immunoglobulin sequences due to, for example, naturally occurring somatic mutations or intentional introduction of substitutions in the framework or antigen binding sites. Typically, a “human antibody” is at least about 80%, 81%, 82%, 83%, 84%, 85%, 86%, 87%, 88%, 89%, 90%, 91%, 92%, 93%, 94%, 95%, 96%, 97%, 98%, 99%, or 100% identical in amino acid sequence to an amino acid sequence encoded by a human germline or rearranged immunoglobulin gene.

The terms “bind,” “binding,” and the like, refer to the ability of the disclosed antigen binding molecules to preferentially bind to its target (e.g. CD3 in the case of anti-CD3 antigen binding molecules or CD19 in the case of anti-CD19 antigen binding molecules) without preferentially binding other molecules in a sample containing a mixed population of molecules. Antigen binding molecules that immunospecifically bind, for example, CD3 are substantially free of other antigen binding molecules having different antigenic specificities (e.g., an anti-CD3 antigen binding molecule is substantially free of antigen binding molecules that specifically bind antigens other than CD3). Antigen binding molecules that immunospecifically bind, for example, CD3 and CD19, however, can have cross-reactivity to other antigens, such as orthologs of human CD3 and CD19, including Macacafascicularis (cynomolgus monkey) CD3 and CD19, respectively. The antigen binding molecules disclosed herein are able to immunospecifically bind both naturally-produced human CD3 and CD19 and to CD3 and CD19 which is recombinantly produced in mammalian or prokaryotic cells.

By “target antigen” as used herein is meant the molecule that is bound specifically by the antigen binding domain comprising the variable regions of a given antibody. As discussed herein, in the target antigens are CD19 and CD3.

The strength, or affinity, of specific binding can be expressed in terms of dissociation constant (K_{D}) of the interaction, wherein a smaller K_{D} represents greater affinity and a larger KD represents lower affinity. Binding properties can be determined by methods well known in the art such as bio-layer interferometry and surface plasmon resonance based methods. One such method entails measuring the rates of antigen-binding site/antigen or receptor/ligand complex association and dissociation, wherein rates depend on the concentration of the complex partners, the affinity of the interaction, and geometric parameters that equally influence the rate in both directions. Thus, both the association rate (ka) and the dissociation rate (kd) can be determined, and the ratio of kd/ka is equal to the dissociation constant K_{D} (See Nature 361:186-187 (1993) and Davies et al. (1990) Annual Rev Biochem 59:439-473).

Specific binding for a particular molecule or an epitope can be exhibited, for example, by an antigen binding molecule having a K_{D} for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰M, at least about 10⁻¹¹ M, at least about 10⁻¹² M, or lesser. Typically, an antigen binding molecule that specifically binds an antigen will have a K_{D} that is 20-, 50-, 100-, 500-, 1000-, 5,000-, 10,000- or more times greater for a control molecule relative to the antigen or epitope.

Also, specific binding for a particular molecule or an epitope can be exhibited, for example, by an antigen binding molecule having a ka or association rate for an antigen or epitope of at least 20-, 50-, 100-, 500-, 1000-, 5,000-, 10,000- or more times greater for the epitope relative to a control.

By “epitope” is herein meant a determinant that interacts with a specific antigen binding domain, for example variable region of an antibody molecule, known as a paratope. Epitopes are groupings of molecules such as amino acids or sugar side chains and usually have specific structural characteristics, as well as specific charge characteristics. A single molecule may have more than one epitope. The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide. Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents. An epitope typically includes at least 3, and more usually, at least 5 or 8-10 amino acids in a unique spatial conformation. Antigen binding molecules that recognize the same epitope can be verified in a simple immunoassay showing the ability of one antigen binding molecule to block the binding of another antigen binding molecule to a target antigen, for example “binning.” As outlined below, the invention not only includes the enumerated antigen binding molecules and antigen binding domains herein, but those that compete for binding with the epitopes bound by the enumerated antigen binding molecules or antigen binding domains.

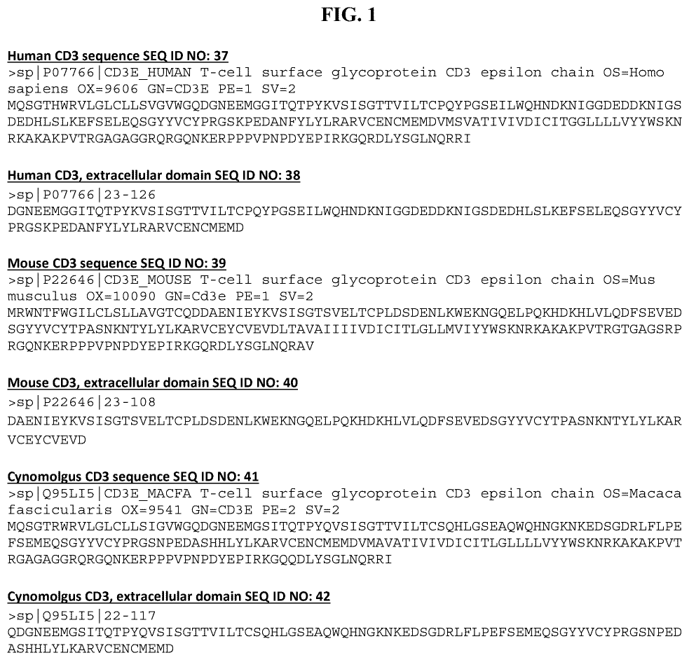

The term “CD3” refers to cluster of differentiation 3 protein complex or CD3 chains that form the CD3 complex, such as CD3 gamma, CD3 delta, CD3 zeta, and CD3 epsilon. In particular variations, CD3 refers to the human CD3-epsilon polypeptide of SEQ ID NO: 37. Example CD3 sequences to which the herein disclosed CD3 antigen binding molecules can bind include, for example, SEQ ID NOs: 37-42, which include human, mouse, and cynomolgus CD3 sequences (FIG. 1).

The term “CD19” refers to Cluster of Differentiation 19, which includes a transmembrane domain, a cytoplasmic C-terminus, and extracellular N-terminus. The herein disclosed CD19 antigen binding molecules can bind the extracellular N-terminus of CD19, which has two C2-type Ig-like domains divided by a smaller potential disulfide linked non-Ig-like domain. Example CD19 sequences to which the herein disclosed CD19 antigen binding molecules can bind include, for example, SEQ ID NOs: 43-48, which include human, mouse, and cynomolgus CD19 sequences (FIG. 1).

“Variant” refers to a polypeptide or a polynucleotide that differs from a reference polypeptide or a reference polynucleotide by one or more modifications for example, substitutions, insertions, or deletions.

By “position” as used herein is meant a location in the sequence of a protein. Positions may be numbered sequentially, or according to an established format, for example the EU index for antibody numbering.

“Parent polypeptide” as used herein is meant a starting polypeptide that is subsequently modified to generate a variant. The parent polypeptide may be a naturally occurring polypeptide, or a variant or engineered version of a naturally occurring polypeptide. Parent polypeptide may refer to the polypeptide itself, compositions that comprise the parent polypeptide, or the amino acid sequence that encodes it.

The term “percent identity” as used herein refers to the percent of amino acids that are the same between two or more polypeptides. Sequence identity between two polypeptides can be determined by aligning the amino acid sequences of the polypeptides and scoring the number of positions in the aligned polypeptides that contain the same amino acid residue and comparing that to the number of positions in the aligned polypeptides that differ. Polypeptides can differ at a position, for example, by containing a different amino acid (i.e. substitution or mutation) or by lacking an amino acid (i.e. amino acid insertion or amino acid deletion in one or both of the polypeptides). Sequences with at least 95% sequence identity to a given sequence also include, for example, sequences with 96%, 97%, 98%, and 99% sequence identity. Sequence identity between two similar sequences can be calculated by algorithms such as Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm (blast.ncbi.nlm.nih.gov/Blast_cgi).

“Treat,” “treatment,” and like terms refer to both therapeutic treatment and prophylactic or preventative measures, and includes reducing the severity and/or frequency of symptoms, eliminating symptoms and/or the underlying cause of the symptoms, reducing the frequency or likelihood of symptoms and/or their underlying cause, and improving or remediating damage caused, directly or indirectly, by, for example, an autoimmune disease. Treatment also includes prolonging survival as compared to the expected survival of a subject not receiving treatment. Subjects to be treated include those that have the condition or disorder as well as those prone to have the condition or disorder or those in which the condition or disorder is to be prevented.

The term “subject” as used herein is intended to mean any animal, in particular, mammals. Any type of mammal can be treated using the disclosed methods. Thus, the methods are applicable to human and nonhuman animals, although preferably used with mice, monkeys, and humans, and most preferably with humans. “Subject” and “patient” are used interchangeably herein.

As used herein, “administering to said patient” and similar terms indicate a procedure by which a herein disclosed antigen binding molecule is injected into a patient such that target cells, tissues, or segments of the body of the subject are contacted with a herein disclosed antigen binding molecule. Suitable routes of administration include, for example, intravenous administration and subcutaneous administration.

As used herein, “cancer” and “cancerous” refers to or describe the physiological condition in mammals that is typically characterized by unregulated cell growth, proliferation, or metastasis, and includes both solid cancers and non-solid cancers. Examples of cancer include but are not limited to carcinoma, lymphoma, blastoma, sarcoma (including liposarcoma), neuroendocrine tumors, mesothelioma, schwanoma, meningioma, adenocarcinoma, melanoma, and leukemia or lymphoid malignancies.

As used herein, the phrase “therapeutically effective amount” refers to an amount of a herein disclosed antigen binding molecule effective to achieve a particular biological or therapeutic result such as, but not limited to, biological or therapeutic results disclosed, described, or exemplified herein. The therapeutically effective amount may vary according to factors such as the disease state, age, sex, and weight of the individual, and the ability of the composition to cause a desired response in a subject.

“Autoimmune diseases” and like terms as used herein include allogenic islet graft rejection, alopecia areata, ankylosing spondylitis, antiphospholipid syndrome, autoimmune Addison's disease, antineutrophil cytoplasmic autoantibodies (ANCA), autoimmune diseases of the adrenal gland, anti-MDA5 syndrome, antisynthetase syndrome, autoimmune hemolytic anemia, autoimmune hepatitis, autoimmune autonomic ganglionopathy (AAG), autoimmune encephalitis (anti-NMDAR, anti-LGI1), autoimmune myocarditis, autoimmune neutropenia, autoimmune oophoritis and orchitis, autoimmune thrombocytopenia, autoimmune urticaria, Behcet's disease, bullous pemphigoid, cardiomyopathy, Castleman's syndrome, celiac spruce-dermatitis, chronic fatigue immune disfunction syndrome, chronic inflammatory demyelinating polyneuropathy, Churg-Strauss syndrome, cicatrical pemphigoid, cold agglutinin disease, CREST syndrome, cold agglutinin disease, Crohn's disease, dermatomyositis, discoid lupus, Evans syndrome, essential mixed cryoglobulinemia, factor VIII deficiency, fibromyalgia-fibromyositis, glomerulonephritis, Grave's disease, Guillain-Barre, Goodpasture's syndrome, graft-versus-host disease (GVHD), Hashimoto's thyroiditis, hemophilia A, idiopathic pulmonary fibrosis, idiopathic thrombocytopenia purpura (ITP), IgA neuropathy, IgM polyneuropathies, IgA vasculitis (Henoch-Schonlein purpura), IgG4-related disease, immune mediated thrombocytopenia, immune thrombocytopenic purpura (ITP) juvenile arthritis, Kawasaki's disease, lichen planus, lupus erthematosis, Meniere's disease, mixed connective tissue disease, multiple sclerosis, type 1 diabetes mellitus, myasthenia gravis, Neuromyelitis optica spectrum disorder (NMOSD), Overlap myositis, pemphigus vulgaris, pernicious anemia, polyarteritis nodosa, polychrondritis, polyglandular syndromes, polymyalgia rheumatica, polymyositis and dermatomyositis, primary agammaglobinulinemia, primary biliary cirrhosis, primary biliary cholangitis (CD19⁺ plasma-cell dominant), psoriasis, psoriatic arthritis, Reynauld's phenomenon, Reiter's syndrome, rheumatoid arthritis, sarcoidosis, scleroderma, Sjogren's syndrome, solid organ transplant rejection, stiff-man syndrome, systemic lupus erythematosus, systemic sclerosis (diffuse cutaneous subtype), type 1 diabetes mellitus (early or new-onset), takayasu arteritis, temporal arteristis/giant cell arteritis, thrombotic thrombocytopenia purpura, ulcerative colitis, uveitis, vasculitides such as dermatitis herpetiformis vasculitis, vitiligo, and Wegner's granulomatosis.

“Inflammatory disorders” and like terms as used herein include acute respiratory distress syndrome (ARDS), acute septic arthritis, adjuvant arthritis (Prakken et al., Springer Semin Immunopathol., 2003 August; 25(1):47-63, incorporated entirely by reference), juvenile idiopathic arthritis (de Kleer et al., Arthritis Rheum. 2003 July; 47(7):2001-10, incorporated entirely by reference), allergic encephalomyelitis, allergic rhinitis, allergic vasculitis, allergy, asthma, atherosclerosis, chronic inflammation due to chronic bacterial or viral infections, chronic obstructive pulmonary disease (COPD), coronary artery disease, encephalitis, inflammatory bowel disease, inflammatory osteolysis, inflammation associated with acute and delayed hypersensitivity reactions, inflammation associated with tumors, peripheral nerve injury or demyelinating diseases, inflammation associated with tissue trauma such as burns and ischemia, inflammation due to meningitis, multiple organ injury syndrome, pulmonary fibrosis, sepsis and septic shock, Stevens-Johnson syndrome, undifferentiated arthropy, and undifferentiated spondyloarthropathy.

### Bi-Specific CD3×CD19 Antigen Binding Molecules

Disclosed herein are bi-specific antigen binding molecules that bind CD3 and CD19. In further variations, antigen binding molecules that specifically bind to CD3 and antigen binding molecules that immunospecifically bind to CD19 are also provided.

As described herein, the bi-specific CD3×CD19 antigen binding molecules combine the antigen binding domains of an anti-CD3 antigen binding molecule and the antigen binding domains of an anti-CD19 antigen binding molecule. Anti-CD3 and anti-CD19 antigen binding molecules are disclosed separately herein, and can be combined in the bi-specific CD3×CD19 antigen binding molecule as described herein.

### CD3 Antigen Binding Molecules

Disclosed herein are anti-CD3 antigen binding molecules with improved stability. Sequence changes that mediate improved stability include, but are not limited to, changes in the framework region and changes in the CDRs. Improvements in stability include increase thermostability of the antigen binding molecules. Improving stability of the antigen binding molecule is important if antigen binding molecules are, for example, to be used in high concentrations such as in a subcutaneous formulation.

### CDRs

Disclosed herein are anti-CD3 antigen binding molecules. In one variation, the anti-CD3 antigen binding molecule comprises an antigen binding domain comprising a first binding subunit comprising the CDR sequences of SEQ ID NO: 9, SEQ ID NO: 10 or 11, and SEQ ID NO: 12, 13, 14, 15, or 16, and a second binding subunit comprising the CDR sequences of SEQ ID NOs: 17, 18, and 19. In some variations, the first binding subunit comprises the CDR sequences of SEQ ID NOs: 9, 10, and 12 and the second binding subunit comprises the CDR sequences of SEQ ID NOs: 17, 18, and 19. In some variations, the first binding subunit comprises the CDR sequences of SEQ ID NOs: 9, 10, and 13 and the second binding subunit comprises the CDR sequences of SEQ ID NOs: 17, 18, and 19. In some variations, the first binding subunit comprises the CDR sequences of SEQ ID NOs: 9, 11, and 12 and the second binding subunit comprises the CDR sequences of SEQ ID NOs: 17, 18, and 19. In some variations, the first binding subunit comprises the CDR sequences of SEQ ID NOs: 9, 11, and 13 and the second binding subunit comprises the CDR sequences of SEQ ID NOs: 17, 18, and 19. In some variations, the first binding subunit comprises the CDR sequences of SEQ ID NOs: 9, 10, and 14 and the second binding subunit comprises the CDR sequences of SEQ ID NOs: 17, 18, and 19. In some variations, the first binding subunit comprises the CDR sequences of SEQ ID NOs: 9, 10, and 15 and the second binding subunit comprises the CDR sequences of SEQ ID NOs: 17, 18, and 19. In some variations, the first binding subunit comprises the CDR sequences of SEQ ID NOs: 9, 11, and 14 and the second binding subunit comprises the CDR sequences of SEQ ID NOs: 17, 18, and 19. In some variations, the first binding subunit comprises the CDR sequences of SEQ ID NOs: 9, 11, and 15 and the second binding subunit comprises the CDR sequences of SEQ ID NOs: 17, 18, and 19. In some variations, the first binding subunit comprises the CDR sequences of SEQ ID NOs: 9, 10, and 16 and the second binding subunit comprises the CDR sequences of SEQ ID NOs: 17, 18, and 19.

### Linkers

The anti-CD3 antigen binding molecules can comprise a linker between the first binding subunit and the second binding subunit. The linker can comprise at least 95% sequence identity to the amino acid sequence of SEQ ID NO: 36 between the first binding subunit and the second binding subunit. The linker can comprise the amino acid sequence of SEQ ID NO: 36 between the first binding subunit and the second binding subunit. The linker can comprise at least 95% sequence identity to the amino acid sequence of any one of SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95. At least 95% sequence identity includes, for example, 96%, 97%, 98%, and 99% sequence identity. The linker can comprise the amino acid sequence of any one of SEQ ID NOs: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95.

### Variable Regions

In some variations of the anti-CD3 antigen binding molecule, the first binding subunit is a heavy chain variable region (VH) and the second binding subunit is a light chain variable region (VL). In some variations, the VH comprises a VH complementarity determining region (HCDR)1, HCDR2, and HCDR3 comprising the amino acid sequences of SEQ ID NOs: 9, 10, and 12, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 10, and 13, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 11, and 12, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 11, and 13, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 10, and 14, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 10, and 15, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 11, and 14, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 11, and 15, respectively, and the VL comprises the VL LCDR1, LCDR2, and LCDR3 comprising the amino acid sequences of SEQ ID NOs: 17, 18, and 19, respectively. In some variations, the VH comprises a VH HCDR1, HCDR2, and HCDR3 comprising the amino acid sequences of SEQ ID NOs: 9, 10, and 16, respectively, and the VL comprises the VL LCDR1, LCDR2, and LCDR3 comprising the amino acid sequences of SEQ ID NOs: 17, 18, and 19, respectively.

The anti-CD3 antigen binding molecules can comprise a heavy chain variable region comprising at least 95% sequence identity to the amino acid sequence of SEQ ID NO: 179, 181, 182, 183, 184, 185, 186, 187, 188, or 189, and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 179 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 181 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 182 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 183 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 184 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 185 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 186 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 187 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 188 and a VL comprising at least 95% sequence identity to the amino acid sequence of SEQ ID NO: 180. In some variations, the anti-CD3 antigen binding molecule comprises a VH comprising at least 95% sequence identity to the amino acid sequence of SEQ ID NO: 189 and a VL comprising at least 95% sequence identity to the amino acid sequence of SEQ ID NO: 180. At least 95% sequence identity includes, for example, 96%, 97%, 98%, and 99% sequence identity.

The anti-CD3 antigen binding molecules can comprise a heavy chain variable region comprising the amino acid sequence of SEQ ID NO: 179, 181, 182, 183, 184, 185, 186, 187, 188, or 189, and a VL comprising the amino acid sequence of SEQ ID NO: 180. In some variations, the anti-CD3 antigen binding molecule comprises a VH comprising the amino acid sequence of SEQ ID NO: 179 and a VL comprising the amino acid sequence of SEQ ID NO: 180. In some variations, the anti-CD3 antigen binding molecule comprises a VH comprising the amino acid sequence of SEQ ID NO: 181 and a VL comprising the amino acid sequence of SEQ ID NO: 180. In some variations, the anti-CD3 antigen binding molecule comprises a VH comprising the amino acid sequence of SEQ ID NO: 182 and a VL comprising the amino acid sequence of SEQ ID NO: 180. In some variations, the anti-CD3 antigen binding molecule comprises a VH comprising the amino acid sequence of SEQ ID NO: 183 and a VL comprising the amino acid sequence of SEQ ID NO: 180. In some variations, the anti-CD3 antigen binding molecule comprises a VH comprising the amino acid sequence of SEQ ID NO: 184 and a VL comprising the amino acid sequence of SEQ ID NO: 180. In some variations, the anti-CD3 antigen binding molecule comprises a VH comprising the amino acid sequence of SEQ ID NO: 185 and a VL comprising the amino acid sequence of SEQ ID NO: 180. In some variations, the anti-CD3 antigen binding molecule comprises a VH comprising the amino acid sequence of SEQ ID NO: 186 and a VL comprising the amino acid sequence of SEQ ID NO: 180. In some variations, the anti-CD3 antigen binding molecule comprises a VH comprising the amino acid sequence of SEQ ID NO: 187 and a VL comprising the amino acid sequence of SEQ ID NO: 180. In some variations, the anti-CD3 antigen binding molecule comprises a VH comprising the amino acid sequence of SEQ ID NO: 188 and a VL comprising the amino acid sequence of SEQ ID NO: 180. In some variations, the anti-CD3 antigen binding molecule comprises a VH comprising the amino acid sequence of SEQ ID NO: 189 and a VL comprising the amino acid sequence of SEQ ID NO: 180.

The anti-CD3 antigen binding molecule can comprise a VH of any one of SEQ ID NOs: 250-2644. The VH of any one of SEQ ID NOs: 250-2644 can comprise a G44C substitution. The VH of any one of SEQ ID NOs: 250-2644 can comprise a N97Q, G96T, G96E, D100G, or any combination thereof, such as G96T/D100G or G96E/D100G. In some embodiments, the substitution is N97Q. In some embodiments, the substitution is D100G. In some embodiments, the substitutions are N97Q and D100G. The anti-CD3 antigen binding molecule can comprise a VL of any one of SEQ ID NOs: 2645-3380. The VL of any one of SEQ ID NOs: 2645-3380 can comprise a G100C substitution. The herein disclosed anti-CD3 antigen binding molecules can comprise any combination of disclosed VH or VL. For example, the anti-CD3 antigen binding molecule can comprise a VH and a VL, wherein the VH comprises any one of SEQ ID NOs: 250-2644 or a variant thereof comprising G44C, N97Q, G96T, G96E, D100G, or a combination thereof, and the VL comprises any one of SEQ ID NOs: 2645-3380 or a variant thereof comprising G100C.

The herein disclosed anti-CD3 antigen binding molecules can comprise a single-chain variable fragment (scFv) comprising at least 95% sequence identity to the amino acid sequence of SEQ ID NO: 190, 191, 192, 193, 194, 195, 196, 197, 198, 199, 200, 201, 202, 203, 204, 205, 206, 207, 208, or 209. In some variations, the scFV comprises at least 95% sequence identity to the amino acid sequence of SEQ ID NO: 190. In some variations, the scFV comprises at least 95% sequence identity to the amino acid sequence of SEQ ID NO: 191. In some variations, the scFV comprises at least 95% sequence identity to the amino acid sequence of SEQ ID NO: 192. In some variations, the scFV comprises at least 95% sequence identity to the amino acid sequence of SEQ ID NO: 193. In some variations, the scFV comprises at least 95% sequence identity to the amino acid sequence of SEQ ID NO: 194. In some variations, the scFV comprises at least 95% sequence identity to the amino acid sequence of SEQ ID NO: 195. In some variations, the scFV comprises at least 95% sequence identity to the amino acid sequence of SEQ ID NO: 196. In some variations, the scFV comprises at least 95% sequence identity to the amino acid sequence of SEQ ID NO: 197. In some variations, the scFV comprises at least 95% sequence identity to the amino acid sequence of SEQ ID NO: 198. In some variations, the scFV comprises at least 95% sequence identity to the amino acid sequence of SEQ ID NO: 199. In some variations, the scFV comprises at least 95% sequence identity to the amino acid sequence of SEQ ID NO: 200. In some variations, the scFV comprises at least 95% sequence identity to the amino acid sequence of SEQ ID NO: 201. In some variations, the scFV comprises at least 95% sequence identity to the amino acid sequence of SEQ ID NO: 202. In some variations, the scFV comprises at least 95% sequence identity to the amino acid sequence of SEQ ID NO: 203. In some variations, the scFV comprises the amino acid sequence of SEQ ID NO: 204. In some variations, the scFV comprises at least 95% sequence identity to the amino acid sequence of SEQ ID NO: 205. In some variations, the scFV comprises at least 95% sequence identity to the amino acid sequence of SEQ ID NO: 206. In some variations, the scFV comprises at least 95% sequence identity to the amino acid sequence of SEQ ID NO: 207. In some variations, the scFV comprises at least 95% sequence identity to the amino acid sequence of SEQ ID NO: 208. In some variations, the scFV comprises at least 95% sequence identity to the amino acid sequence of SEQ ID NO: 209. At least 95% sequence identity includes, for example, 96%, 97%, 98%, and 99% sequence identity.

The herein disclosed anti-CD3 antigen binding molecules can comprise a single-chain variable fragment (scFv) comprising the amino acid sequence of SEQ ID NO: 190, 191, 192, 193, 194, 195, 196, 197, 198, 199, 200, 201, 202, 203, 204, 205, 206, 207, 208, or 209. In some variations, the scFV comprises the amino acid sequence of SEQ ID NO: 190. In some variations, the scFV comprises the amino acid sequence of SEQ ID NO: 191. In some variations, the scFV comprises the amino acid sequence of SEQ ID NO: 192. In some variations, the scFV comprises the amino acid sequence of SEQ ID NO: 193. In some variations, the scFV comprises the amino acid sequence of SEQ ID NO: 194. In some variations, the scFV comprises the amino acid sequence of SEQ ID NO: 195. In some variations, the scFV comprises the amino acid sequence of SEQ ID NO: 196. In some variations, the scFV comprises the amino acid sequence of SEQ ID NO: 197. In some variations, the scFV comprises the amino acid sequence of SEQ ID NO: 198. In some variations, the scFV comprises the amino acid sequence of SEQ ID NO: 199. In some variations, the scFV comprises the amino acid sequence of SEQ ID NO: 200. In some variations, the scFV comprises the amino acid sequence of SEQ ID NO: 201. In some variations, the scFV comprises the amino acid sequence of SEQ ID NO: 202. In some variations, the scFV comprises the amino acid sequence of SEQ ID NO: 203. In some variations, the scFV comprises the amino acid sequence of SEQ ID NO: 204. In some variations, the scFV comprises the amino acid sequence of SEQ ID NO: 205. In some variations, the scFV comprises the amino acid sequence of SEQ ID NO: 206. In some variations, the scFV comprises the amino acid sequence of SEQ ID NO: 207. In some variations, the scFV comprises the amino acid sequence of SEQ ID NO: 208. In some variations, the scFV comprises the amino acid sequence of SEQ ID NO: 209.

### Framework Regions

The anti-CD3 antigen binding molecule can comprise one or more framework regions between CDRs. The framework region described herein can be at specific positions relative to the CDRs of the first binding subunit and the CDRs of the second binding subunit in an antigen binding domain.

Disclosed herein are anti-CD3 antigen binding molecules comprising an antigen binding domain. The antigen binding domain comprises a first binding subunit, a second binding subunit, and a framework region comprising at least 95% identity to an amino acid sequence selected from the group consisting of SEQ ID NOs: 28, 29, 30, 31, 32, 33, 34, and 35. In some variations, the framework region comprises at least 95% identity to the amino acid sequence of SEQ ID NOs: 28. In some variations, the framework region comprises the amino acid sequence of SEQ ID NOs: 28. In some variations, the framework region comprises at least 95% identity to the amino acid sequence of SEQ ID NOs: 29. In some variations, the framework region comprises the amino acid sequence of SEQ ID NOs: 29. In some variations, the framework region comprises at least 95% identity to the amino acid sequence of SEQ ID NOs: 30. In some variations, the framework region comprises the amino acid sequence of SEQ ID NOs: 30. In some variations, the framework region comprises at least 95% identity to the amino acid sequence of SEQ ID NOs: 31. In some variations, the framework region comprises the amino acid sequence of SEQ ID NOs: 31. In some variations, the framework region comprises at least 95% identity to the amino acid sequence of SEQ ID NOs: 32. In some variations, the framework region comprises the amino acid sequence of SEQ ID NOs: 32. In some variations, the framework region comprises at least 95% identity to the amino acid sequence of SEQ ID NOs: 33. In some variations, the framework region comprises the amino acid sequence of SEQ ID NOs: 33. In some variations, the framework region comprises at least 95% identity to the amino acid sequence of SEQ ID NOs: 34. In some variations, the framework region comprises the amino acid sequence of SEQ ID NOs: 34. In some variations, the framework region comprises at least 95% identity to the amino acid sequence of SEQ ID NOs: 35. In some variations, the framework region comprises the amino acid sequence of SEQ ID NOs: 35. At least 95% sequence identity includes, for example, 96%, 97%, 98%, and 99% sequence identity.

The anti-CD3 antigen binding molecules can comprise a framework region 1 (FR1) comprising at least 95% identity to the amino acid sequence of SEQ ID NO: 28, a framework region 2 (FR2) comprising at least 95% identity to the amino acid sequence of SEQ ID NO: 29, a framework region 3 (FR3) comprising at least 95% identity to the amino acid sequence of SEQ ID NO: 30, a framework region 4 (FR4) comprising at least 95% identity to the amino acid sequence of SEQ ID NO: 31, a framework region 5 (FR5) comprising at least 95% identity to the amino acid sequence of SEQ ID NO: 32, a framework region 6 (FR6) comprising at least 95% identity to the amino acid sequence of SEQ ID NO: 33, a framework region 7 (FR7) comprising at least 95% identity to the amino acid sequence of SEQ ID NO: 34, and a framework region 8 (FR8) comprising at least 95% identity to the amino acid sequence of SEQ ID NO: 35. At least 95% sequence identity includes, for example, 96%, 97%, 98%, and 99% sequence identity.

The anti-CD3 antigen binding molecules can comprise FR1 comprising the amino acid sequence of SEQ ID NO: 28, a FR2 comprising the amino acid sequence of SEQ ID NO: 29, a FR3 comprising the amino acid sequence of SEQ ID NO: 30, a FR4 comprising the amino acid sequence of SEQ ID NO: 31, a FR5 comprising the amino acid sequence of SEQ ID NO: 32, a FR6 comprising the amino acid sequence of SEQ ID NO: 33, a FR7 comprising the amino acid sequence of SEQ ID NO: 34, and a FR8 comprising the amino acid sequence of SEQ ID NO: 35.

Disclosed herein are anti-CD3 antigen binding molecules comprising an amino acid sequence comprising at least 95% identity to SEQ ID NO: 28 N-terminal to a first binding subunit CDR1, an amino acid sequence comprising at least 95% identity to SEQ ID NO: 29 between the first binding subunit CDR1 and a first binding subunit CDR2, an amino acid sequence comprising at least 95% identity to SEQ ID NO: 30 between the first binding subunit CDR2 and a first binding subunit CDR3, an amino acid sequence comprising at least 95% identity to SEQ ID NO: 31 C-terminal to the first binding subunit CDR3, an amino acid sequence comprising at least 95% identity to SEQ ID NO: 32 N-terminal to a second binding subunit CDR1, an amino acid sequence comprising at least 95% identity to SEQ ID NO: 33 between the second binding subunit CDR1 and a second binding subunit CDR2, an amino acid sequence comprising at least 95% identity to SEQ ID NO: 34 between the second binding subunit CDR2 and a second binding subunit CDR3; and an amino acid sequence comprising at least 95% identity to SEQ ID NO: 35 C-terminal to the second binding subunit CDR3. At least 95% sequence identity includes, for example, 96%, 97%, 98%, and 99% sequence identity. In some variations, the first binding subunit CDR1 comprises the amino acid sequence of SEQ ID NO: 9, the first binding subunit CDR2 comprises the amino acid sequence of SEQ ID NO: 10 or 11, the first binding subunit CDR3 comprises the amino acid sequence of SEQ ID NO: 12, 13, 14, 15, or 16, the second binding subunit CDR1 comprising the amino acid sequence of SEQ ID NO: 17, the second binding subunit CDR2 comprises the amino acid sequence of SEQ ID NO: 18, and the second binding subunit CDR3 comprises the amino acid sequence of SEQ ID NO: 19.

In some variations, the first binding subunit comprises a CDR1, CDR2, and CDR3 comprising the amino acid sequences of SEQ ID NOs: 9, 10, and 12, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 10, and 13, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 11, and 12, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 11, and 13, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 10, and 14, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 10, and 15, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 11, and 14, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 11, and 15, respectively, and the second binding subunit comprises a CDR1, CDR2, and CDR3 comprising the amino acids sequences of SEQ ID NOs: 17, 18, and 19, respectively. In some variations, the first binding subunit comprises a CDR1, CDR2, and CDR3 comprising the amino acid sequences of SEQ ID NOs: 9, 10, and 16, respectively, and the second binding subunit comprises a CDR1, CDR2, and CDR3 comprising the amino acids sequences of SEQ ID NOs: 17, 18, and 19, respectively.

### Stability

An antigen binding molecule, such as an anti-CD3 antigen binding molecule can include, for example, a non-canonical deamidation motif GNF at positions 96-98 (Kabat numbering) in the first binding subunit CDR3. Consequentially, variants are made to remove this deamidation liability as well as to engineer stabilizing disulfide bridges. N97 is important for binding, but variants at N97 abrogated binding affinity (with the exception of N97Q which retains some binding). Variants at G96 is also important to binding, and G96E or G96T was found to improve binding affinity (albeit with decrease in stability by ˜2-4° C.).

The disulfide bridges can increase the thermodynamic stability of the antigen binding molecule. In some variations, the disulfide bridges can increase the denaturation temperature by at least 2° C.). In some variations, the disulfide bridges can increase the denaturation temperature by at least 3° C.). In some variations, the disulfide bridges can increase the denaturation temperature by at least 4° C.).

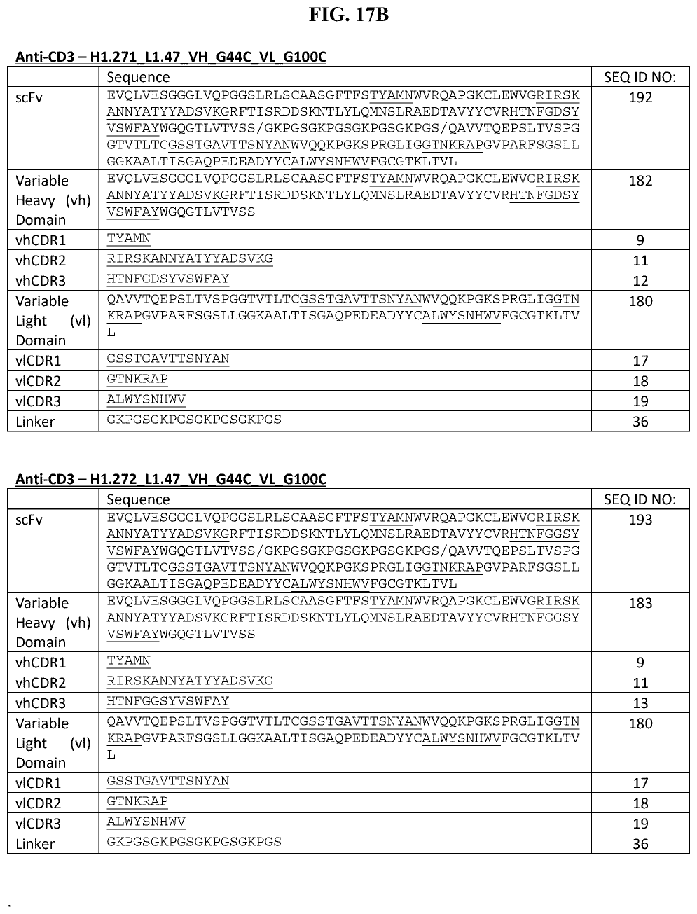

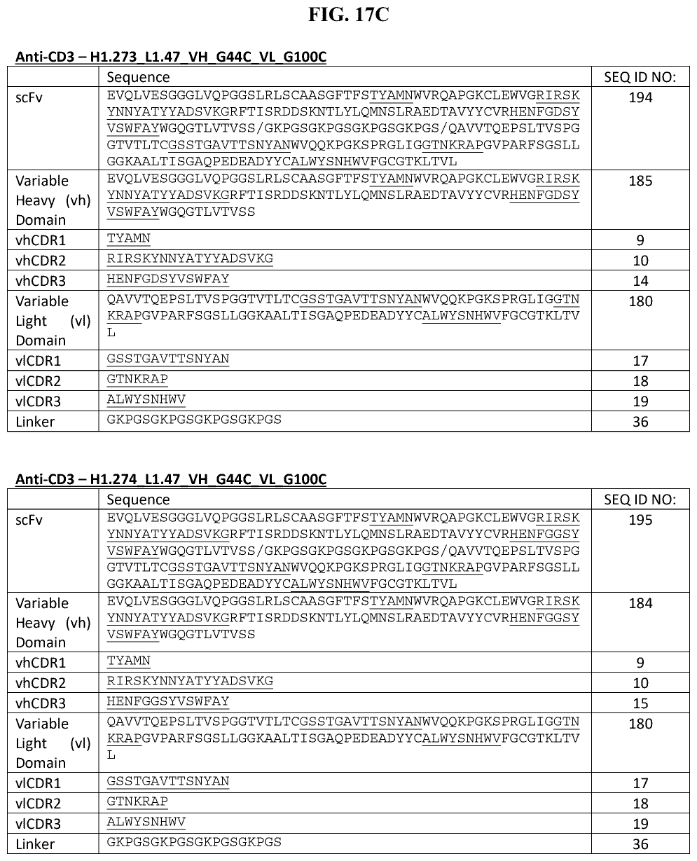

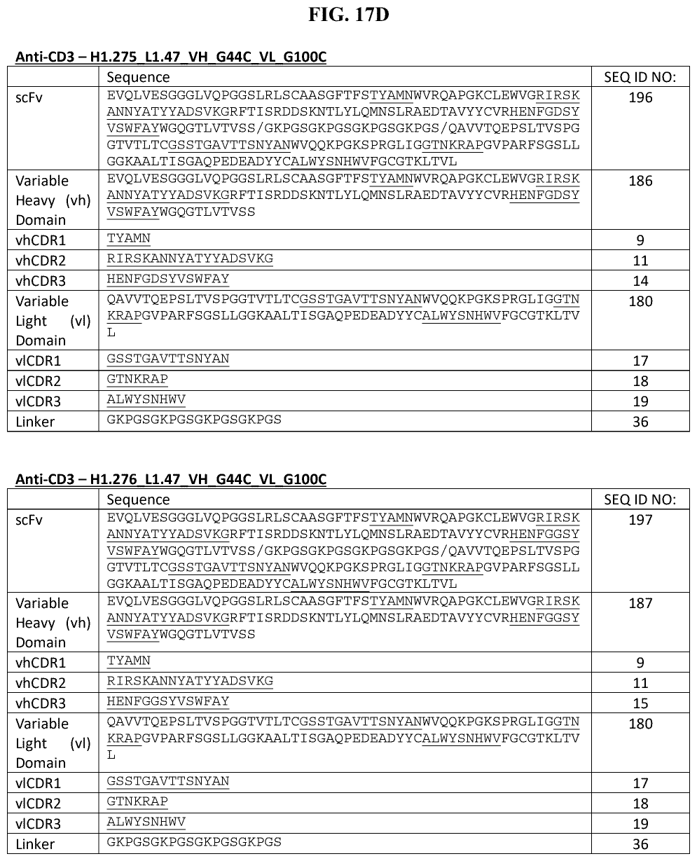

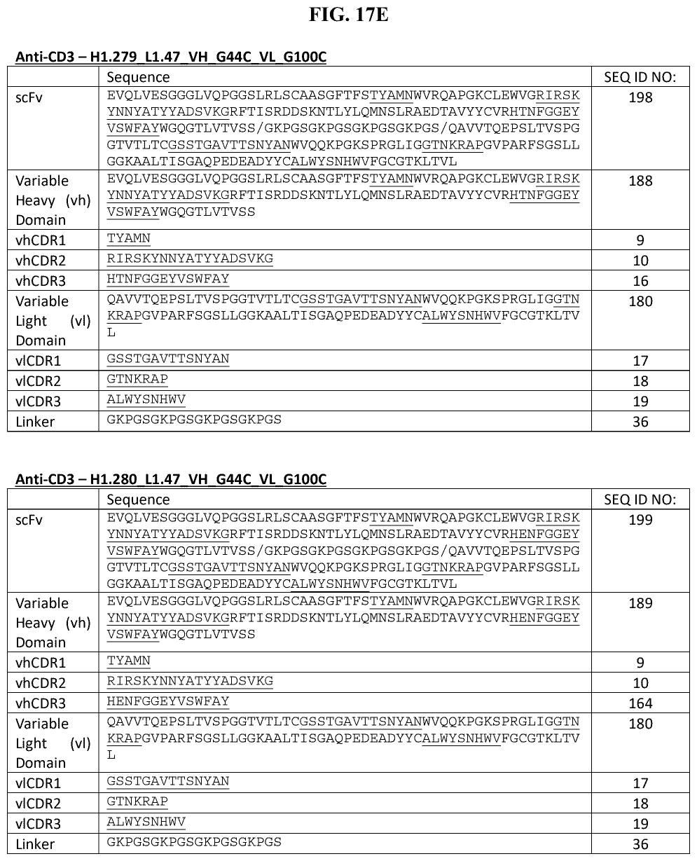

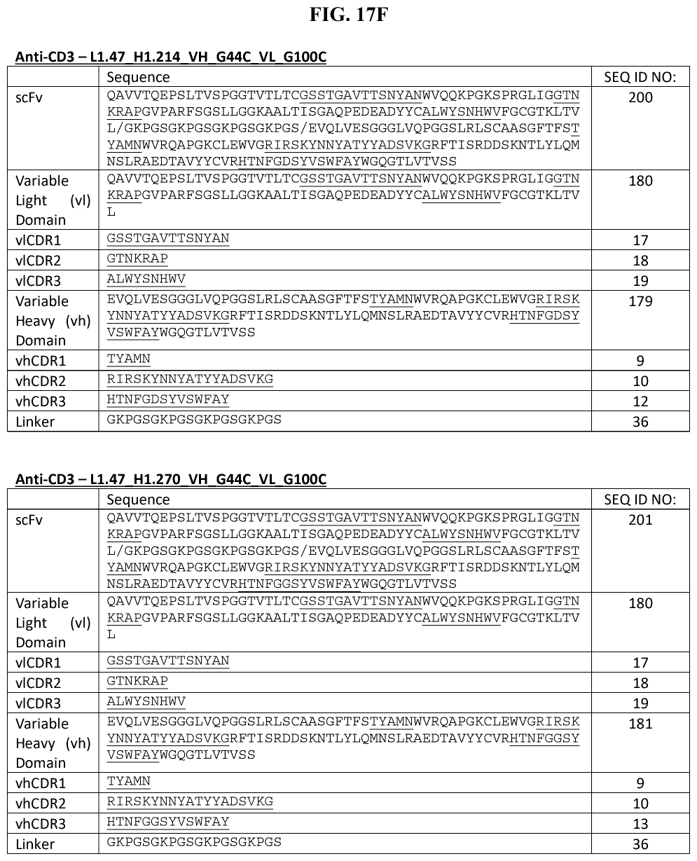

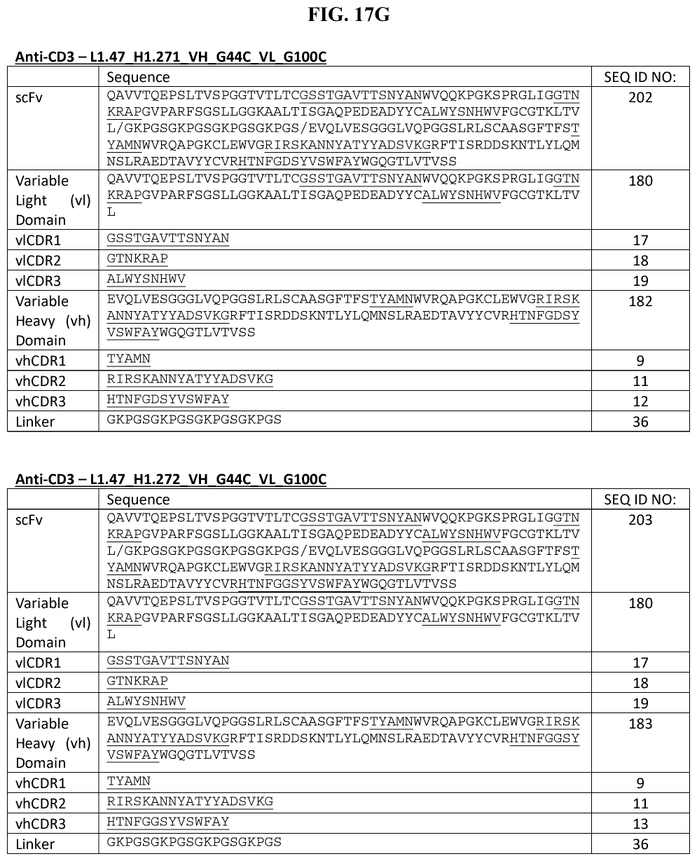

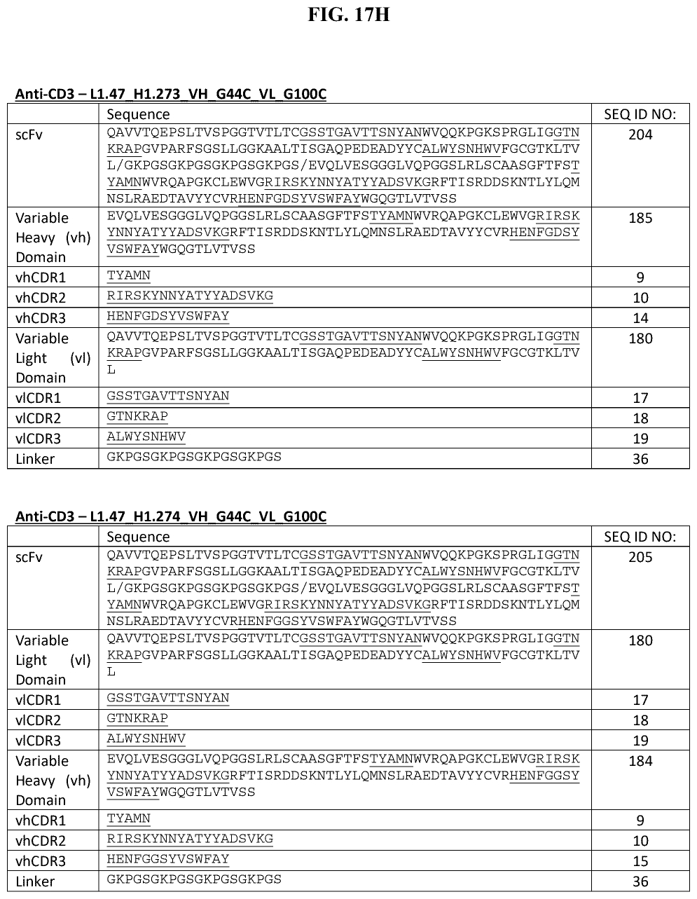

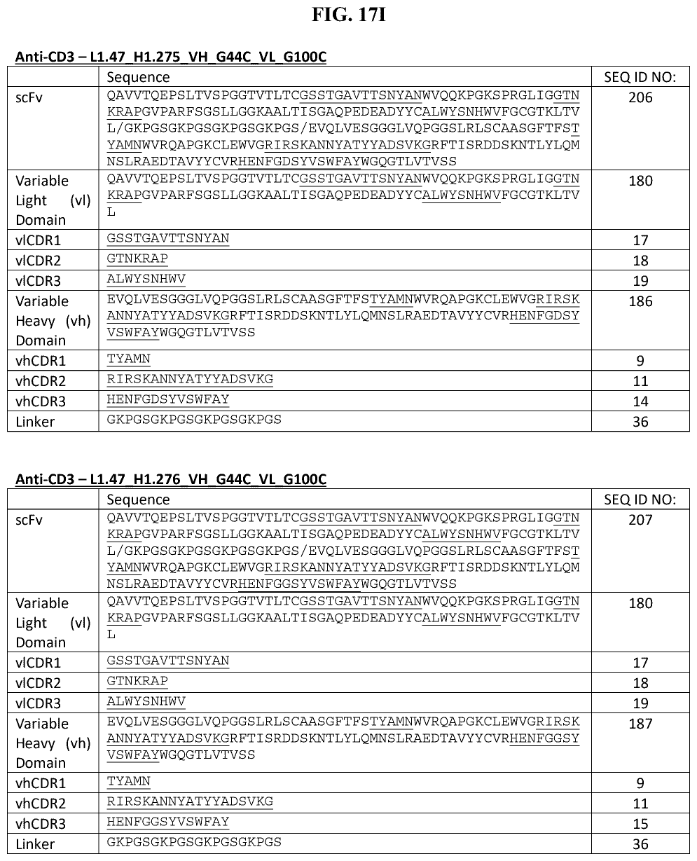

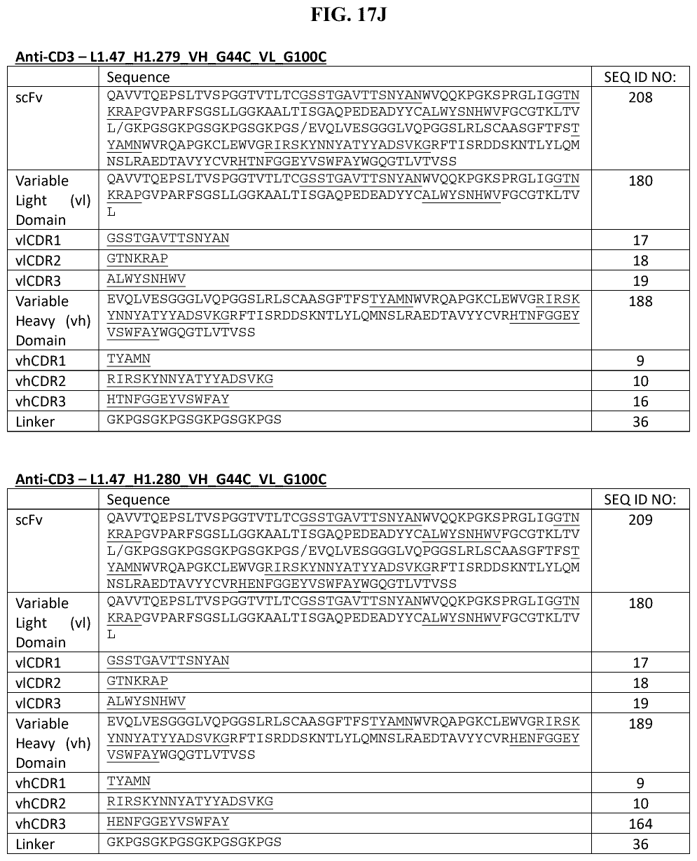

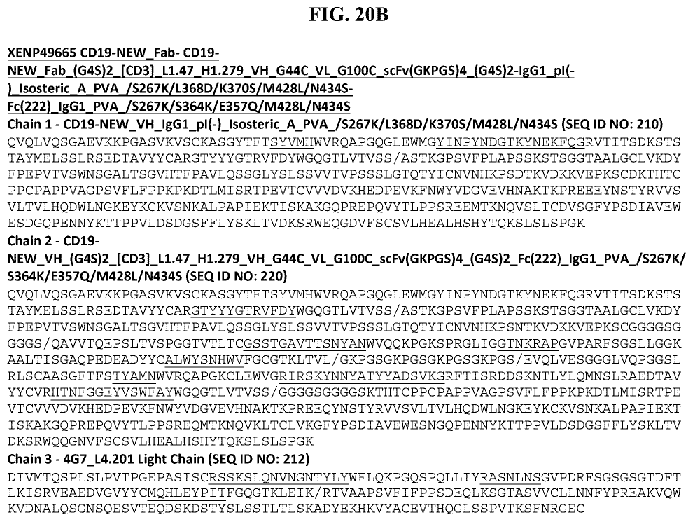

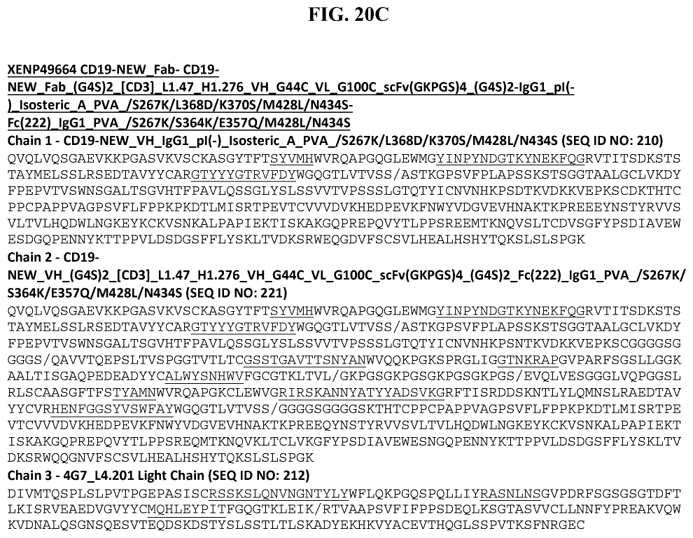

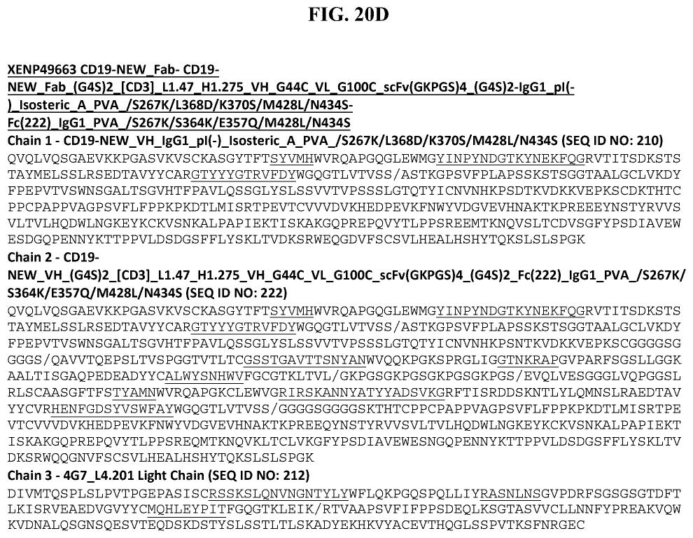

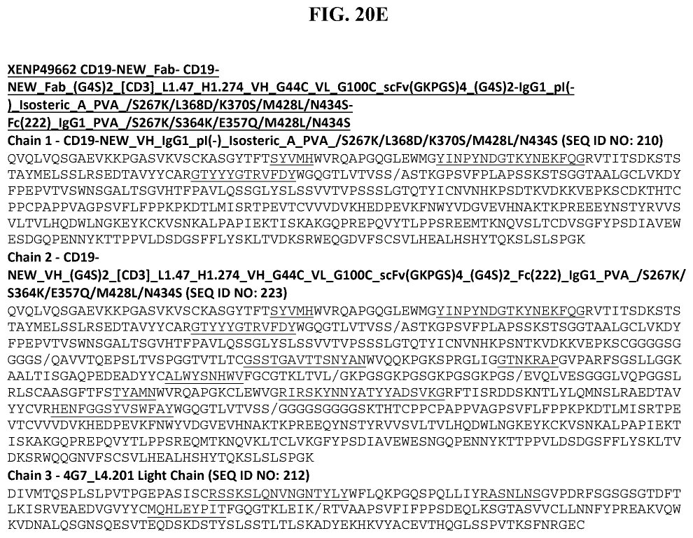

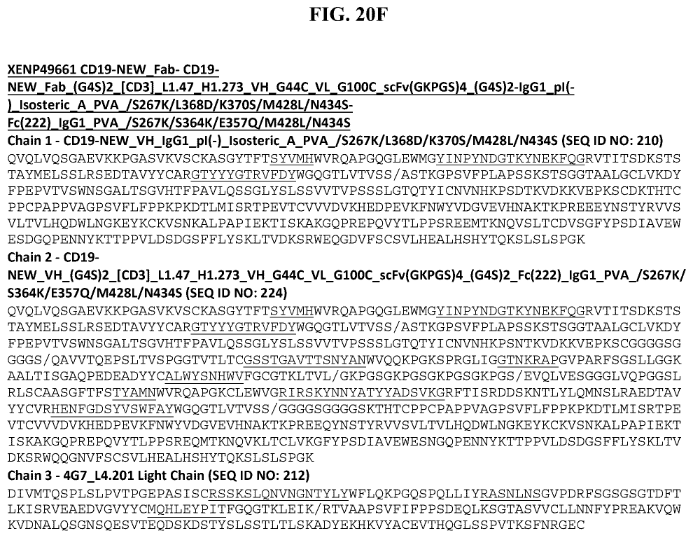

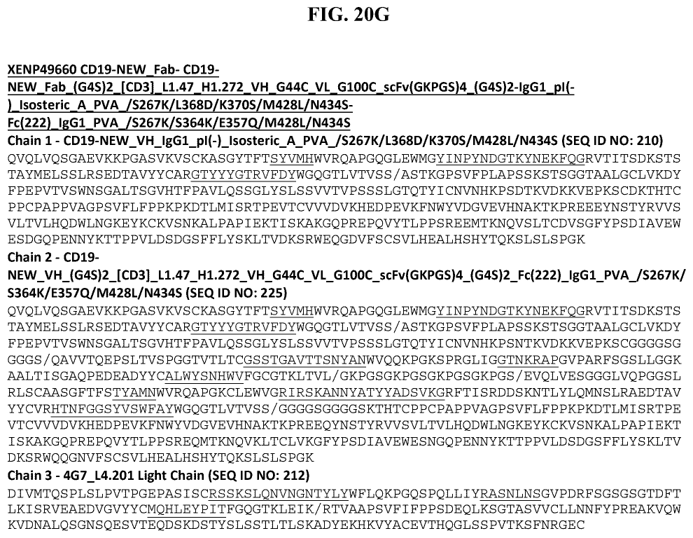

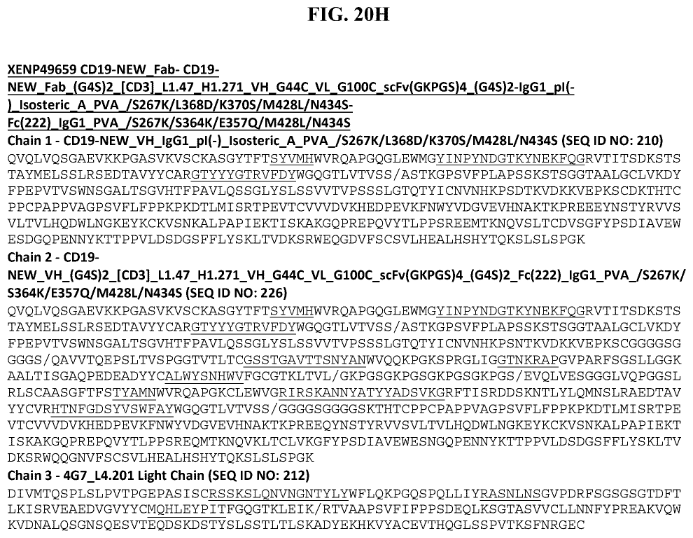

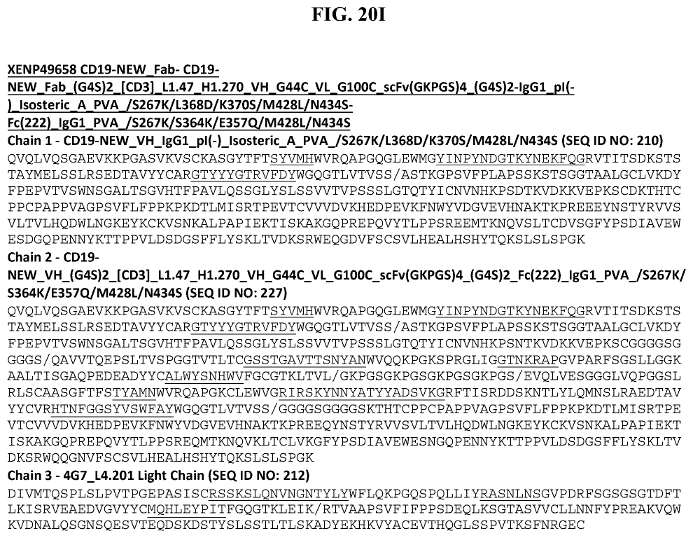

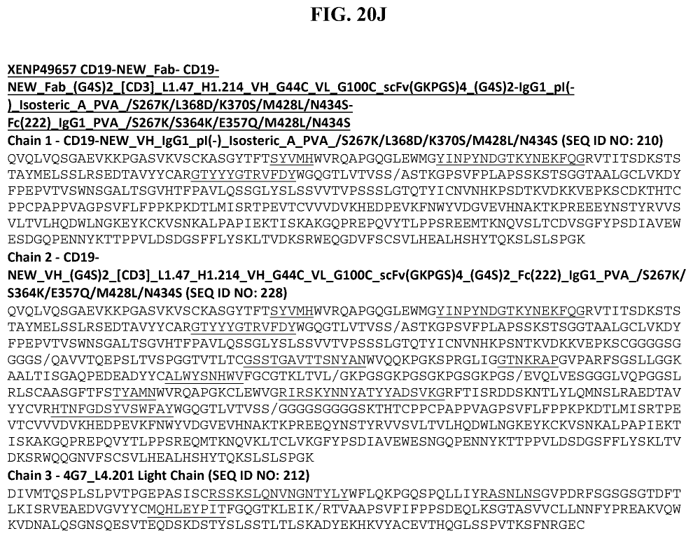

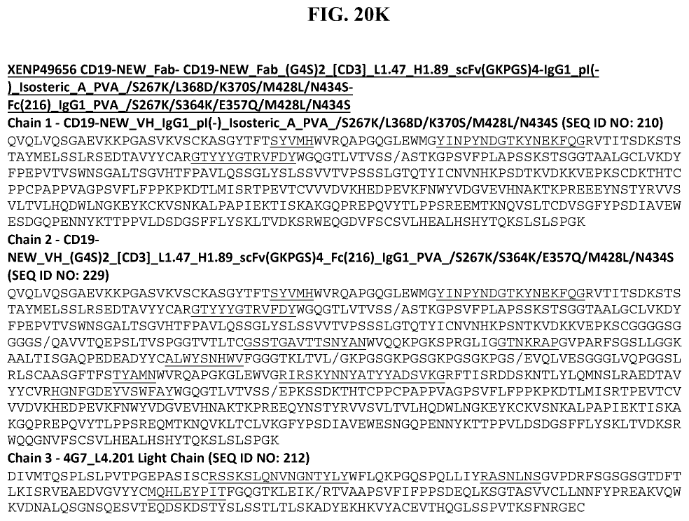

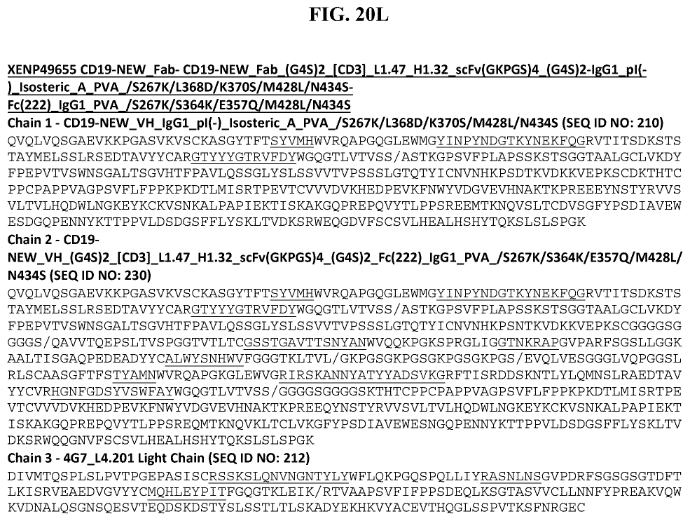

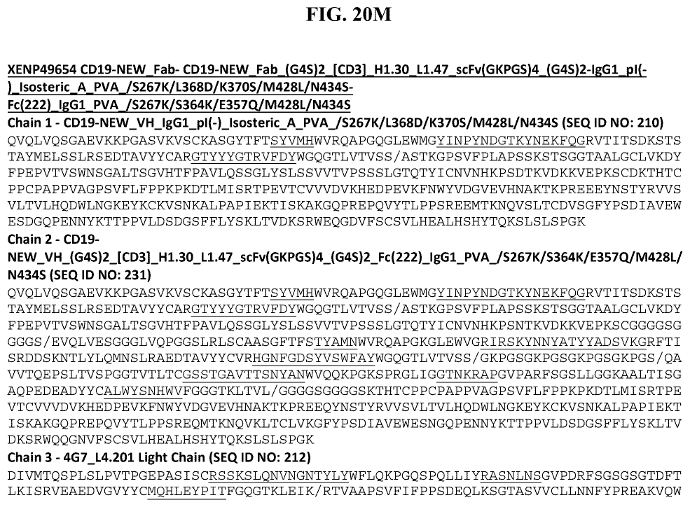

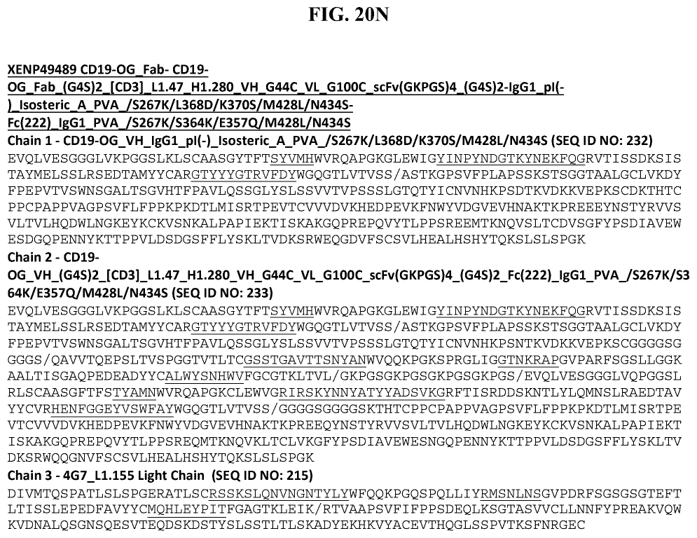

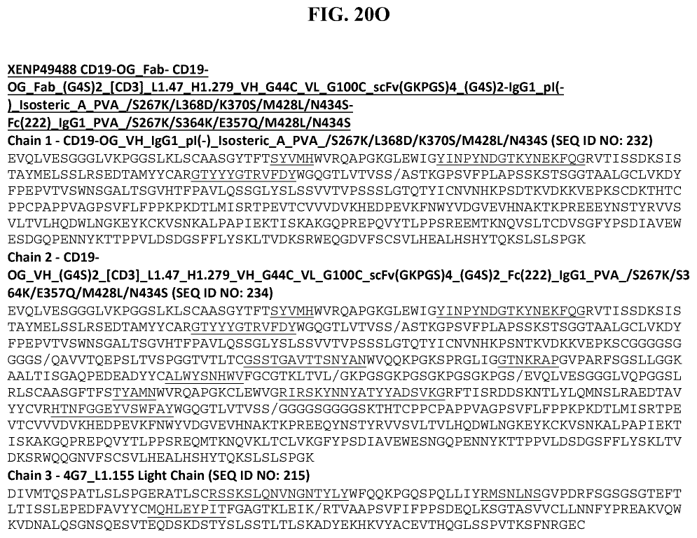

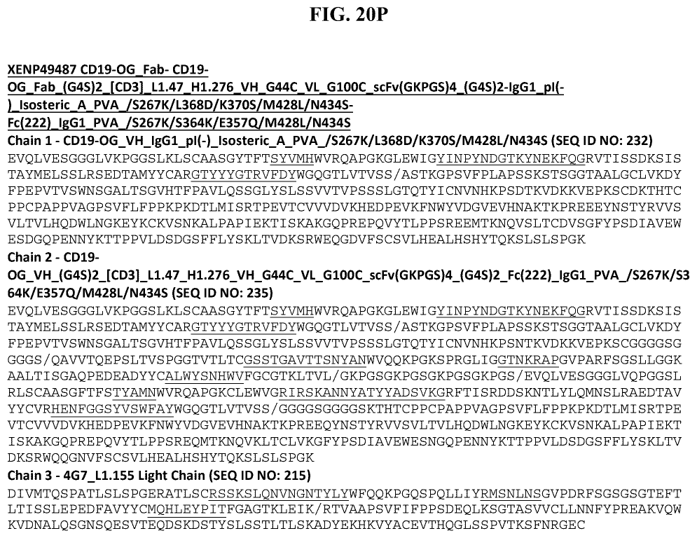

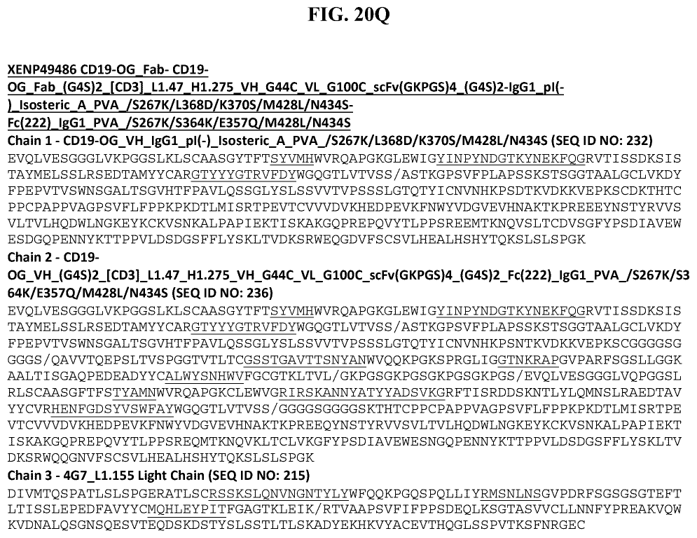

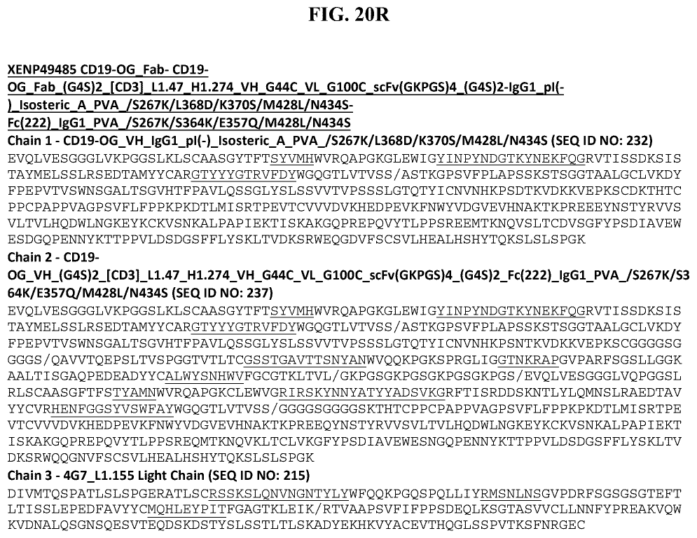

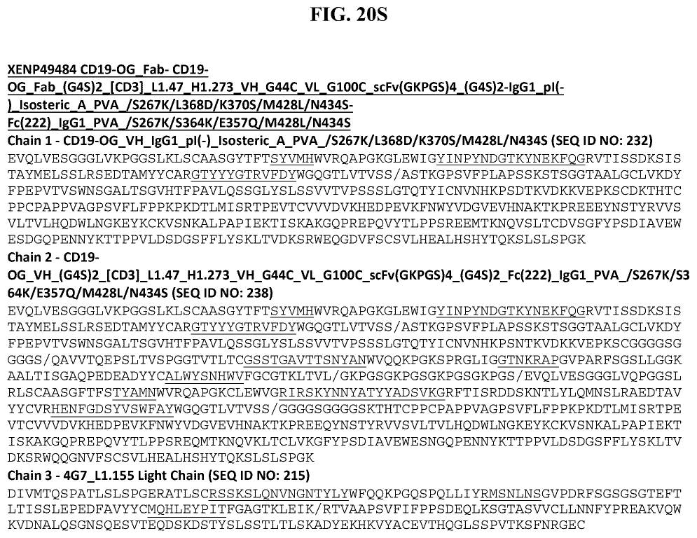

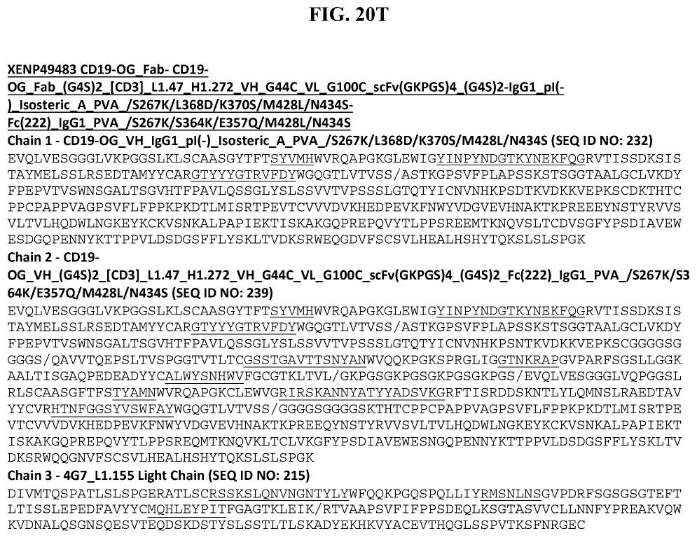

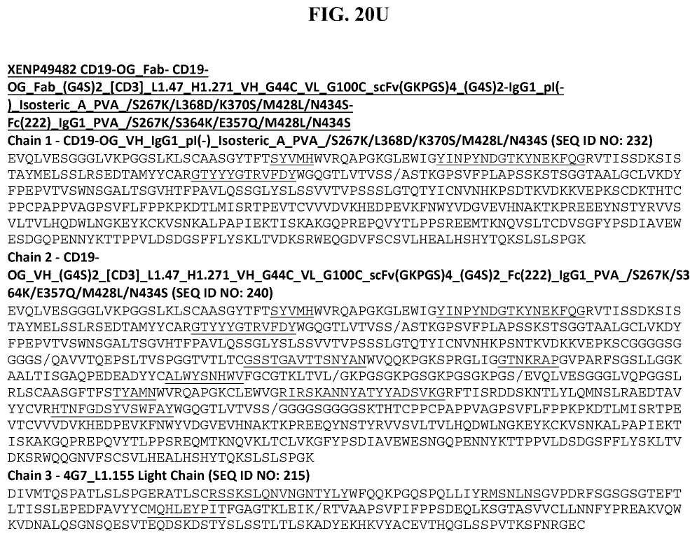

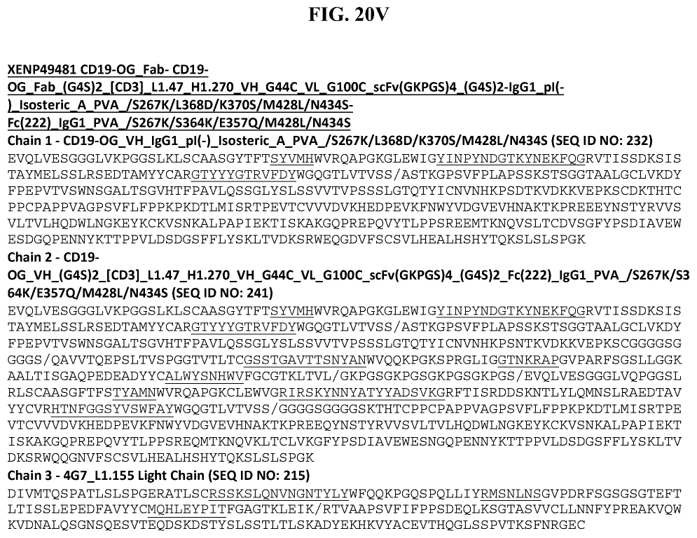

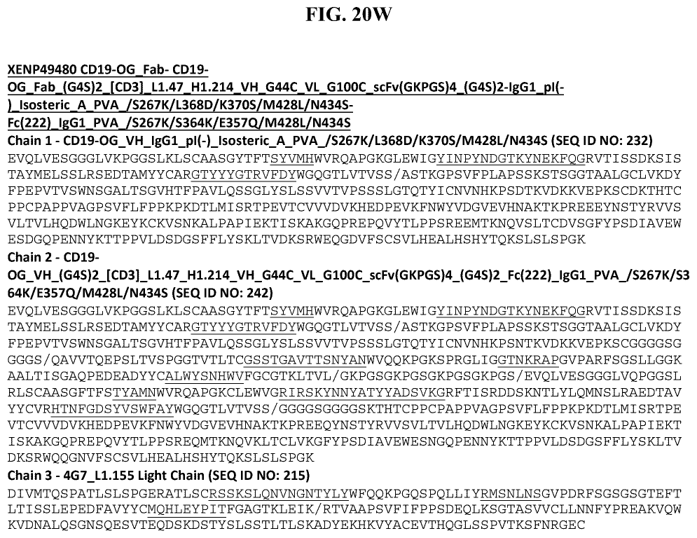

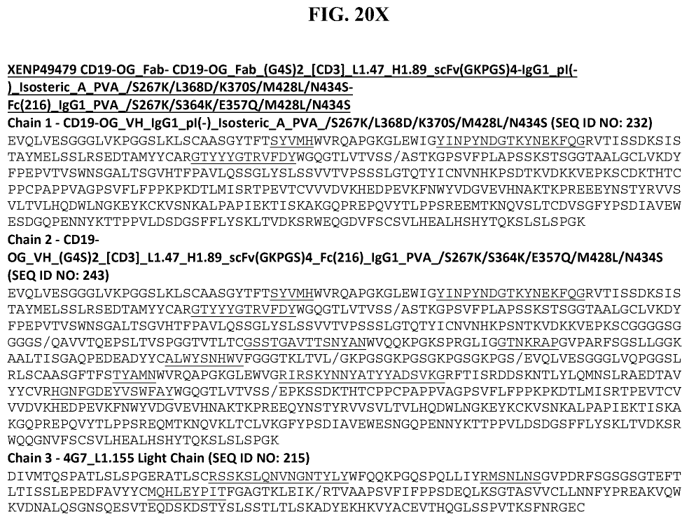

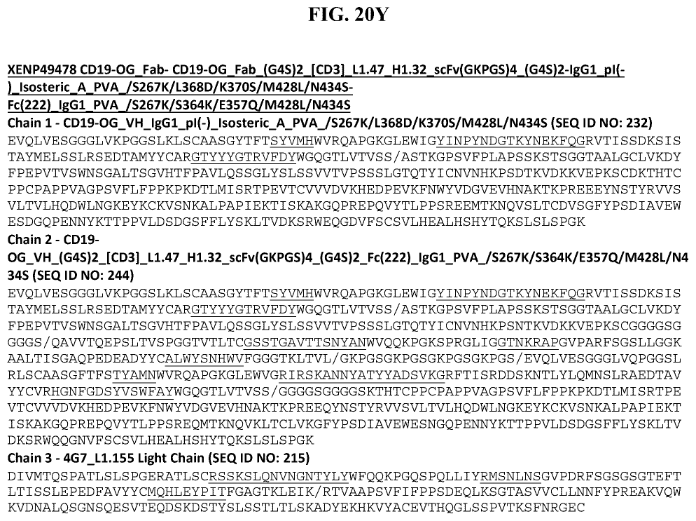

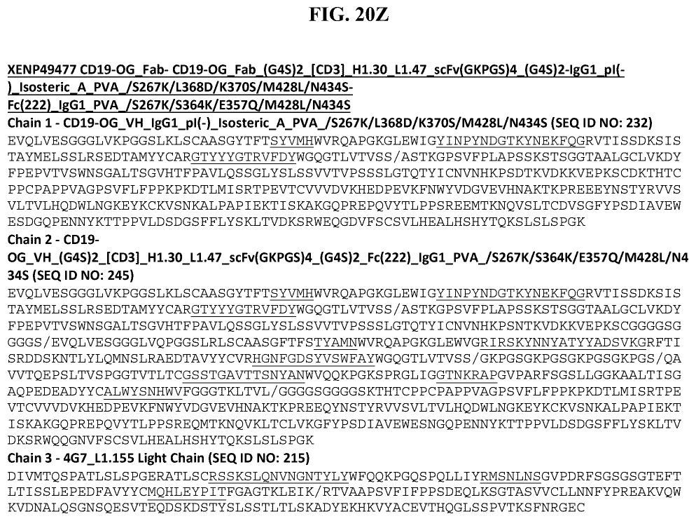

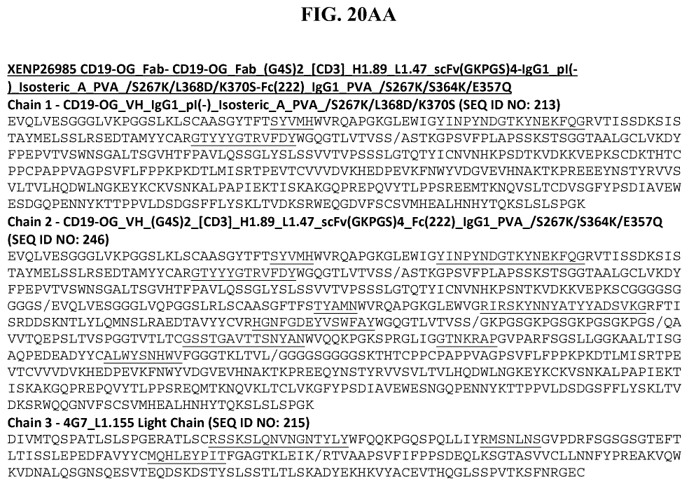

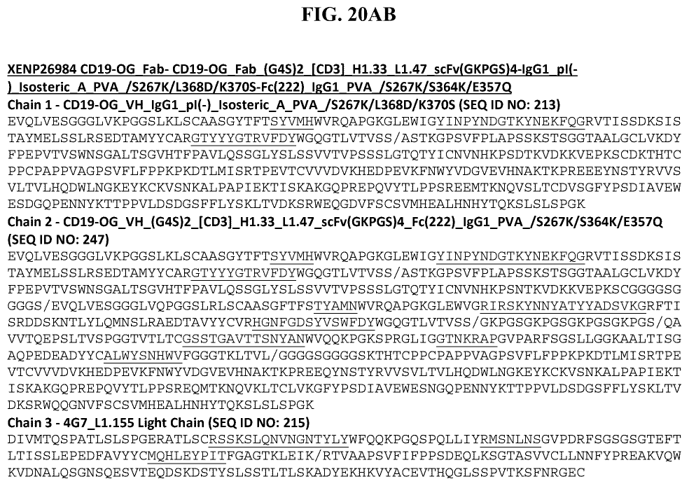

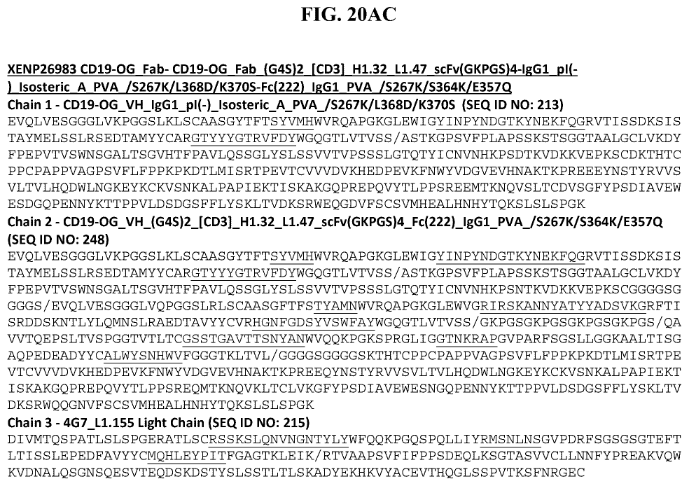

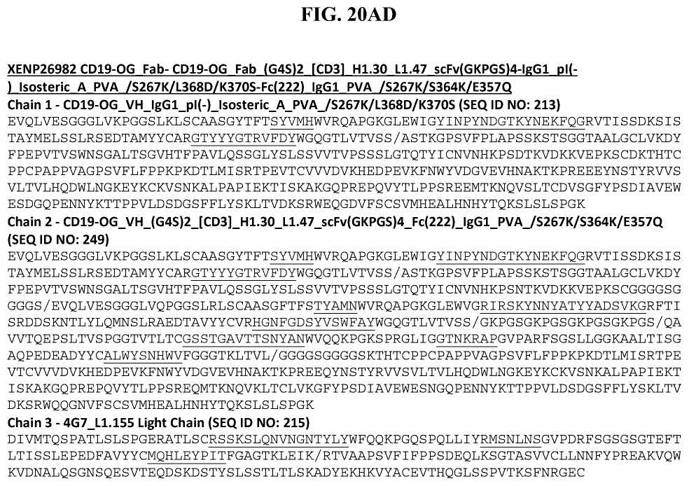

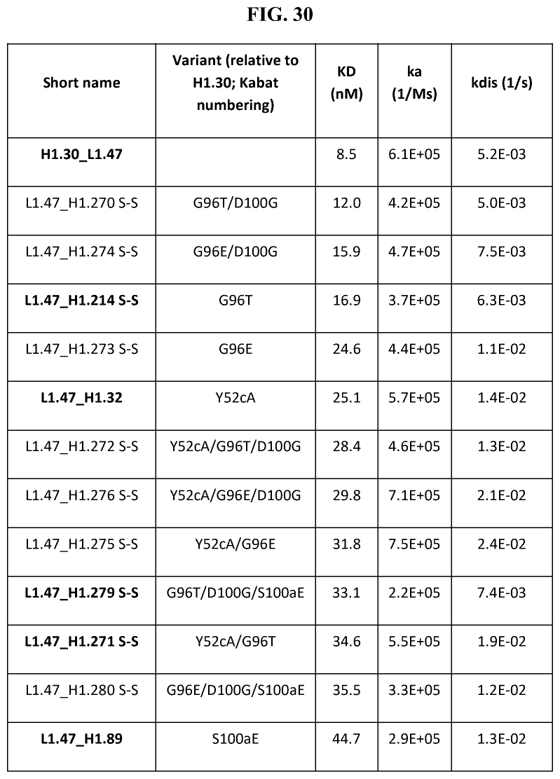

D100G, for example, improves affinity. Further variants are engineered to fix the deamidation and incorporate disulfide bridges while maintaining an affinity ladder and improved stability. Sequences for these variants are depicted in, for example, FIG. 17A-FIG. 17J, and affinity (in the context of 2+1 Fab₂-scFv-Fc molecules of FIG. 20A-FIG. 20AD) are depicted in FIG. 30. The re-engineered anti-CD3 antigen binding molecules can have binding affinity similar to a non-engineered anti-CD3 antigen binding molecule, albeit with removal of the deamidation motif and introduction of stabilizing disulfide bridges.

### CD19 Antigen Binding Molecules

The anti-CD19 antigen binding molecules, for example, are re-humanized and demonstrate enhanced stability.

### CDRs

Disclosed herein are anti-CD19 antigen binding molecules comprising a first binding subunit comprising the CDR sequences of SEQ ID NOs: 1, 2, and 3, and a second binding subunit comprising the CDR sequences of SEQ ID NOs: 4, 5, and 6.

### Linkers

The anti-CD19 antigen binding molecules can comprise a linker between the first binding subunit and the second binding subunit. The linker can comprise at least 95% sequence identity to the amino acid sequence of SEQ ID NO: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95. At least 95% sequence identity includes, for example, 96%, 97%, 98%, and 99% sequence identity. The linker can comprise the amino acid sequence of SEQ ID NO: 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, or 95.

### Variable Regions

The herein disclosed anti-CD19 antigen binding molecules can comprise a heavy chain variable region (VH) and a light chain variable region (VL). In some variations, the disclosed first binding subunit is a VH, and the disclosed second binding subunit is a VL. In some variations, the VH comprises a heavy chain HCDR1 comprising the amino acid sequence of SEQ ID NO: 1, a HCDR2 comprising the amino acid sequence of SEQ ID NO: 2, and a HCDR3 comprising the amino acid sequence of SEQ ID NO: 3, and the VL comprises a light chain LCDR1 comprising the amino acid sequence of SEQ ID NO: 4, a LCDR2 comprising the amino acid sequence of SEQ ID NO: 5, and a LCDR3 comprising the amino acid sequence of SEQ ID NO: 6.

The anti-CD19 antigen binding molecules can comprise a heavy chain variable region comprising at least 95% sequence identity to the amino acid sequence of SEQ ID NO: 7, and a light chain variable region comprising at least 95% sequence identity to the amino acid sequence of SEQ ID NO: 8. At least 95% sequence identity includes, for example, 96%, 97%, 98%, and 99% sequence identity.

The anti-CD19 antigen binding molecules can comprise a heavy chain variable region comprising the amino acid sequence of SEQ ID NO: 7, and a light chain variable region comprising the amino acid sequence of SEQ ID NO: 8.

### Framework Regions

Disclosed herein are anti-CD19 antigen binding molecules comprising an antigen binding domain. The antigen binding domain comprises a first binding subunit, a second binding subunit, and one or more framework regions. In one variation, a framework region comprising at least 95% identity to an amino acid sequence selected from the group consisting of SEQ ID NOs: 20, 21, 22, 23, 24, 25, 26, and 27. In some variations, the framework region comprises at least 95% identity to the amino acid sequence of SEQ ID NOs: 20. In some variations, the framework region comprises the amino acid sequence of SEQ ID NOs: 20. In some variations, the framework region comprises at least 95% identity to the amino acid sequence of SEQ ID NOs: 21. In some variations, the framework region comprises the amino acid sequence of SEQ ID NOs: 21. In some variations, the framework region comprises at least 95% identity to the amino acid sequence of SEQ ID NOs: 22. In some variations, the framework region comprises the amino acid sequence of SEQ ID NOs: 22. In some variations, the framework region comprises at least 95% identity to the amino acid sequence of SEQ ID NOs: 23. In some variations, the framework region comprises the amino acid sequence of SEQ ID NOs: 23. In some variations, the framework region comprises at least 95% identity to the amino acid sequence of SEQ ID NOs: 24. In some variations, the framework region comprises the amino acid sequence of SEQ ID NOs: 24. In some variations, the framework region comprises at least 95% identity to the amino acid sequence of SEQ ID NOs: 25. In some variations, the framework region comprises the amino acid sequence of SEQ ID NOs: 25. In some variations, the framework region comprises at least 95% identity to the amino acid sequence of SEQ ID NOs: 26. In some variations, the framework region comprises the amino acid sequence of SEQ ID NOs: 26. In some variations, the framework region comprises at least 95% identity to the amino acid sequence of SEQ ID NOs: 27. In some variations, the framework region comprises the amino acid sequence of SEQ ID NOs: 27. At least 95% sequence identity includes, for example, 96%, 97%, 98%, and 99% sequence identity.

The anti-CD19 antigen binding molecules can comprise a framework region 1 (FR1) comprising at least 95% identity to an amino acid sequence of SEQ ID NO: 20, a framework region 2 (FR2) comprising at least 95% identity to the amino acid sequence of SEQ ID NO: 21, a framework region 3 (FR3) comprising at least 95% identity to the amino acid sequence of SEQ ID NO: 22, a framework region 4 (FR4) comprising at least 95% identity to the amino acid sequence of SEQ ID NO: 23, a framework region 5 (FR5) comprising at least 95% identity to the amino acid sequence of SEQ ID NO: 24, a framework region 6 (FR6) comprising at least 95% identity to the amino acid sequence of SEQ ID NO: 25, a framework region 7 (FR7) comprising at least 95% identity to the amino acid sequence of SEQ ID NO: 26, and a framework region 8 (FR8) comprising at least 95% identity to the amino acid sequence of SEQ ID NO: 27. At least 95% sequence identity includes, for example, 96%, 97%, 98%, and 99% sequence identity.

The anti-CD19 antigen binding molecules can comprise a framework region 1 (FR1) comprising an amino acid sequence of SEQ ID NO: 20, a framework region 2 (FR2) comprising the amino acid sequence of SEQ ID NO: 21, a framework region 3 (FR3) comprising the amino acid sequence of SEQ ID NO: 22, a framework region 4 (FR4) comprising the amino acid sequence of SEQ ID NO: 23, a framework region 5 (FR5) comprising the amino acid sequence of SEQ ID NO: 24, a framework region 6 (FR6) comprising the amino acid sequence of SEQ ID NO: 25, a framework region 7 (FR7) comprising the amino acid sequence of SEQ ID NO: 26, and a framework region 8 (FR8) comprising the amino acid sequence of SEQ ID NO: 27.

Disclosed herein are anti-CD19 antigen binding molecules comprising an amino acid sequence comprising at least 95% identity to SEQ ID NO: 20 N-terminal to a first binding subunit CDR1, an amino acid sequence comprising at least 95% identity to SEQ ID NO: 21 between the first binding subunit CDR1 and a first binding subunit CDR2, an amino acid sequence comprising at least 95% identity to SEQ ID NO: 22 between the first binding subunit CDR2 and a first binding subunit CDR3, an amino acid sequence comprising at least 95% identity to SEQ ID NO: 23 C-terminal to the first binding subunit CDR3, an amino acid sequence comprising at least 95% identity to SEQ ID NO: 24 N-terminal to a second binding subunit CDR1, an amino acid sequence comprising at least 95% identity to SEQ ID NO: 25 between the second binding subunit CDR1 and a second binding subunit CDR2, an amino acid sequence comprising at least 95% identity to SEQ ID NO: 26 between the second binding subunit CDR2 and a second binding subunit CDR3, and an amino acid sequence comprising at least 95% identity to SEQ ID NO: 27 C-terminal to the second binding subunit CDR3. At least 95% sequence identity includes, for example, 96%, 97%, 98%, and 99% sequence identity.

In some variations, the first binding subunit CDR1 comprises the amino acid sequence of SEQ ID NO: 1, the first binding subunit CDR2 comprises the amino acid sequence of SEQ ID NO: 2, the first binding subunit CDR3 comprises the amino acid sequence of SEQ ID NO: 3, the second binding subunit CDR1 comprising the amino acid sequence of SEQ ID NO: 4, the second binding subunit CDR2 comprises the amino acid sequence of SEQ ID NO: 5, and the second binding subunit CDR3 comprises the amino acid sequence of SEQ ID NO: 6.

The anti-CD19 antigen binding molecules can comprise an amino acid sequence comprising SEQ ID NO: 20 N-terminal to a first binding subunit CDR1, an amino acid sequence comprising SEQ ID NO: 21 between the first binding subunit CDR1 and a first binding subunit CDR2, an amino acid sequence comprising SEQ ID NO: 22 between the first binding subunit CDR2 and a first binding subunit CDR3, an amino acid sequence comprising SEQ ID NO: 23 C-terminal to the first binding subunit CDR3, an amino acid sequence comprising SEQ ID NO: 24 N-terminal to a second binding subunit CDR1, an amino acid sequence comprising SEQ ID NO: 25 between the second binding subunit CDR1 and a second binding subunit CDR2, an amino acid sequence comprising SEQ ID NO: 26 between the second binding subunit CDR2 and a second binding subunit CDR3, and an amino acid sequence comprising SEQ ID NO: 27 C-terminal to the second binding subunit CDR3.

In some variations, the first binding subunit CDR1 comprises the amino acid sequence of SEQ ID NO: 1, the first binding subunit CDR2 comprises the amino acid sequence of SEQ ID NO: 2, the first binding subunit CDR3 comprises the amino acid sequence of SEQ ID NO: 3, the second binding subunit CDR1 comprising the amino acid sequence of SEQ ID NO: 4, the second binding subunit CDR2 comprises the amino acid sequence of SEQ ID NO: 5, and the second binding subunit CDR3 comprises the amino acid sequence of SEQ ID NO: 6.

### Stability

Germline identities and 9mers are increased across both the VH and VL, while maintaining binding affinity (FIG. 29). Further, the re-humanized anti-CD19 antigen binding molecule has +7° C. improved Tₘ (FIG. 29). For example, FIG. 29 shows that the anti-CD19 antigen binding molecule has a Tₘ of 78.5° C. compared to a reference anti-CD19 molecule with a Tₘ of 71.5° C.

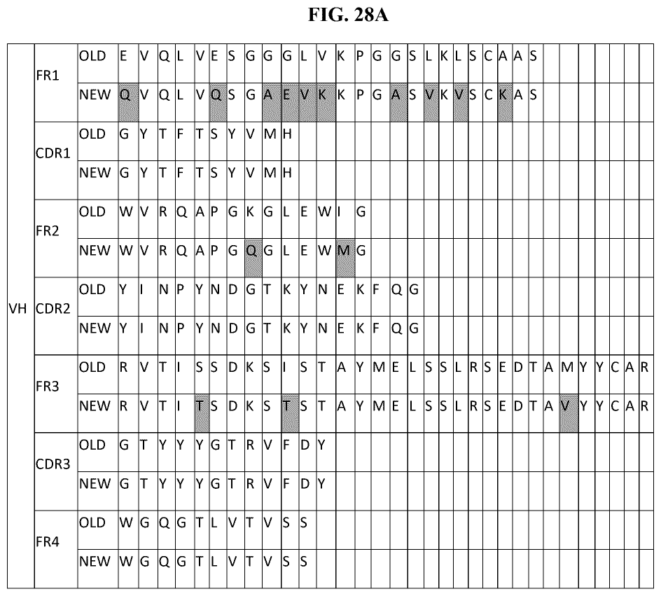

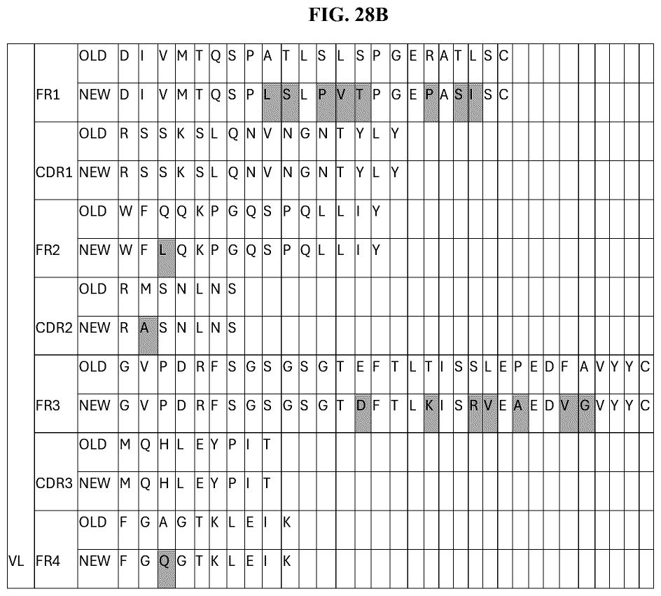

An A100Q substitution in the VL FR4 alone improves stability by 1° C. and removed a predicted MHC-binding peptide (data not shown). Improving stability of the antigen binding molecule is important if antigen binding molecules are, for example, to be used in high concentrations such as in a subcutaneous formulation. Additionally, a VL CDR2 methionine that may be liable to oxidation is substituted with alanine (FIG. 28B). The M51A substitution does not damage binding affinity. The re-engineered anti-CD19 antigen binding molecule can contain, for example, 33 amino acid substitutions relative to the parental sequence.

### Bi-Specific Antigen Binding Molecules

Disclosed herein are anti-CD3 and anti-CD19 bispecific antigen binding molecules comprising an anti-CD19 antigen binding domain with improved stability and an anti-CD3 antigen binding domain with improved stability. Sequence changes that mediate improved stability include, but are not limited to, changes in the framework region. Improvements in stability include increase thermostability of the antigen binding molecules. As described above, improving stability of the antigen binding molecule is important if antigen binding molecules are, for example, to be used in high concentrations such as in a subcutaneous formulation.

### CDRs

Disclosed herein are bispecific anti-CD3 and anti-CD19 antigen binding molecules. In some embodiments, the bispecific anti-CD3 and anti-CD19 antigen binding molecules comprise any of the herein disclosed anti-CD3 antigen binding molecules and means for binding CD19. In some embodiments, the bispecific anti-CD3 and anti-CD19 antigen binding molecules comprise any of the herein disclosed anti-CD19 antigen binding molecules and means for binding CD3.

The means for binding CD19 and means for binding CD3 can correspond to Table 2:

TABLE 2
linking the Group I means clause

| Means for | Corresponding Element Disclosure and/or materials |
|---|---|
| Element | and/or acts disclosed in the Specification |
| means for | Species of CD19 antibodies disclosed in WO2023062188, |
| binding | WO2021055528, WO2020221792, WO2019075366, |
| CD19 | WO2012146394, WO2012055961, WO2011051307, |
| | WO2010052013, WO2010052014, WO2009070642, |
| | WO2007068354, WO2005052004, WO2004106380, |
| | WO2004106381, WO1999054440, WO2024227055, |
| | WO2022254337, WO2021108613, WO2020069409, |
| | WO2019210153, WO2019099639, WO2018023025, |
| | WO2016164731, WO2016164580, WO2016109410, |
| | WO2014153270, WO2012079000, WO2024038115, |
| | WO2023240228, WO2024037594, WO2022117799, |
| | WO2022115762, WO2021259902, WO2022115120, |
| | WO2021084063, WO2021084062, WO2021084064, |
| | WO2020225196, WO2020214937, WO2018220040, |
| | WO2018078123, WO2018002031, WO2017214452, |
| | WO2017032679, WO2016189014, WO2015195498, |
| | WO2014184143, WO2013024095, WO2013024097, |
| | WO2008022152, WO2024155810, WO2024025602, |
| | WO2023019200, WO2022236047, WO2022094334, |
| | WO2020219743, WO2013138244, WO2012067981, |
| | WO2010102276, WO2008031056, WO2023274974, |
| | WO2020249528, WO2020109251, WO2020043878, |
| | WO2018229222, WO2018193105, WO2016166298, |
| | WO2015052534, WO2014057117, WO2018183494, |
| | WO2016180941, WO2014058947, WO2013017540, |
| | WO2012156455, WO2024086190, WO2018200496, |
| | WO2025151484, WO2025076058, WO2024226487, |
| | WO2023235852, WO2023215674, WO2022076573, |
| | WO2008022152, WO2018017928, WO2016130902, |
| | WO2015157297, WO2015157286, WO2009052431, |
| | WO2024182516, WO2019213184, WO2017096329, |
| | WO2016033570, WO2021161197, WO2020219848, |
| | WO2019005642, WO2009054863, WO2007002223, |
| | WO2003025018, WO2017096368, WO2016048938, |
| | WO2020135335, WO2020048525, WO2020132810, |
| | WO2020186974, WO2007076950, WO2019215510, |
| | WO2018083535, WO2022125837, WO2019094498, |
| | WO2019125070, WO2019112347, WO2019175071, |
| | WO2020233589, WO2023245106, WO2006125668, |
| | WO2003048209, WO2010095031, WO2010053716, |
| | WO2005012493, WO2006133450, WO2006121852, |
| | WO2017055541, WO2017055328, WO2011147834, |
| | WO2019011918, WO2012010602, WO2012010562, |
| | WO2012010561, WO2012057765, WO2014163684, |
| | WO2014028560, WO2015109131, WO2020053300, |
| | WO2016023909, WO2023218381, WO2021224629, |
| | WO2019220109, WO2016102965, WO2016112855, |
| | WO2016138846, WO2016139487, WO2020014482, |
| | WO2016149578, WO2018126369, WO2017015783, |
| | WO2021255155, WO2017055314, WO2017064221, |
| | WO2017066136, WO2021034227, WO2017095267, |
| | WO2021216731, WO2018108106, WO2018188612, |
| | WO2024188026, WO2018201794, WO2024044743, |
| | WO2019055842, WO2019028051, WO2019057100, |
| | WO2019057122, WO2019057124, WO2022099026, |
| | WO2020069184, WO2019079249, WO2020204152, |
| | WO2019095641, WO2019119822, WO2019137518, |
| | WO2019159193, WO2020108644, WO2020108646, |
| | WO2020108645, WO2020108643, WO2020108642, |
| | WO2019161796, WO2022097061, WO2022097060, |
| | WO2020236792, WO2019195535, WO2022012683, |
| | WO2022012681, WO2019214332, WO2021119551, |
| | WO2019237081, WO2020011706, WO2022216864, |
| | WO2020018922, WO2020055040, WO2020088605, |
| | WO2021213235, WO2020108090, WO2020114358, |
| | WO2020124021, WO2020125653, WO2024012457, |
| | WO2022026330, WO2020180551, WO2020163222, |
| | WO2024006925, WO2020180882, WO2020210232, |
| | WO2021034952, WO2021165248, WO2021173471, |
| | WO2021170146, WO2024197157, WO2023028159, |
| | WO2023028162, WO2021173844, WO2021178253, |
| | WO2021184673, WO2023073645, WO2021217024, |
| | WO2021223720, WO2021262723, WO2022002154, |
| | WO2022007650, WO2022011846, WO2022081486, |
| | WO2023280297, WO2022105811, WO2022120107, |
| | WO2022121880, WO2022159653, WO2022194264, |
| | WO2023090704, WO2023129937, WO2023199235, |
| | WO2023206985, WO2024094741, WO2024079592, |
| | WO2024131874, WO2025067496, WO2025020672, |
| | WO2025137576, WO2025131125, WO2025157132, |
| | incorporated herein by reference to the extent the |
| | species' therein describe binding to CD19. |
| means for | Species of CD3 antibodies disclosed in WO2023062188, |
| binding | WO2021055528, WO2020221792, WO2019075366, |
| CD3 | WO2012146394, WO2012055961, WO2011051307, |
| | WO2010052013, WO2010052014, WO2009070642, |
| | WO2007068354, WO2005052004, WO2004106380, |
| | WO2004106381, WO1999054440, WO2023250367, |
| | WO2023234933, WO2023201291, WO2023091887, |
| | WO2023279092, WO2022241235, WO2023075798, |
| | WO2018093821, WO2015095392, WO2024124107, |
| | WO2024094741, WO2023198727, WO2023180353, |
| | WO2022228706, WO2022148732, WO2022228705, |
| | WO2021214277, WO2018220099, WO2018114748, |
| | WO2017097723, WO2016020309, WO2022199555, |
| | WO2010119119, WO2009030734, WO2007093630, |
| | WO2024236031, WO2024213754, WO2024094822, |
| | WO2023198839, WO2023144306, WO2023144290, |
| | WO2022053654, WO2022053657, WO2022053655, |
| | WO2022053656, WO2022053658, WO2022053653, |
| | WO2021224499, WO2021028587, WO2019155008, |
| | WO2016110576, WO2025094107, WO2025096717, |
| | WO2024231860, WO2024220687, WO2024220682, |
| | WO2024095173, WO2024044548, WO2023086817, |
| | WO2023081705, WO2021228783, WO2019220368, |
| | WO2017031104, WO2025032521, WO2024121777, |
| | WO2022053990, WO2021229507, WO2016166629, |
| | WO2024206739, WO2024182767, WO2023230476, |
| | WO2023044495, WO2023039295, WO2022251258, |
| | WO2022251253, WO2020232247, WO2008079713, |
| | WO2004052397, WO2025094085, WO2024246856, |
| | WO2024220682, WO2024180469, WO2023081704, |
| | WO2022058445, WO2019220368, WO2018017786, |
| | WO2023164143, WO2022072762, WO2021119135, |
| | WO2020047389, WO2017112762, WO2017112775, |
| | WO2017053856, WO2016081490, WO2015143079, |
| | WO2014047231, WO2025014823, WO2021113701, |
| | WO2020191346, WO2020018825, WO2020018820, |
| | WO2023218100, WO2023064942, WO2022221767, |
| | WO2022023566, WO2021255189, WO2021180821, |
| | WO2020089396, WO2019175658, WO2018226833, |
| | WO2018044948, WO2012098238, WO2010132872, |
| | WO2007033230, WO2005118635, WO2005099755, |
| | WO2014141152, WO2011050106, WO2011050104, |
| | WO2007145941, WO2025117764, WO2022006316, |
| | WO2021222578, WO2021222616, WO2020061478, |
| | WO2019133761, WO2019006072, WO2018237037, |
| | WO2018237006, WO2018119215, WO2018052503, |
| | WO2017223111, WO2025080688, WO2023215829, |
| | WO2023278585, WO2017021349, WO2022117045, |
| | WO2020251781, WO2020210277, WO2015026892, |
| | WO2009106096, WO2022189377, WO2022090439, |
| | WO2021255146, WO2020208124, WO2019115659, |
| | WO2019020745, WO2018219901, WO2017118675, |
| | WO2017097723, WO2017055389, WO2023087255, |
| | WO2020018695, WO2019005642, WO2025120113, |
| | WO2025042742, WO2021163329, WO2020219978, |
| | WO2020089437, WO2019226761, WO2018083204, |
| | WO2016036678, WO2013012414, WO2007071422, |
| | WO2007071426, WO2024073700, WO2016141303, |
| | WO2024190892, WO2021020416, WO2021006199, |
| | WO2019156199, WO2021113701, WO2020191346, |
| | WO2020018820, WO2020018825, WO2025106474, |
| | WO2024263845, WO2024233341, WO2024091991, |
| | WO2024020432, WO2024015897, WO2024011179, |
| | WO2023219613, WO2023191816, WO2022076462, |
| | WO2020252366, WO2017106578, WO2003025018, |
| | WO2021243320, WO2021091906, WO2020025532, |
| | WO2017182427, WO2017129585, WO2015036583, |
| | WO2014144722, WO2022103781, WO2021094000, |
| | WO2020025596, WO2017134134, WO2014140248, |
| | WO2013072415, WO2013072406, WO2015026894, |
| | WO2022099006, WO2020243603, WO2020077258, |
| | WO2019075413, WO2016196230, WO2016004108, |
| | WO2017159287, WO2016047722, WO2017030926, |
| | WO2017096368, WO2016048938, WO2018057802, |
| | WO2025128652, WO2025117869, WO2023177772, |
| | WO2023133280, WO2023097030, WO2022212885, |
| | WO2020257604, WO2019246356, WO2018067331, |
| | WO2020135335, WO2020048525, WO2020132810, |
| | WO2020186974, WO2020127354, WO2013083809, |
| | WO2011033105, WO2004106383, WO2013001065, |
| | WO2022119976, WO2016130819, WO2020204708, |
| | WO2020130829, WO2019108065, WO2017010874, |
| | WO2014051433, WO2015063339, WO2014096051, |
| | WO2014079000, WO2025171124, WO2023224912, |
| | WO2020257681, WO2017023761, WO2016001810, |
| | WO2019224717, WO2017157305, WO2023152581, |
| | WO2022162518, WO2020212947, WO2020212949, |
| | WO2019224717, WO2019224718, WO2016179518, |
| | WO2021092217, WO2017021370, WO2017173410, |
| | WO2017021362, WO2020018556, WO2019157340, |
| | WO2017091656, WO2017136659, WO2022212732, |
| | WO2021231434, WO2018098354, WO2018098356, |
| | WO2022256498, WO2018209298, WO2018209304, |
| | WO2021026074, WO2022097090, WO2020261093, |
| | WO2020236795, WO2019229701, WO2018201051, |
| | WO2025027557, WO2024038165, WO2021078774, |
| | WO2020157210, WO2019224711, WO2022256499, |
| | WO2019075378, WO2019075359, WO2023073225, |
| | WO2021089513, WO2019122052, WO2019133961, |
| | WO2021048423, WO2019175198, WO2023060277, |
| | WO2019222428, WO2024052831, WO2019224717, |
| | WO2019224713, WO2022256500, WO2020069028, |
| | WO2019234220, WO2020025792, WO2020210392, |
| | WO2020076853, WO2023094569, WO2022189380, |
| | WO2020127619, WO2021222578, WO2020206330, |
| | WO2021000530, WO2021104371, WO2022024024, |
| | WO2022234146, WO2021185934, WO2024138072, |
| | WO2022042488, WO2023185732, WO2022068810, |
| | WO2022134645, WO2022167460, WO2024251154, |
| | WO2022174813, WO2022218380, WO2022267702, |
| | WO2023242351, WO2025027511, WO2023236889, |
| | WO2024089551, WO2024050797, WO2021200939, |
| | WO2016020444, WO2004024771, WO2004108158, |
| | WO2022060878, WO2017134158, WO2011121110, |
| | WO2010037836, WO2010037837, WO2014108483, |
| | WO2012143524, WO2019160904, WO2012162067, |
| | WO2014163684, WO2014028560, WO2023279095, |
| | WO2015006482, WO2014131712, WO2014131711, |
| | WO2014131694, WO2013026831, WO2013026833, |
| | WO2013026837, WO2013026835, WO2013026839, |
| | WO2013041687, WO2022096698, WO2021247812, |
| | WO2021183861, WO2021150824, WO2014151910, |
| | WO2007042261, WO2015018527, WO2015172800, |
| | WO2015058861, WO2014153002, WO2014153164, |
| | WO2013186613, WO2013188693, WO2017008169, |
| | WO2015006749, WO2014012085, WO2021173889, |
| | WO2020047176, WO2017079272, WO2014129270, |
| | WO2015126548, WO2016016412, WO2016016415, |
| | WO2014114800, WO2021092060, WO2021092056, |
| | WO2018083204, WO2018060301, WO2016087531, |
| | WO2016079177, WO2016020332, WO2014122144, |
| | WO2014128185, WO2025085781, WO2020186176, |
| | WO2019157308, WO2016205531, WO2016055592, |
| | WO2016055593, WO2014167022, WO2016184570, |
| | WO2015172800, WO2014180577, WO2017009442, |
| | WO2015104346, WO2015001085, WO2013149526, |
| | WO2015109131, WO2023280880, WO2013158856, |
| | WO2015146437, WO2015146438, WO2010109924, |
| | WO2015172341, WO2015181098, WO2015184203, |
| | WO2023192606, WO2016014974, WO2019177970, |
| | WO2018140026, WO2016014942, WO2020053300, |
| | WO2016023909, WO2018224441, WO2018224443, |
| | WO2016068803, WO2016068802, WO2020182984, |
| | WO2016071355, WO2025125386, WO2021255143, |
| | WO2016079076, WO2016079050, WO2018102547, |
| | WO2018027147, WO2016089610, WO2018015340, |
| | WO2016116626, WO2025169945, WO2016166360, |
| | WO2016204966, WO2016179003, WO2016180721, |
| | WO2016205200, WO2017001681, WO2022060901, |
| | WO2017021356, WO2017021354, WO2018184966, |
| | WO2017055388, WO2017055395, WO2017055318, |
| | WO2017055385, WO2017055392, WO2017055393, |
| | WO2021255155, WO2017055314, WO2017055391, |
| | WO2017079121, WO2023071953, WO2017070943, |
| | WO2021034227, WO2017095267, WO2021231976, |
| | WO2017100372, WO2017106453, WO2017127499, |
| | WO2017125831, WO2013092001, WO2017143259, |
| | WO2017150762, WO2023064955, WO2019213444, |
| | WO2019075405, WO2023023489, WO2023023491, |
| | WO2021007266, WO2017176760, WO2017201493, |
| | WO2017205726, WO2024168316, WO2021030484, |
| | WO2019028190, WO2019008379, WO2018058001, |
| | WO2025083076, WO2025082594, WO2018054973, |
| | WO2018054484, WO2018068652, WO2020079280, |
| | WO2018197502, WO2018127473, WO2018114754, |
| | WO2019244107, WO2018117237, WO2018162749, |
| | WO2018188612, WO2018199593, WO2018203567, |
| | WO2024086617, WO2023097219, WO2023044402, |
| | WO2020247929, WO2018208864, WO2020247932, |
| | WO2018220584, WO2018222935, WO2018237341, |
| | WO2019023097, WO2023056391, WO2019033050, |
| | WO2023180346, WO2023006809, WO2019034580, |
| | WO2019045856, WO2019057099, WO2019057122, |
| | WO2019057124, WO2019068633, WO2020204305, |
| | WO2019078697, WO2019086534, WO2019095641, |
| | WO2021195513, WO2019104075, WO2024079010, |
| | WO2024079015, WO2019152705, WO2019143636, |
| | WO2019167962, WO2019175806, WO2019184549, |
| | WO2022097061, WO2022097060, WO2020236792, |
| | WO2019195535, WO2019224715, WO2020156405, |
| | WO2019228406, WO2023031397, WO2022167474, |
| | WO2022069712, WO2021110935, WO2019234241, |
| | WO2021119551, WO2019237081, WO2019241216, |
| | WO2020001344, WO2020041537, WO2020061482, |
| | WO2020088403, WO2020088608, WO2020088437, |
| | WO2020088605, WO2018224443, WO2025131071, |
| | WO2020114478, WO2024184287, WO2020127619, |
| | WO2024012457, WO2020168555, WO2020209559, |
| | WO2020232165, WO2020237173, WO2020252907, |
| | WO2020264200, WO2021003189, WO2021009263, |
| | WO2021022304, WO2021023657, WO2021030586, |
| | WO2021027674, WO2023183758, WO2021030680, |
| | WO2021041300, WO2021043810, WO2021044008, |
| | WO2021048724, WO2021053587, WO2021063330, |
| | WO2021064137, WO2021098822, WO2021104430, |
| | WO2021121215, WO2021127088, WO2022098771, |
| | WO2021151020, WO2021155635, WO2021163366, |
| | WO2021163364, WO2021165248, WO2022170305, |
| | WO2021173783, WO2021188590, WO2021209066, |
| | WO2024196678, WO2021216972, WO2022212831, |
| | WO2021236638, WO2021239987, WO2021237717, |
| | WO2022201053, WO2021240388, WO2025021838, |
| | WO2021255142, WO2021260064, WO2023020507, |
| | WO2022002012, WO2022002112, WO2022013775, |
| | WO2022040417, WO2023051727, WO2022037582, |
| | WO2022105924, WO2022042661, WO2025041836, |
| | WO2022045247, WO2023215789, WO2023164513, |
| | WO2022125583, WO2022046658, WO2022063819, |
| | WO2022068809, WO2022069724, WO2023194807, |
| | WO2022125566, WO2022125576, WO2022125562, |
| | WO2022081822, WO2022077108, WO2022087211, |
| | WO2022094147, WO2022096700, WO2023219121, |
| | WO2023199927, WO2022102768, WO2022100613, |
| | WO2022104267, WO2022105787, WO2022116480, |
| | WO2022117050, WO2022129560, WO2022135468, |
| | WO2023036326, WO2022135536, WO2022150803, |
| | WO2022165171, WO2022166940, WO2022171192, |
| | WO2022170740, WO2022170619, WO2025134050, |
| | WO2025134049, WO2022175255, WO2022192895, |
| | WO2022194264, WO2022197907, WO2022200443, |
| | WO2022229434, WO2022234102, WO2025179012, |
| | WO2024100451, WO2024102723, WO2022240637, |
| | WO2022246244, WO2022262868, WO2022262733, |
| | WO2022266660, WO2022263507, WO2023273595, |
| | WO2023273914, WO2025083076, WO2025082594, |
| | WO2023285560, WO2023001154, WO2023000791, |
| | WO2023002392, WO2023002390, WO2023011338, |
| | WO2023015170, WO2023015169, WO2023014809, |
| | WO2023236099, WO2023029089, WO2023049150, |
| | WO2023057571, WO2023060157, WO2023061419, |
| | WO2023061417, WO2023222027, WO2025140489, |
| | WO2023217289, WO2023078450, WO2023078446, |
| | WO2023079425, WO2025051141, WO2023103335, |
| | WO2023104938, WO2023110788, WO2023125729, |
| | WO2024131751, WO2023125611, WO2023131328, |
| | WO2023213998, WO2023138551, WO2023141856, |
| | WO2023164698, WO2023169419, WO2023183766, |
| | WO2023178357, WO2023173258, WO2023174925, |
| | WO2023179716, WO2023178645, WO2023183477, |
| | WO2023186112, WO2023192850, WO2023187130, |
| | WO2023196954, WO2023060169, WO2023199235, |
| | WO2023208014, WO2023206985, WO2023219120, |
| | WO2023221618, WO2023222024, WO2023230626, |
| | WO2024251242, WO2023250272, WO2024002308, |
| | WO2024041435, WO2024144400, WO2024067222, |
| | WO2024074145, WO2024081918, WO2024092038, |
| | WO2025002014, WO2024087521, WO2024102948, |
| | WO2024108032, WO2024118771, WO2024130032, |
| | WO2024129897, WO2024131861, WO2024131874, |
| | WO2024152963, WO2024156672, WO2024160269, |
| | WO2024165403, WO2024229238, WO2024173852, |
| | WO2024178202, WO2024178414, WO2024184812, |
| | WO2024184811, WO2024184810, WO2024191322, |
| | WO2024191168, WO2024188319, WO2024188965, |
| | WO2024188966, WO2024188355, WO2024199294, |
| | WO2024199454, WO2024214007, WO2024213782, |
| | WO2024220397, WO2024235317, WO2024241206, |
| | WO2024254757, WO2025006794, WO2025024646, |
| | WO2025051309, WO2025056013, WO2025064885, |
| | WO2025064890, WO2025103425, WO2025061171, |
| | WO2025064369, WO2025077737, WO2025080533, |
| | WO2025080534, WO2025087681, WO2025092742, |
| | WO2025092742, WO2025091264, WO2025113615, |
| | WO2025122927, WO2025124266, WO2025124523, |
| | WO2025131075, WO2025131077, WO2025132907, |
| | WO2025140010, WO2025157132, WO2025162453, |
| | WO2025175056, incorporated herein by reference to the |
| | extent the species' therein describe binding to CD3. |

TABLE 3
below links Group 2 means clauses to the
relevant portions of the specification

| Means for Element | Corresponding Element Disclosure |
|---|---|
| means for | Species of CD19 antibodies disclosed in paragraph |
| binding CD19 | [0006], [0009]-[0011]. |
| means for | Species of CD3 antibodies disclosed in paragraph |
| binding CD3 | [0006], [00012]-[0014]. |

The herein disclosed bispecific anti-CD3 and anti-CD19 antigen binding molecules can comprise any of the herein disclosed anti-CD3 antigen binding molecules in combination with any of the herein disclosed anti-CD19 antigen binding molecules. For example, the bi-specific anti-CD19 and anti-CD3 antigen binding molecule can comprise an anti-CD19 antigen binding domain comprising a first binding subunit comprising the CDR sequences of SEQ ID NOs: 1, 2, and 3, and a second binding subunit comprising the CDR sequences of SEQ ID NOs: 4, 5, and 6, and an anti-CD3 antigen binding domain comprising a first binding subunit comprising the CDR sequences of SEQ ID NO: 9, SEQ ID NO: 10 or 11, and SEQ ID NO: 12, 13, 14, 15, or 16, and a second binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 10, and 12 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 10, and 13 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 11, and 12 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 11, and 13 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 10, and 14 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 10, and 15 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 11, and 14 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 11, and 15 and a second anti-CD3 binding subunit comprising the CDR sequences of SEQ ID NOs: 17, 18, and 19. In some variations, the anti-CD19 antigen binding domain comprises a first binding subunit comprising the CDR sequences of SEQ ID NOs: 1, 2, and 3, and a second binding subunit comprising the CDR sequences of SEQ ID NOs: 4, 5, and 6, and the anti-CD3 antigen binding domain comprises a first anti-CD3 binding subunit comprising the CDR sequences of SEQ ID NOs: 9, 10, and 16 and a second anti-CD3 binding subunit comprising the CDR sequences of SEQ ID NOs: 17, 18, and 19.

### Anti-CD19×CD3 Antigen Binding Domain Framework Regions

In some variations, the herein disclosed bi-specific anti-CD19 and anti-CD3 antigen binding molecules comprise an anti-CD19 antigen binding domain comprising a framework region 1 (FR1) comprising at least 95% identity to an amino acid sequence of SEQ ID NO: 20, a framework region 2 (FR2) comprising at least 95% identity to the amino acid sequence of SEQ ID NO: 21, a framework region 3 (FR3) comprising at least 95% identity to the amino acid sequence of SEQ ID NO: 22, a framework region 4 (FR4) comprising at least 95% identity to the amino acid sequence of SEQ ID NO: 23, a framework region 5 (FR5) comprising at least 95% identity to the amino acid sequence of SEQ ID NO: 24, a framework region 6 (FR6) comprising at least 95% identity to the amino acid sequence of SEQ ID NO: 25, a framework region 7 (FR7) comprising at least 95% identity to the amino acid sequence of SEQ ID NO: 26, and a framework region 8 (FR8) comprising at least 95% identity to the amino acid sequence of SEQ ID NO: 27. At least 95% sequence identity includes, for example, 96%, 97%, 98%, and 99% sequence identity.

In some variations, the herein disclosed bi-specific anti-CD19 and anti-CD3 antigen binding molecules comprise an anti-CD19 antigen binding domain comprising an amino acid sequence comprising at least 95% identity to SEQ ID NO: 20 N-terminal to a first binding subunit CDR1, an amino acid sequence comprising at least 95% identity to SEQ ID NO: 21 between the first binding subunit CDR1 and a first binding subunit CDR2, an amino acid sequence comprising at least 95% identity to SEQ ID NO: 22 between the first binding subunit CDR2 and a first binding subunit CDR3, an amino acid sequence comprising at least 95% identity to SEQ ID NO: 23 C-terminal to the first binding subunit CDR3, an amino acid sequence comprising at least 95% identity to SEQ ID NO: 24 N-terminal to a second binding subunit CDR1, an amino acid sequence comprising at least 95% identity to SEQ ID NO: 25 between the second binding subunit CDR1 and a second binding subunit CDR2, an amino acid sequence comprising at least 95% identity to SEQ ID NO: 26 between the second binding subunit CDR2 and a second binding subunit CDR3, and an amino acid sequence comprising at least 95% identity to SEQ ID NO: 27 C-terminal to the second binding subunit CDR3. At least 95% sequence identity includes, for example, 96%, 97%, 98%, and 99% sequence identity.

In some variations, the herein disclosed bi-specific anti-CD19 and anti-CD3 antigen binding molecules comprise an anti-CD19 antigen binding domain comprising a framework region 1 (FR1) comprising an amino acid sequence of SEQ ID NO: 20, a framework region 2 (FR2) comprising the amino acid sequence of SEQ ID NO: 21, a framework region 3 (FR3) comprising the amino acid sequence of SEQ ID NO: 22, a framework region 4 (FR4) comprising the amino acid sequence of SEQ ID NO: 23, a framework region 5 (FR5) comprising the amino acid sequence of SEQ ID NO: 24, a framework region 6 (FR6) comprising the amino acid sequence of SEQ ID NO: 25, a framework region 7 (FR7) comprising the amino acid sequence of SEQ ID NO: 26, and a framework region 8 (FR8) comprising the amino acid sequence of SEQ ID NO: 27.

In some variations, the herein disclosed bi-specific anti-CD19 and anti-CD3 antigen binding molecules comprise an anti-CD19 antigen binding domain comprising an amino acid sequence comprising SEQ ID NO: 20 N-terminal to a first binding subunit CDR1, an amino acid sequence comprising SEQ ID NO: 21 between the first binding subunit CDR1 and a first binding subunit CDR2, an amino acid sequence comprising SEQ ID NO: 22 between the first binding subunit CDR2 and a first binding subunit CDR3, an amino acid sequence comprising SEQ ID NO: 23 C-terminal to the first binding subunit CDR3, an amino acid sequence comprising SEQ ID NO: 24 N-terminal to a second binding subunit CDR1, an amino acid sequence comprising SEQ ID NO: 25 between the second binding subunit CDR1 and a second binding subunit CDR2, an amino acid sequence comprising SEQ ID NO: 26 between the second binding subunit CDR2 and a second binding subunit CDR3, and an amino acid sequence comprising SEQ ID NO: 27 C-terminal to the second binding subunit CDR3.

### Anti-CD19 Antigen Binding Domain Variable Regions

In some variations, the herein disclosed bi-specific anti-CD19 and anti-CD3 antigen binding molecules comprise a first anti-CD19 binding subunit and a first anti-CD3 binding subunit that are heavy chain variable regions, and a second anti-CD19 binding subunit and a second anti-CD3 binding subunit that are light chain variable regions. In some variations, the bi-specific anti-CD19 and anti-CD3 antigen binding molecules comprise an anti-CD19 antigen binding domain comprising a heavy chain variable region comprising at least 95% sequence identity to the amino acid sequence of SEQ ID NO: 7, and a light chain variable region comprising at least 95% sequence identity to the amino acid sequence of SEQ ID NO: 8. At least 95% sequence identity includes, for example, 96%, 97%, 98%, and 99% sequence identity. In some variations, the bi-specific anti-CD19 and anti-CD3 antigen binding molecules comprise an anti-CD19 antigen binding domain comprising a heavy chain variable region comprising the amino acid sequence of SEQ ID NO: 7, and a light chain variable region comprising the amino acid sequence of SEQ ID NO: 8.

Anti-CD3 Antigen binding domain Framework Regions

In some variations, the herein disclosed bi-specific anti-CD19 and anti-CD3 antigen binding molecules comprise an anti-CD3 antigen binding domain comprising a framework region 1 (FR1) comprising at least 95% identity to the amino acid sequence of SEQ ID NO: 28, a framework region 2 (FR2) comprising at least 95% identity to the amino acid sequence of SEQ ID NO: 29, a framework region 3 (FR3) comprising at least 95% identity to the amino acid sequence of SEQ ID NO: 30, a framework region 4 (FR4) comprising at least 95% identity to the amino acid sequence of SEQ ID NO: 31, a framework region 5 (FR5) comprising at least 95% identity to the amino acid sequence of SEQ ID NO: 32, a framework region 6 (FR6) comprising at least 95% identity to the amino acid sequence of SEQ ID NO: 33, a framework region 7 (FR7) comprising at least 95% identity to the amino acid sequence of SEQ ID NO: 34, and a framework region 8 (FR8) comprising at least 95% identity to the amino acid sequence of SEQ ID NO: 35. At least 95% sequence identity includes, for example, 96%, 97%, 98%, and 99% sequence identity.

In some variations, the herein disclosed bi-specific anti-CD19 and anti-CD3 antigen binding molecules comprise an anti-CD3 antigen binding domain comprising an amino acid sequence comprising at least 95% identity to SEQ ID NO: 28 N-terminal to a first binding subunit CDR1, an amino acid sequence comprising at least 95% identity to SEQ ID NO: 29 between the first binding subunit CDR1 and a first binding subunit CDR2, an amino acid sequence comprising at least 95% identity to SEQ ID NO: 30 between the first binding subunit CDR2 and a first binding subunit CDR3, an amino acid sequence comprising at least 95% identity to SEQ ID NO: 31 C-terminal to the first binding subunit CDR3, an amino acid sequence comprising at least 95% identity to SEQ ID NO: 32 N-terminal to a second binding subunit CDR1, an amino acid sequence comprising at least 95% identity to SEQ ID NO: 33 between the second binding subunit CDR1 and a second binding subunit CDR2, an amino acid sequence comprising at least 95% identity to SEQ ID NO: 34 between the second binding subunit CDR2 and a second binding subunit CDR3, and an amino acid sequence comprising at least 95% identity to SEQ ID NO: 35 C-terminal to the second binding subunit CDR3. At least 95% sequence identity includes, for example, 96%, 97%, 98%, and 99% sequence identity.

In some variations, the herein disclosed bi-specific anti-CD19 and anti-CD3 antigen binding molecules comprise an anti-CD3 antigen binding domain comprising a framework region 1 (FR1) comprising the amino acid sequence of SEQ ID NO: 28, a framework region 2 (FR2) comprising the amino acid sequence of SEQ ID NO: 29, a framework region 3 (FR3) comprising the amino acid sequence of SEQ ID NO: 30, a framework region 4 (FR4) comprising the amino acid sequence of SEQ ID NO: 31, a framework region 5 (FR5) comprising the amino acid sequence of SEQ ID NO: 32, a framework region 6 (FR6) comprising the amino acid sequence of SEQ ID NO: 33, a framework region 7 (FR7) comprising the amino acid sequence of SEQ ID NO: 34, and a framework region 8 (FR8) comprising the amino acid sequence of SEQ ID NO: 35.

In some variations, the herein disclosed bi-specific anti-CD19 and anti-CD3 antigen binding molecules comprise an anti-CD3 antigen binding domain comprising an amino acid sequence comprising SEQ ID NO: 28 N-terminal to a first binding subunit CDR1, an amino acid sequence comprising SEQ ID NO: 29 between the first binding subunit CDR1 and a first binding subunit CDR2, an amino acid sequence comprising SEQ ID NO: 30 between the first domain CDR2 and a first binding subunit CDR3, an amino acid sequence comprising SEQ ID NO: 31 C-terminal to the first binding subunit CDR3, an amino acid sequence comprising SEQ ID NO: 32 N-terminal to a second binding subunit CDR1, an amino acid sequence comprising SEQ ID NO: 33 between the second binding subunit CDR1 and a second domain CDR2, an amino acid sequence comprising SEQ ID NO: 34 between the second binding subunit CDR2 and a second binding subunit CDR3, and an amino acid sequence comprising SEQ ID NO: 35 C-terminal to the second binding subunit CDR3.

Anti-CD3 Antigen binding domain Variable Regions

In some variations, the herein disclosed bi-specific anti-CD19 and anti-CD3 antigen binding molecules comprise a first anti-CD19 binding subunit and a first anti-CD3 binding subunit that are heavy chain variable regions, and a second anti-CD19 binding subunit and a second anti-CD3 binding subunit that are light chain variable regions. In some variations, the bi-specific anti-CD19 and anti-CD3 antigen binding molecules comprise an anti-CD3 antigen binding domain comprising a light chain variable region comprising at least 95% sequence identity to the amino acid sequence of SEQ ID NO: 180, and a heavy chain variable region comprising at least 95% sequence identity to the amino acid sequence of SEQ ID NO: 179, 181, 182, 183, 184, 185, 186, 187, 188, or 189. At least 95% sequence identity includes, for example, 96%, 97%, 98%, and 99% sequence identity. In some variations, the bi-specific anti-CD19 and anti-CD3 antigen binding molecules comprise an anti-CD3 antigen binding domain comprising a light chain variable region comprising the amino acid sequence of SEQ ID NO: 180, and a heavy chain variable region comprising the amino acid sequence of SEQ ID NO: 179, 181, 182, 183, 184, 185, 186, 187, 188, or 189.

### Anti-CD3 Antigen Binding Domain Single-Chain Variable Fragments

In some variations, the herein disclosed bi-specific anti-CD19 and anti-CD3 antigen binding molecules comprise an anti-CD3 antigen binding domain comprising a single-chain variable fragment comprising at least 95% sequence identity to the amino acid sequence of SEQ ID NO: 190, 191, 192, 193, 194, 195, 196, 197, 198, 199, 200, 201, 202, 203, 204, 205, 206, 207, 208, or 209. At least 95% sequence identity includes, for example, 96%, 97%, 98%, and 99% sequence identity. In some variations, the bi-specific anti-CD19 and anti-CD3 antigen binding molecules comprise an anti-CD3 antigen binding domain comprising a single-chain variable fragment comprising the amino acid sequence of SEQ ID NO: 190, 191, 192, 193, 194, 195, 196, 197, 198, 199, 200, 201, 202, 203, 204, 205, 206, 207, 208, or 209.

### Bispecific Formats

The herein disclosed bi-specific anti-CD19 and anti-CD3 antigen binding molecules can be a 1+1 Fab-scFv-Fc, 2+1 Fab₂-scFv-Fc, 1+1 Common Light Chain, 2+1 Common Light Chain, 2+1 mAb-scFv, 2+1 stackFab₂-scFv-Fc, Dual scFv, One-arm scFv-mAb, caveman, Bispecific mAb, One-arm central-scFv, mAb-Fv, central-Fv, or Trident. In some variations, the herein disclosed bi-specific anti-CD19 and anti-CD3 antigen binding molecules are a 1+1 Fab-scFv-Fc. In some variations, the herein disclosed bi-specific anti-CD19 and anti-CD3 antigen binding molecules are a 2+1 Fab₂-scFv-Fc. In some variations, the herein disclosed bi-specific anti-CD19 and anti-CD3 antigen binding molecules are a 1+1 Common Light Chain. In some variations, the herein disclosed bi-specific anti-CD19 and anti-CD3 antigen binding molecules are a 2+1 Common Light Chain. In some variations, the herein disclosed bi-specific anti-CD19 and anti-CD3 antigen binding molecules are a 2+1 mAb-scFv. In some variations, the herein disclosed bi-specific anti-CD19 and anti-CD3 antigen binding molecules are a 2+1 stackFab₂-scFv-Fc. In some variations, the herein disclosed bi-specific anti-CD19 and anti-CD3 antigen binding molecules are a Dual scFv. In some variations, the herein disclosed bi-specific anti-CD19 and anti-CD3 antigen binding molecules are a One-arm scFv-mAb. In some variations, the herein disclosed bi-specific anti-CD19 and anti-CD3 antigen binding molecules are a scFv-mAb. In some variations, the herein disclosed bi-specific anti-CD19 and anti-CD3 antigen binding molecules are a Bispecific mAb. In some variations, the herein disclosed bi-specific anti-CD19 and anti-CD3 antigen binding molecules are a One-arm central-scFv. In some variations, the herein disclosed bi-specific anti-CD19 and anti-CD3 antigen binding molecules are a mAb-Fv. In some variations, the herein disclosed bi-specific anti-CD19 and anti-CD3 antigen binding molecules are a central-Fv. In some variations, the herein disclosed bi-specific anti-CD19 and anti-CD3 antigen binding molecules are a Trident.

The 1+1 Fab-scFv-Fc bi-specific anti-CD19 and anti-CD3 antigen binding molecules can comprise the amino acid sequences of SEQ ID NOs: 210, 211, and 212, SEQ ID NOs: 213, 214, and 215, SEQ ID NOs: 213, 216, and 215, SEQ ID NOs: 213, 217, and 215, or SEQ ID NOs: 213, 218, and 215. In some variations, the 1+1 Fab-scFv-Fc bi-specific anti-CD19 and anti-CD3 antigen binding molecule comprises the amino acid sequences of SEQ ID NOs: 210, 211, and 212. In some variations, the 1+1 Fab-scFv-Fc bi-specific anti-CD19 and anti-CD3 antigen binding molecule comprises the amino acid sequences of SEQ ID NOs: 213, 214, and 215. In some variations, the 1+1 Fab-scFv-Fc bi-specific anti-CD19 and anti-CD3 antigen binding molecule comprises the amino acid sequences of SEQ ID NOs: 213, 216, and 215. In some variations, the 1+1 Fab-scFv-Fc bi-specific anti-CD19 and anti-CD3 antigen binding molecule comprises the amino acid sequences of 213, 217, and 215. In some variations, the 1+1 Fab-scFv-Fc bi-specific anti-CD19 and anti-CD3 antigen binding molecule comprises the amino acid sequences of SEQ ID NOs: 213, 218, and 215.

The 2+1 Fab₂-scFv-Fc bi-specific anti-CD19 and anti-CD3 antigen binding molecules can comprise the amino acid sequences of SEQ ID NOs: 210, 219, and 212, SEQ ID NOs: 210, 220, and 212, SEQ ID NOs: 210, 221, and 212, SEQ ID NOs: 210, 222, and 212, SEQ ID NOs: 210, 223, and 212, SEQ ID NOs: 210, 224, and 212, SEQ ID NOs: 210, 225, and 212, SEQ ID NOs: 210, 226, and 212, SEQ ID NOs: 210, 227, and 212, SEQ ID NOs: 210, 228, and 212, SEQ ID NOs: 210, 229, and 212, SEQ ID NOs: 210, 230, and 212, SEQ ID NOs: 210, 231, and 212, SEQ ID NOs: 232, 233, and 215, SEQ ID NOs: 232, 234, and 215, SEQ ID NOs: 232, 235, and 215, SEQ ID NOs: 232, 236, and 215, SEQ ID NOs: 232, 237, and 215, SEQ ID NOs: 232, 238, and 215, SEQ ID NOs: 232, 239, and 215, SEQ ID NOs: 232, 240, and 215, SEQ ID NOs: 232, 241, and 215, SEQ ID NOs: 232, 242, and 215, SEQ ID NOs: 232, 243, and 215, SEQ ID NOs: 232, 244, and 215, SEQ ID NOs: 232, 245, and 215, SEQ ID NOs: 213, 246, and 215, SEQ ID NOs: 213, 247, and 215, SEQ ID NOs: 213, 248, and 215, or SEQ ID NOs: 213, 249, and 215. In some variations, the 2+1 Fab₂-scFv-Fc bi-specific anti-CD19 and anti-CD3 antigen binding molecule comprises the amino acid sequences of SEQ ID NOs: 210, 219, and 212. In some variations, the 2+1 Fab₂-scFv-Fc bi-specific anti-CD19 and anti-CD3 antigen binding molecule comprises the amino acid sequences of SEQ ID NOs: 210, 220, and 212. In some variations, the 2+1 Fab₂-scFv-Fc bi-specific anti-CD19 and anti-CD3 antigen binding molecule comprises the amino acid sequences of SEQ ID NOs: 210, 221, and 212. In some variations, the 2+1 Fab₂-scFv-Fc bi-specific anti-CD19 and anti-CD3 antigen binding molecule comprises the amino acid sequences of SEQ ID NOs: 210, 222, and 212. In some variations, the 2+1 Fab₂-scFv-Fc bi-specific anti-CD19 and anti-CD3 antigen binding molecule comprises the amino acid sequences of SEQ ID NOs: 210, 223, and 212. In some variations, the 2+1 Fab₂-scFv-Fc bi-specific anti-CD19 and anti-CD3 antigen binding molecule comprises the amino acid sequences of SEQ ID NOs: 210, 224, and 212. In some variations, the 2+1 Fab₂-scFv-Fc bi-specific anti-CD19 and anti-CD3 antigen binding molecule comprises the amino acid sequences of SEQ ID NOs: 210, 225, and 212. In some variations, the 2+1 Fab₂-scFv-Fc bi-specific anti-CD19 and anti-CD3 antigen binding molecule comprises the amino acid sequences of SEQ ID NOs: 210, 226, and 212. In some variations, the 2+1 Fab₂-scFv-Fc bi-specific anti-CD19 and anti-CD3 antigen binding molecule comprises the amino acid sequences of SEQ ID NOs: 210, 227, and 212. In some variations, the 2+1 Fab₂-scFv-Fc bi-specific anti-CD19 and anti-CD3 antigen binding molecule comprises the amino acid sequences of SEQ ID NOs: 210, 228, and 212. In some variations, the 2+1 Fab₂-scFv-Fc bi-specific anti-CD19 and anti-CD3 antigen binding molecule comprises the amino acid sequences of SEQ ID NOs: 210, 229, and 212. In some variations, the 2+1 Fab₂-scFv-Fc bi-specific anti-CD19 and anti-CD3 antigen binding molecule comprises the amino acid sequences of SEQ ID NOs: 210, 230, and 212. In some variations, the 2+1 Fab₂-scFv-Fc bi-specific anti-CD19 and anti-CD3 antigen binding molecule comprises the amino acid sequences of SEQ ID NOs: 210, 231, and 212. In some variations, the 2+1 Fab₂-scFv-Fc bi-specific anti-CD19 and anti-CD3 antigen binding molecule comprises the amino acid sequences of SEQ ID NOs: 232, 233, and 215. In some variations, the 2+1 Fab₂-scFv-Fc bi-specific anti-CD19 and anti-CD3 antigen binding molecule comprises the amino acid sequences of SEQ ID NOs: 232, 234, and 215. In some variations, the 2+1 Fab₂-scFv-Fc bi-specific anti-CD19 and anti-CD3 antigen binding molecule comprises the amino acid sequences of SEQ ID NOs: 232, 235, and 215. In some variations, the 2+1 Fab₂-scFv-Fc bi-specific anti-CD19 and anti-CD3 antigen binding molecule comprises the amino acid sequences of SEQ ID NOs: 232, 236, and 215. In some variations, the 2+1 Fab₂-scFv-Fc bi-specific anti-CD19 and anti-CD3 antigen binding molecule comprises the amino acid sequences of SEQ ID NOs: 232, 237, and 215. In some variations, the 2+1 Fab₂-scFv-Fc bi-specific anti-CD19 and anti-CD3 antigen binding molecule comprises the amino acid sequences of SEQ ID NOs: 232, 238, and 215. In some variations, the 2+1 Fab₂-scFv-Fc bi-specific anti-CD19 and anti-CD3 antigen binding molecule comprises the amino acid sequences of SEQ ID NOs: 232, 239, and 215. In some variations, the 2+1 Fab₂-scFv-Fc bi-specific anti-CD19 and anti-CD3 antigen binding molecule comprises the amino acid sequences of SEQ ID NOs: 232, 240, and 215. In some variations, the 2+1 Fab₂-scFv-Fc bi-specific anti-CD19 and anti-CD3 antigen binding molecule comprises the amino acid sequences of SEQ ID NOs: 232, 241, and 215. In some variations, the 2+1 Fab₂-scFv-Fc bi-specific anti-CD19 and anti-CD3 antigen binding molecule comprises the amino acid sequences of SEQ ID NOs: 232, 242, and 215. In some variations, the 2+1 Fab₂-scFv-Fc bi-specific anti-CD19 and anti-CD3 antigen binding molecule comprises the amino acid sequences of SEQ ID NOs: 232, 243, and 215. In some variations, the 2+1 Fab₂-scFv-Fc bi-specific anti-CD19 and anti-CD3 antigen binding molecule comprises the amino acid sequences of SEQ ID NOs: 232, 244, and 215. In some variations, the 2+1 Fab₂-scFv-Fc bi-specific anti-CD19 and anti-CD3 antigen binding molecule comprises the amino acid sequences of SEQ ID NOs: 232, 245, and 215. In some variations, the 2+1 Fab₂-scFv-Fc bi-specific anti-CD19 and anti-CD3 antigen binding molecule comprises the amino acid sequences of SEQ ID NOs: 213, 246, and 215. In some variations, the 2+1 Fab₂-scFv-Fc bi-specific anti-CD19 and anti-CD3 antigen binding molecule comprises the amino acid sequences of SEQ ID NOs: 213, 247, and 215. In some variations, the 2+1 Fab₂-scFv-Fc bi-specific anti-CD19 and anti-CD3 antigen binding molecule comprises the amino acid sequences of SEQ ID NOs: 213, 248, and 215. In some variations, the 2+1 Fab₂-scFv-Fc bi-specific anti-CD19 and anti-CD3 antigen binding molecule comprises the amino acid sequences of SEQ ID NOs: 213, 249, and 215.

### Stability

The anti-CD3 antigen binding domain of the bispecific antigen binding molecule can include, for example, a non-canonical deamidation motif GNF at positions 96-98 (Kabat numbering) in the first binding subunit CDR3. Consequentially, variants are made to remove this deamidation liability as well as to engineer stabilizing disulfide bridges. N97 is important for binding, but variants at N97 abrogated binding affinity (with the exception of N97Q which retains some binding). Variants at G96 is also important to binding, and G96E or G96T was found to improve binding affinity (albeit with decrease in stability by ˜2-4° C.).

The disulfide bridges can increase the thermodynamic stability of the anti-CD3 antigen binding domain of the bispecific antigen binding molecule. In some variations, the disulfide bridges can increase the denaturation temperature by at least 2° C.). In some variations, the disulfide bridges can increase the denaturation temperature by at least 3° C.). In some variations, the disulfide bridges can increase the denaturation temperature by at least 4° C.).

The anti-CD3 antigen binding domain of the bispecific antigen binding molecule can include, for example a D100G substitution to improve affinity. Further variants are engineered to fix the deamidation and incorporate disulfide bridges while maintaining an affinity ladder and improved stability of the anti-CD3 antigen binding domain. Sequences for these variants are depicted in, for example, FIG. 17A-FIG. 17J, and affinity (in the context of 2+1 Fab₂-scFv-Fc molecules of FIG. 20A-FIG. 20AD) are depicted in FIG. 30. The re-engineered anti-CD3 antigen binding domain can have binding affinity similar to a non-engineered anti-CD3 antigen binding molecule, albeit with removal of the deamidation motif and introduction of stabilizing disulfide bridges.

The anti-CD19 antigen binding domain of the bispecific antigen binding molecules are, for example, re-humanized and demonstrate enhanced stability. Germline identities and 9mers were increased across both the VH and VL, while maintaining binding affinity (FIG. 29). Further, the re-humanized anti-CD19 antigen binding domain has +7° C. improved Tₘ (FIG. 29). For example, FIG. 29 shows that the anti-CD19 antigen binding domain has a Tₘ of 78.5° C. compared to a reference anti-CD19 molecule with a Tₘ of 71.5° C.

An A100Q substitution in the VL FR4 alone of the anti-CD19 antigen binding domain of the bispecific antigen binding molecules improves stability by 1° C. and removed a predicted MHC-binding peptide (data not shown). Additionally, a VL CDR2 methionine that may be liable to oxidation is substituted with alanine (FIG. 28B). The M51A substitution does not damage binding affinity. The re-engineered anti-CD19 antigen binding domain can contain, for example, 33 amino acid substitutions relative to the parental sequence.

The herein disclosed anti-CD3 and anti-CD19 bispecific antigen binding molecules exhibit comparable CD19+ Ramos cell killing and T-cell activation compared to the reference bispecific molecules that lack the sequence changes that mediate the improved stability.

### Nucleic Acids, Vectors, and Host Cells

In one embodiment of the present invention, nucleic acids are created that encode the antigen binding molecules, and particularly antibodies described herein, and that may then be cloned into vectors host cells. A nucleic acid composition may refer to one or multiple polynucleotides.

Thus, nucleic acids, and particularly DNA, may be made that encode each protein sequence. These practices are carried out using well-known procedures. For example, a variety of methods that may find use in the present invention are described in Molecular Cloning—A Laboratory Manual, 3rd Ed. (Maniatis, Cold Spring Harbor Laboratory Press, New York, 2012), incorporated herein by reference in its entirety.

The antibodies of the present invention may be produced by culturing a host cell transformed with nucleic acid, preferably an expression vector, containing nucleic acid encoding the antibodies, under the appropriate conditions to induce or cause expression of the protein. The conditions appropriate for expression will vary with the choice of the expression vector and the host cell, and will be easily ascertained by one skilled in the art through routine experimentation. A wide variety of appropriate host cells may be used, including but not limited to mammalian cells, bacteria, insect cells, and yeast. For example, a variety of cell lines that may find use in the present invention are described in the ATCC® cell line catalog, available from the American Type Culture Collection.

The nucleic acids that encode the antibodies may be incorporated into an expression vector in order to express components of the antibody. A variety of expression vectors may be utilized for protein expression. Expression vectors may comprise self-replicating extra-chromosomal vectors or vectors which integrate into a host genome. Expression vectors are constructed to be compatible with the host cell type. Thus expression vectors which find use in the present invention include but are not limited to those which enable protein expression in mammalian cells, bacteria, insect cells, yeast, and in in vitro systems. As is known in the art, a variety of expression vectors are available, commercially or otherwise, that may find use in the present invention for expressing antibodies.

As is known in the art, the nucleic acids encoding the components of the binding domains and antibodies disclosed herein can be incorporated into expression vectors as is known in the art, and depending on the host cells used to produce antibodies, including heterodimeric antibodies. Expression vectors typically comprise a protein operably linked with control or regulatory sequences, selectable markers, any fusion partners, and/or additional elements. Nucleic acids are operably linked to any number of regulatory elements (promoters, origin of replication, selectable markers, ribosomal binding sites, inducers, etc.). The expression vectors can be extra-chromosomal or integrating vectors. Generally, vectors include transcriptional and translational regulatory nucleic acid operably linked to the nucleic acid encoding the antibody, and are typically appropriate to the host cell used to express the protein.

As will be appreciated by those in the art, the nucleic acid compositions will depend on the format and scaffold of the heterodimeric protein. Thus, for example, when the format requires three amino acid sequences, such as for the 1+1 Fab-scFv-Fc or 2+1 Fab₂-scFv-Fc formats, three polynucleotides can be incorporated into one or more expression vectors for expression. In some embodiments, each polynucleotide is incorporated into a different expression vector. In other embodiments, polynucleotides can be incorporated into the same expression vector.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors.

The antibodies provided herein are made by culturing host cells comprising the expression vector(s) as is well known in the art. Once produced, traditional antibody purification steps are done, including an ion exchange chromatography step. As discussed herein, having the pls of the two monomers differ by at least 0.5 can allow separation by ion exchange chromatography or isoelectric focusing, or other methods sensitive to isoelectric point. That is, the inclusion of pl substitutions that alter the isoelectric point (pl) of each monomer so that such that each monomer has a different pl and the heterodimer also has a distinct pl, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, clEF, and analytical IEX columns).

Proteins may be isolated or purified in a variety of ways known to those skilled in the art. Standard purification methods include chromatographic techniques, including ion exchange, hydrophobic interaction, affinity, sizing or gel filtration, and reversed-phase, carried out at atmospheric pressure or at high pressure using systems such as FPLC and HPLC. Purification methods also include electrophoretic, immunological, precipitation, dialysis, and chromatofocusing techniques. Ultrafiltration and diafiltration techniques, in conjunction with protein concentration, are also useful. As is well known in the art, a variety of natural proteins bind Fc and antibodies, and these proteins can find use in the present invention for purification of antibodies. For example, the bacterial proteins A and G bind to the Fc region. Likewise, the bacterial protein L binds to the Fab region of some antibodies, as of course does the antibody's target antigen. Purification can often be enabled by a particular fusion partner. For example, antibodies may be purified using glutathione resin if a GST fusion is employed, Ni+2 affinity chromatography if a His-tag is employed, or immobilized anti-flag antibody if a flag-tag is used. For general guidance in suitable purification techniques, see, e.g. incorporated entirely by reference Protein Purification: Principles and Practice, 3rd Ed., Scopes, Springer-Verlag, NY, 1994, incorporated entirely by reference. The degree of purification necessary will vary depending on the screen or use of the antibodies. In some instances no purification is necessary. If the antibodies are secreted, screening may take place directly from the media. As is well known in the art, some methods of selection do not involve purification of proteins. Thus, for example, if a library of antibodies is made into a phage display library, protein purification may not be performed.

### Uses of the Bispecific Antigen Binding Molecules

Disclosed herein are methods of depleting B-cells in a subject, the method comprising administering any of the herein disclosed bi-specific anti-CD19 and anti-CD3 antigen binding molecules to the subject. Disclosed herein are compositions comprising any of the herein disclosed bispecific antibodies for use in a method of depleting B-cells in a subject, the method comprising administering the composition to the subject. Disclosed herein are any of the herein disclosed bispecific antibodies for use in the preparation of a medicament useful for depleting B-cells in a subject.

The methods for depleting B-cells can be useful in treating B cell-mediated disorders, including but not limited to allergic responses, autoimmune diseases, inflammatory diseases, and hematological malignancies.

The herein disclosed methods and uses can deplete peripheral B-cells, bone marrow B-cells, and lymph node B-cells. In some embodiments, peripheral B-cells are depleted.

In some embodiments, bone marrow B-cells are depleted. In some embodiments, lymph node B-cells are depleted.

The bi-specific binding molecules can be administered to the subject by any suitable means of administration including, for example, subcutaneous administration or intravenous administration. In some embodiments, the bi-specific binding molecules are administered subcutaneously. In some embodiments, the bi-specific binding molecules are administered intravenously.

Disclosed herein are methods of treating an autoimmune, inflammatory disorder, or cancer in a subject, the method comprising administering any of the herein disclosed the bi-specific anti-CD19 and anti-CD3 antigen binding molecules to the subject. In some embodiments, an autoimmune disorder is treated. In some embodiments, an inflammatory disease is treated. In some variations, cancer is treated. Specifically, the treatment methods disclosed herein comprise administration to a patient in need of such administration a therapeutic amount of the antigen binding molecule as described herein.

Disclosed herein are compositions comprising any of the herein disclosed bispecific antibodies for use in a method of treating an autoimmune disorder, inflammatory disease, or cancer in a subject, the method comprising administering the composition to the subject. Disclosed herein are any of the herein disclosed bi-specific antibodies for use in the preparation of a medicament useful for treating an autoimmune disorder in a subject. In some embodiments, an autoimmune disorder is treated. In some embodiments, an inflammatory disease is treated. In some embodiments, cancer is treated.

In some variations, the antibodies disclosed herein can be for use in the treatment of an autoimmune disorder (or any specific autoimmune disorder described herein). In some variations, the antibodies disclosed herein can be for use in the treatment of an inflammatory disease (or any specific inflammatory disease described herein). In some variations, the antibodies disclosed herein can be for use in the treatment of cancer (or any specific cancer described herein).

In one variation, the autoimmune disease is allogenic islet graft rejection. In another variation, the autoimmune disease is alopecia areata. In another variation, the autoimmune disease is ankylosing spondylitis. In another variation, the autoimmune disease is antiphospholipid syndrome. In another variation, the autoimmune disease is autoimmune Addison's disease. In another variation, the autoimmune disease is antineutrophil cytoplasmic autoantibodies (ANCA). In another variation, the autoimmune disease is autoimmune diseases of the adrenal gland. In some variations, the autoimmune disease is anti-MDA5 syndrome. In another variation, the autoimmune disease is antisynthetase syndrome. In another variation, the autoimmune disease is autoimmune autonomic ganglionopathy (AAG). In another variation, the autoimmune disease is autoimmune encephalitis (anti-NMDAR, anti-LGI1). In another variation, the autoimmune disease is autoimmune hemolytic anemia. In another variation, the autoimmune disease is autoimmune hepatitis. In another variation, the autoimmune disease is autoimmune myocarditis. In another variation, the autoimmune disease is autoimmune neutropenia. In another variation, the autoimmune disease is autoimmune oophoritis and orchitis. In another variation, the autoimmune disease is autoimmune thrombocytopenia. In another variation, the autoimmune disease is autoimmune urticaria. In another variation, the autoimmune disease is Behcet's disease. In another variation, the autoimmune disease is bullous pemphigoid. In another variation, the autoimmune disease is cardiomyopathy. In another variation, the autoimmune disease is Castleman's syndrome. In another variation, the autoimmune disease is celiac spruce-dermatitis. In another variation, the autoimmune disease is chronic fatigue immune disfunction syndrome. In another variation, the autoimmune disease is chronic inflammatory demyelinating polyneuropathy. In another variation, the autoimmune disease is Churg-Strauss syndrome. In another variation, the autoimmune disease is cicatrical pemphigoid. In another variation, the autoimmune disease is cold agglutinin disease. In another variation, the autoimmune disease is CREST syndrome. In another variation, the autoimmune disease is cold agglutinin disease. In another variation, the autoimmune disease is Crohn's disease. In another variation, the autoimmune disease is dermatomyositis. In another variation, the autoimmune disease is discoid lupus. In another variation, the autoimmune disease is essential mixed cryoglobulinemia. In another variation, the autoimmune disease is Evans syndrome. In another variation, the autoimmune disease is factor VIII deficiency. In another variation, the autoimmune disease is fibromyalgia-fibromyositis. In another variation, the autoimmune disease is glomerulonephritis. In another variation, the autoimmune disease is Grave's disease. In another variation, the autoimmune disease is Guillain-Barre. In another variation, the autoimmune disease is Goodpasture's syndrome. In another variation, the autoimmune disease is graft-versus-host disease (GVHD). In another variation, the autoimmune disease is Hashimoto's thyroiditis. In another variation, the autoimmune disease is hemophilia A. In another variation, the autoimmune disease is idiopathic pulmonary fibrosis. In another variation, the autoimmune disease is immune mediated thrombocytopenia. In another variation, the autoimmune disease is immune thrombocytopenic purpura (ITP). In another variation, the autoimmune disease is IgA neuropathy. In another variation, the autoimmune disease is IgM polyneuropathies. In another variation, the autoimmune disease is IgA vasculitis (Henoch-Schonlein purpura). In another variation, the autoimmune disease is IgG4-related disease. In another variation, the autoimmune disease is immune mediated thrombocytopenia. In another variation, the autoimmune disease is juvenile arthritis. In another variation, the autoimmune disease is Kawasaki's disease. In another variation, the autoimmune disease is lichen planus. In another variation, the autoimmune disease is lupus erthematosis. In another variation, the autoimmune disease is Meniere's disease. In another variation, the autoimmune disease is mixed connective tissue disease. In another variation, the autoimmune disease is multiple sclerosis. In another variation, the autoimmune disease is type 1 diabetes mellitus. In another variation, the autoimmune disease is myasthenia gravis. In another variation, the autoimmune disease is Neuromyelitis optica spectrum disorder (NMOSD). In another variation, the autoimmune disease is Overlap myositis. In another variation, the autoimmune disease is pemphigus vulgaris. In another variation, the autoimmune disease is pernicious anemia. In another variation, the autoimmune disease is polyarteritis nodosa. In another variation, the autoimmune disease is polychrondritis. In another variation, the autoimmune disease is polyglandular syndromes. In another variation, the autoimmune disease is polymyalgia rheumatica. In another variation, the autoimmune disease is polymyositis and dermatomyositis. In another variation, the autoimmune disease is primary agammaglobinulinemia. In another variation, the autoimmune disease is primary biliary cirrhosis. In another variation, the autoimmune disease is primary biliary cholangitis (CD19⁺ plasma-cell dominant). In another variation, the autoimmune disease is psoriasis. In another variation, the autoimmune disease is psoriatic arthritis. In another variation, the autoimmune disease is Reynauld's phenomenon. In another variation, the autoimmune disease is Reiter's syndrome. In another variation, the autoimmune disease is rheumatoid arthritis. In another variation, the autoimmune disease is sarcoidosis. In another variation, the autoimmune disease is scleroderma. In another variation, the autoimmune disease is Sjogren's syndrome. In another variation, the autoimmune disease is solid organ transplant rejection. In another variation, the autoimmune disease is stiff-man syndrome. In another variation, the autoimmune disease is systemic lupus erythematosus (SLE). in another variation, the autoimmune disease is type 1 diabetes mellitus (early or new-onset). In another variation, the autoimmune disease is systemic sclerosis (diffuse cutaneous subtype). In another variation, the autoimmune disease is takayasu arteritis. In another variation, the autoimmune disease is temporal arteristis/giant cell arteritis. In another variation, the autoimmune disease is thrombotic thrombocytopenia purpura. In another variation, the autoimmune disease is ulcerative colitis. In another variation, the autoimmune disease is uveitis. In another variation, the autoimmune disease is vasculitides such as dermatitis herpetiformis vasculitis. In another variation, the autoimmune disease is vitiligo. In another variation, the autoimmune disease is Wegner's granulomatosis.

In one variation, the inflammatory disorder is acute respiratory distress syndrome (ARDS). In another variation, the inflammatory disorder is acute septic arthritis. In another variation, the inflammatory disorder is adjuvant arthritis (Prakken et al., Springer Semin Immunopathol., 2003 August; 25(1):47-63, incorporated entirely by reference), juvenile idiopathic arthritis (de Kleer et al., Arthritis Rheum. 2003 July; 47(7):2001-10, incorporated entirely by reference). In another variation, the inflammatory disorder is allergic encephalomyelitis. In another variation, the inflammatory disorder is allergic rhinitis. In another variation, the inflammatory disorder is allergic vasculitis. In another variation, the inflammatory disorder is allergy. In another variation, the inflammatory disorder is asthma. In another variation, the inflammatory disorder is atherosclerosis. In another variation, the inflammatory disorder is chronic inflammation due to chronic bacterial or viral infectionis. In another variation, the inflammatory disorder is chronic obstructive pulmonary disease (COPD). In another variation, the inflammatory disorder is coronary artery disease. In another variation, the inflammatory disorder is encephalitis. In another variation, the inflammatory disorder is inflammatory bowel disease. In another variation, the inflammatory disorder is inflammatory osteolysis. In another variation, the inflammatory disorder is inflammation associated with acute and delayed hypersensitivity reactions. In another variation, the inflammatory disorder is inflammation associated with tumors. In another variation, the inflammatory disorder is peripheral nerve injury or demyelinating diseases. In another variation, the inflammatory disorder is inflammation associated with tissue trauma such as burns and ischemia. In another variation, the inflammatory disorder is inflammation due to meningitis. In another variation, the inflammatory disorder is multiple organ injury syndrome. In another variation, the inflammatory disorder is pulmonary fibrosis. In another variation, the inflammatory disorder is sepsis and septic shock. In another variation, the inflammatory disorder is Stevens-Johnson syndrome. In another variation, the inflammatory disorder is undifferentiated arthropy. In another variation, the inflammatory disorder is undifferentiated spondyloarthropathy.

In some variations, a cancer is treated. The cancer can be a non-small cell lung cancer, small cell lung cancer, renal cell cancer, colorectal cancer, ovarian cancer, breast cancer, pancreatic cancer, gastric carcinoma, bladder cancer, esophageal cancer, mesothelioma, melanoma, head and neck cancer, thyroid cancer, sarcoma, prostate cancer, glioblastoma, cervical cancer, thymic carcinoma, leukemia, lymphomas, myelomas, mycoses fungoids, merkel cell cancer, and other hematologic malignancies, such as classical Hodgkin lymphoma (CHL), primary mediastinal large B-cell lymphoma, T-cell/histiocyte-rich B-cell lymphoma, EBV-positive and -negative PTLD, and EBV-associated diffuse large B-cell lymphoma (DLBCL), plasmablastic lymphoma, extranodal NK/T-cell lymphoma, nasopharyngeal carcinoma, and HHV8-associated primary effusion lymphoma, Hodgkin's lymphoma, neoplasm of the central nervous system (CNS), such as primary CNS lymphoma, spinal axis tumor, or brain stem glioma.

Cancers can include hematologic malignancies, such as non-Hodgkin's lymphomas (NHL), including refractory/resistant NHL. NHL cancers include but are not limited to Burkitt's lymphoma (BL), small lymphocytic lymphoma (SLL), chronic lymphocytic leukemia (CLL), mantle cell lymphoma (MCL), follicular lymphoma (FL), marginal zone B-cell lymphoma (MZL), hairy cell leukemia (HCL) and lymphoplasmacytic leukemia (LPL), mucosa-associated lymphoid tissue (MALT), extranodal marginal zone B-cell lymphoma of MALT, nodal marginal zone B cell lymphoma, mediastinal large cell lymphoma, intravascular large cell lymphoma, primary effusion lymphoma, precursor B-lymphoblastic leukemia/lymphoma, precursor T- and NK-cells lymphoma (precursor T lymphoblastic lymphoma, blastic NK cell lymphoma), tumors of the mature T and NK cells, including peripheral T-cell lymphoma and leukemia (PTL), adult T-cell leukemia/T-cell lymphomas and large granular lymphocytic leukemia, T-cell chronic lymphocytic leukemia/prolymphocytic leukemia, T-cell large granular lymphocytic leukemia, aggressive NK-cell leukemia, extranodal T-/NK cell lymphoma, enteropathy-type T-cell lymphoma, hepatosplenic T-cell lymphoma, anaplastic large cell lymphoma (ALCL), angiocetric and angioimmunoblastic T-cell lymphoma, mycosis fungoides/Sezary syndrome, and cutaneous T-cell lymphoma (CTCL).

In some variations, the cancer is a CD19-related cancer, such as B cell lymphoma, optionally Hodgkin lymphoma or non-Hodgkin lymphoma. Non-Hodgkin lymphoma can include large B-cell lymphoma (DLBCL), FL, MLL, MZL, MALT, small lymphocytic lymphoma (such as CLL), MCL, Acute Lymphoblastic Leukemia (ALL), or Waldenstrom's Macroglobulinemia (WM). In some variations, the CD19-related cancer is CD19⁺ macrophage-rich hepatocellular carcinoma (experimental TAM-targeting). In some variations, the CD19-related cancer is CD19⁺ multiple myeloma (subsets with retained B-cell lineage antigens). In some variations, the CD19-related cancer is CD19⁺ testicular lymphoma (relapsed cases). In some variations, the CD19-related cancer is CD19-expressing transformed marginal zone lymphoma.

In some variations, the cancer is a non-small cell lung cancer. In some variations, the cancer is a small cell lung cancer. In some variations, the cancer is a renal cell cancer. In some variations, the cancer is a colorectal cancer. In some variations, the cancer is a ovarian cancer. In some variations, the cancer is a breast cancer. In some variations, the cancer is a pancreatic cancer. In some variations, the cancer is a gastric carcinoma. In some variations, the cancer is a bladder cancer. In some variations, the cancer is a esophageal cancer. In some variations, the cancer is a mesothelioma. In some variations, the cancer is a melanoma. In some variations, the cancer is a head a nd neck cancer. In some variations, the cancer is a thyroid cancer. In some variations, the cancer is a sarcoma. In some variations, the cancer is a prostate cancer. In some variations, the cancer is a glioblastoma. In some variations, the cancer is a cervical cancer. In some variations, the cancer is a thymic carcinoma. In some variations, the cancer is a leukemia. In some variations, the cancer is a lymphomas. In some variations, the cancer is a myeloma. In some variations, the cancer is a mycoses fungoids. In some variations, the cancer is a myeloma. In some variations, the cancer is a myeloid neoplasias. In some variations, the cancer is a merkel cell cancer. In some variations, the cancer is a primary mediastinal large B-cell lymphoma. In some variations, the cancer is a T-cell/histiocyte-rich B-cell lymphoma. In some variations, the cancer is gray zone lymphoma. In some variations, the cancer is diffuse large B-cell lymphoma (DLCL). In some variations, the cancer is diffuse large B-cell lymphoma (DLBCL). In some variations, the cancer is an EBV-positive PTLD. In some variations, the cancer is an EBV-negative PDLL. In some variations, the cancer is an EBV-associated DLBCL. In some variations, the cancer is a plasmablastic lymphoma. In some variations, the cancer is an extranodal NK/T-cell lymphoma. In some variations, the cancer is a nasopharyngeal carcinoma. In some variations, the cancer is an HHV8-associated primary effusion lymphoma. In some variations, the cancer is a Hodgkin's lymphoma. In some variations, the cancer is a Hodgkin's lymphoma including AML with maturation. In some variations, the cancer is a neoplasm of the central nervous system (CNS). In some variations, the cancer is a spinal axis tumor. In some variations, the cancer is a brain stem glioma. In some variations, the cancer is a post-transplant lymphoproliferative disorder (PTLD). In some variations, the cancer is thymoma. In some variations, the cancer is transformed follicular lymphoma. In some variations, the cancer is a tumor of lymphocyte precursor cells.

In some variations, the cancer is a non-Hodgkin's lymphomas (NHL). In some variations, the NHL is a refractory/resistant NHL. In some variations, the NHL is Burkitt's lymphoma (BL). In some variations, the NHL issmall lymphocytic lymphoma (SLL). In some variations, the NHL ischronic lymphocytic leukemia (CLL). In some variations, the NHL is a mantle cell lymphoma (MCL). In some variations, the NHL isfollicular lymphoma (FL). In some variations, the NHL is marginal zone B-cell lymphoma (MZL). In some variations, the NHL is hairy cell leukemia (HCL) and lymphoplasmacytic leukemia (LPL). In some variations, the NHL is mucosa-associated lymphoid tissue (MALT). In some variations, the NHL is extranodal marginal zone B-cell lymphoma of MALT. In some variations, the NHL is nodal marginal zone B cell lymphoma. In some variations, the NHL is mediastinal large cell lymphoma. In some variations, the NHL is intravascular large cell lymphoma. In some variations, the NHL is primary effusion lymphoma. In some variations, the NHL is precursor B-lymphoblastic leukemia/lymphoma. In some variations, the NHL is precursor T- and NK-cells lymphoma (such as precursor T lymphoblastic lymphoma. In some variations, the NHL is blastic NK cell lymphoma). In some variations, the NHL is peripheral T-cell lymphoma and leukemia (PTL). In some variations, the NHL is large granular lymphocytic leukemia. In some variations, the NHL is T-cell chronic lymphocytic leukemia/prolymphocytic leukemia. In some variations, the NHL is T-cell large granular lymphocytic leukemia. In some variations, the NHL is aggressive NK-cell leukemia. In some variations, the NHL is extranodal T-/NK cell lymphoma. In some variations, the NHL is enteropathy-type T-cell lymphoma. In some variations, the NHL is hepatosplenic T-cell lymphoma. In some variations, the NHL is an aplastic large cell lymphoma (ALCL). In some variations, the NHL is an anangiocetric and angioimmunoblastic T-cell lymphoma. In some variations, the NHL is a mycosis fungoides/Sezary syndrome. In some variations, the NHL is a cutaneous T-cell lymphoma (CTCL).

The bi-specific binding molecules can be administered to the subject by any suitable means of administration including, for example, subcutaneous administration or intravenous administration. In some embodiments, the bi-specific binding molecules are administered subcutaneously. In some embodiments, the bi-specific binding molecules are administered intravenously.

### EXAMPLES

The following examples are provided to further describe some of the variations disclosed herein. The examples are intended to illustrate, not to limit, the disclosed variations.

### Example 1: CD19 Binding Domains

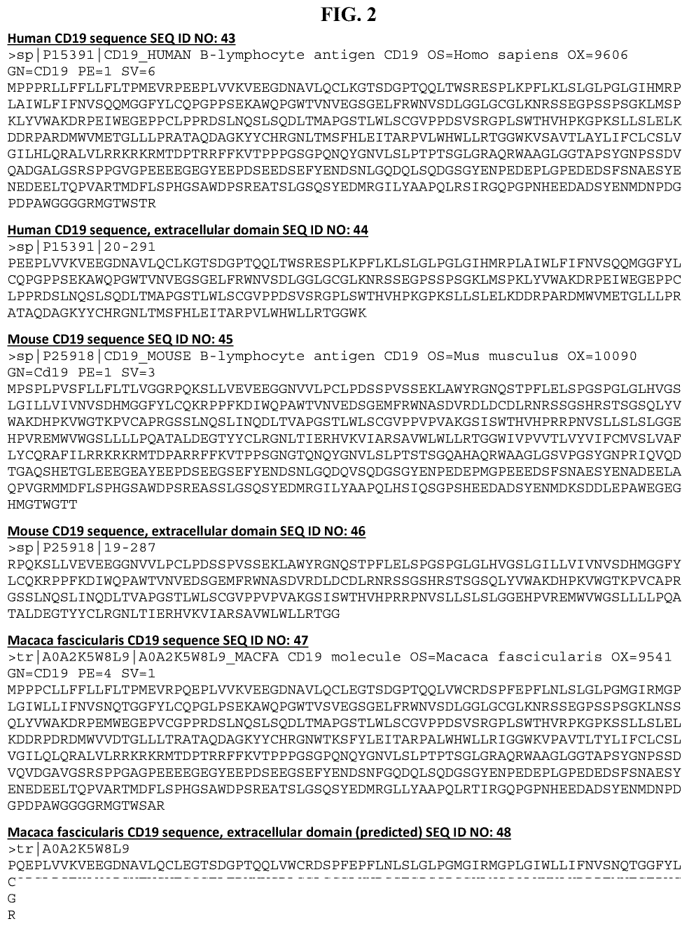

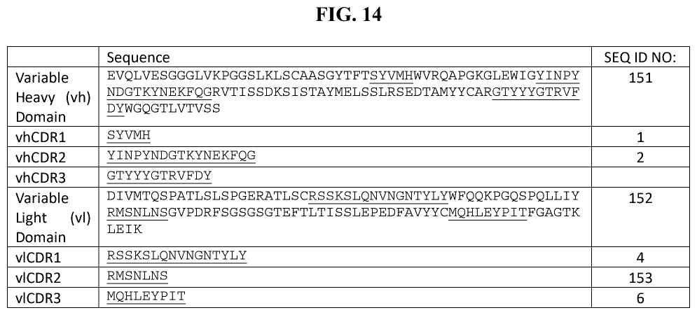

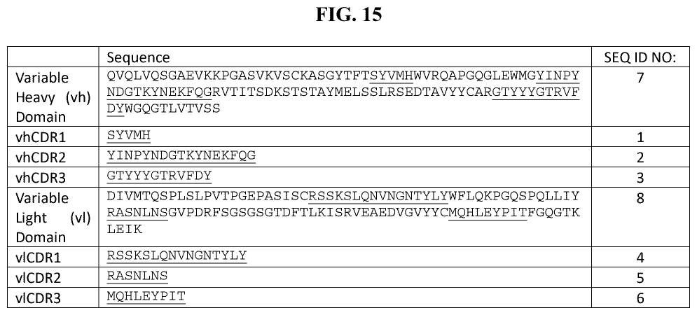

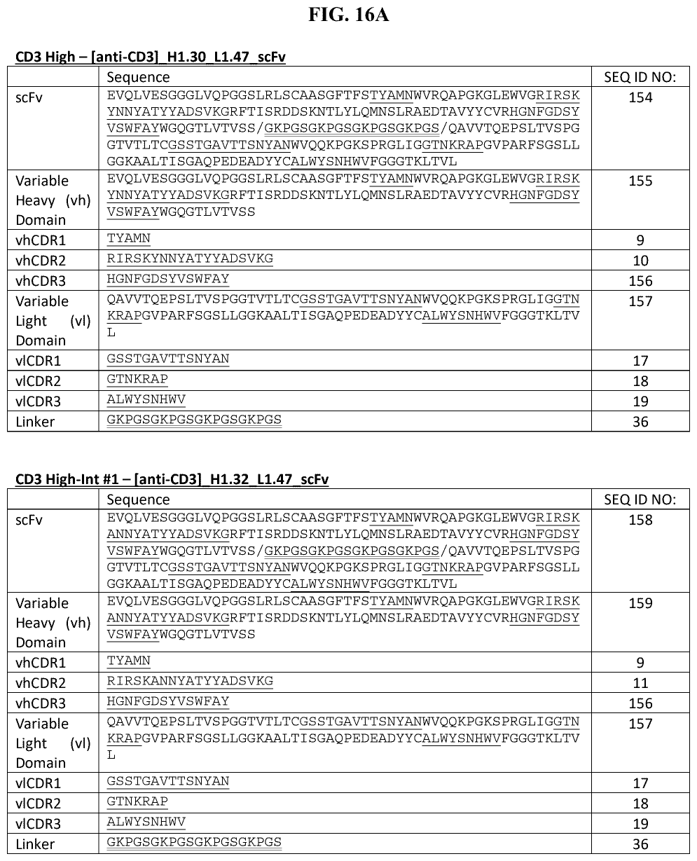

Sequences for human, mouse, and cynomolgus CD19 are depicted in FIG. 2 and are useful for the development of cross-reactive CD19 antigen binding domains for ease of clinical development. Sequences for CD19 binding domains which may find use in the CD19×CD3 bsAbs of the disclosure are known in the art, illustrative sequences which are depicted in FIG. 14. CD19 binding domains were also engineered (as will be described in Example 6) for use in the CD19×CD3 bsAbs of the disclosure, sequences for which are depicted in FIG. 15.

### Example 2: CD3 Binding Domains

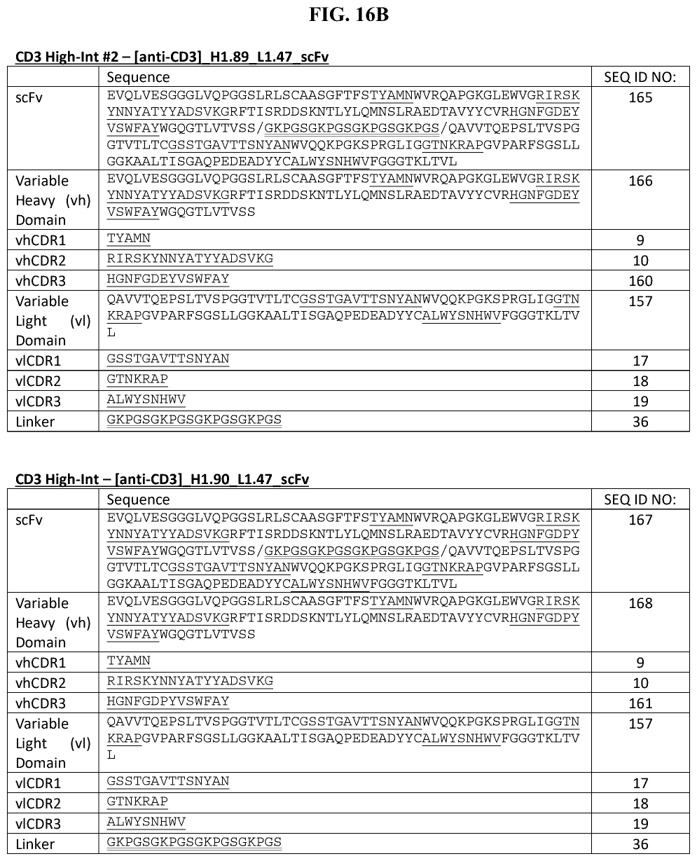

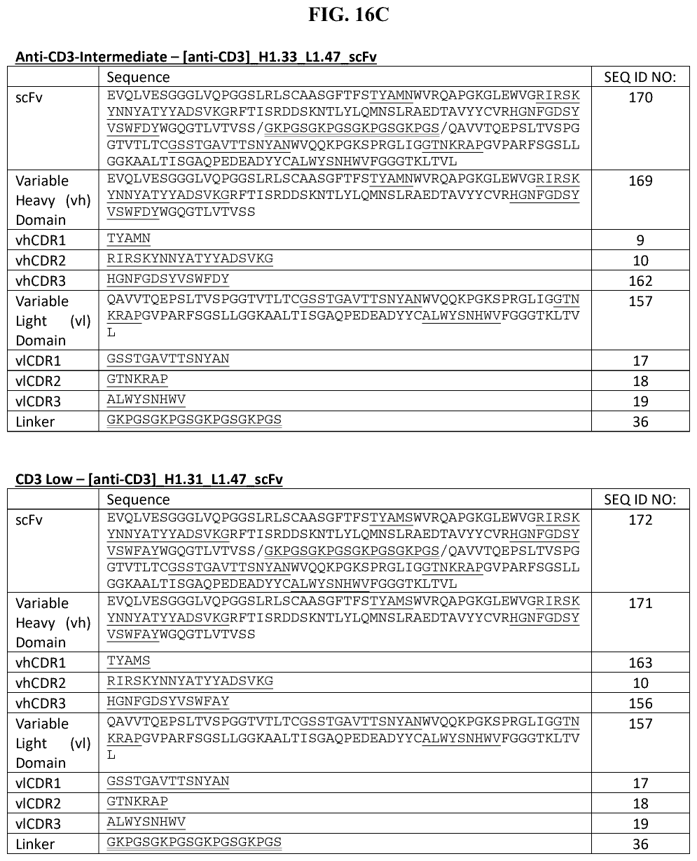

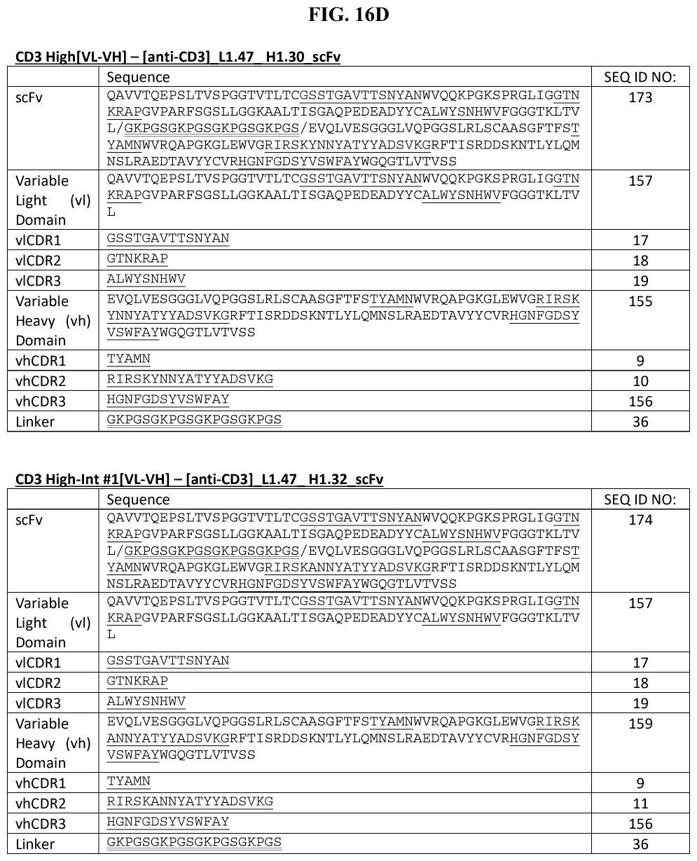

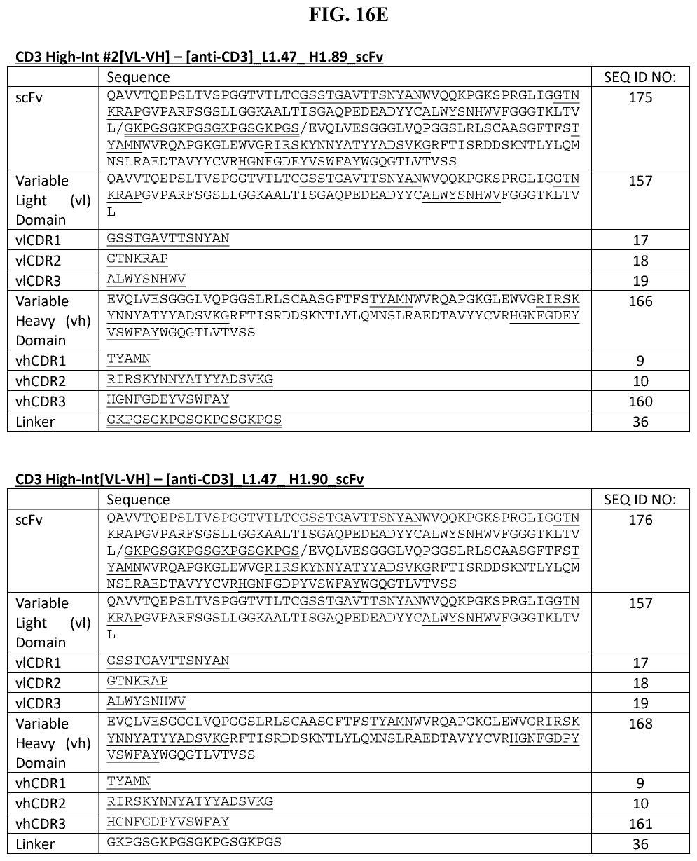

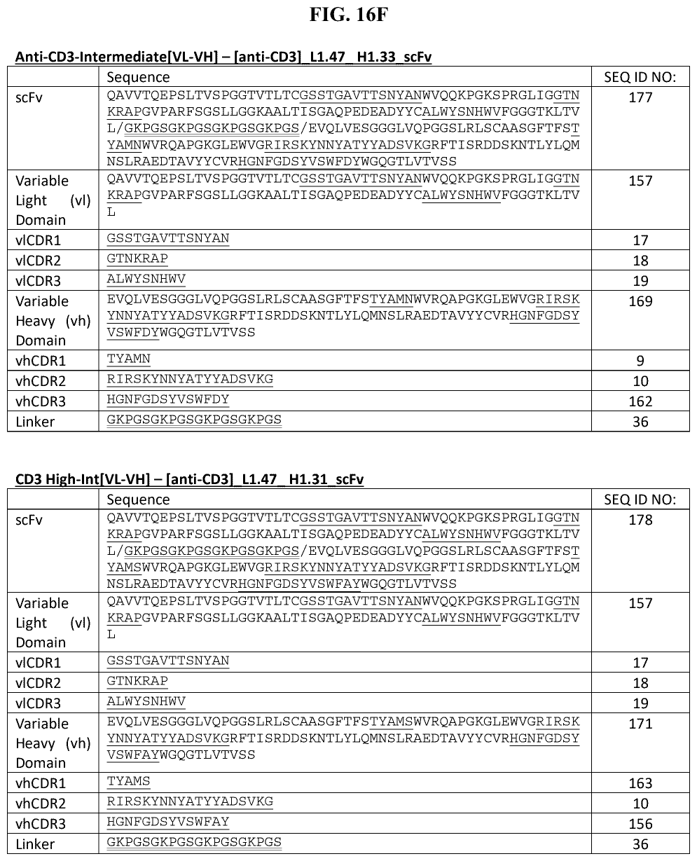

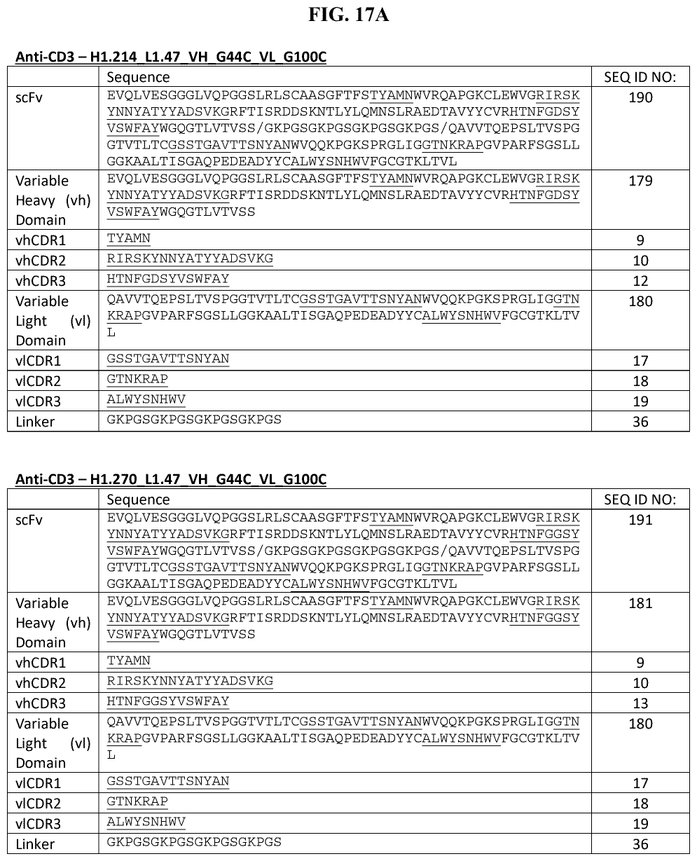

Sequences for human, mouse, and cynomolgus CD3 are depicted in FIG. 1 and are useful for the development of cross-reactive CD3 antigen binding domains for ease of clinical development. Sequences for CD3 binding domains which may find use in the CD19×CD3 bsAbs of the disclosure are known in the art, illustrative sequences which are depicted in FIG. 16A-FIG. 16F. Novel CD3 binding domains were also engineered (as will be described in Example 7) for use in the CD19×CD3 bsAbs of the disclosure, sequences for which are depicted in FIG. 17A-FIG. 17J.

### Example 3: Engineering CD19×CD3 bsAbs

### 3A: 1+1 Fab-scFv-Fc Format

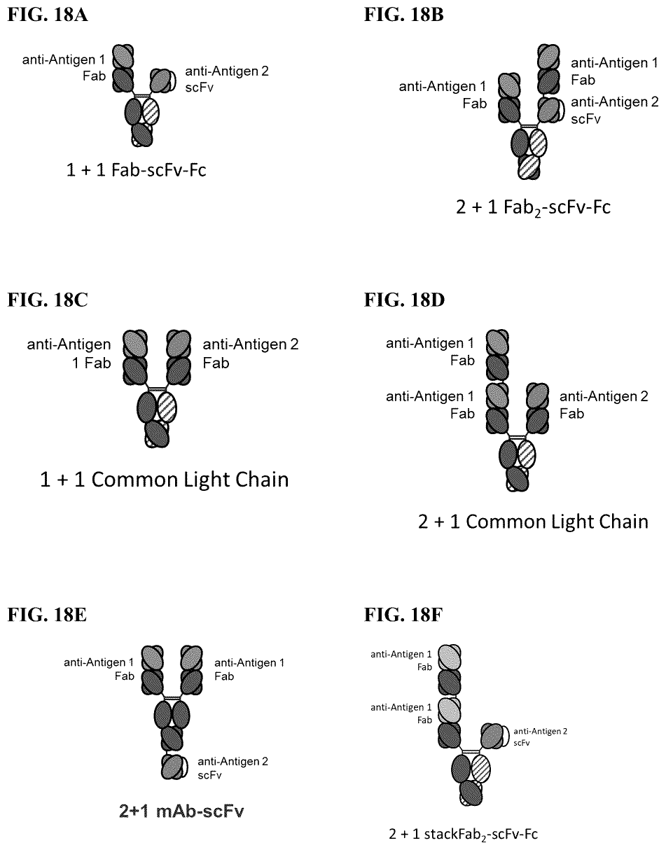

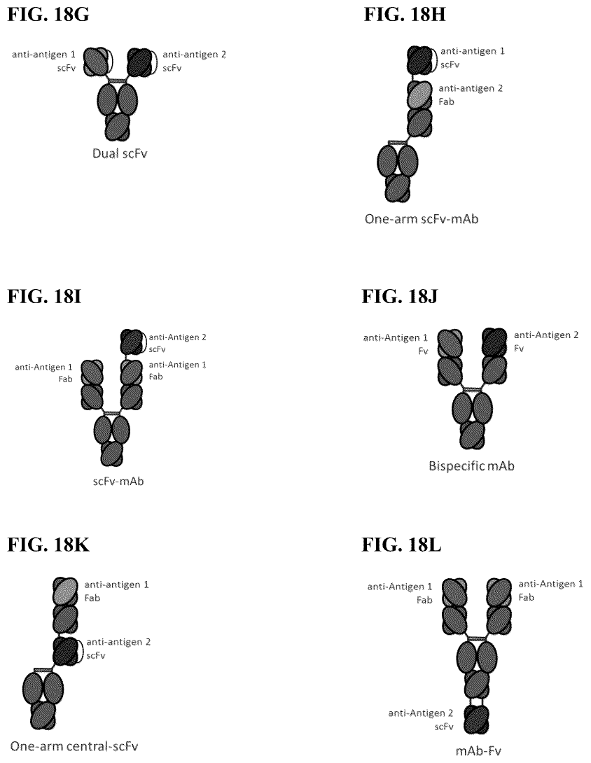

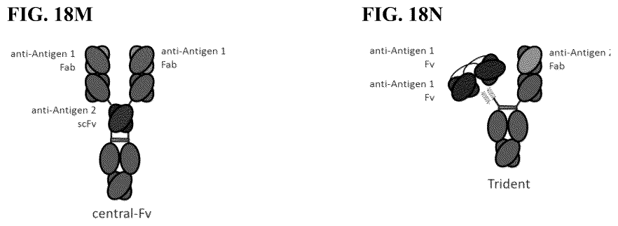

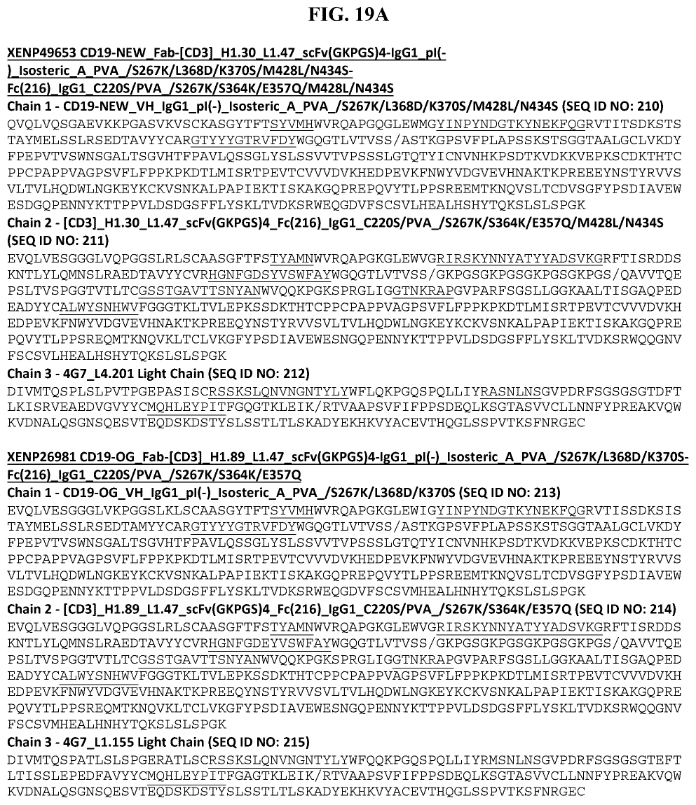

One example format utilizing Fab domains and scFv is the 1+1 Fab-scFv-Fc format (depicted schematically in FIG. 18A) which comprises a first monomer comprising a single-chain Fv (“scFv”) with a first antigen binding specificity covalently attached to a first heterodimeric Fc domain i.e., scFv-domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the heavy chain variable region.

### 3B: 2+1 Fab₂-scFv-Fc Format

Another format utilizing two Fab domains and scFv is the 2+1 Fab2-scFv-Fc format (depicted schematically in FIG. 18B) which comprises a first monomer comprising a VH-CH1 domain covalently attached to an scFv (having a first antigen binding specificity) covalently attached to a first heterodimeric Fc domain i.e., VH-CH1-first domain linked-scFv-second domain linker-CH2-CH3, a second monomer comprising a heavy chain i.e., VH-CH1-hinge-CH2-CH3, wherein the CH2-CH3 is a second heterodimeric Fc domain complementary to the first heterodimeric Fc domain, and a light chain (LC) transfected separately so that a Fab domain having a second antigen binding specificity is formed with the heavy chain variable region.

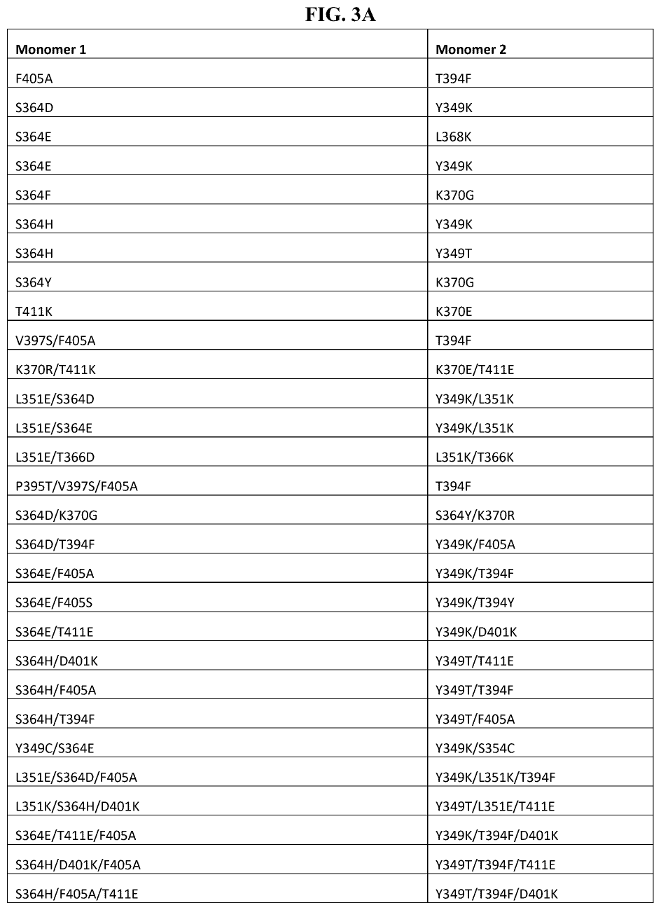

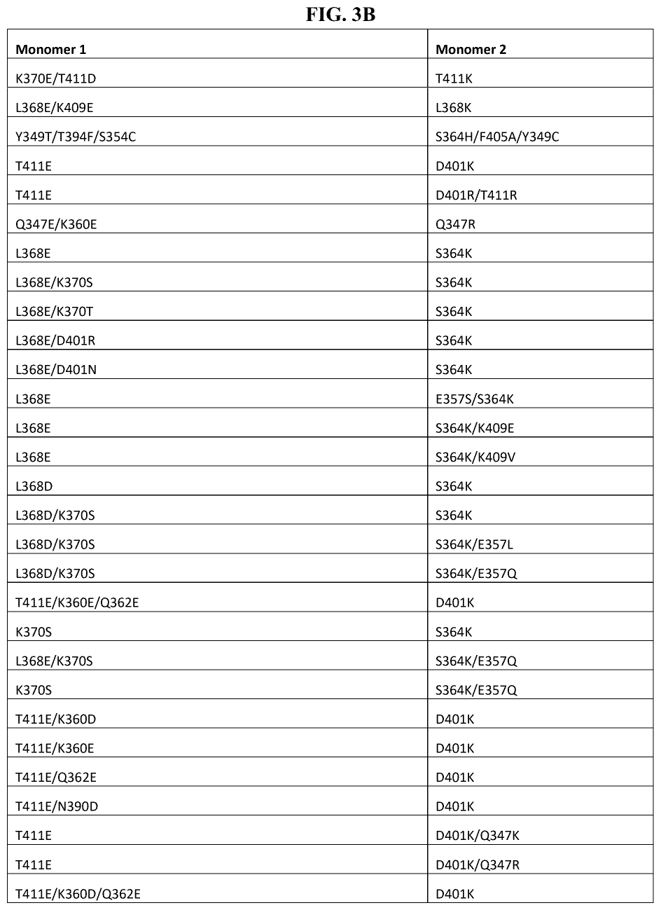

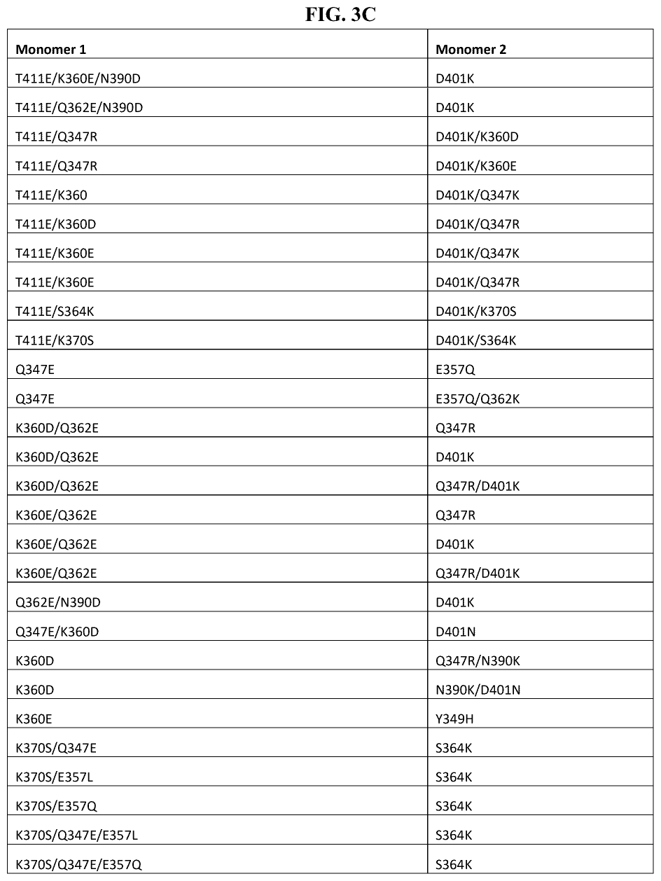

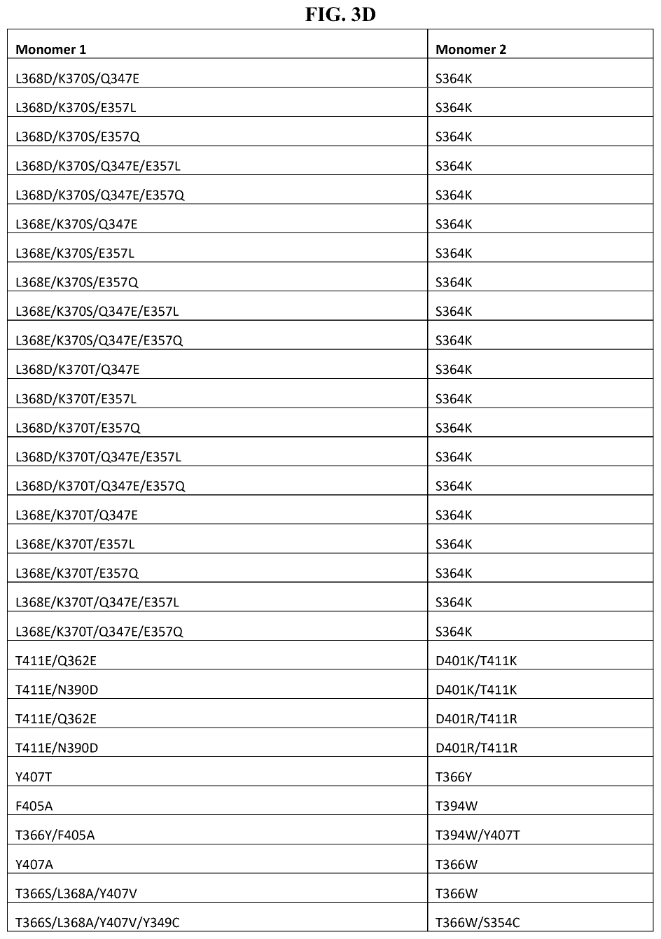

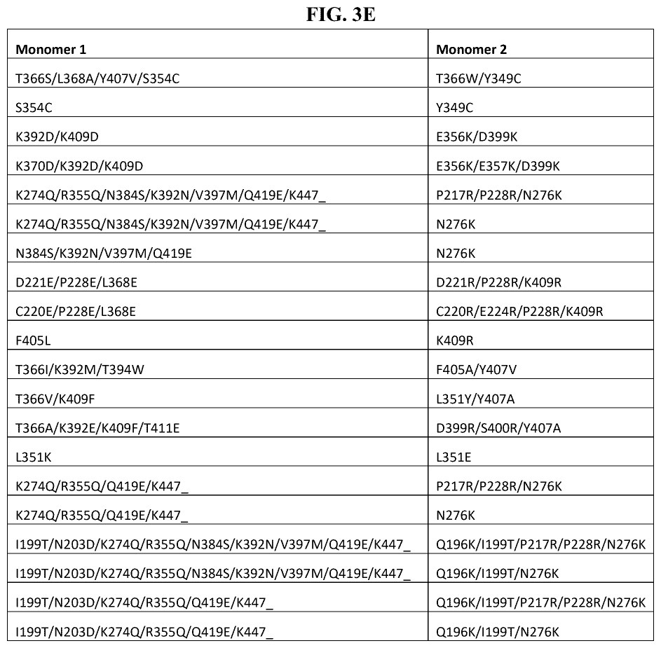

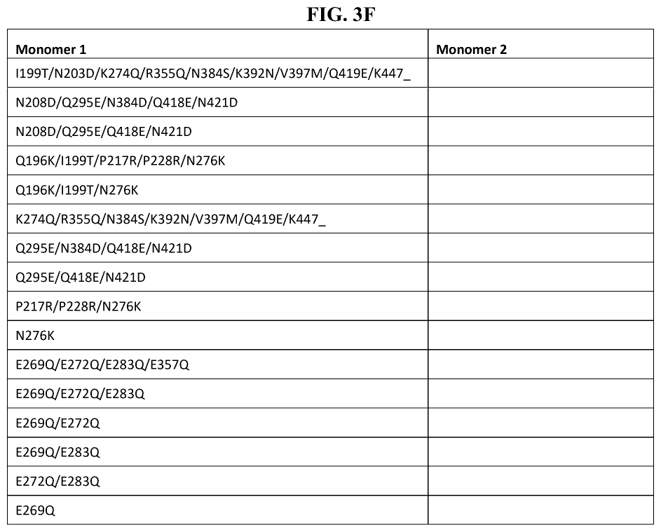

Any number of heterodimerization approaches as is known in the art could find use in this (and other) bispecific formats, in combination with any number of approaches for purifying heterodimers from contaminating homodimers, including those depicted in FIG. A-FIG. 3F. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs.3

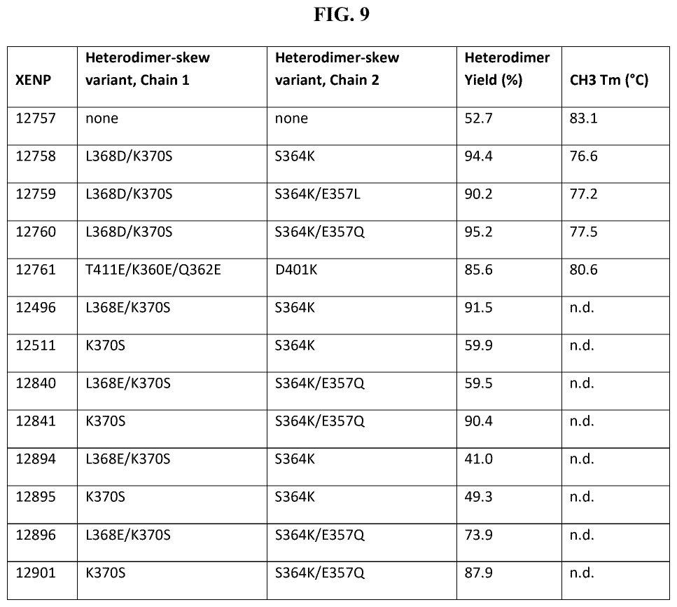

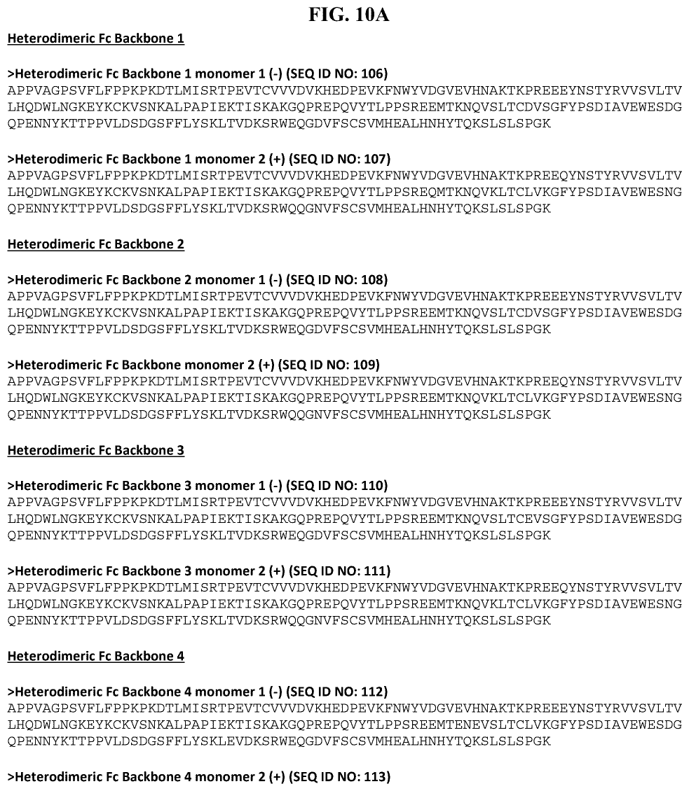

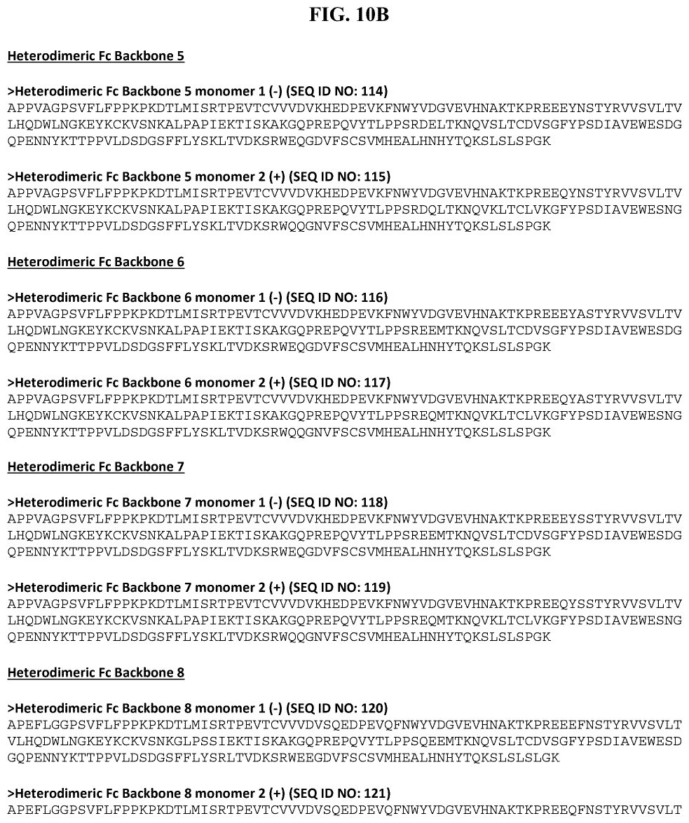

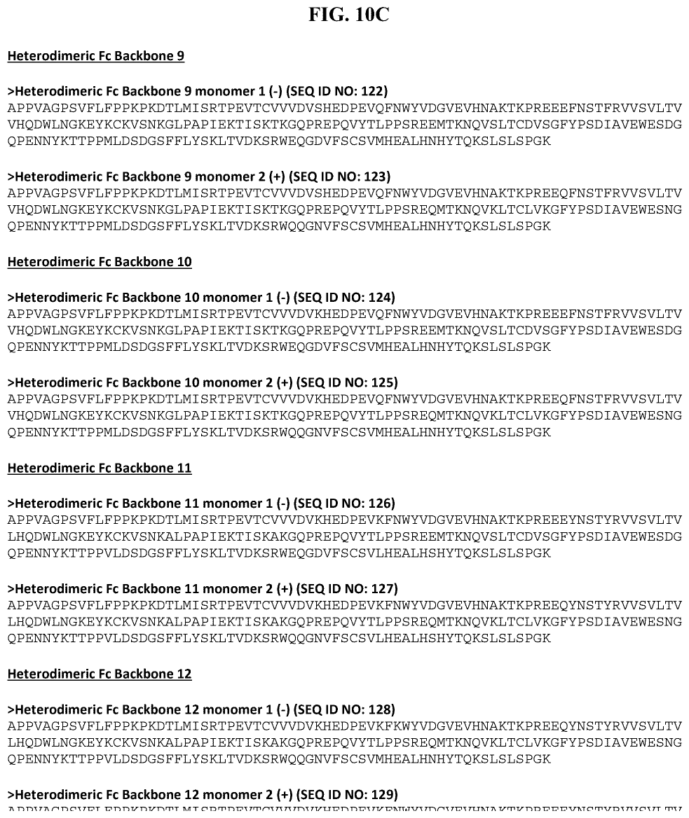

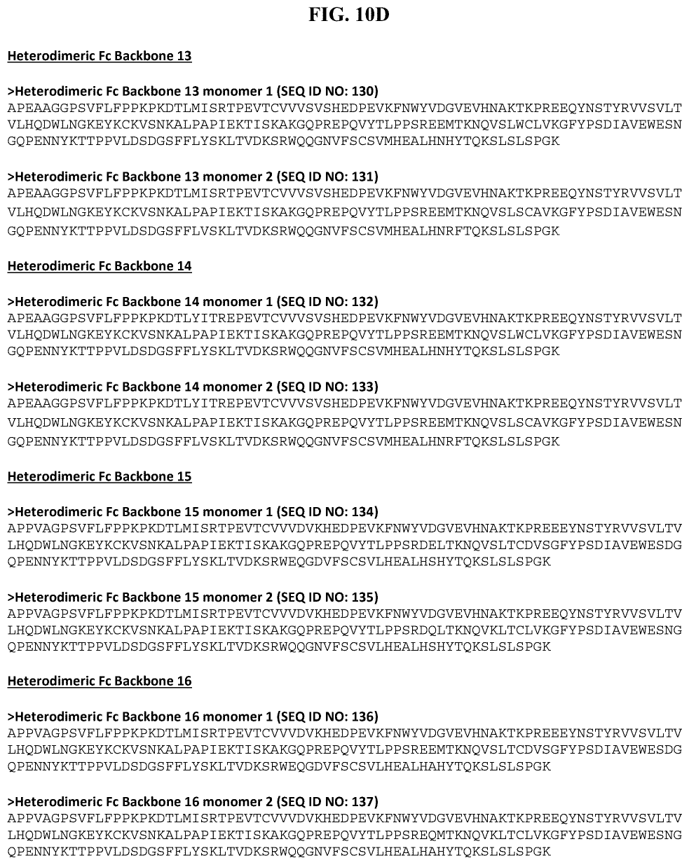

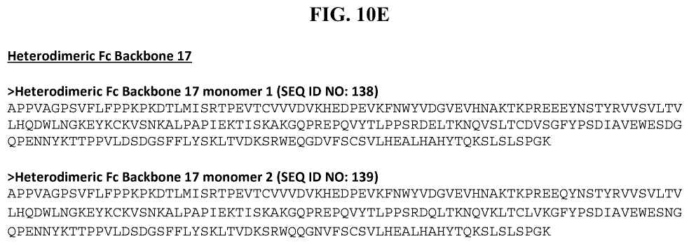

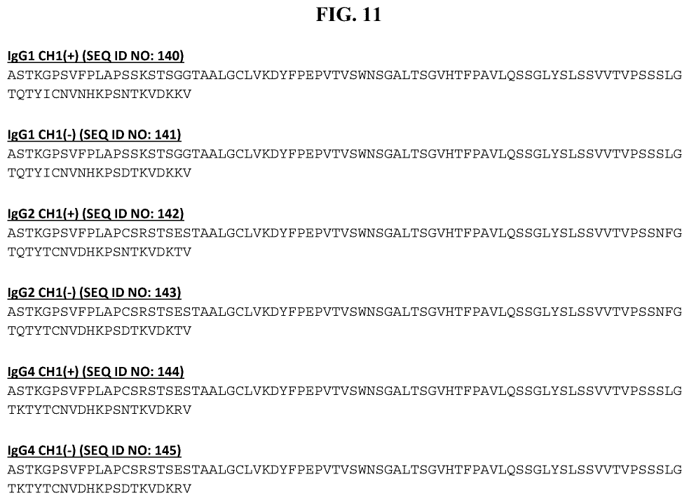

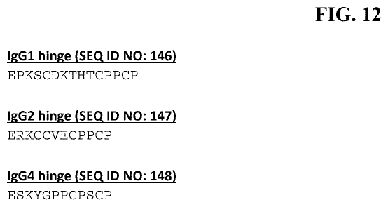

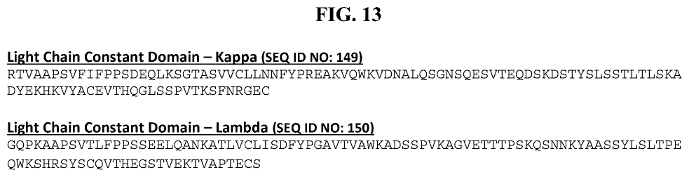

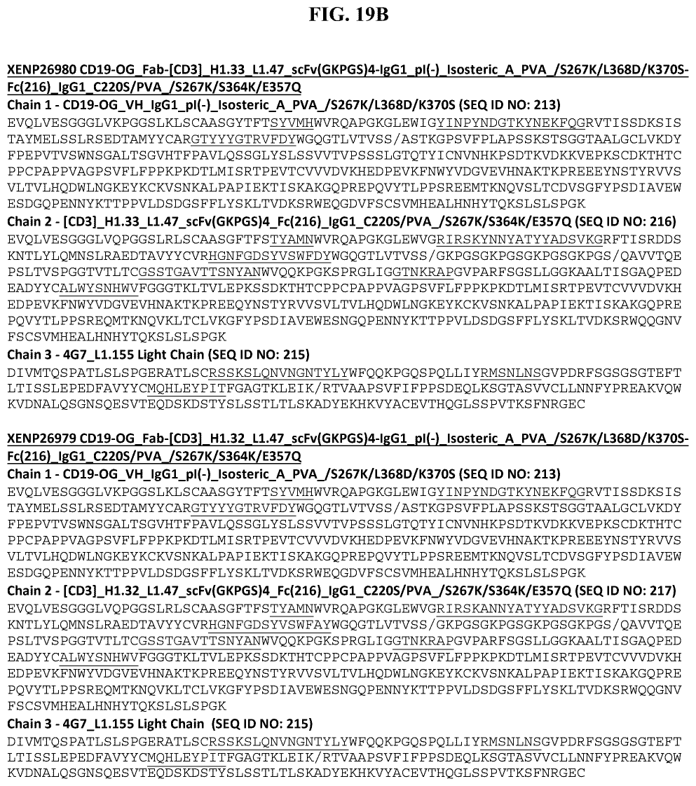

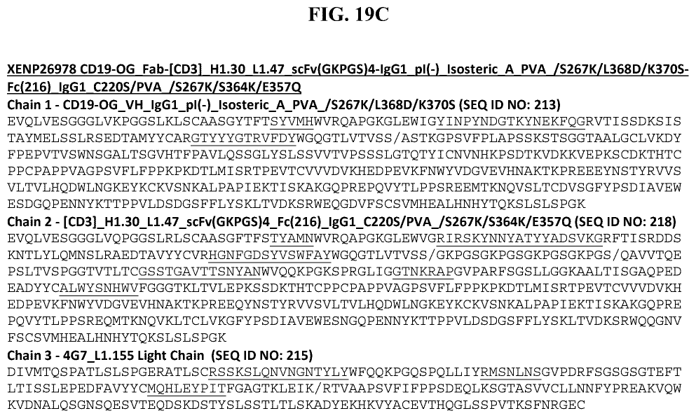

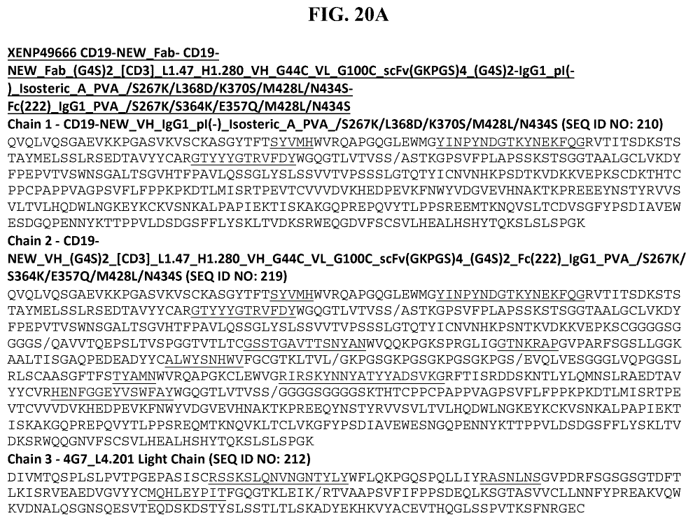

In particular, the bsAbs may utilize Backbones 1 or 11 in FIG. 9. The backbones utilize the L368D/K370S (on the HC) S364K/E357Q (on the VH-scFv-Fc or scFv-Fc) heterodimeric Fc variants. The HC side further includes pI variants N208D/Q295E/N384D/Q418E/N421D to increase negative charge of the heavy chain. The scFv utilizes a positively charged (GKPGS)₄ linker between the VH and VL domains to increase positive charge of the scFv-Fc chain. Collectively, these two approaches enable easy purification of heterodimers from contaminating homodimers. The FcγR ablation variants utilized in this platform are the E233P/L234V/L235A/G236_/S267K substitutions on both the HC and the VH-scFv-Fc or scFv-Fc monomers. In some cases, bsAbs includes the M428L/N434S half-life extension variants. Sequences for illustrative CD19×CD3 bsAbs in the 1+1 Fab-scFv-Fc and in the 2+1 Fab₂-scFv-Fc formats are depicted respectively in FIG. 19A-FIG. 19C and FIG. 20A-FIG. 20AD.

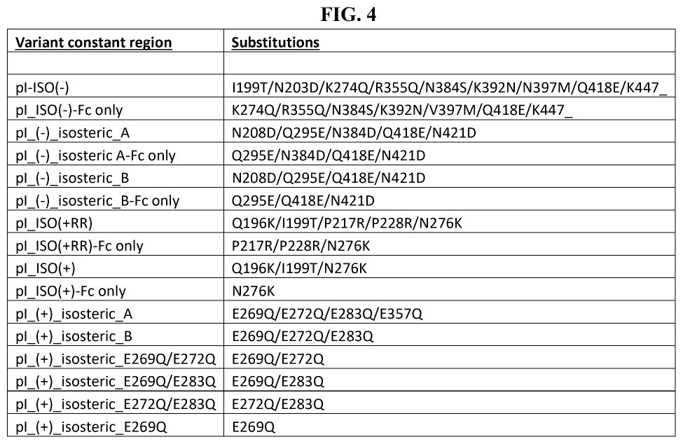

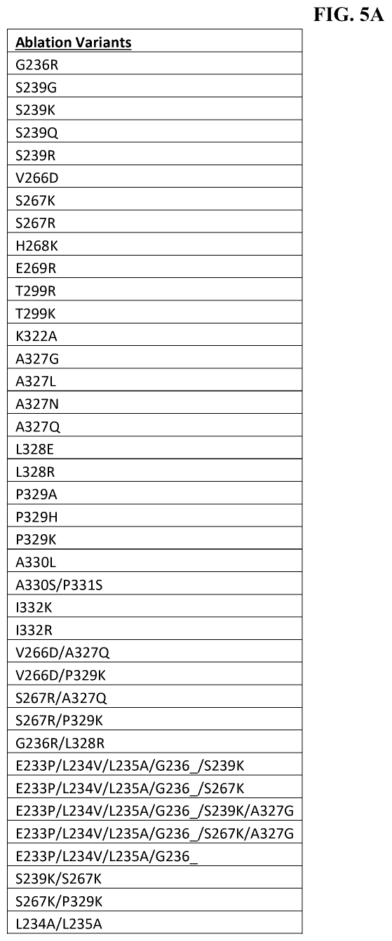

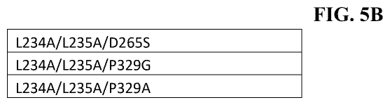

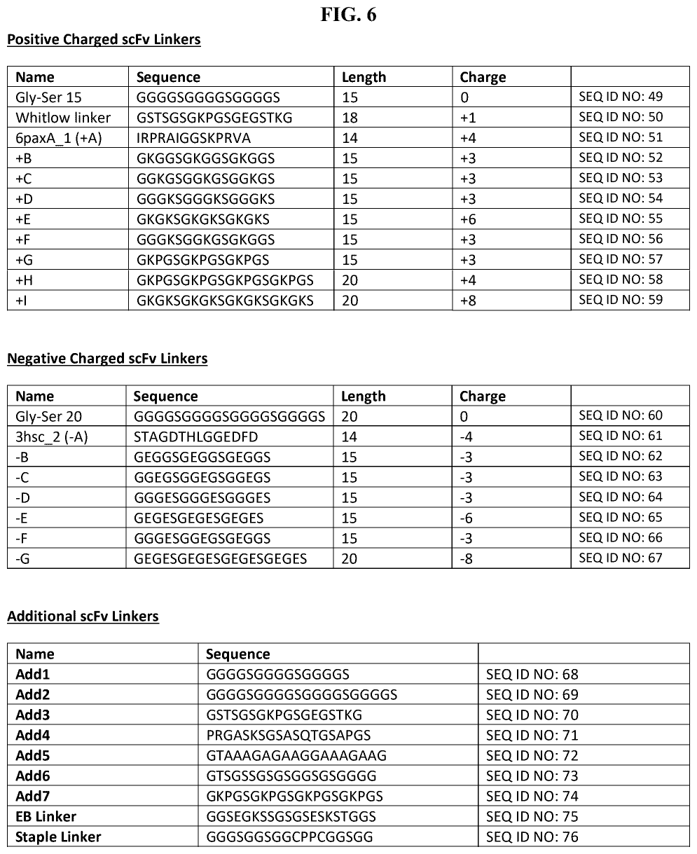

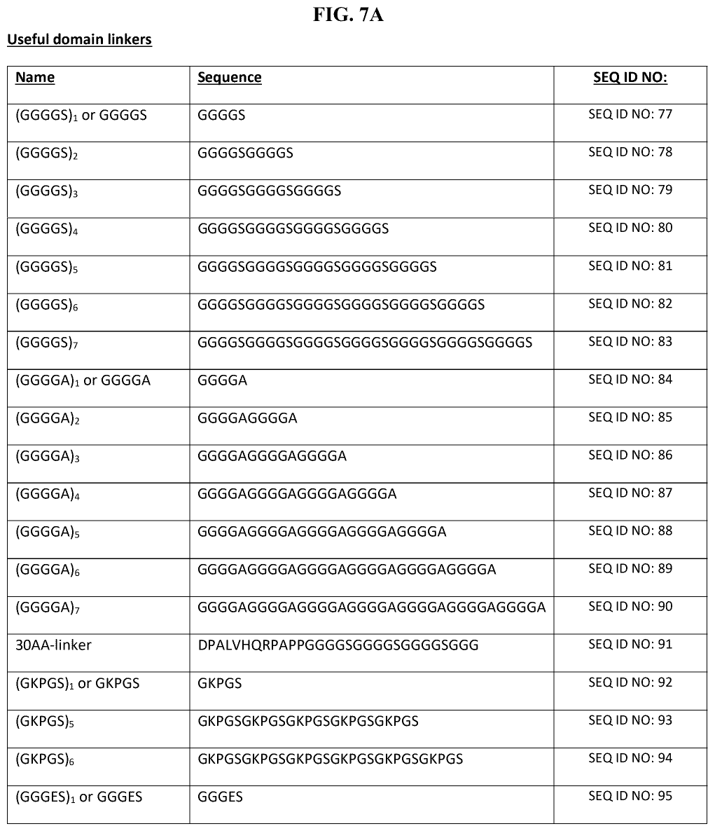

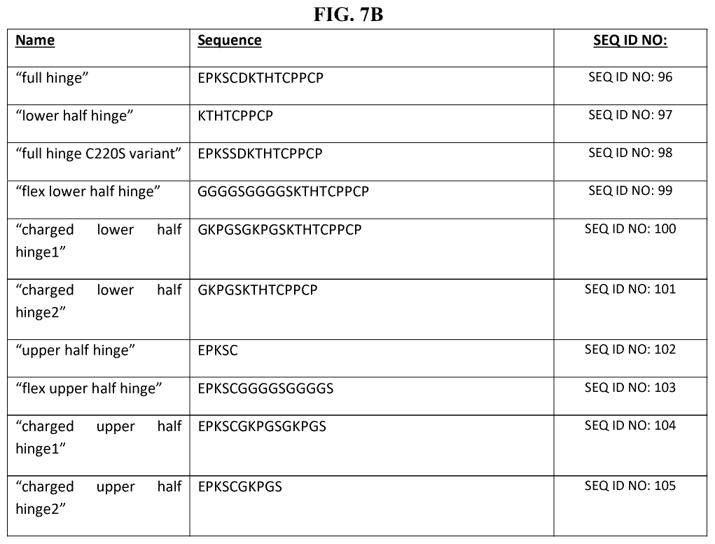

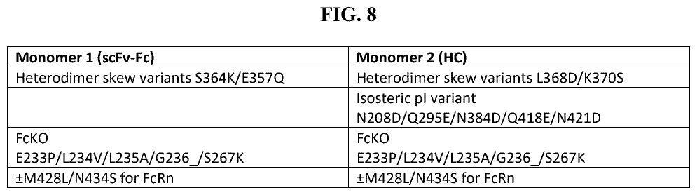

Although the above provide particularly useful backbones, any number of heterodimerization approaches as is known in the art could find use in the bispecific formats, in combination with any number of approaches for purifying heterodimers from contaminating homodimers, including those depicted in FIG. 3A-FIG. 3F and FIG. 4. Any of the number of linkers as is known in the art may find use in linking the VH and VL domains of the scFv. Finally, it may be useful to maximize serum half-life of the bsAbs, and any of the number of half-life extending variants as is known in the art may find use in these bsAbs. Regardless of bsAb format, the CD28 bispecific antibodies are monovalent for CD28 and incorporate Fc variants engineered to ablate FcγR binding to avoid potential superagonism. Such Fc variants include those depicted in FIG. 5A and FIG. 5B.

### Example 4: CD19×CD3 bsAbs Deplete CD19⁺ Cells In Vitro

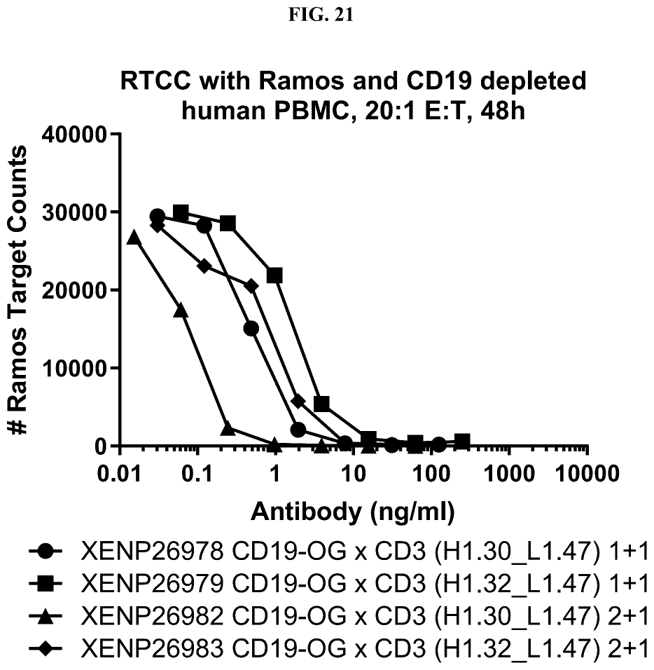

In a first experiment, first generation CD19×CD3 bsAbs (utilizing the CD19-OG binding domain and the H1.30_L1.47 or H1.32_L1.47 CD3 binding domains; sequences for which are depicted in FIG. 19A-FIG. 19C and FIG. 20A-FIG. 20AD) were incubated with CD19⁺ Ramos lymphoma cell line and CD19 depleted human PBMCs at a 10:1 effector:target ratio for 48 hours. The data as depicted in FIG. 21 show that the CD19×CD3 bsAbs exhibit highly efficacious cytotoxicity for CD19⁺ human lymphoma line.

### Example 5: CD19×CD3 bsAbs Deplete Peripheral B Cell In Vivo

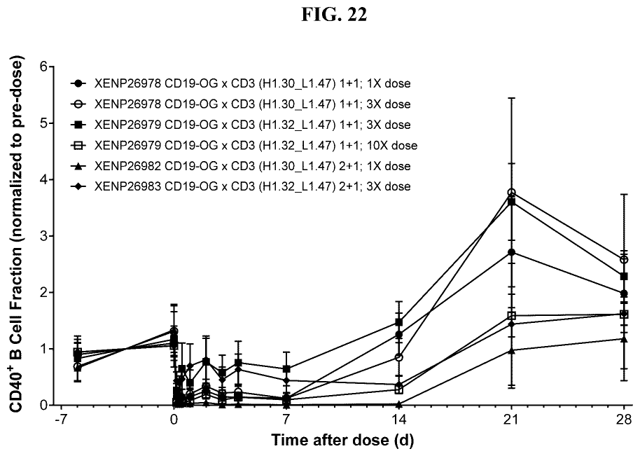

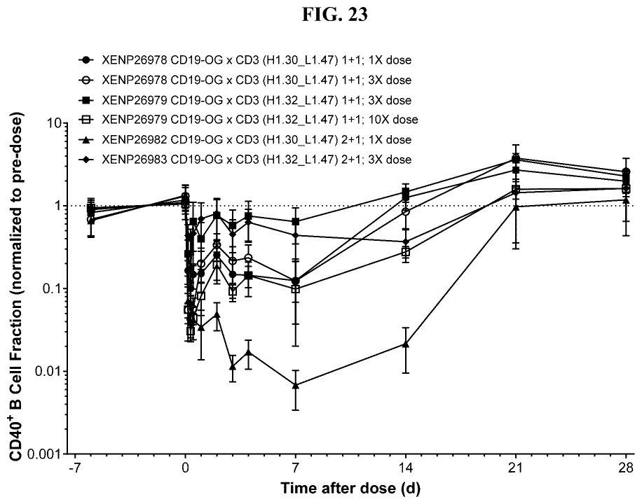

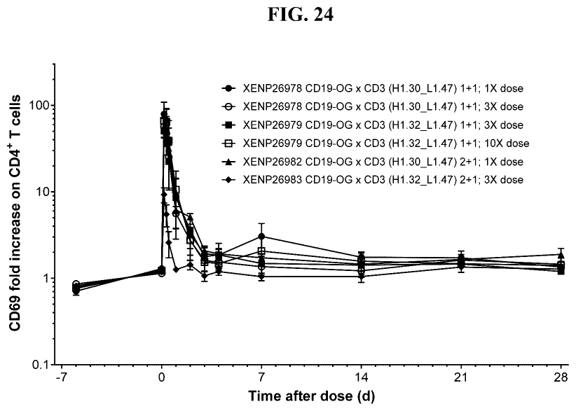

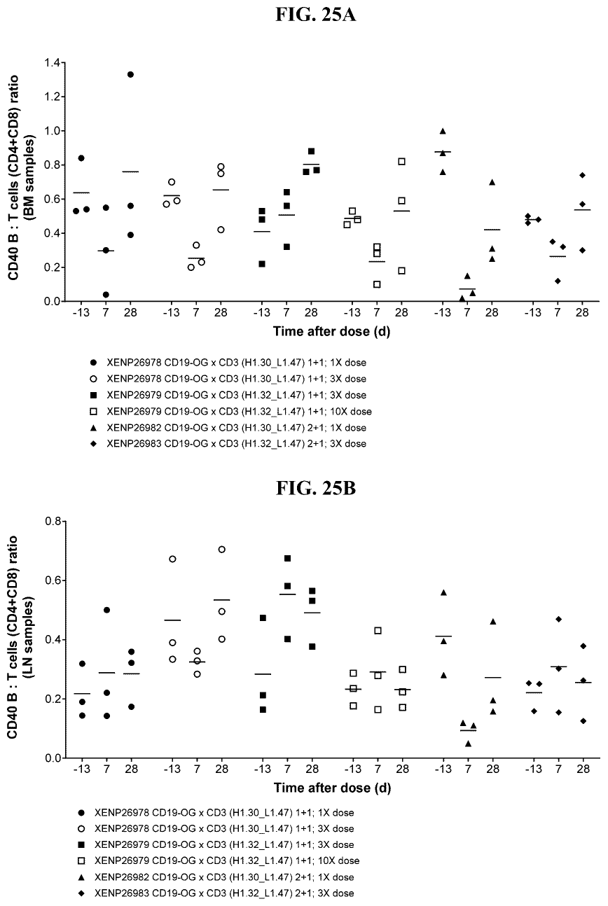

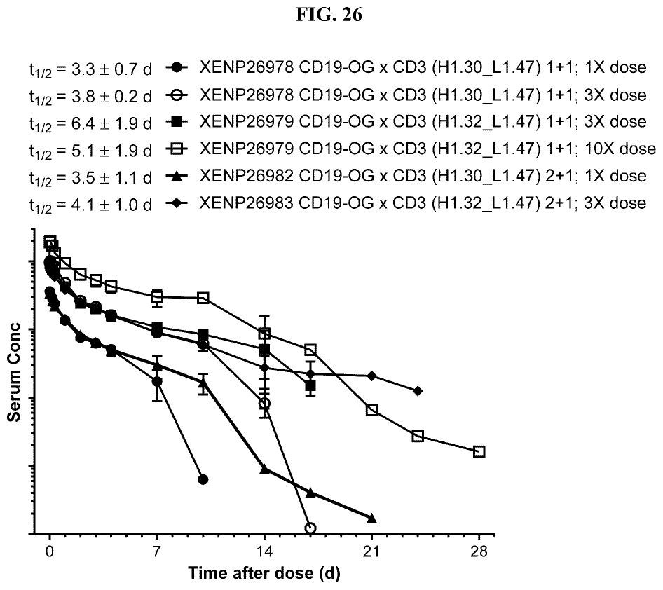

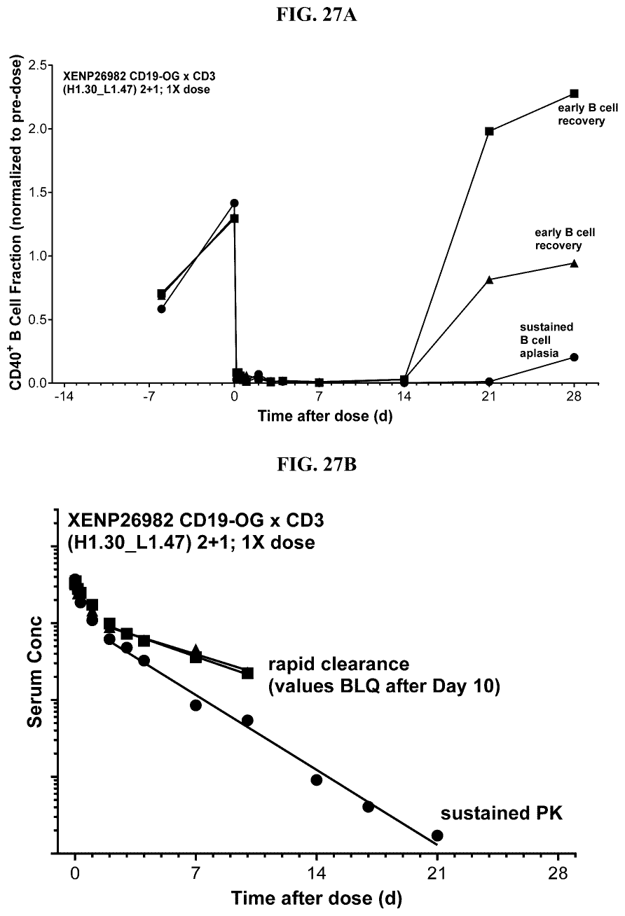

Next, activity of the first generation CD19×CD3 bsAbs were investigated in vivo. Cynomolgus monkeys were dosed with various concentrations of the bsAbs, and peripheral, bone marrow, and lymph node B cell depletion was assessed (along with peripheral T cell activation, IL-6 release, and pharmacokinetics). The data as depicted in FIG. 22, FIG. 23, FIG. 25A, and FIG. 25B, show that B cell depletion was achieved with all of the CD19×CD3 bsAbs, although most robust with the 2+1 Fab₂-scFv-Fc format with CD3 High (XENP26982) which almost completely depletes B cells out to 14 days after dosing. T cell activation was similar among the bsAbs, although IL-6 release was consistently higher for bsAbs utilizing CD3 High (FIG. 24A and FIG. 24B). In data not shown, both CD4+ and CD8⁺ T cells exhibit similar trends in activation (CD69 and CD25). As depicted in FIG. 26, bsAbs could be measured in serum up to 4 weeks after dosing, although pharmacokinetics showed signs of ADA- or TMDD-mediated rapid clearance at ˜7-14 days. Notably as depicted in FIG. 27A and FIG. 27B, rapid clearance of CD19×CD3 bsAb coincide with early B cell recovery.

### Example 6: Re-Engineering CD19 Binding Domain

As described in Example 5, PK showed potential signs of ADA (albeit in cynomolgus monkeys), and so it may be important to modulate immunogenicity of the bsAbs. Accordingly, the CD19-OG binding domain was re-humanized (FIG. 28A and FIG. 28B). As depicted in FIG. 29, germline identities and 9mers were increased across both the VH and VL, while maintaining binding affinity. Another important property is the stability of the binding domains, especially if the CD19×CD3 bsAbs are to be used in a high concentration subcutaneous formulation. As depicted in FIG. 29, the re-humanized CD19 binding domain had +7° C. improved Tₘ. Notably, the A100Q substitution in the VL FR4 alone improved stability by 1° C. and removed a predicted MHC-binding peptide (data not shown). Additionally, a VL CDR2 methionine that may be liable to oxidation was substituted with alanine. Notably, the M51A substitution did not damage binding affinity. Collectively, the re-engineered CD19 binding domain contains 33 amino acid substitutions relative to the parental sequence.

### Example 7: Re-engineering CD3 Binding Domain

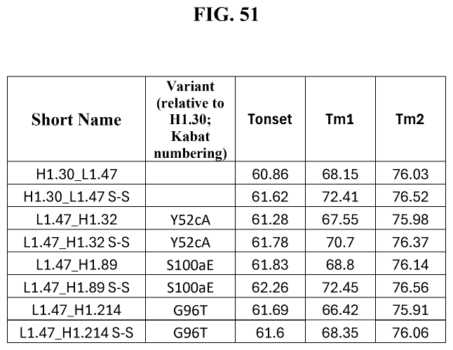

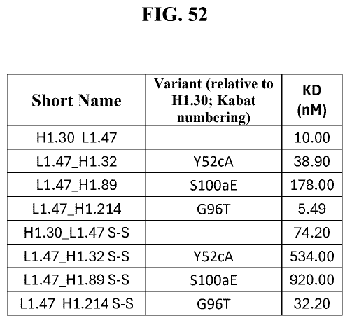

As described above, improving stability of the binding domains is important if the CD19×CD3 bsAbs are to be used in high concentrations subcutaneous formulations. Accordingly, the CD3 binding domains were engineered with disulfide bridges to improve stability in the context of an scFv. Data as depicted in FIG. 51 show that the engineered disulfide bridges increase Tm1 by ˜2-4° C. While the engineered disulfide bridges did result in increased stability, there was also some loss of affinity as depicted in FIG. 52.

Additionally, the CD3 binding domains utilized in Examples 4 and 5 include a non-canonical deamidation motif GNF at positions 96-98 (Kabat numbering) in the VH CDR3. In a first round of engineering, variants were made to remove this deamidation liability as well as to engineer stabilizing disulfide bridges. In data not shown from initial engineering, N97 was determined to be important for binding, but it was found that variants at N97 abrogated binding affinity (with the exception of N97Q which retains some binding). Variants at G96 was also important to binding, and G96E or G96T was found to improve binding affinity (albeit with decrease in stability by ˜2-4° C., as depicted in FIG. 51). And finally it was found that D100G improves affinity. Based on these (and the baseline that Y52cA and S100aE were affinity modulating variants already included in H1.32 and H1.89 respectively relative to H1.30), further variants were engineered to fix the deamidation and incorporate disulfide bridges while maintaining an affinity ladder similar to the original 3×CD3 binding domains and improved stability. Sequences for these variants are depicted in FIG. 17A-FIG. 17J, and affinity (in the context of 2+1 Fab₂-scFv-Fc molecules of FIG. 20A-FIG. 20AD) are depicted in FIG. 30. The data show that some of the re-engineered CD3 binding domains had binding affinity similar to the original CD3 binding domains, albeit with removal of the deamidation motif and introduction of stabilizing disulfide bridges.

### Example 8: Optimizing CD19×CD3 bsAbs

CD19×CD3 bsAbs were enhanced using the novel CD19 binding domains described in Example 6, novel CD3 binding domains in Example 7, and half-life extension Fc variants, sequences for which are depicted in FIG. 19A-FIG. 19C and FIG. 20A-FIG. 20AD.

### Example 8A: In Vitro Assessment

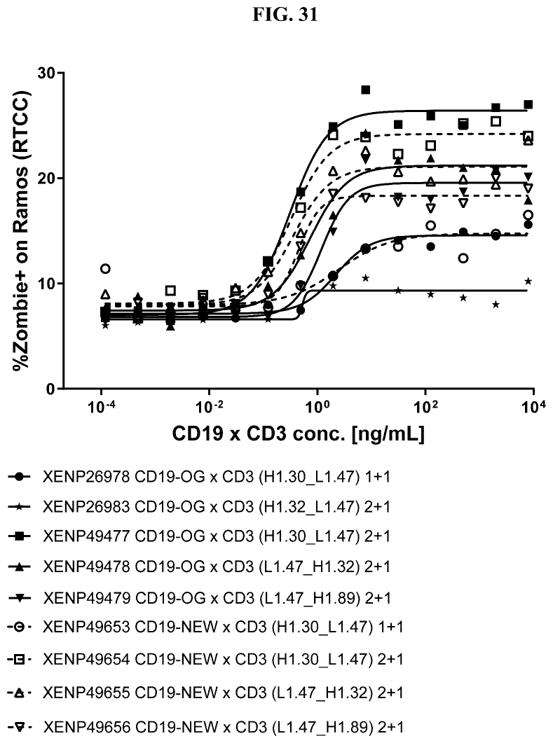

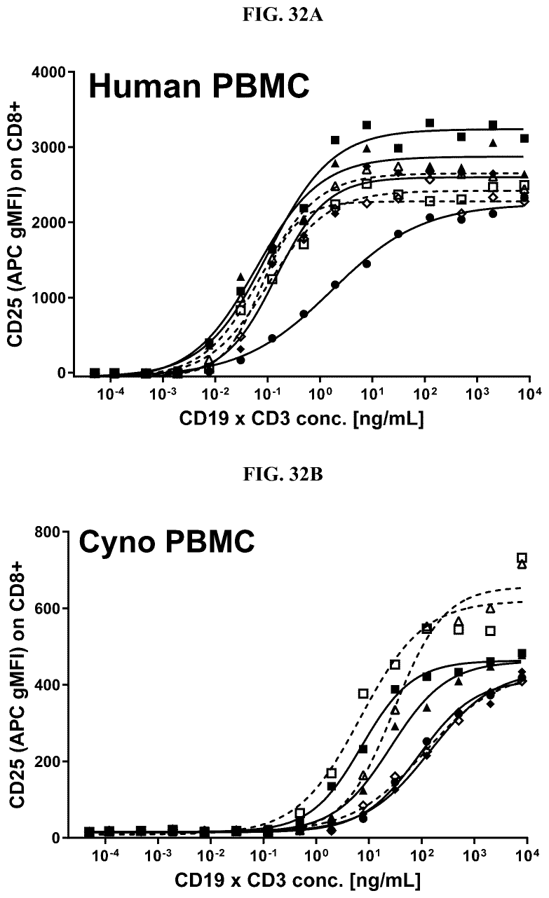

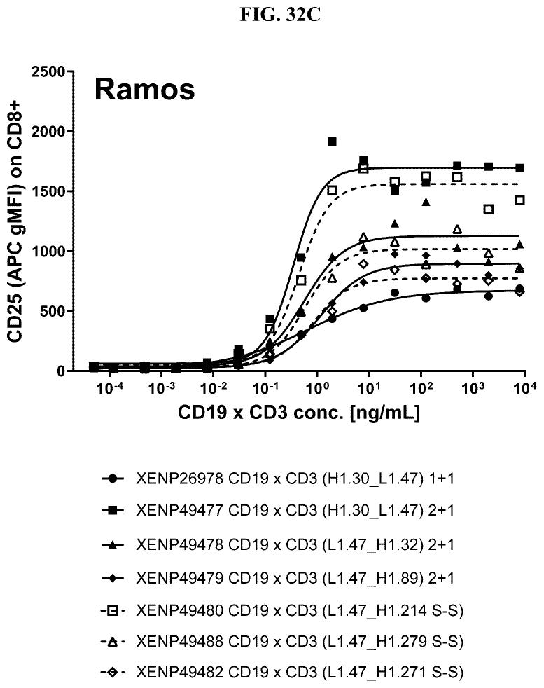

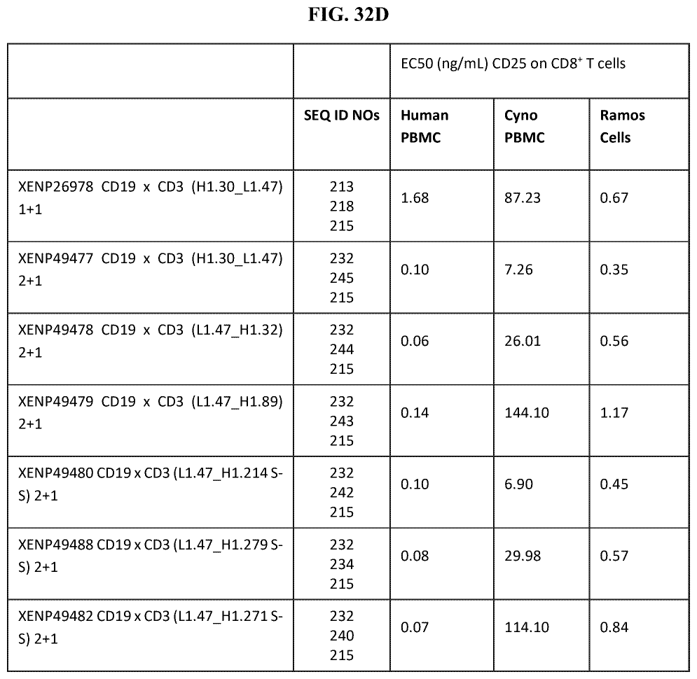

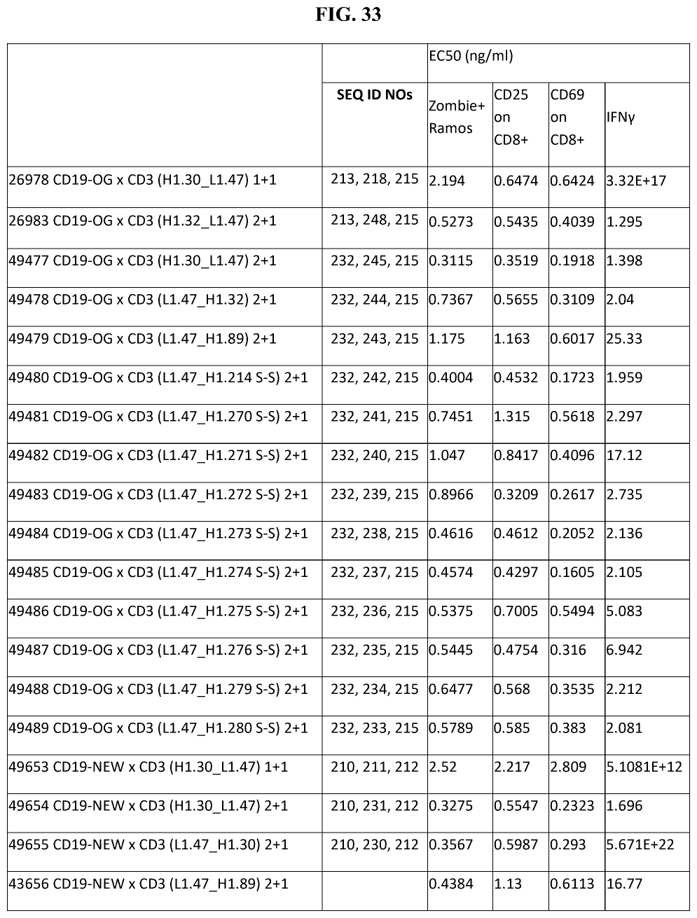

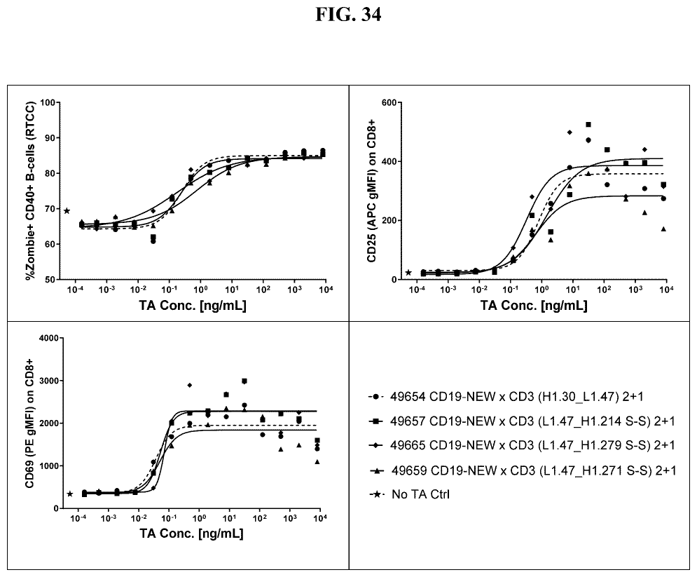

Activity of the first generation and enhanced CD19×CD3 bsAbs were assessed to determine whether the re-engineered CD19 and CD3 binding domains impacted activity. As before, the bsAbs were incubated with CD19⁺ Ramos cells as well as human or cynomolgus PBMCs. Data as depicted in FIG. 31, FIG. 32A-FIG. 32D, FIG. 33, and FIG. 34 show that bsAbs with the re-engineered binding domains have equivalent activity to the first generation bsAbs (with respect to target cell kill and T cell activation).

### Example 8B: In Vivo Assessment

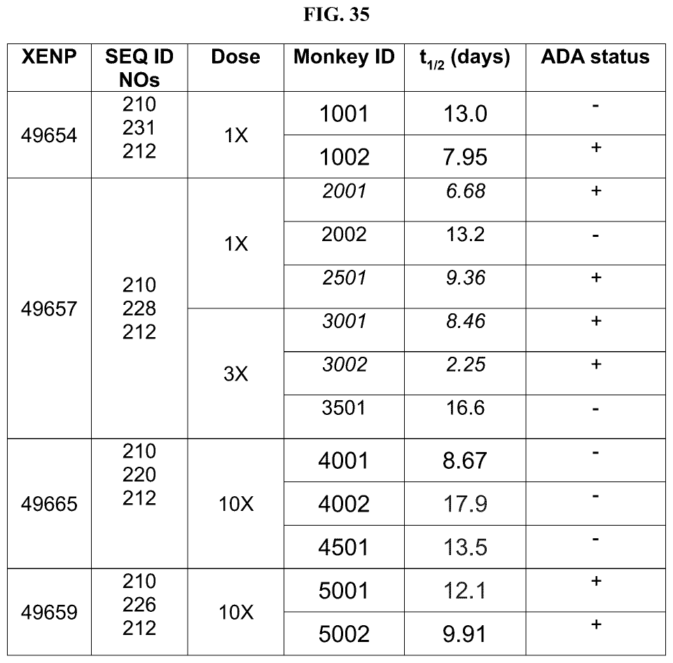

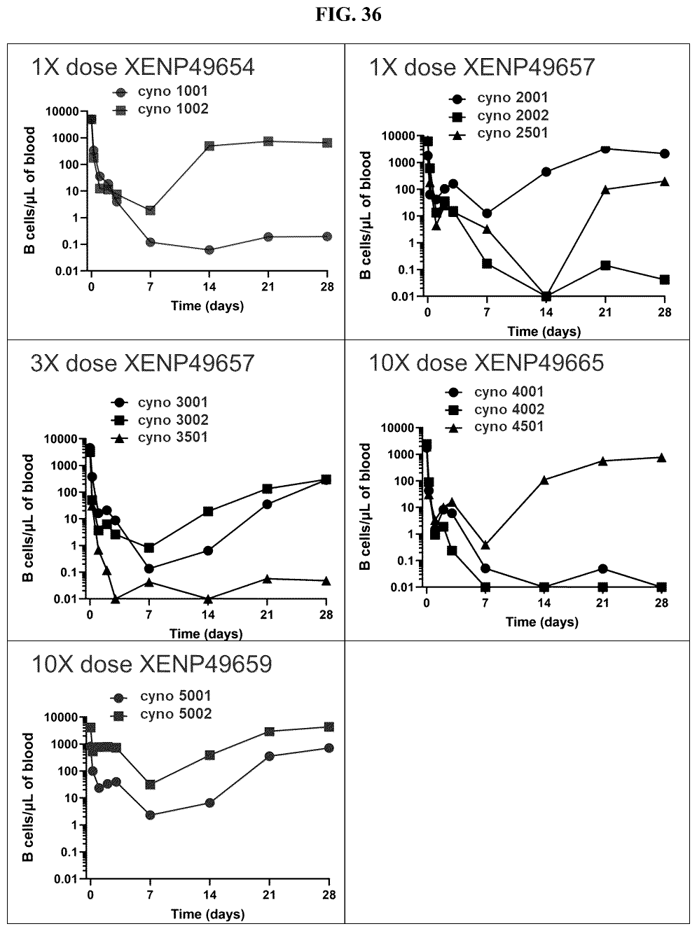

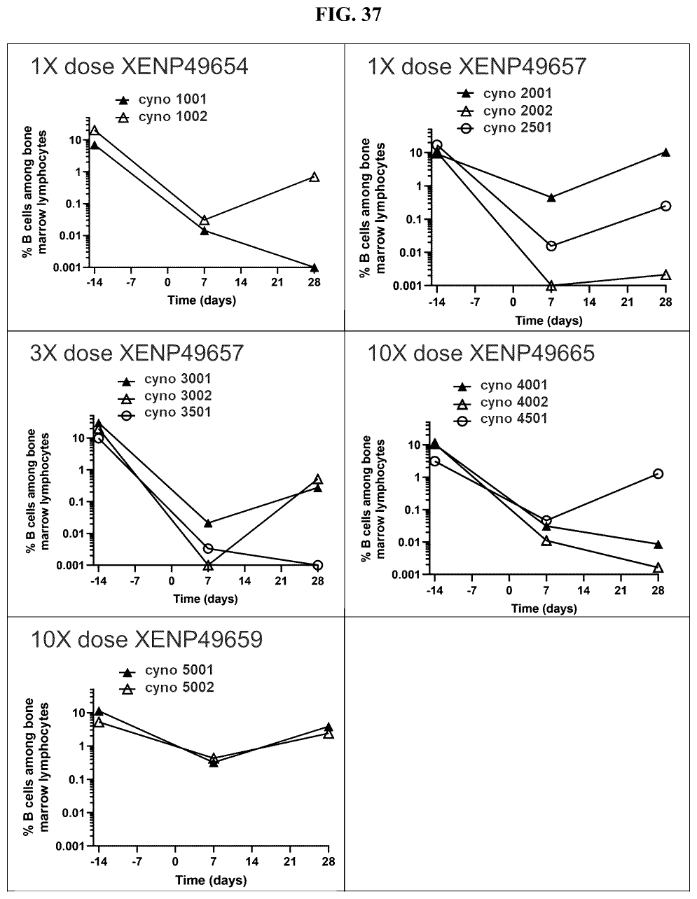

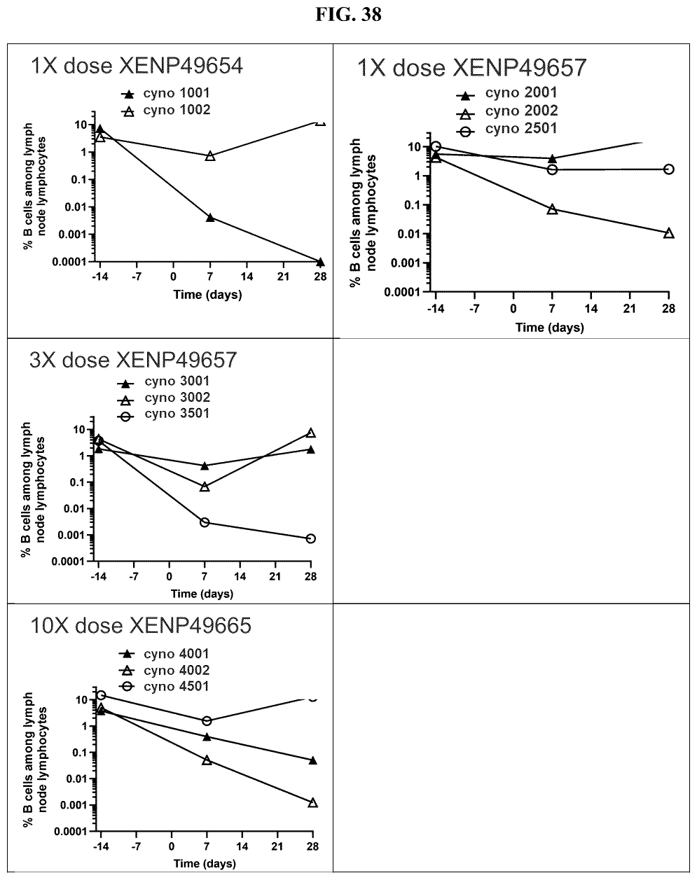

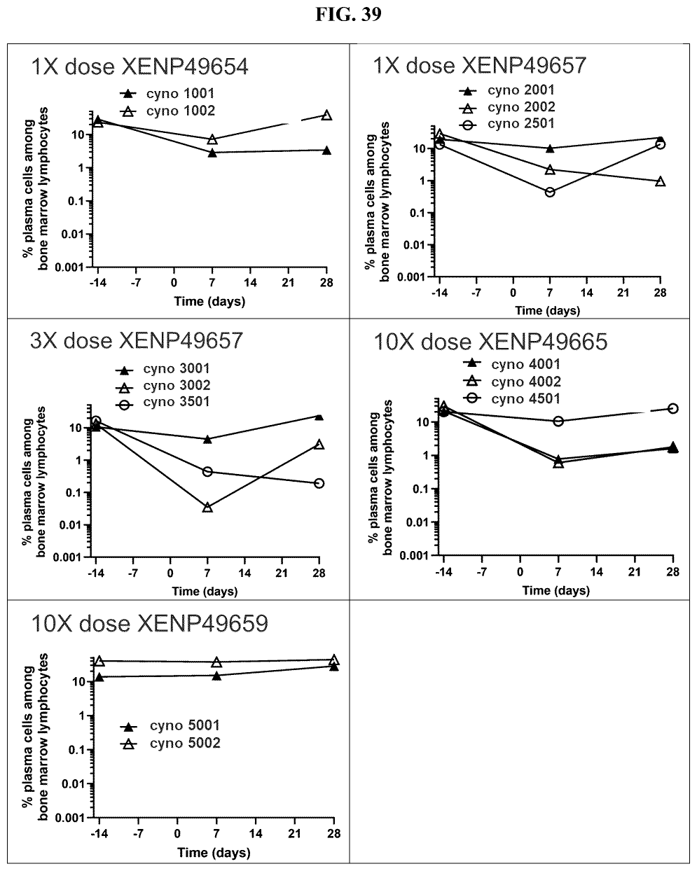

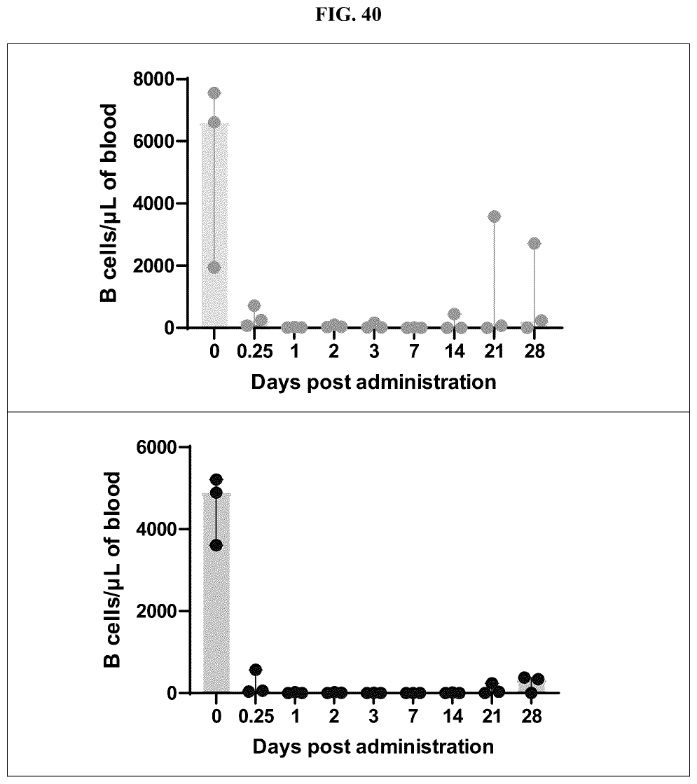

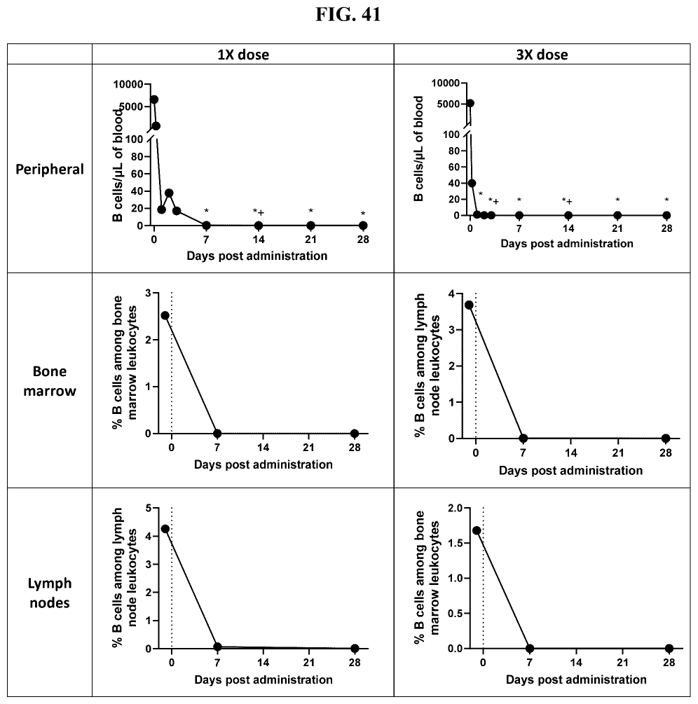

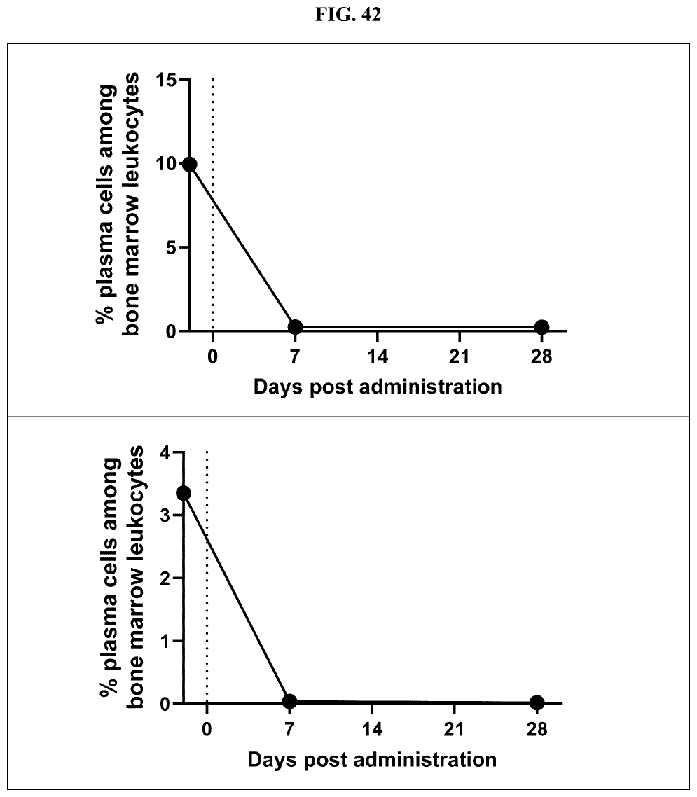

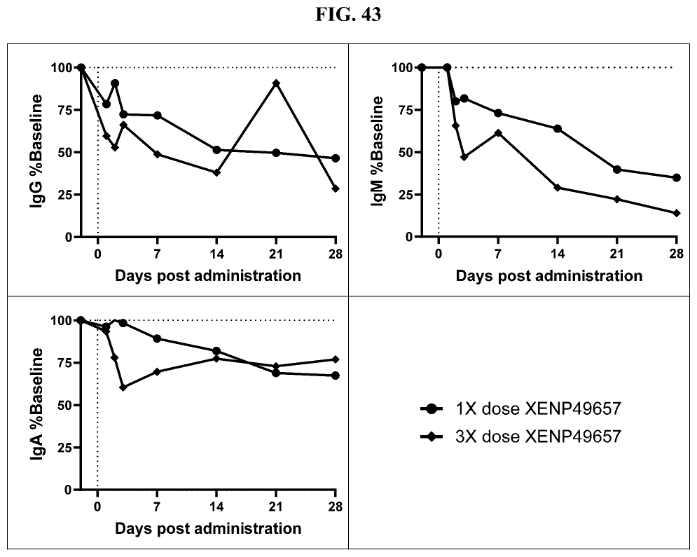

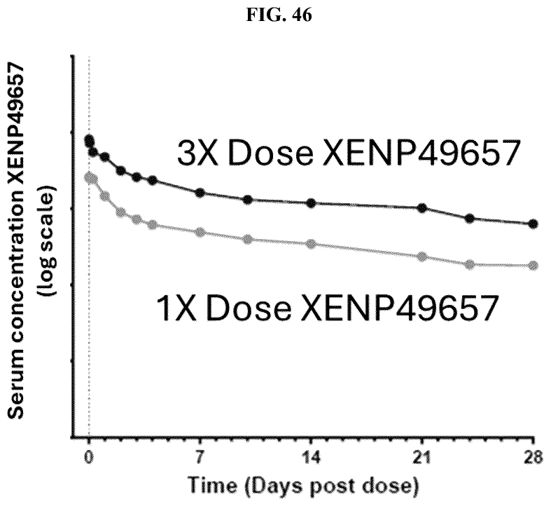

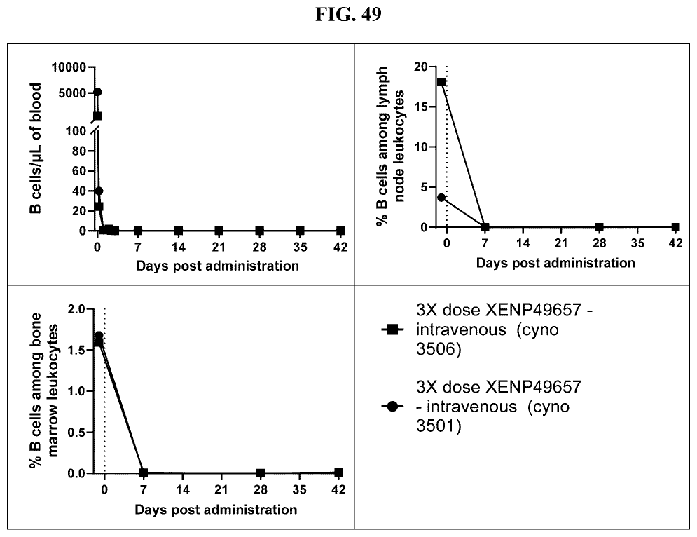

The activity of the enhanced CD19×CD3 bsAbs were investigated in vivo. In a first study, cynomolgus monkeys were dosed intravenously with various concentrations (low 1× dose and high 3× dose) of the bsAbs, and peripheral, bone marrow, and lymph node B cell plasma depletion and pharmacokinetics was assessed, data for which are depicted in FIG. 35-FIGS. 43, 46, and 49 (cyno 3501 and 3506 are from separate studies). As before, the study was impacted by ADA across the bispecific antibodies, so animals impacted by ADA are denoted in FIG. 35. Half-life estimated were generally consistent between compartmental and non-compartmental analysis. Noting only the animals without ADA, XENP49657 has better half-life than XENP49654 (13.2 and 16.6 days vs. 13.0 days). Notably in cynomolgus monkey 1001 and 2002 which were not impacted by ADA, respectively dosed with bsAbs having old and new CD3 binding domain, depletion of B cells and plasma cells were similar. For cynomolgus monkeys 2002 and 3501 which were not impacted by ADA, both dosed with XENP49657 respectively at 1× and 3× dose, B cell depletion in peripheral blood was sustained for at least 28 days. Although XENP49665 also demonstrated effective depletion of B cells, it was dosed at much higher relative concentration, while XENP49659 demonstrated less B cell depletion despite 10× dose. Further as depicted in FIG. 38 and FIG. 39, the CD19×CD3 bsAbs, particularly XENP49657, promoted deep B cell depletion in bone marrow and lymph nodes. As depicted in FIG. 42, the bsAbs also deplete CD19+ plasma cells in bone marrow. As depicted in FIG. 43, XENP49657 significantly reduces serum immunoglobulin. As depicted in FIG. 46, XENP49657 exhibited half-life up to ˜15 days. Notably as depicted in FIG. 49, single intravenous dose XENP49657 results in deep B-cell depletion for at least 42 days.

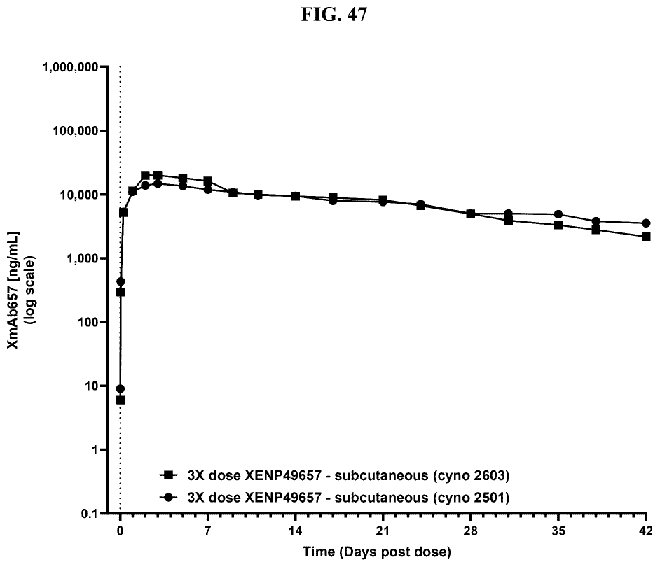

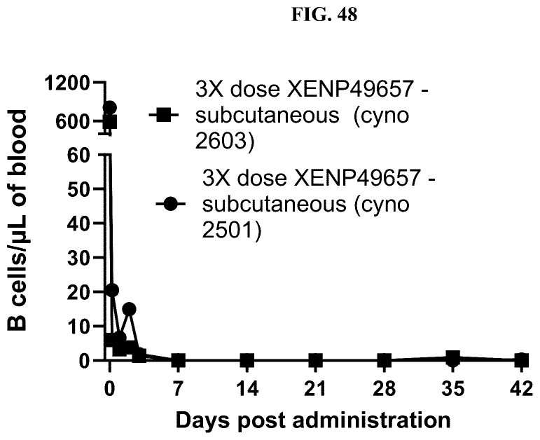

In a second study, cynomolgus monkeys were dosed subcutaneously with high 3× dose of XENP49657, and peripheral B cell depletion and pharmacokinetics were assessed, data for which are depicted in FIG. 47 and FIG. 48. Consistent with intravenous dosing, single subcutaneous dose XENP49657 results in deep B-cell depletion sustained for at least 42 days, and half-life up to ˜15 days.

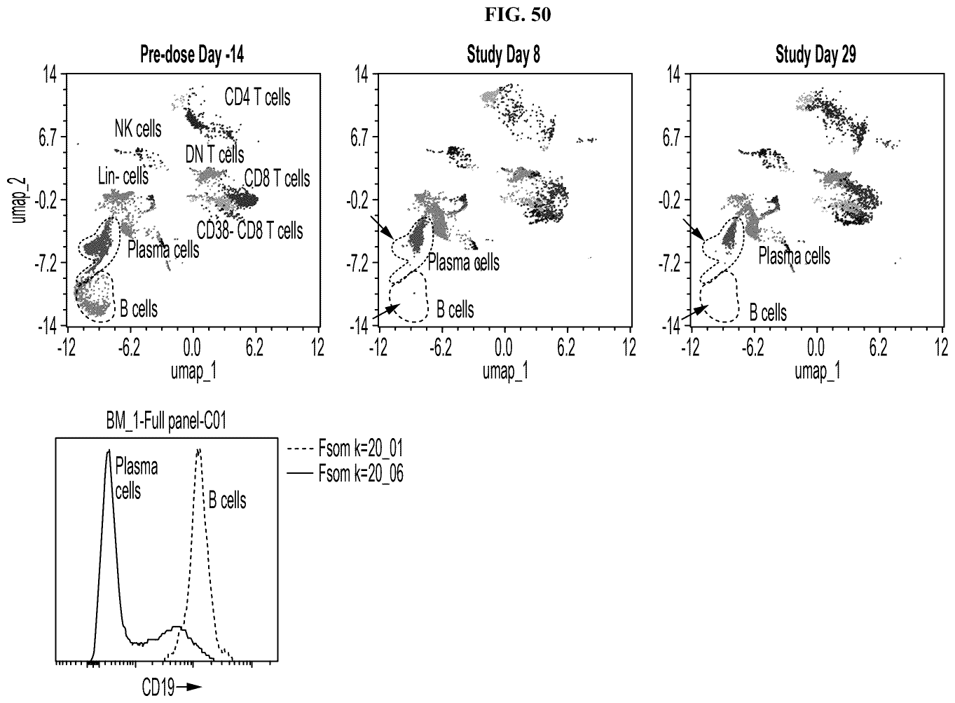

As depicted in FIG. 50, XENP49657 promotes depletion of CD19+ plasma cells in bone marrow.

### Example 9: Further Biophysical Characterization of Enhanced CD19×CD3 bsAbs

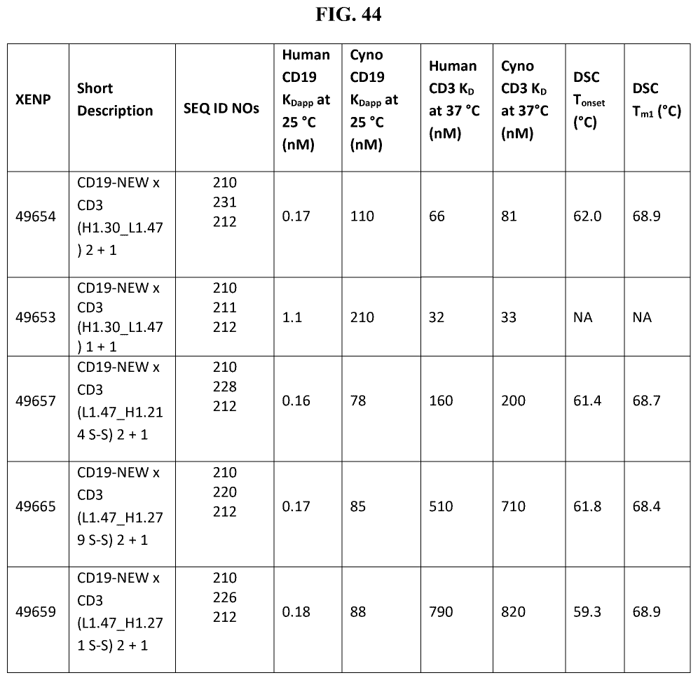

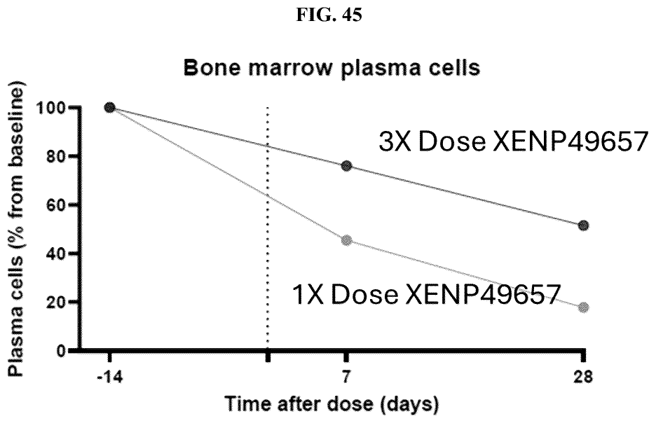

Affinity for human and cyno CD19 and CD3 were determined as follows. For CD19, human and cyno CD19-His were captured onto a CD5 chip pre-conjugated with anti-His capture mAb. Each antigen was captured at different density in order to achieve the desired response ranges suitable for kinetics. Dilution series of antibody were followed over each surface at 25° C. For CD3, amine couple human and cyno cd3/e were used, and bispecific antibodies were flowed at various concentrations. Melting temperature for the bispecific antibodies were also determined using DSC methods. Temperature was ramped from 25° C. to 95° C. at a rate of 1° C./min, and date were fit to a non-2 state model, with 3 transitions when appropriate. Data are collectively shown in FIG. 44.

Finally (data not shown), CD19×CD3 bsAb XENP49654 having the old CD3 binding domain was found to aggregate at higher concentrations, while XENP49657 and XENP49665 having stability-enhanced CD3 binding domains did not show such behavior.

### Example 10: CD19×CD3 bsAbs Deplete B Cells from Patients with RA

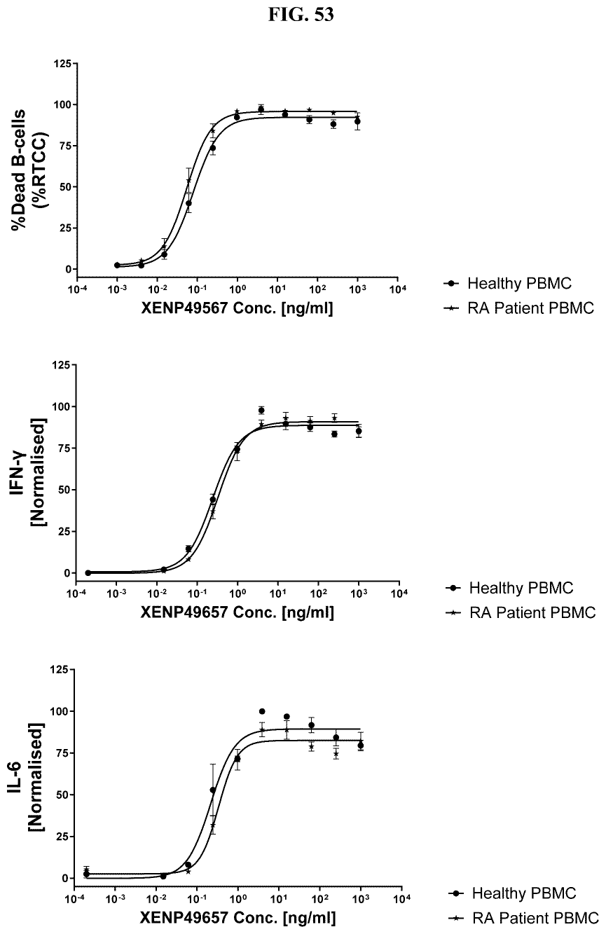

0.3×10⁶ human PBMCs from healthy donors or donors with rheumatoid arthritis were incubated with varying concentrations of XENP49657 for 24 hours at 37° C. Cells were stained for 45 minutes at 4° C. followed by fixation and analysis by flow cytometry. Supernatant was used for cytokine analysis. Data, as depicted in FIG. 53, show that XENP49657 effectively depleted B cells from both healthy donors and donors with rheumatoid arthritis.

### Example 11: CD19×CD3 bsAbs Deplete B Cells from Patients with SLE

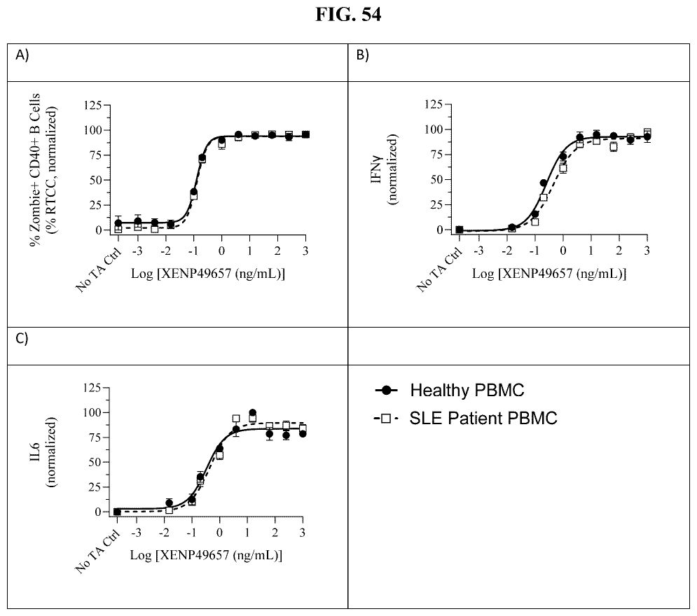

0.3×10⁶ human PBMCs from healthy donors or donors with systemic lupus erythematosus were incubated with varying concentrations of XENP49657 for 24 hours at 37° C. Cells were stained for 45 minutes at 4° C. followed by fixation and analysis by flow cytometry. Supernatant was used for cytokine analysis. Data, as depicted in FIG. 54, show that XENP49657 effectively depleted B cells from both healthy donors and donors with systemic lupus erythematosus.

Those skilled in the art will appreciate that numerous changes and modifications can be made to the preferred variations or embodiments disclosed herein and that such changes and modifications can be made without departing from the spirit of the disclosure. It is, therefore, intended that the appended claims cover all such equivalent variations as fall within the true spirit and scope of the disclosure.

Sequence Table

| | | |
|---|---|---|
| Anti-CD19 | SEQ ID | SYVMH |
| First Subunit | NO: 1 | |
| CDR1 | | |
| Anti-CD19 | SEQ ID | YINPYNDGTKYNEKFQG |
| First Subunit | NO: 2 | |
| CDR2 | | |
| Anti-CD19 | SEQ ID | GTYYYGTRVFDY |
| First Subunit | NO: 3 | |
| CDR3 | | |
| Anti-CD19 | SEQ ID | RSSKSLQNVNGNTYLY |
| Second | NO: 4 | |
| Subunit CDR1 | | |
| Anti-CD19 | SEQ ID | RASNLNS |
| Second | NO: 5 | |
| Subunit CDR2 | | |
| Anti-CD19 | SEQ ID | MQHLEYPIT |
| Second | NO: 6 | |
| Subunit CDR3 | | |
| Anti-CD19 | SEQ ID | QVQLVQSGAEVKKPGASVKVSCKASGYTFTSYVMHWVR |
| First Subunit | NO: 7 | QAPGQGLEWMGYINPYNDGTKYNEKFQGRVTITSDKSTS |
| | | TAYMELSSLRSEDTAVYYCARGTYYYGTRVFDYWGQGT |
| | | LVTVSS |
| Anti-CD19 | SEQ ID | DIVMTQSPLSLPVTPGEPASISCRSSKSLQNVNGNTYLYWF |
| Second | NO: 8 | LQKPGQSPQLLIYRASNLNSGVPDRFSGSGSGTDFTLKISR |
| Subunit | | VEAEDVGVYYCMQHLEYPITFGQGTKLEIK |
| Anti-CD3 First | SEQ ID | TYAMN |
| Subunit CDR1 | NO: 9 | |
| Anti-CD3 | SEQ ID | RIRSKYNNYATYYADSVKG |
| First Subunit | NO: 10 | |
| CDR2a | | |
| Anti-CD3 | SEQ ID | RIRSKANNYATYYADSVKG |
| First Subunit | NO: 11 | |
| CDR2b | | |
| Anti-CD3 First | SEQ ID | HTNFGDSYVSWFAY |
| Subunit | NO: 12 | |
| CDR3a | | |
| Anti-CD3 First | SEQ ID | HTNFGGSYVSWFAY |
| Subunit | NO: 13 | |
| CDR3b | | |
| Anti-CD3 First | SEQ ID | HENFGDSYVSWFAY |
| Subunit | NO: 14 | |
| CDR3c | | |
| Anti-CD3 First | SEQ ID | HENFGGSYVSWFAY |
| Subunit | NO: 15 | |
| CDR3d | | |
| Anti-CD3 First | SEQ ID | HTNFGGEYVSWFAY |
| Subunit | NO: 16 | |
| CDR3e | | |
| Anti-CD3 | SEQ ID | GSSTGAVTTSNYAN |
| Second | NO: 17 | |
| Subunit CDR1 | | |
| Anti-CD3 | SEQ ID | GTNKRAP |
| Second | NO: 18 | |
| Subunit CDR2 | | |
| Anti-CD3 | SEQ ID | ALWYSNHWV |
| Second | NO: 19 | |
| Subunit CDR3 | | |
| Anti-CD19 | SEQ ID | QVQLVQSGAEVKKPGASVKVSCKASGYTFT |
| FR1 | NO: 20 | |
| Anti-CD19 | SEQ ID | WVRQAPGQGLEWMG |
| FR2 | NO: 21 | |
| Anti-CD19 | SEQ ID | RVTITSDKSTSTAYMELSSLRSEDTAVYYCAR |
| FR3 | NO: 22 | |
| Anti-CD19 | SEQ ID | WGQGTLVTVSS |
| FR4 | NO: 23 | |
| Anti-CD19 | SEQ ID | DIVMTQSPLSLPVTPGEPASISC |
| FR5 | NO: 24 | |
| Anti-CD19 | SEQ ID | WFLQKPGQSPQLLIY |
| FR6 | NO: 25 | |
| Anti-CD19 | SEQ ID | GVPDRFSGSGSGTDFTLKISRVEAEDVGVYYC |
| FR7 | NO: 26 | |
| Anti-CD19 | SEQ ID | FGQGTKLEIK |
| FR8 | NO: 27 | |
| Anti-CD3 FR1 | SEQ ID | EVQLVESGGGLVQPGGSLRLSCAASGFTFS |
| | NO: 28 | |
| Anti-CD3 FR2 | SEQ ID | WVRQAPGKCLEWVG |
| | NO: 29 | |
| Anti-CD3 FR3 | SEQ ID | RFTISRDDSKNTLYLQMNSLRAEDTAVYYCVR |
| | NO: 30 | |
| Anti-CD3 FR4 | SEQ ID | WGQGTLVTVSS |
| | NO: 31 | |
| Anti-CD3 FR5 | SEQ ID | QAVVTQEPSLTVSPGGTVTLTC |
| | NO: 32 | |
| Anti-CD3 FR6 | SEQ ID | WVQQKPGKSPRGLIG |
| | NO: 33 | |
| Anti-CD3 FR7 | SEQ ID | GVPARFSGSLLGGKAALTISGAQPEDEADYYC |
| | NO: 34 | |
| Anti-CD3 FR8 | SEQ ID | FGCGTKLTVL |
| | NO: 35 | |
| Anti-CD3 | SEQ ID | GKPGSGKPGSGKPGSGKPGS |
| Linker | NO: 36 | |
| Human CD3 | SEQ ID | MQSGTHWRVLGLCLLSVGVWGQDGNEEMGGITQTPYKVS |
| sequence | NO: 37 | ISGTTVILTCPQYPGSEILWQHNDKNIGGDEDDKNIGSDE |
| | | DHLSLKEFSELEQSGYYVCYPRGSKPEDANFYLYLRARVC |
| | | ENCMEMDVMSVATIVIVDICITGGLLLLVYYWSKNRKAK |
| | | AKPVTRGAGAGGRQRGQNKERPPPVPNPDYEPIRKGQRD |
| | | LYSGLNQRRI |
| Human CD3, | SEQ ID | DGNEEMGGITQTPYKVSISGTTVILTCPQYPGSEILWQHND |
| extracellular | NO: 38 | KNIGGDEDDKNIGSDEDHLSLKEFSELEQSGYYVCYPRGS |
| domain | | KPEDANFYLYLRARVCENCMEMD |
| Mouse CD3 | SEQ ID | MRWNTFWGILCLSLLAVGTCQDDAENIEYKVSISGTSVEL |
| sequence | NO: 39 | TCPLDSDENLKWEKNGQELPQKHDKHLVLQDFSEVEDSGY |
| | | YVCYTPASNKNTYLYLKARVCEYCVEVDLTAVAIIIIVDI |
| | | CITLGLLMVIYYWSKNRKAKAKPVTRGTGAGSRPRGQNK |
| | | ERPPPVPNPDYEPIRKGQRDLYSGLNQRAV |
| Mouse CD3, | SEQ ID | DAENIEYKVSISGTSVELTCPLDSDENLKWEKNGQELPQK |
| extracellular | NO: 40 | HDKHLVLQDFSEVEDSGYYVCYTPASNKNTYLYLKARVC |
| domain | | EYCVEVD |
| Cynomolgus | SEQ ID | MQSGTRWRVLGLCLLSIGVWGQDGNEEMGSITQTPYQVS |
| CD3 sequence | NO: 41 | ISGTTVILTCSQHLGSEAQWQHNGKNKEDSGDRLFLPEFS |
| | | EMEQSGYYVCYPRGSNPEDASHHLYLKARVCENCMEMDV |
| | | MAVATIVIVDICITLGLLLLVYYWSKNRKAKAKPVTRGAG |
| | | AGGRQRGQNKERPPPVPNPDYEPIRKGQQDLYSGLNQRRI |
| Cynomolgus | SEQ ID | QDGNEEMGSITQTPYQVSISGTTVILTCSQHLGSEAQWQH |
| CD3, | NO: 42 | NGKNKEDSGDRLFLPEFSEMEQSGYYVCYPRGSNPEDASH |
| extracellular | | HLYLKARVCENCMEMD |
| domain | | |
| Human CD19 | SEQ ID | MPPPRLLFFLLFLTPMEVRPEEPLVVKVEEGDNAVLQCLK |
| sequence | NO: 43 | GTSDGPTQQLTWSRESPLKPFLKLSLGLPGLGIHMRPLAI |
| | | WLFIFNVSQQMGGFYLCQPGPPSEKAWQPGWTVNVEGSGE |
| | | LFRWNVSDLGGLGCGLKNRSSEGPSSPSGKLMSPKLYVW |
| | | AKDRPEIWEGEPPCLPPRDSLNQSLSQDLTMAPGSTLWLS |
| | | CGVPPDSVSRGPLSWTHVHPKGPKSLLSLELKDDRPARDM |
| | | WVMETGLLLPRATAQDAGKYYCHRGNLTMSFHLEITARP |
| | | VLWHWLLRTGGWKVSAVTLAYLIFCLCSLVGILHLQRAL |
| | | VLRRKRKRMTDPTRRFFKVTPPPGSGPQNQYGNVLSLPTP |
| | | TSGLGRAQRWAAGLGGTAPSYGNPSSDVQADGALGSRSP |
| | | PGVGPEEEEGEGYEEPDSEEDSEFYENDSNLGQDQLSQDG |
| | | SGYENPEDEPLGPEDEDSFSNAESYENEDEELTQPVARTM |
| | | DFLSPHGSAWDPSREATSLGSQSYEDMRGILYAAPQLRSI |
| | | RGQPGPNHEEDADSYENMDNPDGPDPAWGGGGRMGTWST |
| | | R |
| Human CD19 | SEQ ID | PEEPLVVKVEEGDNAVLQCLKGTSDGPTQQLTWSRESPLK |
| sequence, | NO: 44 | PFLKLSLGLPGLGIHMRPLAIWLFIFNVSQQMGGFYLCQPG |
| extracellular | | PPSEKAWQPGWTVNVEGSGELFRWNVSDLGGLGCGLKN |
| domain | | RSSEGPSSPSGKLMSPKLYVWAKDRPEIWEGEPPCLPPRDS |
| | | LNQSLSQDLTMAPGSTLWLSCGVPPDSVSRGPLSWTHVHP |
| | | KGPKSLLSLELKDDRPARDMWVMETGLLLPRATAQDAG |
| | | KYYCHRGNLTMSFHLEITARPVLWHWLLRTGGWK |
| Mouse CD19 | SEQ ID | MPSPLPVSFLLFLTLVGGRPQKSLLVEVEEGGNVVLPCLPD |
| sequence | NO: 45 | SSPVSSEKLAWYRGNQSTPFLELSPGSPGLGLHVGSLGILL |
| | | VIVNVSDHMGGFYLCQKRPPFKDIWQPAWTVNVEDSGEM |
| | | FRWNASDVRDLDCDLRNRSSGSHRSTSGSQLYVWAKDHP |
| | | KVWGTKPVCAPRGSSLNQSLINQDLTVAPGSTLWLSCGVP |
| | | PVPVAKGSISWTHVHPRRPNVSLLSLSLGGEHPVREMWV |
| | | WGSLLLLPQATALDEGTYYCLRGNLTIERHVKVIARSAV |
| | | WLWLLRTGGWIVPVVTLVYVIFCMVSLVAFLYCQRAFILR |
| | | RKRKRMTDPARRFFKVTPPSGNGTQNQYGNVLSLPTSTSG |
| | | QAHAQRWAAGLGSVPGSYGNPRIQVQDTGAQSHETGLEE |
| | | EGEAYEEPDSEEGSEFYENDSNLGQDQVSQDGSGYENPED |
| | | EPMGPEEEDSFSNAESYENADEELAQPVGRMMDFLSPHGS |
| | | AWDPSREASSLGSQSYEDMRGILYAAPQLHSIQSGPSHEE |
| | | DADSYENMDKSDDLEPAWEGEGHMGTWGTT |
| Mouse CD19 | SEQ ID | RPQKSLLVEVEEGGNVVLPCLPDSSPVSSEKLAWYRGNQS |
| sequence, | NO: 46 | TPFLELSPGSPGLGLHVGSLGILLVIVNVSDHMGGFYLCQ |
| extracellular | | KRPPFKDIWQPAWTVNVEDSGEMFRWNASDVRDLDCDLR |
| domain | | NRSSGSHRSTSGSQLYVWAKDHPKVWGTKPVCAPRGSSL |
| | | NQSLINQDLTVAPGSTLWLSCGVPPVPVAKGSISWTHVHP |
| | | RRPNVSLLSLSLGGEHPVREMWVWGSLLLLPQATALDEG |
| | | TYYCLRGNLTIERHVKVIARSAVWLWLLRTGG |
| Macaca | SEQ ID | MPPPCLLFFLLFLTPMEVRPQEPLVVKVEEGDNAVLQCLE |
| fascicularis | NO: 47 | GTSDGPTQQLVWCRDSPFEPFLNLSLGLPGMGIRMGPLGI |
| CD19 | | WLLIFNVSNQTGGFYLCQPGLPSEKAWQPGWTVSVEGSG |
| sequence | | ELFRWNVSDLGGLGCGLKNRSSEGPSSPSGKLNSSQLYVW |
| | | AKDRPEMWEGEPVCGPPRDSLNQSLSQDLTMAPGSTLWL |
| | | SCGVPPDSVSRGPLSWTHVRPKGPKSSLLSLELKDDRPDR |
| | | DMWVVDTGLLLTRATAQDAGKYYCHRGNWTKSFYLEIT |
| | | ARPALWHWLLRIGGWKVPAVTLTYLIFCLCSLVGILQLQR |
| | | ALVLRRKRKRMTDPTRRFFKVTPPPGSGPQNQYGNVLSLP |
| | | TPTSGLGRAQRWAAGLGGTAPSYGNPSSDVQVDGAVGSR |
| | | SPPGAGPEEEEGEGYEEPDSEEGSEFYENDSNFGQDQLSQD |
| | | GSGYENPEDEPLGPEDEDSFSNAESYENEDEELTQPVART |
| | | MDFLSPHGSAWDPSREATSLGSQSYEDMRGLLYAAPQLR |
| | | TIRGQPGPNHEEDADSYENMDNPDGPDPAWGGGGRMGT |
| | | WSAR |
| Macaca | SEQ ID | PQEPLVVKVEEGDNAVLQCLEGTSDGPTQQLVWCRDSPF |
| fascicularis | NO: 48 | EPFLNLSLGLPGMGIRMGPLGIWLLIFNVSNQTGGFYLCQ |
| CD19 | | PGLPSEKAWQPGWTVSVEGSGELFRWNVSDLGGLGCGLK |
| sequence, | | NRSSEGPSSPSGKLNSSQLYVWAKDRPEMWEGEPVCGPPR |
| extracellular | | DSLNQSLSQDLTMAPGSTLWLSCGVPPDSVSRGPLSWTHV |
| domain | | RPKGPKSSLLSLELKDDRPDRDMWVVDTGLLLTRATAQD |
| (predicted) | | AGKYYCHRGNWTKSFYLEITARPALWHWLLRIGGWK |
| Gly-Ser 15 | SEQ ID | GGGGSGGGGSGGGGS |
| linker | NO: 49 | |
| Whitlow linker | SEQ ID | GSTSGSGKPGSGEGSTKG |
| | NO: 50 | |
| 6paxA_1 (+A) | SEQ ID | IRPRAIGGSKPRVA |
| linker | NO: 51 | |
| +B linker | SEQ ID | GKGGSGKGGSGKGGS |
| | NO: 52 | |
| +C linker | SEQ ID | GGKGSGGKGSGGKGS |
| | NO: 53 | |
| +D linker | SEQ ID | GGGKSGGGKSGGGKS |
| | NO: 54 | |
| +E linker | SEQ ID | GKGKSGKGKSGKGKS |
| | NO: 55 | |
| +F linker | SEQ ID | GGGKSGGKGSGKGGS |
| | NO: 56 | |
| +G linker | SEQ ID | GKPGSGKPGSGKPGS |
| | NO: 57 | |
| +H linker | SEQ ID | GKPGSGKPGSGKPGSGKPGS |
| | NO: 58 | |
| +I linker | SEQ ID | GKGKSGKGKSGKGKSGKGKS |
| | NO: 59 | |
| Gly-Ser 20 | SEQ ID | GGGGSGGGGSGGGGSGGGGS |
| linker | NO: 60 | |
| 3hsc_2 (−A) | SEQ ID | STAGDTHLGGEDFD |
| linker | NO: 61 | |
| −B linker | SEQ ID | GEGGSGEGGSGEGGS |
| | NO: 62 | |
| −C linker | SEQ ID | GEGGSGEGGSGEGGS |
| | NO: 63 | |
| −D linker | SEQ ID | GGGESGGGESGGGES |
| | NO: 64 | |
| −E linker | SEQ ID | GEGESGEGESGEGES |
| | NO: 65 | |
| −F linker | SEQ ID | GGGESGGEGSGEGGS |
| | NO: 66 | |
| −G linker | SEQ ID | GEGESGEGESGEGESGEGES |
| | NO: 67 | |
| Add1 linker | SEQ ID | GGGGSGGGGSGGGGS |
| | NO: 68 | |
| Add2 linker | SEQ ID | GGGGSGGGGSGGGGSGGGGS |
| | NO: 69 | |
| Add3 linker | SEQ ID | GSTSGSGKPGSGEGSTKG |
| | NO: 70 | |
| Add4 linker | SEQ ID | PRGASKSGSASQTGSAPGS |
| | NO: 71 | |
| Add5 linker | SEQ ID | GTAAAGAGAAGGAAAGAAG |
| | NO: 72 | |
| Add6 linker | SEQ ID | GTSGSSGSGSGGSGSGGGG |
| | NO: 73 | |
| Add7 linker | SEQ ID | GKPGSGKPGSGKPGSGKPGS |
| | NO: 74 | |
| EB linker | SEQ ID | GGSEGKSSGSGSESKSTGGS |
| | NO: 75 | |
| Staple linker | SEQ ID | GGGSGGSGGCPPCGGSGG |
| | NO: 76 | |
| (GGGGS)₁ | SEQ ID | GGGGS |
| | NO: 77 | |
| (GGGGS)₂ | SEQ ID | GGGGSGGGGS |
| | NO: 78 | |
| (GGGGS)₃ | SEQ ID | GGGGSGGGGSGGGGS |
| | NO: 79 | |
| (GGGGS)₄ | SEQ ID | GGGGSGGGGSGGGGSGGGGS |
| | NO: 80 | |
| (GGGGS)₅ | SEQ ID | GGGGSGGGGSGGGGSGGGGSGGGGS |
| | NO: 81 | |
| (GGGGS)₆ | SEQ ID | GGGGSGGGGSGGGGSGGGGSGGGGSGGGGS |
| | NO: 82 | |
| (GGGGS)₇ | SEQ ID | GGGGSGGGGSGGGGSGGGGSGGGGSGGGGSGGGGS |
| | NO: 83 | |
| (GGGGA)₁ | SEQ ID | GGGGA |
| | NO: 84 | |
| (GGGGA)₂ | SEQ ID | GGGGAGGGGA |
| | NO: 85 | |
| (GGGGA)₃ | SEQ ID | GGGGAGGGGAGGGGA |
| | NO: 86 | |
| (GGGGA)₄ | SEQ ID | GGGGAGGGGAGGGGAGGGGA |
| | NO: 87 | |
| (GGGGA)₅ | SEQ ID | GGGAGGGGAGGGGAGGGGAGGGGA |
| | NO: 88 | |
| (GGGGA)₆ | SEQ ID | GGGGAGGGGAGGGGAGGGGAGGGGAGGGGA |
| | NO: 89 | |
| (GGGGA)₇ | SEQ ID | GGGGAGGGGAGGGGAGGGGAGGGGAGGGGAGGGGA |
| | NO: 90 | |
| 30AA-linker | SEQ ID | DPALVHQRPAPPGGGGSGGGGSGGGGSGGG |
| | NO: 91 | |
| (GKPGS)₁ | SEQ ID | GKPGS |
| | NO: 92 | |
| (GKPGS)₅ | SEQ ID | GKPGSGKPGSGKPGSGKPGSGKPGS |
| | NO: 93 | |
| (GKPGS)₆ | SEQ ID | GKPGSGKPGSGKPGSGKPGSGKPGSGKPGS |
| | NO: 94 | |
| (GGGES)₁ | SEQ ID | GGGES |
| | NO: 95 | |
| “Full hinge” | SEQ ID | EPKSCDKTHTCPPCP |
| | NO: 96 | |
| “Lower half | SEQ ID | KTHTCPPCP |
| hinge” | NO: 97 | |
| “Full hinge | SEQ ID | EPKSSDKTHTCPPCP |
| C220S variant” | NO: 98 | |
| “Flex lower | SEQ ID | GGGGSGGGGSKTHTCPPCP |
| half hinge” | NO: 99 | |
| “Charged | SEQ ID | GKPGSGKPGSKTHTCPPCP |
| lower half | NO: 100 | |
| hinge1” | | |
| “Charged | SEQ ID | GKPGSKTHTCPPCP |
| lower half | NO: 101 | |
| hinge2” | | |
| “Upper half | SEQ ID | EPKSC |
| hinge” | NO: 102 | |
| “Flex upper | SEQ ID | EPKSCGGGGSGGGGS |
| half hinge” | NO: 103 | |
| “Charged | SEQ ID | EPKSCGKPGSGKPGS |
| upper half | NO: 104 | |
| hinge1” | | |
| “Charged | SEQ ID | EPKSCGKPGS |
| upper half | NO: 105 | |
| hinge2” | | |
| Heterodimeric | SEQ ID | APPVAGPSVFLFPPKPKDTLMISRTPEVTCVVVDVKHEDP |
| Fc Backbone 1 | NO: 106 | EVKFNWYVDGVEVHNAKTKPREEEYNSTYRVVSVLTVLH |
| monomer 1 (−) | | QDWLNGKEYKCKVSNKALPAPIEKTISKAKGQPREPQVYT |
| | | LPPSREEMTKNQVSLTCDVSGFYPSDIAVEWESDGQPENN |
| | | YKTTPPVLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone 1 | NO: 107 | VKFNWYVDGVEVHNAKTKPREEQYNSTYRVVSVLTVLH |
| monomer 2 (+) | | QDWLNGKEYKCKVSNKALPAPIEKTISKAKGQPREPQVYT |
| | | LPPSREQMTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone 2 | NO: 108 | VKFNWYVDGVEVHNAKTKPREEEYNSTYRVVSVLTVLH |
| monomer 1 (−) | | QDWLNGKEYKCKVSNKALPAPIEKTISKAKGQPREPQVYT |
| | | LPPSREEMTKNQVSLTCDVSGFYPSDIAVEWESDGQPENN |
| | | YKTTPPVLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone | NO: 109 | VKFNWYVDGVEVHNAKTKPREEQYNSTYRVVSVLTVLH |
| monomer 2 (+) | | QDWLNGKEYKCKVSNKALPAPIEKTISKAKGQPREPQVYT |
| | | LPPSREEMTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone 3 | NO: 110 | VKFNWYVDGVEVHNAKTKPREEEYNSTYRVVSVLTVLH |
| monomer 1 (−) | | QDWLNGKEYKCKVSNKALPAPIEKTISKAKGQPREPQVYT |
| | | LPPSREEMTKNQVSLTCEVSGFYPSDIAVEWESDGQPENN |
| | | YKTTPPVLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone 3 | NO: 111 | VKFNWYVDGVEVHNAKTKPREEQYNSTYRVVSVLTVLH |
| monomer 2 (+) | | QDWLNGKEYKCKVSNKALPAPIEKTISKAKGQPREPQVYT |
| | | LPPSREEMTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone 4 | NO: 112 | VKFNWYVDGVEVHNAKTKPREEEYNSTYRVVSVLTVLH |
| monomer 1 (−) | | QDWLNGKEYKCKVSNKALPAPIEKTISKAKGQPREPQVYT |
| | | LPPSREEMTENEVSLTCLVKGFYPSDIAVEWESDGQPENN |
| | | YKTTPPVLDSDGSFFLYSKLEVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone 4 | NO: 113 | VKFNWYVDGVEVHNAKTKPREEQYNSTYRVVSVLTVLH |
| monomer 2 (+) | | QDWLNGKEYKCKVSNKALPAPIEKTISKAKGQPREPQVYT |
| | | LPPSREEMTKNQVSLTCLVKGFYPSDIAVEWESNGQPENN |
| | | YKTTPPVLDSKGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone 5 | NO: 114 | VKFNWYVDGVEVHNAKTKPREEEYNSTYRVVSVLTVLH |
| monomer 1 (−) | | QDWLNGKEYKCKVSNKALPAPIEKTISKAKGQPREPQVYT |
| | | LPPSRDELTKNQVSLTCDVSGFYPSDIAVEWESDGQPENN |
| | | YKTTPPVLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone 5 | NO: 115 | VKFNWYVDGVEVHNAKTKPREEQYNSTYRVVSVLTVLH |
| monomer 2 (+) | | QDWLNGKEYKCKVSNKALPAPIEKTISKAKGQPREPQVYT |
| | | LPPSRDQLTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone 6 | NO: 116 | VKFNWYVDGVEVHNAKTKPREEEYASTYRVVSVLTVLH |
| monomer 1 (−) | | QDWLNGKEYKCKVSNKALPAPIEKTISKAKGQPREPQVYT |
| | | LPPSREEMTKNQVSLTCDVSGFYPSDIAVEWESDGQPENN |
| | | YKTTPPVLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone 6 | NO: 117 | VKFNWYVDGVEVHNAKTKPREEQYASTYRVVSVLTVLH |
| monomer 2 (+) | | QDWLNGKEYKCKVSNKALPAPIEKTISKAKGQPREPQVYT |
| | | LPPSREQMTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone 7 | NO: 118 | VKFNWYVDGVEVHNAKTKPREEEYSSTYRVVSVLTVLHQ |
| monomer 1 (−) | | DWLNGKEYKCKVSNKALPAPIEKTISKAKGQPREPQVYTL |
| | | PPSREEMTKNQVSLTCDVSGFYPSDIAVEWESDGQPENNY |
| | | KTTPPVLDSDGSFFLYSKLTVDKSRWEQGDVFSCSVMHEA |
| | | LHNHYTQKSLSLSPGK |
| Heterodimeric | SEQ ID | APPVAGPSVFLFPPKPKDTLMISRTPEVTCVVVDVKHEDPE |
| Fc Backbone 7 | NO: 119 | VKFNWYVDGVEVHNAKTKPREEQYSSTYRVVSVLTVLH |
| monomer 2 (+) | | QDWLNGKEYKCKVSNKALPAPIEKTISKAKGQPREPQVYT |
| | | LPPSREQMTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APEFLGGPSVFLFPPKPKDTLMISRTPEVTCVVVDVSQEDP |
| Fc Backbone 8 | NO: 120 | EVQFNWYVDGVEVHNAKTKPREEEFNSTYRVVSVLTVLH |
| monomer 1 (−) | | QDWLNGKEYKCKVSNKGLPSSIEKTISKAKGQPREPQVYT |
| | | LPPSQEEMTKNQVSLTCDVSGFYPSDIAVEWESDGQPENN |
| | | YKTTPPVLDSDGSFFLYSRLTVDKSRWEEGDVFSCSVMHE |
| | | ALHNHYTQKSLSLSLGK |
| Heterodimeric | SEQ ID | APEFLGGPSVFLFPPKPKDTLMISRTPEVTCVVVDVSQEDP |
| Fc Backbone 8 | NO: 121 | EVQFNWYVDGVEVHNAKTKPREEQFNSTYRVVSVLTVLH |
| monomer 2 (+) | | QDWLNGKEYKCKVSNKGLPSSIEKTISKAKGQPREPQVYT |
| | | LPPSQEQMTKNQVKLTCLVKGFYPSDIAVEWESNGQPEN |
| | | NYKTTPPVLDSDGSFFLYSRLTVDKSRWQEGNVFSCSVM |
| | | HEALHNHYTQKSLSLSLGK |
| Heterodimeric | SEQ ID | APPVAGPSVFLFPPKPKDTLMISRTPEVTCVVVDVSHEDPE |
| Fc Backbone 9 | NO: 122 | VQFNWYVDGVEVHNAKTKPREEEFNSTFRVVSVLTVVHQ |
| monomer 1 (−) | | DWLNGKEYKCKVSNKGLPAPIEKTISKTKGQPREPQVYTL |
| | | PPSREEMTKNQVSLTCDVSGFYPSDIAVEWESDGQPENNY |
| | | KTTPPMLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVSHEDPE |
| Fc Backbone 9 | NO: 123 | VQFNWYVDGVEVHNAKTKPREEQFNSTFRVVSVLTVVH |
| monomer 2 (+) | | QDWLNGKEYKCKVSNKGLPAPIEKTISKTKGQPREPQVYT |
| | | LPPSREQMTKNQVKLTCLVKGFYPSDIAVEWESNGQPENN |
| | | YKTTPPMLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone | NO: 124 | VQFNWYVDGVEVHNAKTKPREEEFNSTFRVVSVLTVVHQ |
| 10 monomer 1 | | DWLNGKEYKCKVSNKGLPAPIEKTISKTKGQPREPQVYTL |
| (−) | | PPSREEMTKNQVSLTCDVSGFYPSDIAVEWESDGQPENNY |
| | | KTTPPMLDSDGSFFLYSKLTVDKSRWEQGDVFSCSVMHE |
| | | ALHNHYTQKSLSLSPGK |
| Heterodimeric | SEQ ID | APPVAGPSVFLFPPKPKDTLMISRTPEVTCVVVDVKHEDPE |
| Fc Backbone | NO: 125 | VQFNWYVDGVEVHNAKTKPREEQFNSTFRVVSVLTVVH |
| 10 monomer 2 | | QDWLNGKEYKCKVSNKGLPAPIEKTISKTKGQPREPQVYT |
| (+) | | LPPSREQMTKNQVKLTCLVKGFYPSDIAVEWESNGQPENN |
| | | YKTTPPMLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone | NO: 126 | VKFNWYVDGVEVHNAKTKPREEEYNSTYRVVSVLTVLH |
| 11 monomer 1 | | QDWLNGKEYKCKVSNKALPAPIEKTISKAKGQPREPQVYT |
| (−) | | LPPSREEMTKNQVSLTCDVSGFYPSDIAVEWESDGQPENN |
| | | YKTTPPVLDSDGSFFLYSKLTVDKSRWEQGDVFSCSVLHE |
| | | ALHSHYTQKSLSLSPGK |
| Heterodimeric | SEQ ID | APPVAGPSVFLFPPKPKDTLMISRTPEVTCVVVDVKHEDPE |
| Fc Backbone | NO: 127 | VKFNWYVDGVEVHNAKTKPREEQYNSTYRVVSVLTVLH |
| 11 monomer 2 | | QDWLNGKEYKCKVSNKALPAPIEKTISKAKGQPREPQVYT |
| (+) | | LPPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Heterodimeric | SEQ ID | APPVAGPSVFLFPPKPKDTLMISRTPEVTCVVVDVKHEDPE |
| Fc Backbone | NO: 128 | VKFKWYVDGVEVHNAKTKPREEQYNSTYRVVSVLTVLH |
| 12 monomer 1 | | QDWLNGKEYKCKVSNKALPAPIEKTISKAKGQPREPQVYT |
| (−) | | LPPSREQMTKNQVKLTCLVKGFYPSDIAVEWESNGQPENN |
| | | YKTTPPVLDSDGSFFLYSKLTVDKSRWQQGNVFSCSVMH |
| | | EALHNHYTQKSLSLSPGK |
| Heterodimeric | SEQ ID | APPVAGPSVFLFPPKPKDTLMISRTPEVTCVVVDVKHEDPE |
| Fc Backbone | NO: 129 | VKFNWYVDGVEVHNAKTKPREEEYNSTYRVVSVLTVLH |
| 12 monomer 2 | | QDWLNGKEYKCKVSNKALPAPIEKTISKAKGQPREPQVYT |
| (+) | | LPPSREEMTKNQVSLTCDVSGFYPSDIAVEWESDGQPENN |
| | | YKTTPPVLDSDGSFFLYSKLTVDKSRWEQGDVFSCSVMHE |
| | | ALHNHYTQKSLSLSPGK |
| Heterodimeric | SEQ ID | APEAAGGPSVFLFPPKPKDTLMISRTPEVTCVVVSVSHEDP |
| Fc Backbone | NO: 130 | EVKFNWYVDGVEVHNAKTKPREEQYNSTYRVVSVLTVL |
| 13 monomer 1 | | HQDWLNGKEYKCKVSNKALPAPIEKTISKAKGQPREPQV |
| | | YTLPPSREEMTKNQVSLWCLVKGFYPSDIAVEWESNGQPE |
| | | NNYKTTPPVLDSDGSFFLYSKLTVDKSRWQQGNVFSCSV |
| | | MHEALHNHYTQKSLSLSPGK |
| Heterodimeric | SEQ ID | APEAAGGPSVFLFPPKPKDTLMISRTPEVTCVVVSVSHEDP |
| Fc Backbone | NO: 131 | EVKFNWYVDGVEVHNAKTKPREEQYNSTYRVVSVLTVL |
| 13 monomer 2 | | HQDWLNGKEYKCKVSNKALPAPIEKTISKAKGQPREPQV |
| | | YTLPPSREEMTKNQVSLSCAVKGFYPSDIAVEWESNGQPE |
| | | NNYKTTPPVLDSDGSFFLVSKLTVDKSRWQQGNVFSCSV |
| | | MHEALHNRFTQKSLSLSPGK |
| Heterodimeric | SEQ ID | APEAAGGPSVFLFPPKPKDTLYITREPEVTCVVVSVSHEDP |
| Fc Backbone | NO: 132 | EVKFNWYVDGVEVHNAKTKPREEQYNSTYRVVSVLTVL |
| 14 monomer 1 | | HQDWLNGKEYKCKVSNKALPAPIEKTISKAKGQPREPQV |
| | | YTLPPSREEMTKNQVSLWCLVKGFYPSDIAVEWESNGQPE |
| | | NNYKTTPPVLDSDGSFFLYSKLTVDKSRWQQGNVFSCSV |
| | | MHEALHNHYTQKSLSLSPGK |
| Heterodimeric | SEQ ID | APEAAGGPSVFLFPPKPKDTLYITREPEVTCVVVSVSHEDP |
| Fc Backbone | NO: 133 | EVKFNWYVDGVEVHNAKTKPREEQYNSTYRVVSVLTVL |
| 14 monomer 2 | | HQDWLNGKEYKCKVSNKALPAPIEKTISKAKGQPREPQV |
| | | YTLPPSREEMTKNQVSLSCAVKGFYPSDIAVEWESNGQPE |
| | | NNYKTTPPVLDSDGSFFLVSKLTVDKSRWQQGNVFSCSV |
| | | MHEALHNRFTQKSLSLSPGK |
| Heterodimeric | SEQ ID | APPVAGPSVFLFPPKPKDTLMISRTPEVTCVVVDVKHEDPE |
| Fc Backbone | NO: 134 | VKFNWYVDGVEVHNAKTKPREEEYNSTYRVVSVLTVLH |
| 15 monomer 1 | | QDWLNGKEYKCKVSNKALPAPIEKTISKAKGQPREPQVYT |
| | | LPPSRDELTKNQVSLTCDVSGFYPSDIAVEWESDGQPENN |
| | | YKTTPPVLDSDGSFFLYSKLTVDKSRWEQGDVFSCSVLHE |
| | | ALHSHYTQKSLSLSPGK |
| Heterodimeric | SEQ ID | APPVAGPSVFLFPPKPKDTLMISRTPEVTCVVVDVKHEDPE |
| Fc Backbone | NO: 135 | VKFNWYVDGVEVHNAKTKPREEQYNSTYRVVSVLTVLH |
| 15 monomer 2 | | QDWLNGKEYKCKVSNKALPAPIEKTISKAKGQPREPQVYT |
| | | LPPSRDQLTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Heterodimeric | SEQ ID | APPVAGPSVFLFPPKPKDTLMISRTPEVTCVVVDVKHEDPE |
| Fc Backbone | NO: 136 | VKFNWYVDGVEVHNAKTKPREEEYNSTYRVVSVLTVLH |
| 16 monomer 1 | | QDWLNGKEYKCKVSNKALPAPIEKTISKAKGQPREPQVYT |
| | | LPPSREEMTKNQVSLTCDVSGFYPSDIAVEWESDGQPENN |
| | | YKTTPPVLDSDGSFFLYSKLTVDKSRWEQGDVFSCSVLHE |
| | | ALHAHYTQKSLSLSPGK |
| Heterodimeric | SEQ ID | APPVAGPSVFLFPPKPKDTLMISRTPEVTCVVVDVKHEDPE |
| Fc Backbone | NO: 137 | VKFNWYVDGVEVHNAKTKPREEQYNSTYRVVSVLTVLH |
| 16 monomer 2 | | QDWLNGKEYKCKVSNKALPAPIEKTISKAKGQPREPQVYT |
| | | LPPSREQMTKNQVKLTCLVKGFYPSDIAVEWESNGQPENN |
| | | YKTTPPVLDSDGSFFLYSKLTVDKSRWQQGNVFSCSVLHE |
| | | ALHAHYTQKSLSLSPGK |
| Heterodimeric | SEQ ID | APPVAGPSVFLFPPKPKDTLMISRTPEVTCVVVDVKHEDPE |
| Fc Backbone | NO: 138 | VKFNWYVDGVEVHNAKTKPREEEYNSTYRVVSVLTVLH |
| 17 monomer 1 | | QDWLNGKEYKCKVSNKALPAPIEKTISKAKGQPREPQVYT |
| | | LPPSRDELTKNQVSLTCDVSGFYPSDIAVEWESDGQPENN |
| | | YKTTPPVLDSDGSFFLYSKLTVDKSRWEQGDVFSCSVLHE |
| | | ALHAHYTQKSLSLSPGK |
| Heterodimeric | SEQ ID | APPVAGPSVFLFPPKPKDTLMISRTPEVTCVVVDVKHEDPE |
| Fc Backbone | NO: 139 | VKFNWYVDGVEVHNAKTKPREEQYNSTYRVVSVLTVLH |
| 17 monomer 2 | | QDWLNGKEYKCKVSNKALPAPIEKTISKAKGQPREPQVYT |
| | | LPPSRDQLTKNQVKLTCLVKGFYPSDIAVEWESNGQPENN |
| | | YKTTPPVLDSDGSFFLYSKLTVDKSRWQQGNVFSCSVLHE |
| | | ALHAHYTQKSLSLSPGK |
| IgG1 CH1(+) | SEQ ID | ASTKGPSVFPLAPSSKSTSGGTAALGCLVKDYFPEPVTVS |
| | NO: 140 | WNSGALTSGVHTFPAVLQSSGLYSLSSVVTVPSSSLGTQT |
| | | YICNVNHKPSNTKVDKKV |
| IgG1 CH1(−) | SEQ ID | ASTKGPSVFPLAPSSKSTSGGTAALGCLVKDYFPEPVTVS |
| | NO: 141 | WNSGALTSGVHTFPAVLQSSGLYSLSSVVTVPSSSLGTQT |
| | | YICNVNHKPSDTKVDKKV |
| IgG2 CH1(+) | SEQ ID | ASTKGPSVFPLAPCSRSTSESTAALGCLVKDYFPEPVTVSW |
| | NO: 142 | NSGALTSGVHTFPAVLQSSGLYSLSSVVTVPSSNFGTQTYT |
| | | CNVDHKPSNTKVDKTV |
| IgG2 CH1(−) | SEQ ID | ASTKGPSVFPLAPCSRSTSESTAALGCLVKDYFPEPVTVSW |
| | NO: 143 | NSGALTSGVHTFPAVLQSSGLYSLSSVVTVPSSNFGTQTYT |
| | | CNVDHKPSDTKVDKTV |
| IgG4 CH1(+) | SEQ ID | ASTKGPSVFPLAPCSRSTSESTAALGCLVKDYFPEPVTVSW |
| | NO: 144 | NSGALTSGVHTFPAVLQSSGLYSLSSVVTVPSSSLGTKTYT |
| | | CNVDHKPSNTKVDKRV |
| IgG4 CH1(−) | SEQ ID | ASTKGPSVFPLAPCSRSTSESTAALGCLVKDYFPEPVTVSW |
| | NO: 145 | NSGALTSGVHTFPAVLQSSGLYSLSSVVTVPSSSLGTKTYT |
| | | CNVDHKPSDTKVDKRV |
| IgG1 hinge | SEQ ID | EPKSCDKTHTCPPCP |
| | NO: 146 | |
| IgG2 hinge | SEQ ID | ERKCCVECPPCP |
| | NO: 147 | |
| IgG4 hinge | SEQ ID | ESKYGPPCPSCP |
| | NO: 148 | |
| Light Chain | SEQ ID | RTVAAPSVFIFPPSDEQLKSGTASVVCLLNNFYPREAKVQ |
| Constant | NO: 149 | WKVDNALQSGNSQESVTEQDSKDSTYSLSSTLTLSKADYE |
| Domain- | | KHKVYACEVTHQGLSSPVTKSFNRGEC |
| Kappa | | |
| Light Chain | SEQ ID | GQPKAAPSVTLFPPSSEELQANKATLVCLISDFYPGAVTVA |
| Constant | NO: 150 | WKADSSPVKAGVETTTPSKQSNNKYAASSYLSLTPEQWK |
| Domain- | | SHRSYSCQVTHEGSTVEKTVAPTECS |
| Lambda | | |
| Anti-CD19- | SEQ ID | EVQLVESGGGLVKPGGSLKLSCAASGYTFTSYVMHWVRQ |
| OG First | NO: 151 | APGKGLEWIGYINPYNDGTKYNEKFQGRVTISSDKSISTAY |
| Subunit | | MELSSLRSEDTAMYYCARGTYYYGTRVFDYWGQGTLVT |
| | | VSS |
| Anti-CD19- | SEQ ID | DIVMTQSPATLSLSPGERATLSCRSSKSLQNVNGNTYLYW |
| OG Second | NO: 152 | FQQKPGQSPQLLIYRMSNLNSGVPDRFSGSGSGTEFTLTISS |
| Subunit | | LEPEDFAVYYCMQHLEYPITFGAGTKLEIK |
| Anti-CD19- | SEQ ID | RMSNLNS |
| OG Second | NO: 153 | |
| Subunit CDR2 | | |
| CD3 High- | SEQ ID | EVQLVESGGGLVQPGGSLRLSCAASGFTFSTYAMNWVRQ |
| [anti- | NO: 154 | APGKGLEWVGRIRSKYNNYATYYADSVKGRFTISRDDSK |
| CD3]_H1.30 | | NTLYLQMNSLRAEDTAVYYCVRHGNFGDSYVSWFAYWG |
| L1.47 scFv | | QGTLVTVSSGKPGSGKPGSGKPGSGKPGSQAVVTQEPSLT |
| | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVL |
| Anti-CD3 VHa | SEQ ID | EVQLVESGGGLVQPGGSLRLSCAASGFTFSTYAMNWVRQ |
| | NO: 155 | APGKGLEWVGRIRSKYNNYATYYADSVKGRFTISRDDSK |
| | | NTLYLQMNSLRAEDTAVYYCVRHGNFGDSYVSWFAYWG |
| | | QGTLVTVSS |
| Anti-CD3 scFv | SEQ ID | HGNFGDSYVSWFAY |
| vhCDR3a | NO: 156 | |
| Anti-CD3 VLa | SEQ ID | QAVVTQEPSLTVSPGGTVTLTCGSSTGAVTTSNYANWVQ |
| | NO: 157 | QKPGKSPRGLIGGTNKRAPGVPARFSGSLLGGKAALTISG |
| | | AQPEDEADYYCALWYSNHWVFGGGTKLTVL |
| CD3 High-Int | SEQ ID | EVQLVESGGGLVQPGGSLRLSCAASGFTFSTYAMNWVRQ |
| #1-[anti- | NO: 158 | APGKGLEWVGRIRSKANNYATYYADSVKGRFTISRDDSK |
| CD3]_H1.32 | | NTLYLQMNSLRAEDTAVYYCVRHGNFGDSYVSWFAYWG |
| L1.47 scFv | | QGTLVTVSSGKPGSGKPGSGKPGSGKPGSQAVVTQEPSLT |
| | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVL |
| Anti-CD3 VHb | SEQ ID | EVQLVESGGGLVQPGGSLRLSCAASGFTFSTYAMNWVRQ |
| | NO: 159 | APGKGLEWVGRIRSKANNYATYYADSVKGRFTISRDDSK |
| | | NTLYLQMNSLRAEDTAVYYCVRHGNFGDSYVSWFAYWG |
| | | QGTLVTVSS |
| Anti-CD3 scFv | SEQ ID | HGNFGDEYVSWFAY |
| vhCDR3b | NO: 160 | |
| Anti-CD3 scFv | SEQ ID | HGNFGDPYVSWFAY |
| vhCDR3c | NO: 161 | |
| Anti-CD3 scFv | SEQ ID | HGNFGDSYVSWFDY |
| vhCDR3d | NO: 162 | |
| Anti-CD3 scFv | SEQ ID | TYAMS |
| vhCDR1 | NO: 163 | |
| Anti-CD3 scFv | SEQ ID | HENFGGEYVSWFAY |
| vhCDR3e | NO: 164 | |
| CD3 High-Int | SEQ ID | EVQLVESGGGLVQPGGSLRLSCAASGFTFSTYAMNWVRQ |
| #2-[anti- | NO: 165 | APGKGLEWVGRIRSKYNNYATYYADSVKGRFTISRDDSK |
| CD3]_H1.89 | | NTLYLQMNSLRAEDTAVYYCVRHGNFGDEYVSWFAYWG |
| L1.47 scFv | | QGTLVTVSSGKPGSGKPGSGKPGSGKPGSQAVVTQEPSLT |
| | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVL |
| Anti-CD3 VHc | SEQ ID | EVQLVESGGGLVQPGGSLRLSCAASGFTFSTYAMNWVRQ |
| | NO: 166 | APGKGLEWVGRIRSKYNNYATYYADSVKGRFTISRDDSK |
| | | NTLYLQMNSLRAEDTAVYYCVRHGNFGDEYVSWFAYWG |
| | | QGTLVTVSS |
| CD3 High-Int | SEQ ID | EVQLVESGGGLVQPGGSLRLSCAASGFTFSTYAMNWVRQ |
| -[anti- | NO: 167 | APGKGLEWVGRIRSKYNNYATYYADSVKGRFTISRDDSK |
| CD3]_H1.90 | | NTLYLQMNSLRAEDTAVYYCVRHGNFGDPYVSWFAYWG |
| L1.47 scFv | | QGTLVTVSSGKPGSGKPGSGKPGSGKPGSQAVVTQEPSLT |
| | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVL |
| Anti-CD3 VHd | SEQ ID | EVQLVESGGGLVQPGGSLRLSCAASGFTFSTYAMNWVRQ |
| | NO: 168 | APGKGLEWVGRIRSKYNNYATYYADSVKGRFTISRDDSK |
| | | NTLYLQMNSLRAEDTAVYYCVRHGNFGDPYVSWFAYWG |
| | | QGTLVTVSS |
| Anti-CD3 VHe | SEQ ID | EVQLVESGGGLVQPGGSLRLSCAASGFTFSTYAMNWVRQ |
| | NO: 169 | APGKGLEWVGRIRSKYNNYATYYADSVKGRFTISRDDSK |
| | | NTLYLQMNSLRAEDTAVYYCVRHGNFGDSYVSWFDYWG |
| | | QGTLVTVSS |
| Anti-CD3- | SEQ ID | EVQLVESGGGLVQPGGSLRLSCAASGFTFSTYAMNWVRQ |
| Intermediate- | NO: 170 | APGKGLEWVGRIRSKYNNYATYYADSVKGRFTISRDDSK |
| [anti- | | NTLYLQMNSLRAEDTAVYYCVRHGNFGDSYVSWFDYWG |
| CD3]_H1.33 | | QGTLVTVSSGKPGSGKPGSGKPGSGKPGSQAVVTQEPSLT |
| L1.47 scFv | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVL |
| Anti-CD3 VHf | SEQ ID | EVQLVESGGGLVQPGGSLRLSCAASGFTFSTYAMSWVRQ |
| | NO: 171 | APGKGLEWVGRIRSKYNNYATYYADSVKGRFTISRDDSK |
| | | NTLYLQMNSLRAEDTAVYYCVRHGNFGDSYVSWFAYWG |
| | | QGTLVTVSS |
| CD3 Low- | SEQ ID | EVQLVESGGGLVQPGGSLRLSCAASGFTFSTYAMSWVRQ |
| [anti- | NO: 172 | APGKGLEWVGRIRSKYNNYATYYADSVKGRFTISRDDSK |
| CD3]_H1.31 | | NTLYLQMNSLRAEDTAVYYCVRHGNFGDSYVSWFAYWG |
| L1.47 scFv | | QGTLVTVSSGKPGSGKPGSGKPGSGKPGSQAVVTQEPSLT |
| | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVL |
| CD3 High[VL- | SEQ ID | QAVVTQEPSLTVSPGGTVTLTCGSSTGAVTTSNYANWVQ |
| VH]-[anti- | NO: 173 | QKPGKSPRGLIGGTNKRAPGVPARFSGSLLGGKAALTISG |
| CD3]_L1.47 | | AQPEDEADYYCALWYSNHWVFGGGTKLTVLGKPGSGKP |
| H1.30 scFv | | GSGKPGSGKPGSEVQLVESGGGLVQPGGSLRLSCAASGFT |
| | | FSTYAMNWVRQAPGKGLEWVGRIRSKYNNYATYYADSV |
| | | KGRFTISRDDSKNTLYLQMNSLRAEDTAVYYCVRHGNFG |
| | | DSYVSWFAYWGQGTLVTVSS |
| CD3 High-Int | SEQ ID | QAVVTQEPSLTVSPGGTVTLTCGSSTGAVTTSNYANWVQ |
| #1[VL-VH]- | NO: 174 | QKPGKSPRGLIGGTNKRAPGVPARFSGSLLGGKAALTISG |
| [anti- | | AQPEDEADYYCALWYSNHWVFGGGTKLTVLGKPGSGKP |
| CD3]_L1.47 | | GSGKPGSGKPGSEVQLVESGGGLVQPGGSLRLSCAASGFT |
| H1.32 scFv | | FSTYAMNWVRQAPGKGLEWVGRIRSKANNYATYYADSV |
| | | KGRFTISRDDSKNTLYLQMNSLRAEDTAVYYCVRHGNFG |
| | | DSYVSWFAYWGQGTLVTVSS |
| CD3 High-Int | SEQ ID | QAVVTQEPSLTVSPGGTVTLTCGSSTGAVTTSNYANWVQ |
| #2[VL-VH]- | NO: 175 | QKPGKSPRGLIGGTNKRAPGVPARFSGSLLGGKAALTISG |
| [anti- | | AQPEDEADYYCALWYSNHWVFGGGTKLTVLGKPGSGKP |
| CD3]_L1.47 | | GSGKPGSGKPGSEVQLVESGGGLVQPGGSLRLSCAASGFT |
| H1.89 scFv | | FSTYAMNWVRQAPGKGLEWVGRIRSKYNNYATYYADSV |
| | | KGRFTISRDDSKNTLYLQMNSLRAEDTAVYYCVRHGNFG |
| | | DEYVSWFAYWGQGTLVTVSS |
| CD3 High- | SEQ ID | QAVVTQEPSLTVSPGGTVTLTCGSSTGAVTTSNYANWVQ |
| Int[VL-VH]- | NO: 176 | QKPGKSPRGLIGGTNKRAPGVPARFSGSLLGGKAALTISG |
| [anti- | | AQPEDEADYYCALWYSNHWVFGGGTKLTVLGKPGSGKP |
| CD3]_L1.47 | | GSGKPGSGKPGSEVQLVESGGGLVQPGGSLRLSCAASGFT |
| H1.90 scFv | | FSTYAMNWVRQAPGKGLEWVGRIRSKYNNYATYYADSV |
| | | KGRFTISRDDSKNTLYLQMNSLRAEDTAVYYCVRHGNFG |
| | | DPYVSWFAYWGQGTLVTVSS |
| Anti-CD3- | SEQ ID | QAVVTQEPSLTVSPGGTVTLTCGSSTGAVTTSNYANWVQ |
| Intermediate | NO: 177 | QKPGKSPRGLIGGTNKRAPGVPARFSGSLLGGKAALTISG |
| [VL-VH]-[anti- | | AQPEDEADYYCALWYSNHWVFGGGTKLTVLGKPGSGKP |
| CD3]_L1.47 | | GSGKPGSGKPGSEVQLVESGGGLVQPGGSLRLSCAASGFT |
| H1.33 scFv | | FSTYAMNWVRQAPGKGLEWVGRIRSKYNNYATYYADSV |
| | | KGRFTISRDDSKNTLYLQMNSLRAEDTAVYYCVRHGNFG |
| | | DSYVSWFDYWGQGTLVTVSS |
| CD3 High- | SEQ ID | QAVVTQEPSLTVSPGGTVTLTCGSSTGAVTTSNYANWVQ |
| Int[VL-VH]- | NO: 178 | QKPGKSPRGLIGGTNKRAPGVPARFSGSLLGGKAALTISG |
| [anti- | | AQPEDEADYYCALWYSNHWVFGGGTKLTVLGKPGSGKP |
| CD3]_L1.47 | | GSGKPGSGKPGSEVQLVESGGGLVQPGGSLRLSCAASGFT |
| H1.31 scFv | | FSTYAMSWVRQAPGKGLEWVGRIRSKYNNYATYYADSV |
| | | KGRFTISRDDSKNTLYLQMNSLRAEDTAVYYCVRHGNFG |
| | | DSYVSWFAYWGQGTLVTVSS |
| Anti-CD3 VHg | SEQ ID | EVQLVESGGGLVQPGGSLRLSCAASGFTFSTYAMNWVRQ |
| | NO: 179 | APGKCLEWVGRIRSKYNNYATYYADSVKGRFTISRDDSK |
| | | NTLYLQMNSLRAEDTAVYYCVRHTNFGDSYVSWFAYWG |
| | | QGTLVTVSS |
| Anti-CD3 VLb | SEQ ID | QAVVTQEPSLTVSPGGTVTLTCGSSTGAVTTSNYANWVQ |
| | NO: 180 | QKPGKSPRGLIGGTNKRAPGVPARFSGSLLGGKAALTISG |
| | | AQPEDEADYYCALWYSNHWVFGCGTKLTVL |
| Anti-CD3 VHh | SEQ ID | EVQLVESGGGLVQPGGSLRLSCAASGFTFSTYAMNWVRQ |
| | NO: 181 | APGKCLEWVGRIRSKYNNYATYYADSVKGRFTISRDDSK |
| | | NTLYLQMNSLRAEDTAVYYCVRHTNFGGSYVSWFAYWG |
| | | QGTLVTVSS |
| Anti-CD3 VHi | SEQ ID | EVQLVESGGGLVQPGGSLRLSCAASGFTFSTYAMNWVRQ |
| | NO: 182 | APGKCLEWVGRIRSKANNYATYYADSVKGRFTISRDDSK |
| | | NTLYLQMNSLRAEDTAVYYCVRHTNFGDSYVSWFAYWG |
| | | QGTLVTVSS |
| Anti-CD3 VHj | SEQ ID | EVQLVESGGGLVQPGGSLRLSCAASGFTFSTYAMNWVRQ |
| | NO: 183 | APGKCLEWVGRIRSKANNYATYYADSVKGRFTISRDDSK |
| | | NTLYLQMNSLRAEDTAVYYCVRHTNFGGSYVSWFAYWG |
| | | QGTLVTVSS |
| Anti-CD3 VHk | SEQ ID | EVQLVESGGGLVQPGGSLRLSCAASGFTFSTYAMNWVRQ |
| | NO: 184 | APGKCLEWVGRIRSKYNNYATYYADSVKGRFTISRDDSK |
| | | NTLYLQMNSLRAEDTAVYYCVRHENFGGSYVSWFAYWG |
| | | QGTLVTVSS |
| Anti-CD3 VHl | SEQ ID | EVQLVESGGGLVQPGGSLRLSCAASGFTFSTYAMNWVRQ |
| | NO: 185 | APGKCLEWVGRIRSKYNNYATYYADSVKGRFTISRDDSK |
| | | NTLYLQMNSLRAEDTAVYYCVRHENFGDSYVSWFAYWG |
| | | QGTLVTVSS |
| Anti-CD3 | SEQ ID | EVQLVESGGGLVQPGGSLRLSCAASGFTFSTYAMNWVRQ |
| VHm | NO: 186 | APGKCLEWVGRIRSKANNYATYYADSVKGRFTISRDDSK |
| | | NTLYLQMNSLRAEDTAVYYCVRHENFGDSYVSWFAYWG |
| | | QGTLVTVSS |
| Anti-CD3 VHn | SEQ ID | EVQLVESGGGLVQPGGSLRLSCAASGFTFSTYAMNWVRQ |
| | NO: 187 | APGKCLEWVGRIRSKANNYATYYADSVKGRFTISRDDSK |
| | | NTLYLQMNSLRAEDTAVYYCVRHENFGGSYVSWFAYWG |
| | | QGTLVTVSS |
| Anti-CD3 VHo | SEQ ID | EVQLVESGGGLVQPGGSLRLSCAASGFTFSTYAMNWVRQ |
| | NO: 188 | APGKCLEWVGRIRSKYNNYATYYADSVKGRFTISRDDSK |
| | | NTLYLQMNSLRAEDTAVYYCVRHTNFGGEYVSWFAYWG |
| | | QGTLVTVSS |
| Anti-CD3 VHp | SEQ ID | EVQLVESGGGLVQPGGSLRLSCAASGFTFSTYAMNWVRQ |
| | NO: 189 | APGKCLEWVGRIRSKYNNYATYYADSVKGRFTISRDDSK |
| | | NTLYLQMNSLRAEDTAVYYCVRHENFGGEYVSWFAYWG |
| | | QGTLVTVSS |
| Anti-CD3- | SEQ ID | EVQLVESGGGLVQPGGSLRLSCAASGFTFSTYAMNWVRQ |
| H1.214_L1.47 | NO: 190 | APGKCLEWVGRIRSKYNNYATYYADSVKGRFTISRDDSK |
| _VH_G44C_V | | NTLYLQMNSLRAEDTAVYYCVRHTNFGDSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.270_L1.47 | NO: 191 | APGKCLEWVGRIRSKYNNYATYYADSVKGRFTISRDDSK |
| _VH_G44C_V | | NTLYLQMNSLRAEDTAVYYCVRHTNFGGSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.271_L1.47 | NO: 192 | APGKCLEWVGRIRSKANNYATYYADSVKGRFTISRDDSK |
| _VH_G44C_V | | NTLYLQMNSLRAEDTAVYYCVRHTNFGDSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.272_L1.47 | NO: 193 | APGKCLEWVGRIRSKANNYATYYADSVKGRFTISRDDSK |
| _VH_G44C_V | | NTLYLQMNSLRAEDTAVYYCVRHTNFGGSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.273_L1.47 | NO: 194 | APGKCLEWVGRIRSKYNNYATYYADSVKGRFTISRDDSK |
| _VH_G44C_V | | NTLYLQMNSLRAEDTAVYYCVRHENFGDSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.274_L1.47 | NO: 195 | APGKCLEWVGRIRSKYNNYATYYADSVKGRFTISRDDSK |
| _VH_G44C_V | | NTLYLQMNSLRAEDTAVYYCVRHENFGGSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.275_L1.47 | NO: 196 | APGKCLEWVGRIRSKANNYATYYADSVKGRFTISRDDSK |
| _VH_G44C_V | | NTLYLQMNSLRAEDTAVYYCVRHENFGDSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.276_L1.47 | NO: 197 | APGKCLEWVGRIRSKANNYATYYADSVKGRFTISRDDSK |
| _VH_G44C_V | | NTLYLQMNSLRAEDTAVYYCVRHENFGGSYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.279_L1.47 | NO: 198 | APGKCLEWVGRIRSKYNNYATYYADSVKGRFTISRDDSK |
| _VH_G44C_V | | NTLYLQMNSLRAEDTAVYYCVRHTNFGGEYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | EVQLVESGGGLVQPGGSLRLSCAASGFTFSTYAMNWVRQ |
| H1.280_L1.47 | NO: 199 | APGKCLEWVGRIRSKYNNYATYYADSVKGRFTISRDDSK |
| _VH_G44C_V | | NTLYLQMNSLRAEDTAVYYCVRHENFGGEYVSWFAYWG |
| L_G100C_scF | | QGTLVTVSSGKPGSGKPGSGKPGSGKPGSQAVVTQEPSLT |
| V | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGCGTKLTVL |
| Anti-CD3- | SEQ ID | QAVVTQEPSLTVSPGGTVTLTCGSSTGAVTTSNYANWVQ |
| L1.47_H1.214 | NO: 200 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKYNNYATYYADSV |
| | | KGRFTISRDDSKNTLYLQMNSLRAEDTAVYYCVRHTNFG |
| | | DSYVSWFAYWGQGTLVTVSS |
| Anti-CD3- | SEQ ID | QAVVTQEPSLTVSPGGTVTLTCGSSTGAVTTSNYANWVQ |
| L1.47_H1.270 | NO: 201 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKYNNYATYYADSV |
| | | KGRFTISRDDSKNTLYLQMNSLRAEDTAVYYCVRHTNFG |
| | | GSYVSWFAYWGQGTLVTVSS |
| Anti-CD3- | SEQ ID | QAVVTQEPSLTVSPGGTVTLTCGSSTGAVTTSNYANWVQ |
| L1.47_H1.271 | NO: 202 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKANNYATYYADSV |
| | | KGRFTISRDDSKNTLYLQMNSLRAEDTAVYYCVRHTNFG |
| | | DSYVSWFAYWGQGTLVTVSS |
| Anti-CD3- | SEQ ID | QAVVTQEPSLTVSPGGTVTLTCGSSTGAVTTSNYANWVQ |
| L1.47_H1.272 | NO: 203 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKANNYATYYADSV |
| | | KGRFTISRDDSKNTLYLQMNSLRAEDTAVYYCVRHTNFG |
| | | GSYVSWFAYWGQGTLVTVSS |
| Anti-CD3- | SEQ ID | QAVVTQEPSLTVSPGGTVTLTCGSSTGAVTTSNYANWVQ |
| L1.47_H1.273 | NO: 204 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKYNNYATYYADSV |
| | | KGRFTISRDDSKNTLYLQMNSLRAEDTAVYYCVRHENFG |
| | | DSYVSWFAYWGQGTLVTVSS |
| Anti-CD3- | SEQ ID | QAVVTQEPSLTVSPGGTVTLTCGSSTGAVTTSNYANWVQ |
| L1.47_H1.274 | NO: 205 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKYNNYATYYADSV |
| | | KGRFTISRDDSKNTLYLQMNSLRAEDTAVYYCVRHENFG |
| | | GSYVSWFAYWGQGTLVTVSS |
| Anti-CD3- | SEQ ID | QAVVTQEPSLTVSPGGTVTLTCGSSTGAVTTSNYANWVQ |
| L1.47_H1.275 | NO: 206 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKANNYATYYADSV |
| | | KGRFTISRDDSKNTLYLQMNSLRAEDTAVYYCVRHENFG |
| | | DSYVSWFAYWGQGTLVTVSS |
| Anti-CD3- | SEQ ID | QAVVTQEPSLTVSPGGTVTLTCGSSTGAVTTSNYANWVQ |
| L1.47_H1.276 | NO: 207 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| V | | FSTYAMNWVRQAPGKCLEWVGRIRSKANNYATYYADSV |
| | | KGRFTISRDDSKNTLYLQMNSLRAEDTAVYYCVRHENFG |
| | | GSYVSWFAYWGQGTLVTVSS |
| Anti-CD3- | SEQ ID | QAVVTQEPSLTVSPGGTVTLTCGSSTGAVTTSNYANWVQ |
| L1.47_H1.279 | NO: 208 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_scF | | GSGKPGSGKPGSEVQLVESGGGLVQPGGSLRLSCAASGFT |
| c | | FSTYAMNWVRQAPGKCLEWVGRIRSKYNNYATYYADSV |
| | | KGRFTISRDDSKNTLYLQMNSLRAEDTAVYYCVRHTNFG |
| | | GEYVSWFAYWGQGTLVTVSS |
| Anti-CD3- | SEQ ID | QAVVTQEPSLTVSPGGTVTLTCGSSTGAVTTSNYANWVQ |
| L1.47_H1.280 | NO: 209 | QKPGKSPRGLIGGTNKRAPGVPARFSGSLLGGKAALTISG |
| _VH_G44C_V | | AQPEDEADYYCALWYSNHWVFGCGTKLTVLGKPGSGKP |
| L_G100C_ | | GSGKPGSGKPGSEVQLVESGGGLVQPGGSLRLSCAASGFT |
| scFc | | FSTYAMNWVRQAPGKCLEWVGRIRSKYNNYATYYADSV |
| | | KGRFTISRDDSKNTLYLQMNSLRAEDTAVYYCVRHENFG |
| | | GEYVSWFAYWGQGTLVTVSS |
| Chain 1- | SEQ ID | QVQLVQSGAEVKKPGASVKVSCKASGYTFTSYVMHWVR |
| CD19- | NO: 210 | QAPGQGLEWMGYINPYNDGTKYNEKFQGRVTITSDKSTS |
| NEW_VH_Ig | | TAYMELSSLRSEDTAVYYCARGTYYYGTRVFDYWGQGT |
| G1_pI(−)_ | | LVTVSSASTKGPSVFPLAPSSKSTSGGTAALGCLVKDYFPE |
| Isosteric_A_ | | PVTVSWNSGALTSGVHTFPAVLQSSGLYSLSSVVTVPSSSL |
| PVA/S267K/ | | GTQTYICNVNHKPSDTKVDKKVEPKSCDKTHTCPPCPAPP |
| L368D/K370S/ | | VAGPSVFLFPPKPKDTLMISRTPEVTCVVVDVKHEDPEVK |
| M428L/N434S | | FNWYVDGVEVHNAKTKPREEEYNSTYRVVSVLTVLHQD |
| | | WLNGKEYKCKVSNKALPAPIEKTISKAKGQPREPQVYTLP |
| | | PSREEMTKNQVSLTCDVSGFYPSDIAVEWESDGQPENNYK |
| | | TTPPVLDSDGSFFLYSKLTVDKSRWEQGDVFSCSVLHEAL |
| | | HSHYTQKSLSLSPGK |
| Chain 2- | SEQ ID | EVQLVESGGGLVQPGGSLRLSCAASGFTFSTYAMNWVRQ |
| [CD3]_H1.30_ | NO: 211 | APGKGLEWVGRIRSKYNNYATYYADSVKGRFTISRDDSK |
| L1.47_scFv(G | | NTLYLQMNSLRAEDTAVYYCVRHGNFGDSYVSWFAYWG |
| KPGS)4_Fc(2 | | QGTLVTVSSGKPGSGKPGSGKPGSGKPGSQAVVTQEPSLT |
| 16)_IgG1_C22 | | VSPGGTVTLTCGSSTGAVTTSNYANWVQQKPGKSPRGLIG |
| OS/PVA/S267 | | GTNKRAPGVPARFSGSLLGGKAALTISGAQPEDEADYYCA |
| K/S364K/E357 | | LWYSNHWVFGGGTKLTVLEPKSSDKTHTCPPCPAPPVAG |
| Q/M428L/N43 | | PSVFLFPPKPKDTLMISRTPEVTCVVVDVKHEDPEVKFNW |
| 4S | | YVDGVEVHNAKTKPREEQYNSTYRVVSVLTVLHQDWLN |
| | | GKEYKCKVSNKALPAPIEKTISKAKGQPREPQVYTLPPSRE |
| | | QMTKNQVKLTCLVKGFYPSDIAVEWESNGQPENNYKTTP |
| | | PVLDSDGSFFLYSKLTVDKSRWQQGNVFSCSVLHEALHSH |
| | | YTQKSLSLSPGK |
| Chain 3- | SEQ ID | DIVMTQSPLSLPVTPGEPASISCRSSKSLQNVNGNTYLYWF |
| 4G7 L4.201 | NO: 212 | LQKPGQSPQLLIYRASNLNSGVPDRFSGSGSGTDFTLKISR |
| Light Chain | | VEAEDVGVYYCMQHLEYPITFGQGTKLEIKRTVAAPSVFI |
| | | FPPSDEQLKSGTASVVCLLNNFYPREAKVQWKVDNALQS |
| | | GNSQESVTEQDSKDSTYSLSSTLTLSKADYEKHKVYACEV |
| | | THQGLSSPVTKSFNRGEC |
| Chain 1- | SEQ ID | EVQLVESGGGLVKPGGSLKLSCAASGYTFTSYVMHWVRQ |
| CD19- | NO: 213 | APGKGLEWIGYINPYNDGTKYNEKFQGRVTISSDKSISTAY |
| OG_VHa_IgG | | MELSSLRSEDTAMYYCARGTYYYGTRVFDYWGQGTLVT |
| 1 | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSDTKVDKKVEPKSCDKTHTCPPCPAPPVA |
| | | GPSVFLFPPKPKDTLMISRTPEVTCVVVDVKHEDPEVKFN |
| | | WYVDGVEVHNAKTKPREEEYNSTYRVVSVLTVLHQDWL |
| | | NGKEYKCKVSNKALPAPIEKTISKAKGQPREPQVYTLPPSR |
| | | EEMTKNQVSLTCDVSGFYPSDIAVEWESDGQPENNYKTTP |
| | | PVLDSDGSFFLYSKLTVDKSRWEQGDVFSCSVMHEALHN |
| | | HYTQKSLSLSPGK |
| Chain 2- | SEQ ID | EVQLVESGGGLVQPGGSLRLSCAASGFTFSTYAMNWVRQ |
| [CD3]_H1.89 | NO: 214 | APGKGLEWVGRIRSKYNNYATYYADSVKGRFTISRDDSK |
| L1.47_scFv(G | | NTLYLQMNSLRAEDTAVYYCVRHGNFGDEYVSWFAYWG |
| KPGS)4_Fc(2 | | QGTLVTVSSGKPGSGKPGSGKPGSGKPGSQAVVTQEPSLT |
| 16)_IgG1 | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVLEPKSSDKTHTCPPCPAPPVAG |
| | | PSVFLFPPKPKDTLMISRTPEVTCVVVDVKHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRE |
| | | QMTKNQVKLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| Chain 3- | SEQ ID | DIVMTQSPATLSLSPGERATLSCRSSKSLQNVNGNTYLYW |
| 4G7 L1.155 | NO: 215 | FQQKPGQSPQLLIYRMSNLNSGVPDRFSGSGSGTEFTLTISS |
| Light Chain | | LEPEDFAVYYCMQHLEYPITFGAGTKLEIKRTVAAPSVFIF |
| | | PPSDEQLKSGTASVVCLLNNFYPREAKVQWKVDNALQSG |
| | | NSQESVTEQDSKDSTYSLSSTLTLSKADYEKHKVYACEVT |
| | | HQGLSSPVTKSFNRGEC |
| Chain 2- | SEQ ID | EVQLVESGGGLVQPGGSLRLSCAASGFTFSTYAMNWVRQ |
| [CD3]_H1.33 | NO: 216 | APGKGLEWVGRIRSKYNNYATYYADSVKGRFTISRDDSK |
| L1.47_scFv(G | | NTLYLQMNSLRAEDTAVYYCVRHGNFGDSYVSWFDYWG |
| KPGS)4_Fc(2 | | QGTLVTVSSGKPGSGKPGSGKPGSGKPGSQAVVTQEPSLT |
| 16)_IgG1 | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVLEPKSSDKTHTCPPCPAPPVAG |
| | | PSVFLFPPKPKDTLMISRTPEVTCVVVDVKHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRE |
| | | QMTKNQVKLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| Chain_2- | SEQ ID | EVQLVESGGGLVQPGGSLRLSCAASGFTFSTYAMNWVRQ |
| [CD3]_H1.32 | NO: 217 | APGKGLEWVGRIRSKANNYATYYADSVKGRFTISRDDSK |
| L1.47_scFv(G | | NTLYLQMNSLRAEDTAVYYCVRHGNFGDSYVSWFAYWG |
| KPGS)4_Fc(2 | | QGTLVTVSSGKPGSGKPGSGKPGSGKPGSQAVVTQEPSLT |
| 16)_IgG1 | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVLEPKSSDKTHTCPPCPAPPVAG |
| | | PSVFLEPPKPKDTLMISRTPEVTCVVVDVKHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRE |
| | | QMTKNQVKLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| Chain_2- | SEQ ID | EVQLVESGGGLVQPGGSLRLSCAASGFTFSTYAMNWVRQ |
| [CD3]_H1.30 | NO: 218 | APGKGLEWVGRIRSKYNNYATYYADSVKGRFTISRDDSK |
| L1.47_scFv(G | | NTLYLQMNSLRAEDTAVYYCVRHGNFGDSYVSWFAYWG |
| KPGS)4_Fc(2 | | QGTLVTVSSGKPGSGKPGSGKPGSGKPGSQAVVTQEPSLT |
| 16)_IgG1 | | VSPGGTVTLTCGSSTGAVTTSNYANWVQQKPGKSPRGLIG |
| | | GTNKRAPGVPARFSGSLLGGKAALTISGAQPEDEADYYCA |
| | | LWYSNHWVFGGGTKLTVLEPKSSDKTHTCPPCPAPPVAG |
| | | PSVFLFPPKPKDTLMISRTPEVTCVVVDVKHEDPEVKFNW |
| | | YVDGVEVHNAKTKPREEQYNSTYRVVSVLTVLHQDWLN |
| | | GKEYKCKVSNKALPAPIEKTISKAKGQPREPQVYTLPPSRE |
| | | QMTKNQVKLTCLVKGFYPSDIAVEWESNGQPENNYKTTP |
| | | PVLDSDGSFFLYSKLTVDKSRWQQGNVFSCSVMHEALHN |
| | | HYTQKSLSLSPGK |
| Chain_2- | SEQ ID | QVQLVQSGAEVKKPGASVKVSCKASGYTFTSYVMHWVR |
| CD19- | NO: 219 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.280 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_ | | EDEADYYCALWYSNHWVFGCGTKLTVLGKPGSGKPGSG |
| IgG1 | | KPGSGKPGSEVQLVESGGGLVQPGGSLRLSCAASGFTFST |
| | | YAMNWVRQAPGKCLEWVGRIRSKYNNYATYYADSVKG |
| | | RFTISRDDSKNTLYLQMNSLRAEDTAVYYCVRHENFGGE |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 220 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.279 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKYNNYATYYADSVKG |
| | | RFTISRDDSKNTLYLQMNSLRAEDTAVYYCVRHTNFGGE |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 221 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.276 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKANNYATYYADSVKG |
| | | RFTISRDDSKNTLYLQMNSLRAEDTAVYYCVRHENFGGS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 222 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.275 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKANNYATYYADSVKG |
| | | RFTISRDDSKNTLYLQMNSLRAEDTAVYYCVRHENFGDS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 223 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.274 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKYNNYATYYADSVKG |
| | | RFTISRDDSKNTLYLQMNSLRAEDTAVYYCVRHENFGGS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 224 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.273 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKYNNYATYYADSVKG |
| | | RFTISRDDSKNTLYLQMNSLRAEDTAVYYCVRHENFGDS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 225 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.272 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKANNYATYYADSVKG |
| | | RFTISRDDSKNTLYLQMNSLRAEDTAVYYCVRHTNFGGS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 226 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.271 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKANNYATYYADSVKG |
| | | RFTISRDDSKNTLYLQMNSLRAEDTAVYYCVRHTNFGDS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 227 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.270 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKYNNYATYYADSVKG |
| | | RFTISRDDSKNTLYLQMNSLRAEDTAVYYCVRHTNFGGS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 228 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.214 | | PVTVSWNSGALTSGVHTFPAVLQSSGLYSLSSVVTVPSSSL |
| VH_G44C_VL | | GTQTYICNVNHKPSNTKVDKKVEPKSCGGGGSGGGGSQA |
| G100C_scFv( | | VVTQEPSLTVSPGGTVTLTCGSSTGAVTTSNYANWVQQK |
| GKPGS)4_(G4 | | PGKSPRGLIGGTNKRAPGVPARFSGSLLGGKAALTISGAQP |
| S)2_Fc(222)_I | | EDEADYYCALWYSNHWVFGCGTKLTVLGKPGSGKPGSG |
| gG1 | | KPGSGKPGSEVQLVESGGGLVQPGGSLRLSCAASGFTFST |
| | | YAMNWVRQAPGKCLEWVGRIRSKYNNYATYYADSVKG |
| | | RFTISRDDSKNTLYLQMNSLRAEDTAVYYCVRHTNFGDS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 229 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.89_sc | | PVTVSWNSGALTSGVHTFPAVLQSSGLYSLSSVVTVPSSSL |
| Fv(GKPGS)4 | | GTQTYICNVNHKPSNTKVDKKVEPKSCGGGGSGGGGSQA |
| Fc(216)_IgG1 | | VVTQEPSLTVSPGGTVTLTCGSSTGAVTTSNYANWVQQK |
| | | PGKSPRGLIGGTNKRAPGVPARFSGSLLGGKAALTISGAQP |
| | | EDEADYYCALWYSNHWVFGGGTKLTVLGKPGSGKPGSG |
| | | KPGSGKPGSEVQLVESGGGLVQPGGSLRLSCAASGFTFST |
| | | YAMNWVRQAPGKGLEWVGRIRSKYNNYATYYADSVKG |
| | | RFTISRDDSKNTLYLQMNSLRAEDTAVYYCVRHGNFGDE |
| | | YVSWFAYWGQGTLVTVSSEPKSSDKTHTCPPCPAPPVAGP |
| | | SVFLFPPKPKDTLMISRTPEVTCVVVDVKHEDPEVKFNWY |
| | | VDGVEVHNAKTKPREEQYNSTYRVVSVLTVLHQDWLNG |
| | | KEYKCKVSNKALPAPIEKTISKAKGQPREPQVYTLPPSREQ |
| | | MTKNQVKLTCLVKGFYPSDIAVEWESNGQPENNYKTTPP |
| | | VLDSDGSFFLYSKLTVDKSRWQQGNVFSCSVLHEALHSH |
| | | YTQKSLSLSPGK |
| Chain_2- | SEQ ID | QVQLVQSGAEVKKPGASVKVSCKASGYTFTSYVMHWVR |
| CD19- | NO: 230 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_L | | LVTVSSASTKGPSVFPLAPSSKSTSGGTAALGCLVKDYFPE |
| 1.47_H1.32_sc | | PVTVSWNSGALTSGVHTFPAVLQSSGLYSLSSVVTVPSSSL |
| Fv(GKPGS)4 | | GTQTYICNVNHKPSNTKVDKKVEPKSCGGGGSGGGGSQA |
| (G4S)2_Fc(22 | | VVTQEPSLTVSPGGTVTLTCGSSTGAVTTSNYANWVQQK |
| 2)_IgG1 | | PGKSPRGLIGGTNKRAPGVPARFSGSLLGGKAALTISGAQP |
| | | EDEADYYCALWYSNHWVFGGGTKLTVLGKPGSGKPGSG |
| | | KPGSGKPGSEVQLVESGGGLVQPGGSLRLSCAASGFTFST |
| | | YAMNWVRQAPGKGLEWVGRIRSKANNYATYYADSVKG |
| | | RFTISRDDSKNTLYLQMNSLRAEDTAVYYCVRHGNFGDS |
| | | YVSWFAYWGQGTLVTVSSGGGGSGGGGSKTHTCPPCPAP |
| | | PVAGPSVFLFPPKPKDTLMISRTPEVTCVVVDVKHEDPEV |
| | | KFNWYVDGVEVHNAKTKPREEQYNSTYRVVSVLTVLHQ |
| | | DWLNGKEYKCKVSNKALPAPIEKTISKAKGQPREPQVYTL |
| | | PPSREQMTKNQVKLTCLVKGFYPSDIAVEWESNGQPENN |
| | | YKTTPPVLDSDGSFFLYSKLTVDKSRWQQGNVFSCSVLHE |
| | | ALHSHYTQKSLSLSPGK |
| Chain_2- | SEQ ID | QVQLVQSGAEVKKPGASVKVSCKASGYTFTSYVMHWVR |
| CD19- | NO: 231 | QAPGQGLEWMGYINPYNDGTKYNEKFQGRVTITSDKSTS |
| NEW_VH_(G | | TAYMELSSLRSEDTAVYYCARGTYYYGTRVFDYWGQGT |
| 4S)2_[CD3]_H | | LVTVSSASTKGPSVFPLAPSSKSTSGGTAALGCLVKDYFPE |
| 1.30_L1.47_sc | | PVTVSWNSGALTSGVHTFPAVLQSSGLYSLSSVVTVPSSSL |
| Fv(GKPGS)4 | | GTQTYICNVNHKPSNTKVDKKVEPKSCGGGGSGGGGSEV |
| (G4S)2_Fc(22 | | QLVESGGGLVQPGGSLRLSCAASGFTFSTYAMNWVRQAP |
| 2)_IgG1 | | GKGLEWVGRIRSKYNNYATYYADSVKGRFTISRDDSKNT |
| | | LYLQMNSLRAEDTAVYYCVRHGNFGDSYVSWFAYWGQ |
| | | GTLVTVSSGKPGSGKPGSGKPGSGKPGSQAVVTQEPSLTV |
| | | SPGGTVTLTCGSSTGAVTTSNYANWVQQKPGKSPRGLIGG |
| | | TNKRAPGVPARFSGSLLGGKAALTISGAQPEDEADYYCAL |
| | | WYSNHWVFGGGTKLTVLGGGGSGGGGSKTHTCPPCPAPP |
| | | VAGPSVFLFPPKPKDTLMISRTPEVTCVVVDVKHEDPEVK |
| | | FNWYVDGVEVHNAKTKPREEQYNSTYRVVSVLTVLHQD |
| | | WLNGKEYKCKVSNKALPAPIEKTISKAKGQPREPQVYTLP |
| | | PSREQMTKNQVKLTCLVKGFYPSDIAVEWESNGQPENNY |
| | | KTTPPVLDSDGSFFLYSKLTVDKSRWQQGNVFSCSVLHEA |
| | | LHSHYTQKSLSLSPGK |
| Chain_1- | SEQ ID | EVQLVESGGGLVKPGGSLKLSCAASGYTFTSYVMHWVRQ |
| CD19- | NO: 232 | APGKGLEWIGYINPYNDGTKYNEKFQGRVTISSDKSISTAY |
| OG_VHb_IgG | | MELSSLRSEDTAMYYCARGTYYYGTRVFDYWGQGTLVT |
| 1 | | VSSASTKGPSVFPLAPSSKSTSGGTAALGCLVKDYFPEPVT |
| | | VSWNSGALTSGVHTFPAVLQSSGLYSLSSVVTVPSSSLGT |
| | | QTYICNVNHKPSDTKVDKKVEPKSCDKTHTCPPCPAPPVA |
| | | GPSVFLFPPKPKDTLMISRTPEVTCVVVDVKHEDPEVKEN |
| | | WYVDGVEVHNAKTKPREEEYNSTYRVVSVLTVLHQDWL |
| | | NGKEYKCKVSNKALPAPIEKTISKAKGQPREPQVYTLPPSR |
| | | EEMTKNQVSLTCDVSGFYPSDIAVEWESDGQPENNYKTTP |
| | | PVLDSDGSFFLYSKLTVDKSRWEQGDVFSCSVLHEALHSH |
| | | YTQKSLSLSPGK |
| Chain_2- | SEQ ID | EVQLVESGGGLVKPGGSLKLSCAASGYTFTSYVMHWVRQ |
| CD19- | NO: 233 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.280_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_ | | EADYYCALWYSNHWVFGCGTKLTVLGKPGSGKPGSGKP |
| IgG1 | | GSGKPGSEVQLVESGGGLVQPGGSLRLSCAASGFTFSTYA |
| | | MNWVRQAPGKCLEWVGRIRSKYNNYATYYADSVKGRFT |
| | | ISRDDSKNTLYLQMNSLRAEDTAVYYCVRHENFGGEYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 234 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.279_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_ | | EADYYCALWYSNHWVFGCGTKLTVLGKPGSGKPGSGKP |
| IgG1 | | GSGKPGSEVQLVESGGGLVQPGGSLRLSCAASGFTFSTYA |
| | | MNWVRQAPGKCLEWVGRIRSKYNNYATYYADSVKGRFT |
| | | ISRDDSKNTLYLQMNSLRAEDTAVYYCVRHTNFGGEYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 235 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.276_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_ | | EADYYCALWYSNHWVFGCGTKLTVLGKPGSGKPGSGKP |
| IgG1 | | GSGKPGSEVQLVESGGGLVQPGGSLRLSCAASGFTFSTYA |
| | | MNWVRQAPGKCLEWVGRIRSKANNYATYYADSVKGRFT |
| | | ISRDDSKNTLYLQMNSLRAEDTAVYYCVRHENFGGSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 236 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.275_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKANNYATYYADSVKGRFT |
| | | ISRDDSKNTLYLQMNSLRAEDTAVYYCVRHENFGDSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 237 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.274_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKYNNYATYYADSVKGRFT |
| | | ISRDDSKNTLYLQMNSLRAEDTAVYYCVRHENFGGSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 238 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.273_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKYNNYATYYADSVKGRFT |
| | | ISRDDSKNTLYLQMNSLRAEDTAVYYCVRHENFGDSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 239 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.272_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKANNYATYYADSVKGRFT |
| | | ISRDDSKNTLYLQMNSLRAEDTAVYYCVRHTNFGGSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 240 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.271_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKANNYATYYADSVKGRFT |
| | | ISRDDSKNTLYLQMNSLRAEDTAVYYCVRHTNFGDSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKFN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 241 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.270_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKYNNYATYYADSVKGRFT |
| | | ISRDDSKNTLYLQMNSLRAEDTAVYYCVRHTNFGGSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 242 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.214_V | | VSWNSGALTSGVHTFPAVLQSSGLYSLSSVVTVPSSSLGT |
| H_G44C_VL | | QTYICNVNHKPSNTKVDKKVEPKSCGGGGSGGGGSQAVV |
| G100C_scFv( | | TQEPSLTVSPGGTVTLTCGSSTGAVTTSNYANWVQQKPG |
| GKPGS)4_(G4 | | KSPRGLIGGTNKRAPGVPARFSGSLLGGKAALTISGAQPED |
| S)2_Fc(222)_I | | EADYYCALWYSNHWVFGCGTKLTVLGKPGSGKPGSGKP |
| gG1 | | GSGKPGSEVQLVESGGGLVQPGGSLRLSCAASGFTFSTYA |
| | | MNWVRQAPGKCLEWVGRIRSKYNNYATYYADSVKGRFT |
| | | ISRDDSKNTLYLQMNSLRAEDTAVYYCVRHTNFGDSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 243 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.89_scF | | VSWNSGALTSGVHTFPAVLQSSGLYSLSSVVTVPSSSLGT |
| v(GKPGS)4_F | | QTYICNVNHKPSNTKVDKKVEPKSCGGGGSGGGGSQAVV |
| c(216)_IgG1 | | TQEPSLTVSPGGTVTLTCGSSTGAVTTSNYANWVQQKPG |
| | | KSPRGLIGGTNKRAPGVPARFSGSLLGGKAALTISGAQPED |
| | | EADYYCALWYSNHWVFGGGTKLTVLGKPGSGKPGSGKP |
| | | GSGKPGSEVQLVESGGGLVQPGGSLRLSCAASGFTFSTYA |
| | | MNWVRQAPGKGLEWVGRIRSKYNNYATYYADSVKGRFT |
| | | ISRDDSKNTLYLQMNSLRAEDTAVYYCVRHGNFGDEYVS |
| | | WFAYWGQGTLVTVSSEPKSSDKTHTCPPCPAPPVAGPSVF |
| | | LFPPKPKDTLMISRTPEVTCVVVDVKHEDPEVKFNWYVD |
| | | GVEVHNAKTKPREEQYNSTYRVVSVLTVLHQDWLNGKE |
| | | YKCKVSNKALPAPIEKTISKAKGQPREPQVYTLPPSREQMT |
| | | KNQVKLTCLVKGFYPSDIAVEWESNGQPENNYKTTPPVL |
| | | DSDGSFFLYSKLTVDKSRWQQGNVFSCSVLHEALHSHYT |
| | | QKSLSLSPGK |
| Chain_2- | SEQ ID | EVQLVESGGGLVKPGGSLKLSCAASGYTFTSYVMHWVRQ |
| CD19- | NO: 244 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_L1. | | VSSASTKGPSVFPLAPSSKSTSGGTAALGCLVKDYFPEPVT |
| 47_H1.32_scF | | VSWNSGALTSGVHTFPAVLQSSGLYSLSSVVTVPSSSLGT |
| v(GKPGS)4_( | | QTYICNVNHKPSNTKVDKKVEPKSCGGGGSGGGGSQAVV |
| G4S)2_Fc(222 | | TQEPSLTVSPGGTVTLTCGSSTGAVTTSNYANWVQQKPG |
| )_IgG1 | | KSPRGLIGGTNKRAPGVPARFSGSLLGGKAALTISGAQPED |
| | | EADYYCALWYSNHWVFGGGTKLTVLGKPGSGKPGSGKP |
| | | GSGKPGSEVQLVESGGGLVQPGGSLRLSCAASGFTFSTYA |
| | | MNWVRQAPGKGLEWVGRIRSKANNYATYYADSVKGRFT |
| | | ISRDDSKNTLYLQMNSLRAEDTAVYYCVRHGNFGDSYVS |
| | | WFAYWGQGTLVTVSSGGGGSGGGGSKTHTCPPCPAPPVA |
| | | GPSVFLFPPKPKDTLMISRTPEVTCVVVDVKHEDPEVKEN |
| | | WYVDGVEVHNAKTKPREEQYNSTYRVVSVLTVLHQDWL |
| | | NGKEYKCKVSNKALPAPIEKTISKAKGQPREPQVYTLPPSR |
| | | EQMTKNQVKLTCLVKGFYPSDIAVEWESNGQPENNYKTT |
| | | PPVLDSDGSFFLYSKLTVDKSRWQQGNVFSCSVLHEALHS |
| | | HYTQKSLSLSPGK |
| Chain_2- | SEQ ID | EVQLVESGGGLVKPGGSLKLSCAASGYTFTSYVMHWVRQ |
| CD19- | NO: 245 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_H1. | | VSSASTKGPSVFPLAPSSKSTSGGTAALGCLVKDYFPEPVT |
| 30_L1.47_scF | | VSWNSGALTSGVHTFPAVLQSSGLYSLSSVVTVPSSSLGT |
| v(GKPGS)4_( | | QTYICNVNHKPSNTKVDKKVEPKSCGGGGSGGGGSEVQL |
| G4S)2_Fc(222 | | VESGGGLVQPGGSLRLSCAASGFTFSTYAMNWVRQAPGK |
| )_IgG1 | | GLEWVGRIRSKYNNYATYYADSVKGRFTISRDDSKNTLY |
| | | LQMNSLRAEDTAVYYCVRHGNFGDSYVSWFAYWGQGTL |
| | | VTVSSGKPGSGKPGSGKPGSGKPGSQAVVTQEPSLTVSPG |
| | | GTVTLTCGSSTGAVTTSNYANWVQQKPGKSPRGLIGGTN |
| | | KRAPGVPARFSGSLLGGKAALTISGAQPEDEADYYCALW |
| | | YSNHWVFGGGTKLTVLGGGGSGGGGSKTHTCPPCPAPPV |
| | | AGPSVFLFPPKPKDTLMISRTPEVTCVVVDVKHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REQMTKNQVKLTCLVKGFYPSDIAVEWESNGQPENNYKT |
| | | TPPVLDSDGSFFLYSKLTVDKSRWQQGNVFSCSVLHEALH |
| | | SHYTQKSLSLSPGK |
| Chain_2- | SEQ ID | EVQLVESGGGLVKPGGSLKLSCAASGYTFTSYVMHWVRQ |
| CD19- | NO: 246 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_H1. | | VSSASTKGPSVFPLAPSSKSTSGGTAALGCLVKDYFPEPVT |
| 89_L1.47_scF | | VSWNSGALTSGVHTFPAVLQSSGLYSLSSVVTVPSSSLGT |
| v(GKPGS)4_F | | QTYICNVNHKPSNTKVDKKVEPKSCGGGGSGGGGSEVQL |
| c(222)_IgG1 | | VESGGGLVQPGGSLRLSCAASGFTFSTYAMNWVRQAPGK |
| | | GLEWVGRIRSKYNNYATYYADSVKGRFTISRDDSKNTLY |
| | | LQMNSLRAEDTAVYYCVRHGNFGDEYVSWFAYWGQGTL |
| | | VTVSSGKPGSGKPGSGKPGSGKPGSQAVVTQEPSLTVSPG |
| | | GTVTLTCGSSTGAVTTSNYANWVQQKPGKSPRGLIGGTN |
| | | KRAPGVPARFSGSLLGGKAALTISGAQPEDEADYYCALW |
| | | YSNHWVFGGGTKLTVLGGGGSGGGGSKTHTCPPCPAPPV |
| | | AGPSVFLFPPKPKDTLMISRTPEVTCVVVDVKHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REQMTKNQVKLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| Chain_2- | SEQ ID | EVQLVESGGGLVKPGGSLKLSCAASGYTFTSYVMHWVRQ |
| CD19- | NO: 247 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_H1. | | VSSASTKGPSVFPLAPSSKSTSGGTAALGCLVKDYFPEPVT |
| 33_L1.47_scFv | | VSWNSGALTSGVHTFPAVLQSSGLYSLSSVVTVPSSSLGT |
| (GKPGS)4_ | | QTYICNVNHKPSNTKVDKKVEPKSCGGGGSGGGGSEVQL |
| Fc(222)_IgG1 | | VESGGGLVQPGGSLRLSCAASGFTFSTYAMNWVRQAPGK |
| | | GLEWVGRIRSKYNNYATYYADSVKGRFTISRDDSKNTLY |
| | | LQMNSLRAEDTAVYYCVRHGNFGDSYVSWFDYWGQGTL |
| | | VTVSSGKPGSGKPGSGKPGSGKPGSQAVVTQEPSLTVSPG |
| | | GTVTLTCGSSTGAVTTSNYANWVQQKPGKSPRGLIGGTN |
| | | KRAPGVPARFSGSLLGGKAALTISGAQPEDEADYYCALW |
| | | YSNHWVFGGGTKLTVLGGGGSGGGGSKTHTCPPCPAPPV |
| | | AGPSVFLFPPKPKDTLMISRTPEVTCVVVDVKHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REQMTKNQVKLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| Chain_2- | SEQ ID | EVQLVESGGGLVKPGGSLKLSCAASGYTFTSYVMHWVRQ |
| CD19- | NO: 248 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_H1. | | VSSASTKGPSVFPLAPSSKSTSGGTAALGCLVKDYFPEPVT |
| 32_L1.47_scF | | VSWNSGALTSGVHTFPAVLQSSGLYSLSSVVTVPSSSLGT |
| v(GKPGS)4_F | | QTYICNVNHKPSNTKVDKKVEPKSCGGGGSGGGGSEVQL |
| c(222)_IgG1 | | VESGGGLVQPGGSLRLSCAASGFTFSTYAMNWVRQAPGK |
| | | GLEWVGRIRSKANNYATYYADSVKGRFTISRDDSKNTLY |
| | | LQMNSLRAEDTAVYYCVRHGNFGDSYVSWFAYWGQGTL |
| | | VTVSSGKPGSGKPGSGKPGSGKPGSQAVVTQEPSLTVSPG |
| | | GTVTLTCGSSTGAVTTSNYANWVQQKPGKSPRGLIGGTN |
| | | KRAPGVPARFSGSLLGGKAALTISGAQPEDEADYYCALW |
| | | YSNHWVFGGGTKLTVLGGGGSGGGGSKTHTCPPCPAPPV |
| | | AGPSVFLFPPKPKDTLMISRTPEVTCVVVDVKHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REQMTKNQVKLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |
| Chain_2- | SEQ ID | EVQLVESGGGLVKPGGSLKLSCAASGYTFTSYVMHWVRQ |
| CD19- | NO: 249 | APGKGLEWIGYINPYNDGTKYNEKFQGRVTISSDKSISTAY |
| OG_VH_(G4S | | MELSSLRSEDTAMYYCARGTYYYGTRVFDYWGQGTLVT |
| )2_[CD3]_H1. | | VSSASTKGPSVFPLAPSSKSTSGGTAALGCLVKDYFPEPVT |
| 30_L1.47_scF | | VSWNSGALTSGVHTFPAVLQSSGLYSLSSVVTVPSSSLGT |
| v(GKPGS)4_F | | QTYICNVNHKPSNTKVDKKVEPKSCGGGGSGGGGSEVQL |
| c(222)_IgG1 | | VESGGGLVQPGGSLRLSCAASGFTFSTYAMNWVRQAPGK |
| | | GLEWVGRIRSKYNNYATYYADSVKGRFTISRDDSKNTLY |
| | | LQMNSLRAEDTAVYYCVRHGNFGDSYVSWFAYWGQGTL |
| | | VTVSSGKPGSGKPGSGKPGSGKPGSQAVVTQEPSLTVSPG |
| | | GTVTLTCGSSTGAVTTSNYANWVQQKPGKSPRGLIGGTN |
| | | KRAPGVPARFSGSLLGGKAALTISGAQPEDEADYYCALW |
| | | YSNHWVFGGGTKLTVLGGGGSGGGGSKTHTCPPCPAPPV |
| | | AGPSVFLFPPKPKDTLMISRTPEVTCVVVDVKHEDPEVKF |
| | | NWYVDGVEVHNAKTKPREEQYNSTYRVVSVLTVLHQDW |
| | | LNGKEYKCKVSNKALPAPIEKTISKAKGQPREPQVYTLPPS |
| | | REQMTKNQVKLTCLVKGFYPSDIAVEWESNGQPENNYKT |
| | | TPPVLDSDGSFFLYSKLTVDKSRWQQGNVFSCSVMHEAL |
| | | HNHYTQKSLSLSPGK |

1. A bi-specific anti-CD19 and anti-CD3 antigen binding molecule comprising:
an anti-CD19 antigen binding domain comprising
a first anti-CD19 binding subunit comprising CDR amino acid sequences of SEQ ID NOs: 1, 2, and 3, and
a second anti-CD19 binding subunit comprising CDR amino acid sequences of SEQ ID NOs: 4, 5, and 6; and
an anti-CD3 antigen binding domain comprising
a first anti-CD3 binding subunit comprising the CDR amino acid sequences of SEQ ID NO: 9, SEQ ID NO: 10 or 11, and SEQ ID NO: 12, 13, 14, 15, or 16, and
an anti-CD3 second binding subunit comprising the CDR amino acid sequences of SEQ ID NOs: 17, 18, and 19.

2.-3. (canceled)

4. A bi-specific anti-CD19 and anti-CD3 antigen binding molecule, wherein the anti-CD19 antigen binding domain comprises:
a first anti-CD19 binding subunit comprising at least 95% sequence identity to the amino acid sequence of SEQ ID NO: 7; and
a second anti-CD19 binding subunit comprising at least 95% sequence identity to the amino acid sequence of SEQ ID NO: 8; and
a light chain variable region comprising at least 95% sequence identity to an amino acid sequence of SEQ ID NO: 180; and
a heavy chain variable region comprising at least 95% sequence identity to an amino acid sequence selected from the group consisting of SEQ ID NO: 179, 181, 182, 183, 184, 185, 186, 187, 188, and 189.

5. The bi-specific anti-CD19 and anti-CD3 antigen binding molecule of claim 4, wherein the anti-CD19 antigen binding domain comprises:
a first anti-CD19 binding subunit comprising the amino acid sequence of SEQ ID NO: 7; and
a second anti-CD19 binding subunit comprising the amino acid sequence of SEQ ID NO: 8.

6. (canceled)

7. The bi-specific anti-CD19 and anti-CD3 antigen binding molecule of claim 4, wherein the anti-CD19 antigen binding domain comprises:
the light chain variable region comprising an amino acid sequence of SEQ ID NO: 180; and
the heavy chain variable region comprising an amino acid sequence selected from the group consisting of SEQ ID NO: 179, 181, 182, 183, 184, 185, 186, 187, 188, and 189.

8.-9. (canceled)

10. The bi-specific anti-CD19 and anti-CD3 antigen binding molecule of claim 1, wherein the bi-specific antibody is a 1+1 Fab-scFv-Fc, 2+1 Fab₂-scFv-Fc, 1+1 Common Light Chain, 2+1 Common Light Chain, 2+1 mAb-scFv, 2+1 stackFab₂-scFv-Fc, Dual scFv, One-arm scFv-mAb, scFv-mAb, Bispecific mAb, One-arm central-scFv, mAb-Fv, central-Fv, or Trident.

11. (canceled)

12. The bi-specific anti-CD19 and anti-CD3 antigen binding molecule of claim 9, comprising:
a first monomer comprising, from N- to C-terminus, a VH-CH1-first domain linker-scFv-second domain linker-CH2-CH3, wherein
the scFv comprises an antigen binding domain that binds CD3; and
the CH2-CH3 is first variant Fc domain;
a second monomer comprising a VH-CH1-hinge-CH2-CH3, wherein the CH2-CH3 is a second variant Fc domain;
a first light chain that together with the first monomer forms a Fab domain that comprises an antigen binding domain that binds CD19; and
a second light chain that together with the second monomer forms a Fab domain that comprises an antigen binding domain that binds CD19.

13.-36. (canceled)

37. A bi-specific anti-CD19 and anti-CD3 antigen binding molecule, wherein the binding molecule is a 1+1 Fab-scFv-Fc and comprises an amino acid sequence selected from the group consisting of:
SEQ ID NOs: 210, 211, and 212;
SEQ ID NOs: 213, 214, and 215;
SEQ ID NOs: 213, 216, and 215;
SEQ ID NOs: 213, 217, and 215; and
SEQ ID NOs: 213, 218, and 215.

38. The bi-specific anti-CD19 and anti-CD3 antigen binding molecule of claim 12, wherein the binding molecule is a 2+1 Fab₂-scFv-Fc and comprises an amino acid sequence selected from the group consisting of:
SEQ ID NOs: 210, 219, and 212;
SEQ ID NOs: 210, 220, and 212;
SEQ ID NOs: 210, 221, and 212;
SEQ ID NOs: 210, 222, and 212;
SEQ ID NOs: 210, 223, and 212;
SEQ ID NOs: 210, 224, and 212;
SEQ ID NOs: 210, 225, and 212;
SEQ ID NOs: 210, 226, and 212;
SEQ ID NOs: 210, 227, and 212;
SEQ ID NOs: 210, 228, and 212;
SEQ ID NOs: 210, 229, and 212;
SEQ ID NOs: 210, 230, and 212;
SEQ ID NOs: 210, 231, and 212;
SEQ ID NOs: 232, 233, and 215;
SEQ ID NOs: 232, 234, and 215;
SEQ ID NOs: 232, 235, and 215;
SEQ ID NOs: 232, 236, and 215;
SEQ ID NOs: 232, 237, and 215;
SEQ ID NOs: 232, 238, and 215;
SEQ ID NOs: 232, 239, and 215;
SEQ ID NOs: 232, 240, and 215;
SEQ ID NOs: 232, 241, and 215;
SEQ ID NOs: 232, 242, and 215;
SEQ ID NOs: 232, 243, and 215;
SEQ ID NOs: 232, 244, and 215;
SEQ ID NOs: 232, 245, and 215;
SEQ ID NOs: 213, 246, and 215;
SEQ ID NOs: 213, 247, and 215;
SEQ ID NOs: 213, 248, and 215; and
SEQ ID NOs: 213, 249, and 215.

39. A bi-specific anti-CD19 and anti-CD3 antigen binding molecule comprising:
a means for binding CD19; and
a first anti-CD3 binding subunit comprising the CDR amino acid sequences of SEQ ID NO: 9, SEQ ID NO: 10 or 11, and SEQ ID NO: 12, 13, 14, 15, or 16, and an anti-CD3 second binding subunit comprising the CDR amino acid sequences of SEQ ID NOs: 17, 18, and 19.

40. A bi-specific anti-CD19 and anti-CD3 antigen binding molecule comprising:
a means for binding CD3; and
an anti-CD19 antigen binding domain comprising:
a first binding subunit comprising CDR amino acid sequences of SEQ ID NOs: 1, 2, and 3; and
a second binding subunit comprising CDR amino acid sequences of SEQ ID NOs: 4, 5, and 6.

41. A method of depleting B-cells in a subject, the method comprising administering a bi-specific anti-CD19 and anti-CD3 antigen binding molecule to the subject,
wherein the anti-CD19 antigen binding domain comprises:
a first anti-CD19 binding subunit comprising at least 95% sequence identity to the amino acid sequence of SEQ ID NO: 7; and
a second anti-CD19 binding subunit comprising at least 95% sequence identity to the amino acid sequence of SEQ ID NO: 8; and
a light chain variable region comprising at least 95% sequence identity to an amino acid sequence of SEQ ID NO: 180; and
a heavy chain variable region comprising at least 95% sequence identity to an amino acid sequence selected from the group consisting of SEQ ID NO: 179, 181, 182, 183, 184, 185, 186, 187, 188, and 189.

42.-43. (canceled)

44. A method of treating an autoimmune disorder in a subject, the method comprising administering a bi-specific anti-CD19 and anti-CD3 antigen binding molecule to the subject,
wherein the anti-CD19 antigen binding domain comprises:
a first anti-CD19 binding subunit comprising at least 95% sequence identity to the amino acid sequence of SEQ ID NO: 7; and
a second anti-CD19 binding subunit comprising at least 95% sequence identity to the amino acid sequence of SEQ ID NO: 8; and
a light chain variable region comprising at least 95% sequence identity to an amino acid sequence of SEQ ID NO: 180; and
a heavy chain variable region comprising at least 95% sequence identity to an amino acid sequence selected from the group consisting of SEQ ID NO: 179, 181, 182, 183, 184, 185, 186, 187, 188, and 189.

45. (canceled)

46. A method of treating an inflammatory disease in a subject, the method comprising administering a bi-specific anti-CD19 and anti-CD3 antigen binding molecule to the subject,
wherein the anti-CD19 antigen binding domain comprises:
a first anti-CD19 binding subunit comprising at least 95% sequence identity to the amino acid sequence of SEQ ID NO: 7; and
a second anti-CD19 binding subunit comprising at least 95% sequence identity to the amino acid sequence of SEQ ID NO: 8; and
a light chain variable region comprising at least 95% sequence identity to an amino acid sequence of SEQ ID NO: 180; and
a heavy chain variable region comprising at least 95% sequence identity to an amino acid sequence selected from the group consisting of SEQ ID NO: 179, 181, 182, 183, 184, 185, 186, 187, 188, and 189.

47. A method of treating cancer in a subject, the method comprising administering a bi-specific anti-CD19 and anti-CD3 antigen binding molecule to the subject,
wherein the anti-CD19 antigen binding domain comprises:
a first anti-CD19 binding subunit comprising at least 95% sequence identity to the amino acid sequence of SEQ ID NO: 7; and
a second anti-CD19 binding subunit comprising at least 95% sequence identity to the amino acid sequence of SEQ ID NO: 8; and
a light chain variable region comprising at least 95% sequence identity to an amino acid sequence of SEQ ID NO: 180; and
a heavy chain variable region comprising at least 95% sequence identity to an amino acid sequence selected from the group consisting of SEQ ID NO: 179, 181, 182, 183, 184, 185, 186, 187, 188, and 189.

48. (canceled)

49. An anti-CD3 antigen binding molecule, comprising an antigen binding domain comprising:
a first binding subunit comprising complementarity determining region (CDR) amino acid sequences of SEQ ID NO: 9, SEQ ID NO: 10 or 11, and SEQ ID NO: 12, 13, 14, 15, or 16; and
a second binding subunit comprising the CDR amino acid sequences of SEQ ID NOs: 17, 18, and 19.

50.-57. (canceled)

58. An anti-CD3 antigen binding molecule, comprising:
a first binding subunit and a second binding subunit;
wherein the first binding subunit comprises a framework region having at least 95% identity to an amino acid sequence selected from the group consisting of SEQ ID NO: 28, 29, 30, and 31, and/or
wherein the second binding subunit comprises a framework region having at least 95% identity to an amino acid sequence selected from the group consisting of 32, 33, 34, and 35.

59.-65. (canceled)

66. An anti-CD19 antigen binding molecule, comprising an antigen binding domain comprising:
a first binding subunit comprising CDR amino acid sequences of SEQ ID NOs: 1, 2, and 3; and
a second binding subunit comprising CDR amino acid sequences of SEQ ID NOs: 4, 5, and 6.

67.-71. (canceled)

72. An anti-CD19 antigen binding molecule, comprising:
a first binding subunit and a second binding subunit; and
wherein the first binding subunit comprises a framework region having at least 95% identity to an amino acid sequence selected from the group consisting of SEQ ID NO: 20, 21, 22, and 23, and/or
wherein the second binding subunit comprises a framework region having at least 95% identity to an amino acid sequence selected from the group consisting of 24, 25, 26, and 27.

73.-78. (canceled)

79. A bi-specific anti-CD19 and anti-CD3 antigen binding molecule comprising:
a first anti-CD19 binding subunit comprising:
a framework region 1 (FR1) comprising at least 95% identity to an amino acid sequence of SEQ ID NO: 20;
a framework region 2 (FR2) comprising at least 95% identity to the amino acid sequence of SEQ ID NO: 21;
a framework region 3 (FR3) comprising at least 95% identity to the amino acid sequence of SEQ ID NO: 22; and
a framework region 4 (FR4) comprising at least 95% identity to the amino acid sequence of SEQ ID NO: 23; and
a second anti-CD19 binding subunit comprising:
a framework region 5 (FR5) comprising at least 95% identity to the amino acid sequence of SEQ ID NO: 24;
a framework region 6 (FR6) comprising at least 95% identity to the amino acid sequence of SEQ ID NO: 25;
a framework region 7 (FR7) comprising at least 95% identity to the amino acid sequence of SEQ ID NO: 26; and
a framework region 8 (FR8) comprising at least 95% identity to the amino acid sequence of SEQ ID NO: 27.

80.-86. (canceled)

87. A nucleic acid encoding a first anti-CD19 binding subunit, the first anti-CD19 binding subunit comprising CDR amino acid sequences of SEQ ID NOs: 1, 2, and 3.

88. (canceled)

89. A nucleic acid encoding a second anti-CD19 binding subunit comprising CDR amino acid sequences of SEQ ID NOs: 4, 5, and 6.

90. A nucleic acid encoding a first anti-CD3 binding subunit,
the first binding subunit comprising complementarity determining region (CDR) amino acid sequences of SEQ ID NO: 9, SEQ ID NO: 10 or 11, and SEQ ID NO: 12, 13, 14, 15, or 16.

91. (canceled)

92. A nucleic acid encoding a second anti-CD3 binding subunit, the second binding subunit comprising the CDR amino acid sequences of SEQ ID NOs: 17, 18, and 19.

93.-97. (canceled)

98. A host cell comprising the nucleic acid of claim 87.
